(19) United States
(12) Patent Application Publication
(10) Pub. No.: US 2023/0016929 A1
(43) Pub. Date: Jan. 19, 2023

(54) EXTRAHEPATIC DELIVERY

(71) Applicant: ALNYLAM PHARMACEUTICALS, INC., Cambridge, MA (US)

(72) Inventors: Jayaprakash K. NAIR, Cambridge, MA (US); Martin A. MAIER, Cambridge, MA (US); Juan C. SALINAS, Cambridge, MA (US); Shigeo MATSUDA, Cambridge, MA (US); Alexander V. KEL'IN, Cambridge, MA (US); Scott P. LENTINI, Cambridge, MA (US); Guo HE, Cambridge, MA (US); Michelle H. JUNG, Cambridge, MA (US); Justin M. PIERSON, Cambridge, MA (US); Muthiah MANOHARAN, Cambridge, MA (US); Dale C. GUENTHER, Cambridge, MA (US); Ivan ZLATEV, Cambridge, MA (US); Christopher S. THEILE, Cambridge, MA (US); Vasant R. JADHAV, Cambridge, MA (US); Stuart MILSTEIN, Cambridge, MA (US); Maja JANAS, Cambridge, MA (US); Dhrubajyoti DATTA, Cambridge, MA (US)

(21) Appl. No.: 17/774,477

(22) PCT Filed: Nov. 6, 2020
(86) PCT No.: PCT/US2020/059399

§ 371 Date: May 4, 2022

Related U.S. Application Data
(60) Provisional application No. 62/931,631, filed on Nov. 6, 2019.

(51) Int. Cl. A61K 31/713; A61K 48/00; C12N 15/113; A61P 25/28

(52) U.S. Cl. CPC A61K 31/713; A61K 48/0075; C12N 15/113; A61P 25/28

(57) ABSTRACT

One aspect of the present invention relates to a compound comprising an antisense strand which is complementary to a target gene; a sense strand which is complementary to said antisense strand; and one or more lipophilic monomers, containing one or more lipophilic moieties, conjugated to one or more positions on at least one strand, optionally via a linker or carrier. Another aspect of the invention relates to a method of gene silencing, comprising administering to a cell or a subject in need thereof a therapeutically effective amount of the lipophilic monomer-conjugated compound.

### BACKGROUND

Efficient delivery of an iRNA agent to cells in vivo requires specific targeting and substantial protection from the extracellular environment, particularly serum proteins. RNAi-based therapeutics show promising clinical data for treatment of liver-associated disorders. However, siRNA delivery into extra-hepatic tissues remains an obstacle, limiting the use of siRNA-based therapies.

One of the factors that limit the experimental and therapeutic application of iRNA agents in vivo is the ability to deliver intact siRNA efficiently. Particular difficulties have been associated with non-viral gene transfer into the retina in vivo. One of the challenges is to overcome the inner limiting membrane, which impedes the transfection of the retina. Additionally, negatively charged sugars of the vitreous have been shown to interact with positive DNA-transfection reagent complexes, promoting their aggregation, which impedes diffusion and cellular uptake.

Delivery of oligonucleotides to the central nervous system (CNS) poses particular problems due to the blood brain barrier (BBB) that free oligonucleotides cannot cross. One means to deliver oligonucleotides into the CNS is by intrathecal delivery. However, the oligonucleotides need also to be efficiently internalized into target cells of the CNS to achieve the desired therapeutic effect. Previous work has typically used delivery reagents such as liposomes, cationic lipids, and nanoparticles forming complexes to aid the intracellular internalization of oligonucleotides into cells of neuronal origin.

Thus, there is a continuing need for new and improved methods for delivering siRNA molecules in vivo, without the use of tissue delivery reagents, to achieve and enhance the therapeutic potential of iRNA agents.

### SUMMARY

One aspect of the invention provides a compound (e.g., an oligonucleotide that can be either single-stranded or double-stranded) comprising one or more lipophilic monomers, containing one or more lipophilic moieties, conjugated to one or more positions on at least one strand of the oligonucleotide, optionally via a linker or carrier. For instance, some embodiments of the invention provide a compound (e.g., a double-stranded iRNA agent) comprising: an antisense strand which is complementary to a target gene; a sense strand which is complementary to said antisense strand; and one or more lipophilic monomers, containing one or more lipophilic moieties, conjugated to one or more positions on at least one strand, optionally via a linker or carrier.

In some embodiments, the lipophilicity of the lipophilic moiety, measured by octanol-water partition coefficient, logK_{ow}, exceeds 0. The lipophilic moiety may possess a logK_{ow} exceeding 1, exceeding 1.5, exceeding 2, exceeding 3, exceeding 4, exceeding 5, or exceeding 10.

In some embodiments, the hydrophobicity of the compound, measured by the unbound fraction in the plasma protein binding assay of the compound, exceeds 0.2. In one embodiment, the plasma protein binding assay determined is an electrophoretic mobility shift assay (EMSA) using human serum albumin protein. The hydrophobicity of the compound, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

In some embodiments, the lipophilic moiety is an aliphatic, cyclic such as alicyclic, or polycyclic such as polyalicyclic compound, such as a steroid (e.g., sterol) or a linear or branched aliphatic hydrocarbon. Exemplary lipophilic moieties are lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

Suitable lipophilic moieties also include those containing a saturated or unsaturated C₄-C₃₀ hydrocarbon chain (e.g., C₄-C₃₀ alkyl or alkenyl), and an optional functional group selected from the group consisting of hydroxyl, amine, carboxylic acid, sulfonate, phosphate, thiol, azide, and alkyne. The functional groups are useful to attach the lipophilic moiety to the iRNA agent. In some embodiments, the lipophilic moiety contains a saturated or unsaturated C₆-C₁₈ hydrocarbon chain (e.g., a linear C₆-C₁₈ alkyl or alkenyl). In one embodiment, the lipophilic moiety contains a saturated or unsaturated C₁₆ hydrocarbon chain (e.g., a linear C₁₆ alkyl or alkenyl). In some embodiments, the lipophilic moiety contains two or more carbon-carbon double bonds.

In some embodiments, the lipophilic moiety is a C₆-C₃₀ moiety having a free terminal carboxylic acid functionality (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, cis-4,7,10,13,16,19-docosahexaenoic acid).

In some embodiments, the lipophilic moiety is a C₆-C₃₀ acid (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, cis-4,7,10,13,16,19-docosahexaenoic acid, vitamin A, vitamin E, cholesterol etc.) or a C₆-C₃₀ alcohol (e.g., hexanol, heptanol, octanol, nonanol, decanol, undecanol, dodcanol, tridecanol, tetradecanol, pentadecanol, hexadecanol, heptadecanol, octadecanol, oleyl alcohol, linoleyl alcohol, arachidonic alcohol, cis-4,7,10,13,16,19-docosahexanol, retinol, vitamin E, cholesterol etc.).

The lipophilic monomer may comprise a lipophilic moiety conjugated to any part of the iRNA agent, e.g., a nucleobase, sugar moiety, or internucleosidic linkage. When the lipophilic moiety is conjugated to the iRNA agent via a direct attachment to the nucleobase, ribosugar, or internucleosidic linkage of the iRNA agent, the lipophilic monomer then comprises the nucleobase, ribosugar, or internucleosidic linkage, and the lipophilic moiety. Alternatively, the lipophilic monomer may comprise a lipophilic moiety conjugated to a non-ribose replacement unit, such as a linker or carrier. When the lipophilic moiety is conjugated to the iRNA agent via a non-ribose replacement unit, such as a linker or a carrier, the lipophilic monomer then comprises the non-ribose replacement unit, such as the linker or carrier, and the lipophilic moiety.

In certain embodiments, the lipophilic monomer does not contain a nucleobase.

In certain embodiments, the lipophilic monomer comprises the lipophilic moiety conjugated to the compound via one or more linkers (tethers).

In some embodiments, the lipophilic monomer comprises the lipophilic moiety conjugated to the compound via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, at least one of the linkers (tethers) is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., a phosphate group), or a peptidase cleavable linker (e.g., a peptide bond).

In other embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, mannose, and combinations thereof.

In certain embodiments, the lipophilic monomer comprises the lipophilic moiety conjugated to the compound via a non-ribose replacement unit, i.e., a carrier that replaces one or more nucleotide(s). The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone, a glycerol backbone, or a diethanolamine backbone.

In some embodiments, the carrier replaces one or more nucleotide(s) in the double-stranded iRNA agent. In some embodiments, the carrier replaces one or more nucleotide(s) in the internal position(s) of the double-stranded iRNA agent. In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

In some embodiments, the lipophilic monomer may be represented by one of the following formulae:

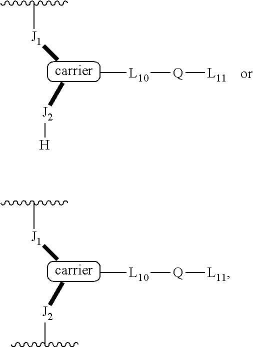

wherein:

J₁ and J₂ are each independently O, S, NR^{N}, optionally substituted alkyl, OC(O)NH, NHC(O)O, C(O)NH, NHC(O), OC(O), C(O)O, OC(O)O, NHC(O)NH, NHC(S)NH, OC(S)NH, OP(N(R^{P})₂)O, or OP(N(R)₂);

[symbol] is a cyclic group or an acyclic group;

R^{N} is H, optionally substituted alkyl, optionally substituted alkenyl, optionally substituted alkynyl, optionally substituted aryl, optionally substituted cycloalkyl, optionally substituted aralkyl, optionally substituted heteroaryl, or an amino protecting group;

R^{P} is independently for each occurrence H, optionally substituted alkyl, optionally substituted alkenyl, optionally substituted alkynyl, optionally substituted aryl, optionally substituted cycloalkyl, or optionally substituted heteroaryl;

L₁₀ is substituted or unsubstituted, saturated or unsaturated C3-C8 hydrocarbon, (e.g., C3-C8 alkyl, alkenyl, or alkynyl, or C3-C8 hydrocarbon containing two or more double bonds); the substituted groups include those already described herein for “substituted” hydrocarbon, alkyl, alkenyl, or alkynyl;

L₁₁ is substituted or unsubstituted, saturated or unsaturated C6-C26 hydrocarbon, (e.g., C6-C26 alkyl, alkenyl, or alkynyl, or C3-C8 hydrocarbon containing two or more double bonds); the substituted groups include those already described herein for “substituted” hydrocarbon, alkyl, alkenyl, or alkynyl; and

Q is absent when there is no nucleobase on the carrier, or a cleavable group that will cleave L₁₀ from L₁₁ at least 10% in vivo. For instance, Q may be a cleavable group that can be cleaved in vivo to cleave Lₙ off the lipophilic monomer by about 10-70%, about 15-50%, about 20-40%, or about 20-30%. Exemplary cleavable groups include —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)N(R⁵)—, —N(R⁵)C(O)—, —C(S)N(R⁵)—, —N(R⁵)C(S)—, —N(R⁵)C(O)N(R⁵)—, —N(R⁵)C(O)C(R³)(R⁴)OC(O)—, —C(O)OC(R³)(R⁴)C(O)N(R⁵)—, —OC(O)O—, —OSi(R⁵)₂O—, —C(O)(CR³R⁴)C(O)O—, —OC(O)(CR³R⁴)C(O)—,

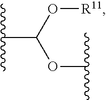

or combinations thereof, R¹¹ is a C₂-C₈ alkyl or alkenyl. For each occurrence, R³, R⁴, and R⁵ are each independently H or C₁-C₄ alkyl.

In one embodiment, the cleavability of Q is determined by the stability of ligands in cerebral spinal fluid (CSF), the stability of ligands in plasma, the stability of ligands in brain homogenate or tissue homogenate (liver, ocular etc.), or the stability of ligands in vitreous humor.

The cyclic and acyclic groups include those already described herein.

In one embodiment, the acyclic group is a serinol, glycerol, or diethanolamine backbone.

In one embodiment, the cyclic group is selected from the group consisting of pyrrolidinyl, hydroxyprolinyl, cyclopentyl, cyclohexyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

In one embodiment, the cyclic group is a ribose or a ribose analog. Examples of ribose analogs include arabinose, 4′-thio ribose, 2′-O-methyl ribose, GNA, UNA, and LNA analogs.

In some embodiments, the lipophilic monomer conjugated to one or more positions of a strand of the compound has a structure of:

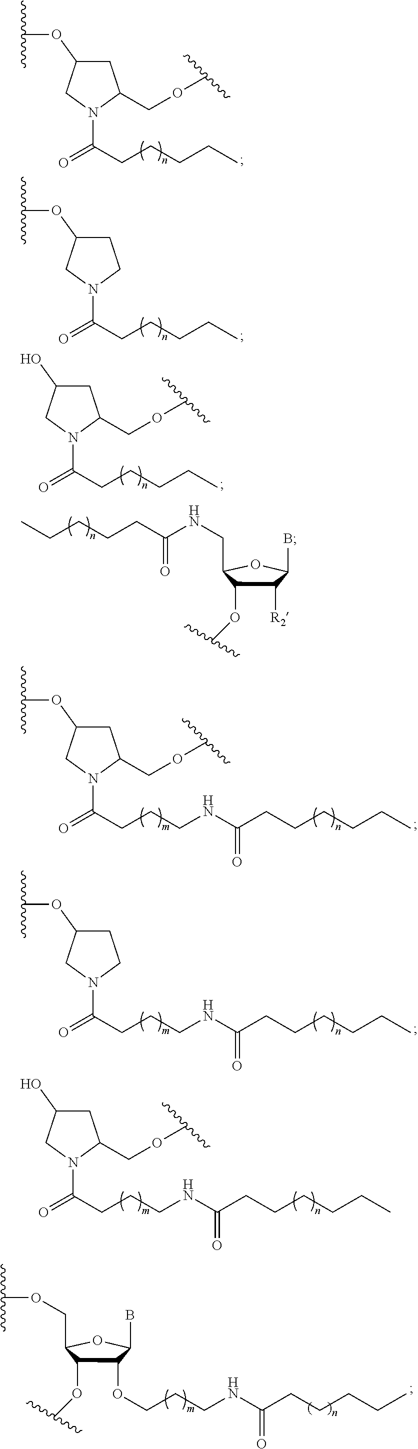

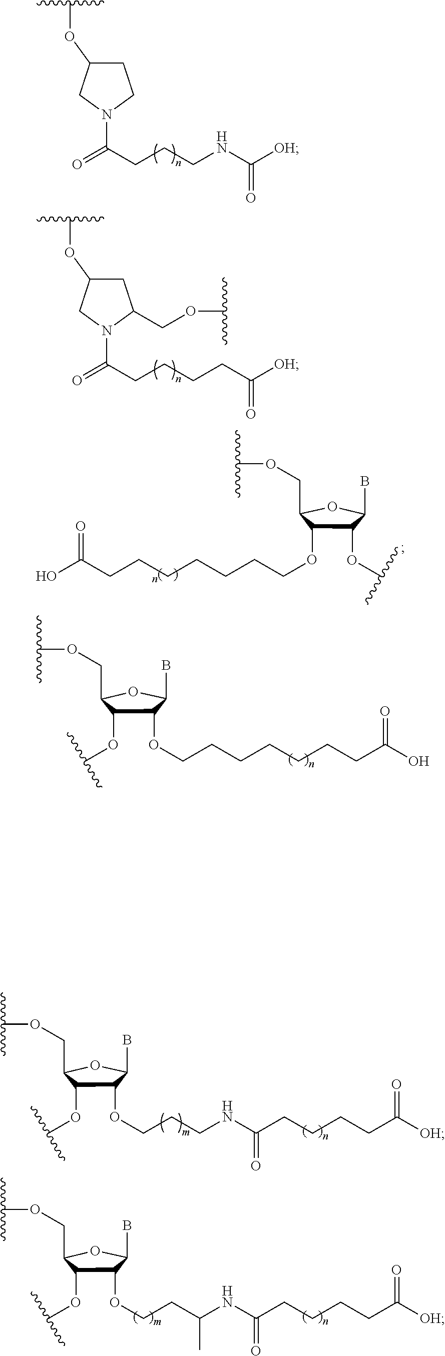

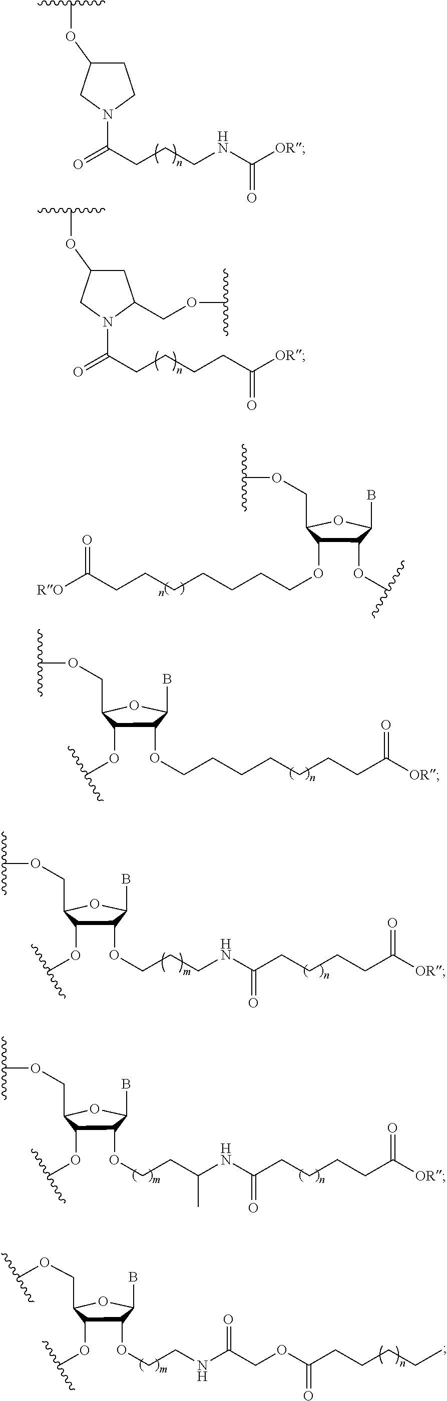

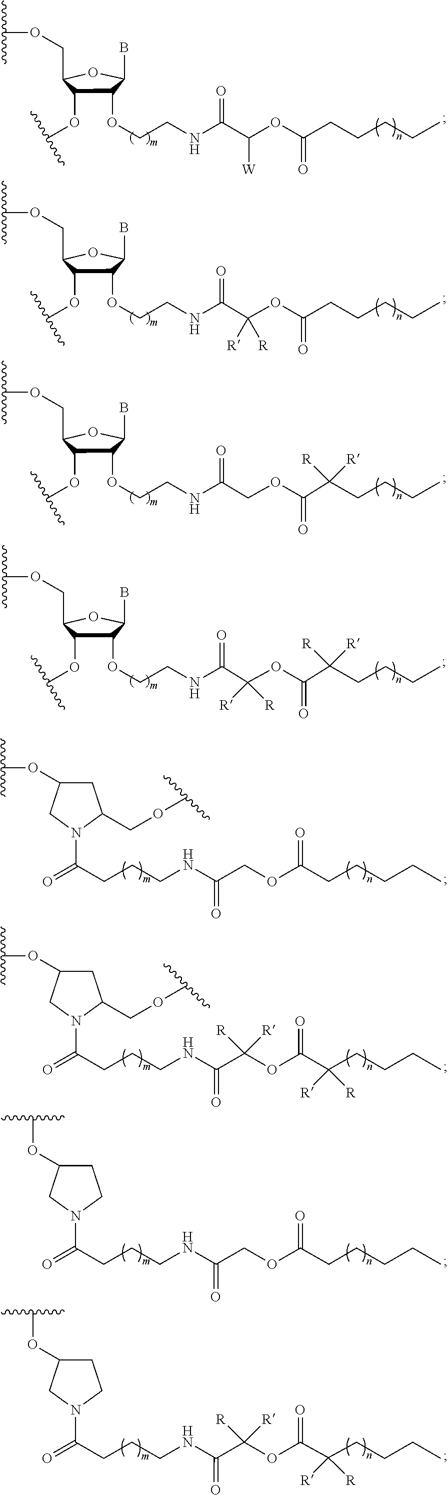

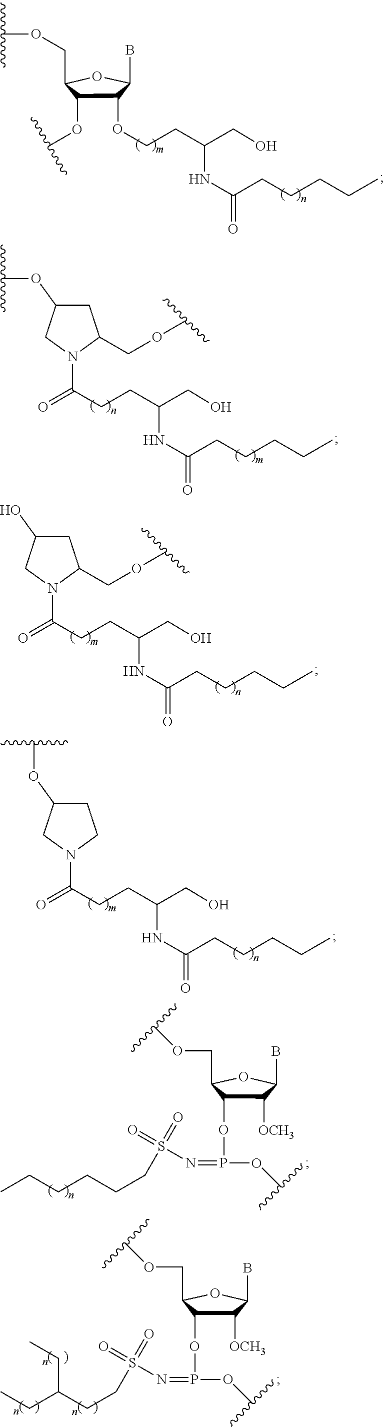

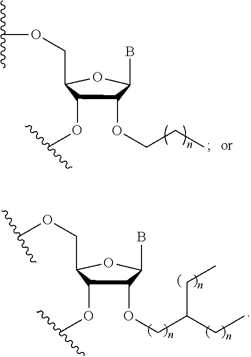

In the above structures for the lipophilic monomers, the monomers may also contain one or more asymmetric centers and thus occur as racemates and racemic mixtures, single enantiomers, individual diastereomers and diastereomeric mixtures. All such isomeric forms of the monomers are expressly included.

In the above structures for the lipophilic monomers, the alkylene chain can contain one or more unsaturated bonds.

Integer m is 0-8. Integer n is 1-21. R₂′ may be any functional group that is an acceptable 2′-modification for a ribose sugar, such as a 2′-O-methoxyalkyl (e.g., 2′-O-methoxymethyl, 2′-O-methoxyethyl, or 2′-O-2-methoxypropanyl) modification, 2′-O-allyl modification, 2′-C-allyl modification, 2′-fluoro modification, 2′-O—N-methylacetamido (2′-O-NMA) modification, 2′-O-dimethylaminoethoxyethyl (2′-O-DMAEOE) modification, 2′-O-aminopropyl (2′-O-AP) modification, or 2′-ara-F modification. For instance, R₂′ may be H, OH, F, OMe, O-methoxyalkyl, O-allyl, O—N-methylacetamido, O-dimethylaminoethoxyethyl, or O-aminopropyl. B is a modified or unmodified nucleobase. W is an alkyl group such as a C₁-C₄ alkyl (e.g., methyl, ethyl, propyl, isopropyl, butyl, isobutyl, t-butyl). R, R′, and R″ are each independently H or an alkyl group such as a C₁-C₄ alkyl (e.g., methyl, ethyl, propyl, isopropyl, t-butyl).

In some embodiments, the lipophilic monomer conjugated to one or more positions of a strand of the compound has a structure of:

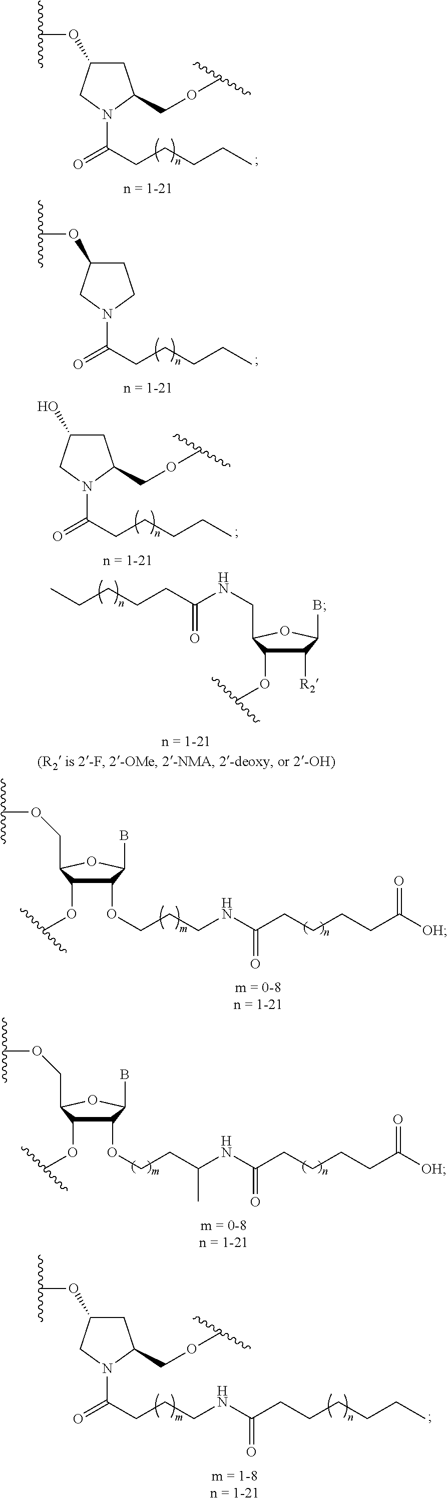

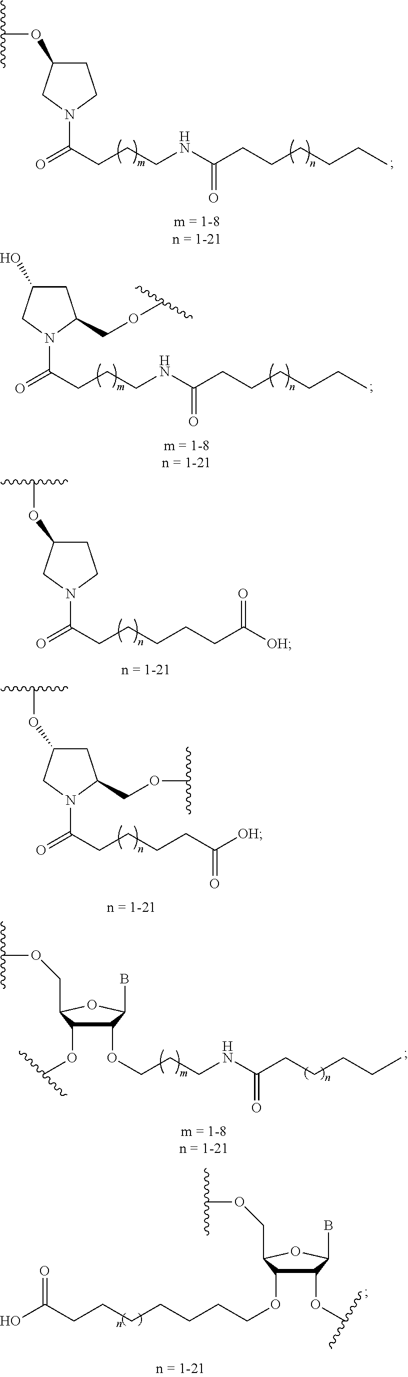

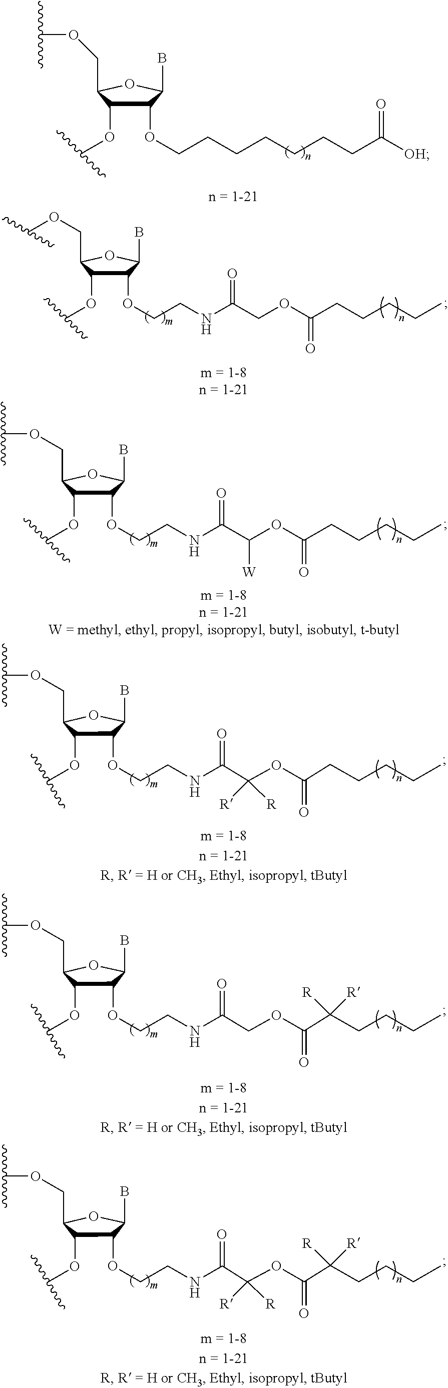

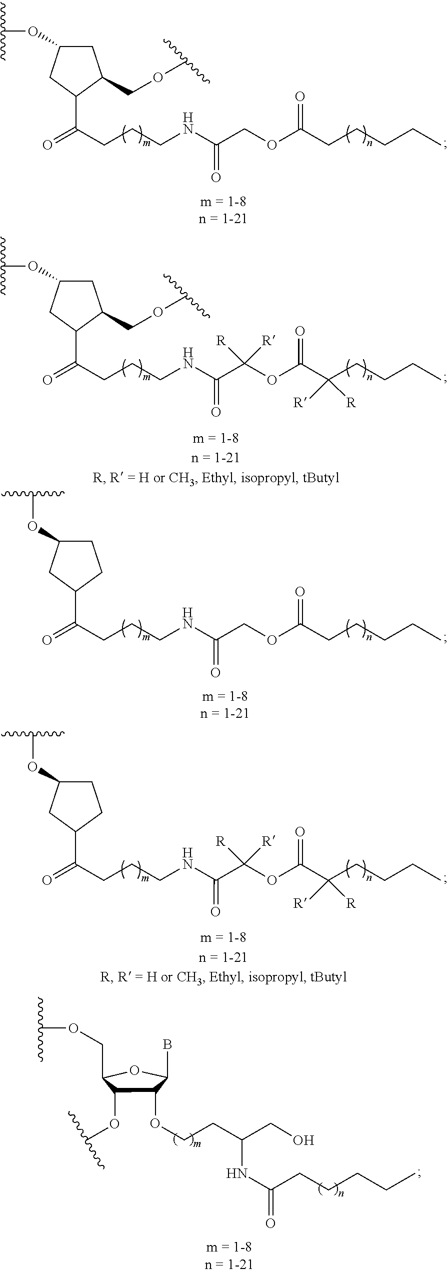

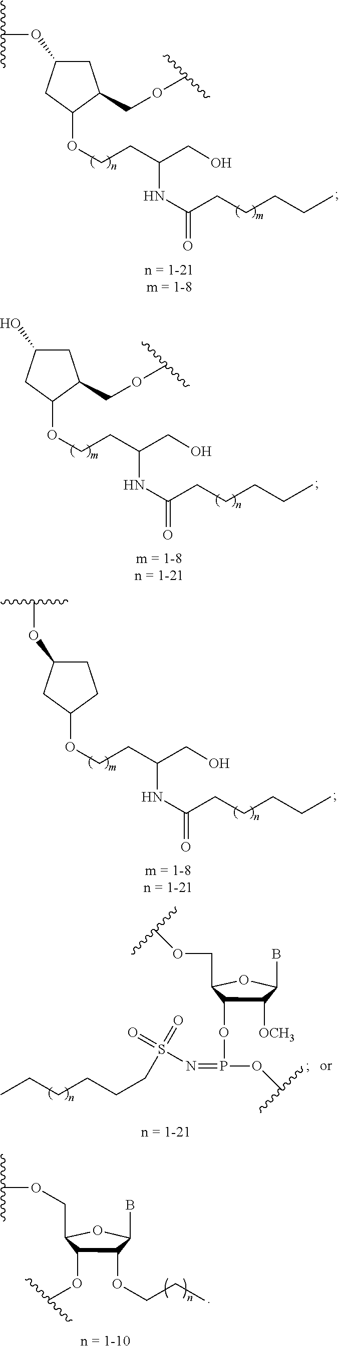

In these structures, B is a modified or unmodified nucleobase.

Specific embodiments of the lipophilic monomers include:

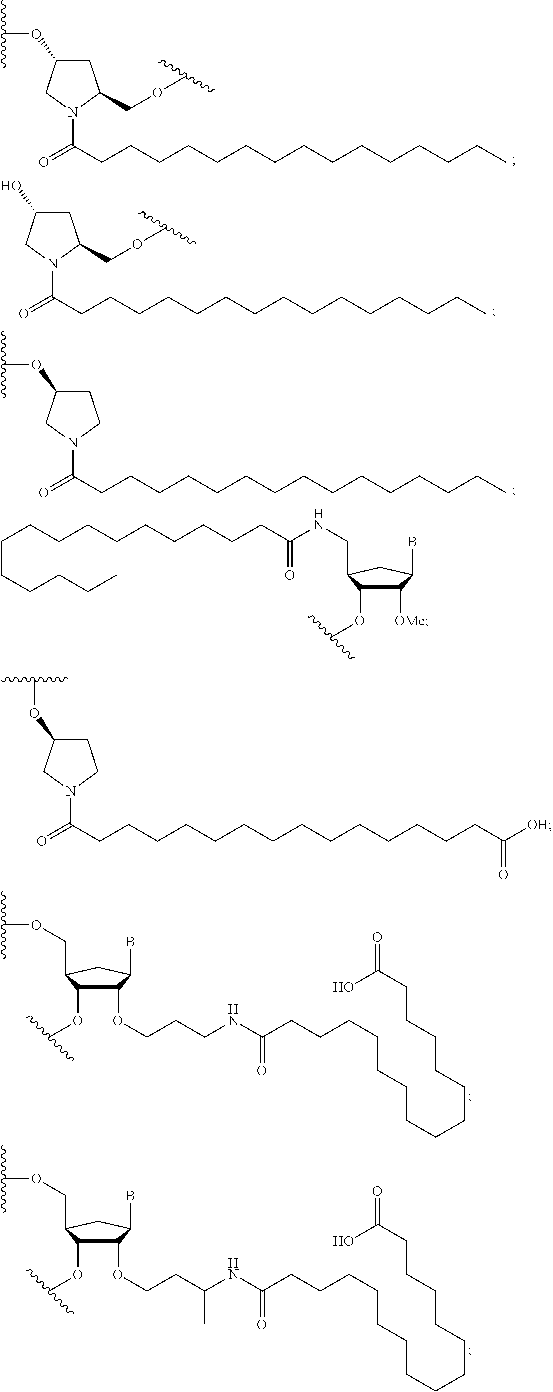

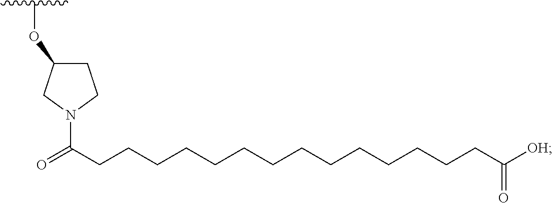 [SMILES: *O[C@H]1CCN(C(=O)CCCCCCCCCCCCCCC(=O)O)C1]

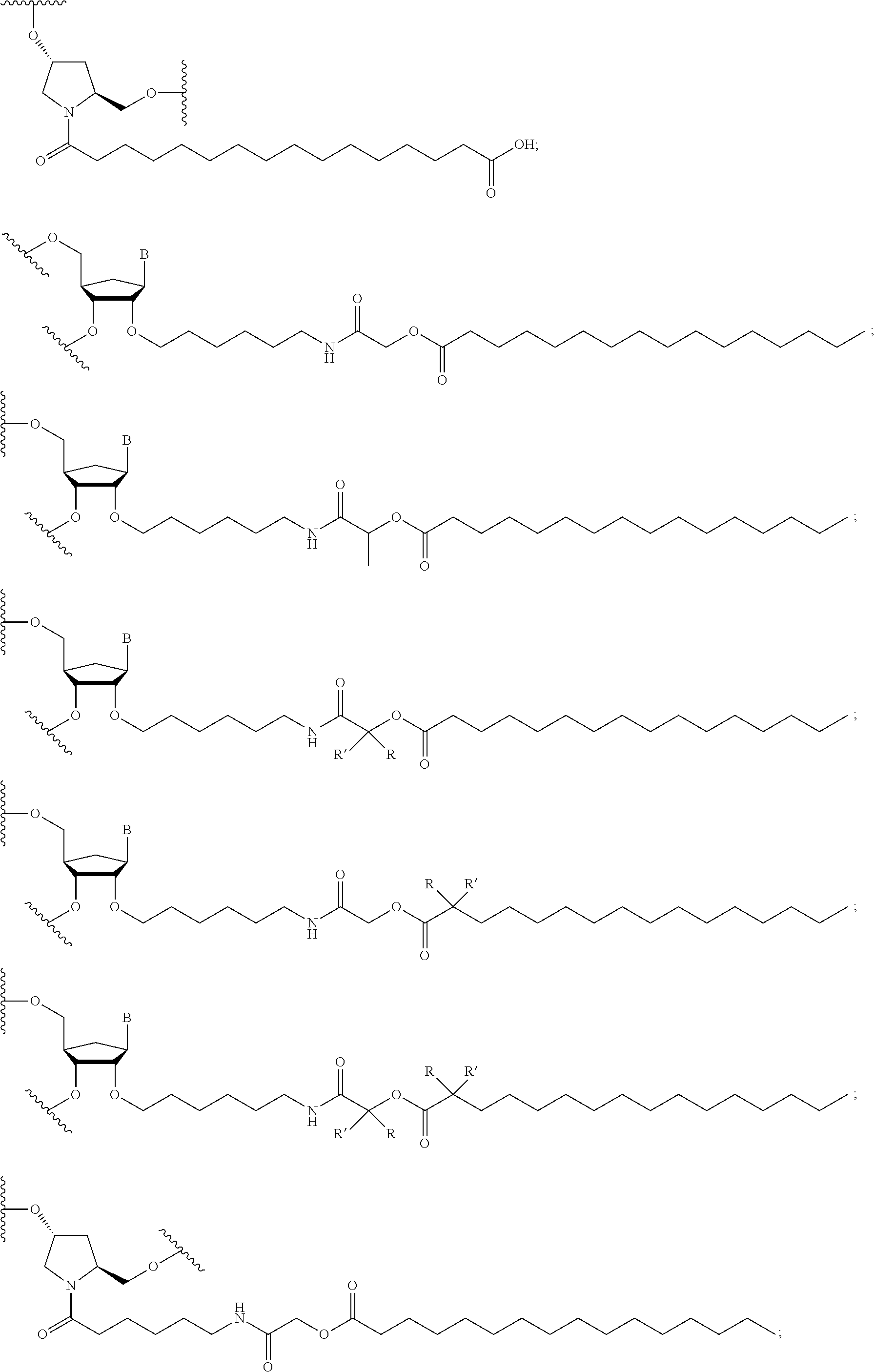

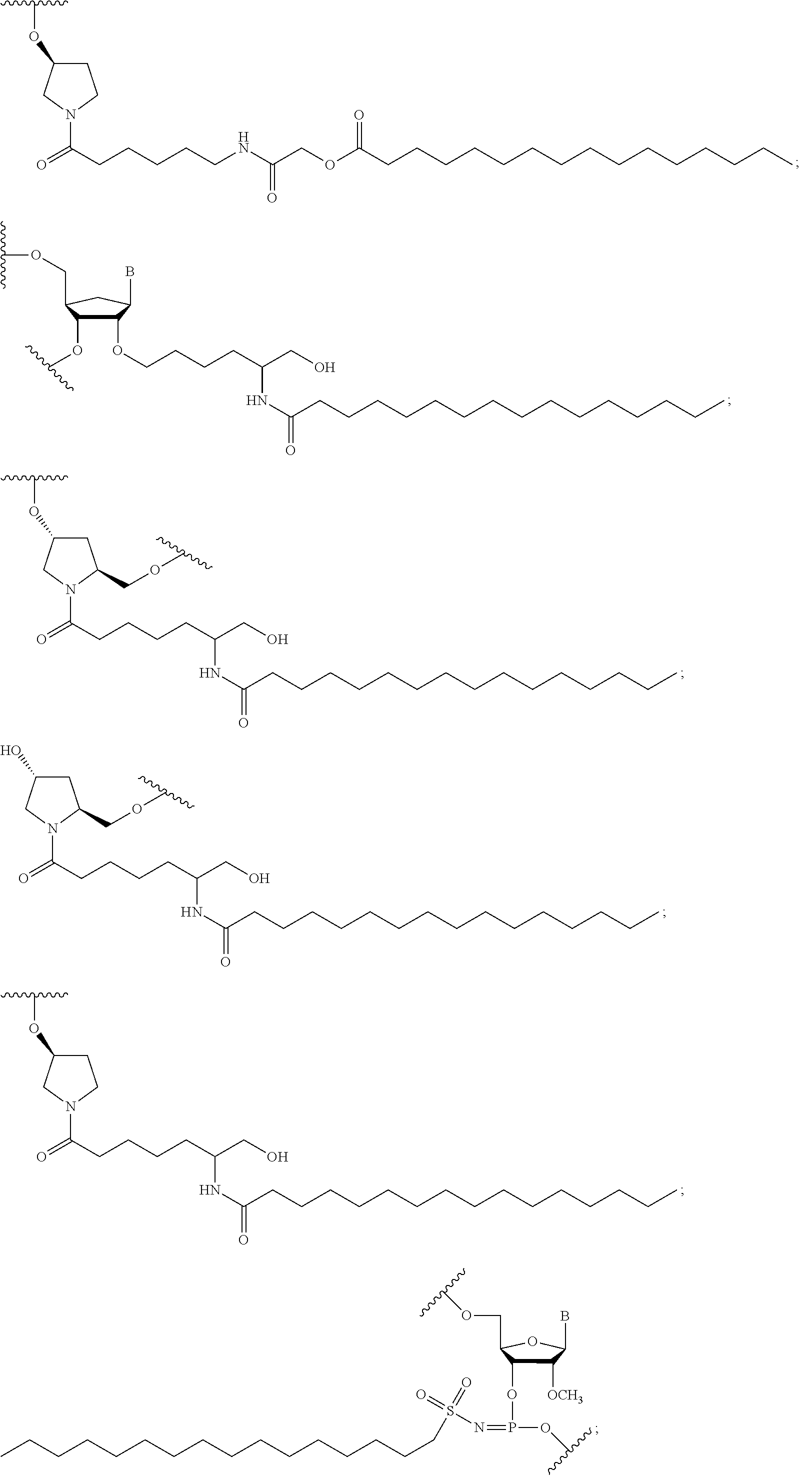

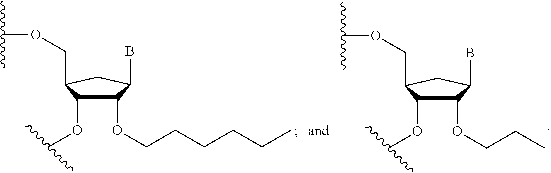

In these structures, B is a modified or unmodified nucleobase; and R and R′ are each independently H, methyl, ethyl, isopropyl, or t-butyl.

In some embodiments, the lipophilic monomer contains a lipophilic moiety conjugated to a strand of the compound (a single strand of a single-stranded oligonucleotide; or sense strand and/or antisense strand of a double-stranded oligonucleotide) via a carrier of:

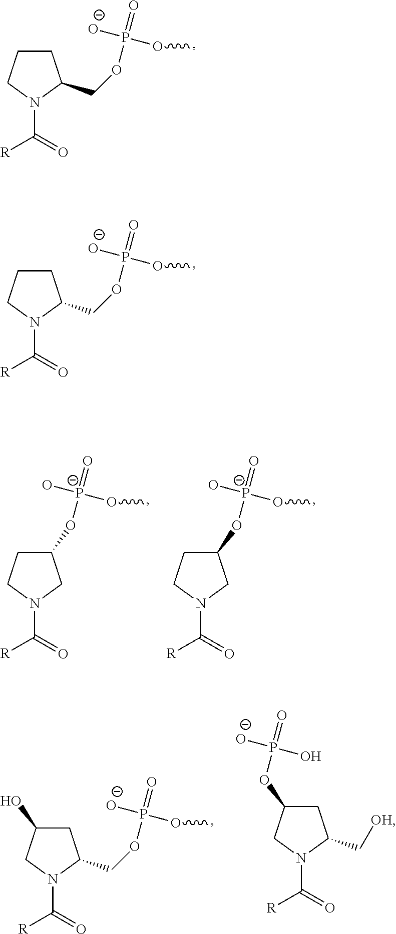

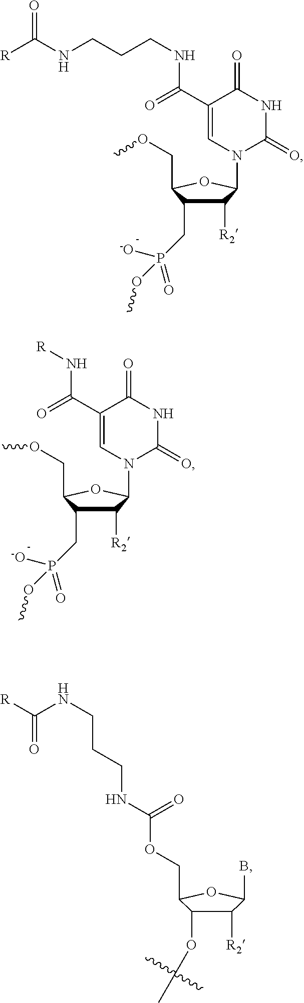

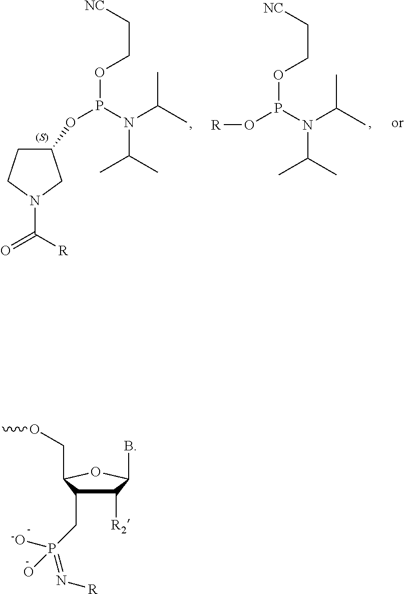

In these embodiments, R is the lipophilic moiety as defined herein. R₂′ is H, OH, F, OMe, O-methoxyalkyl, O-allyl, O—N-methylacetamido, O-dimethylaminoethoxyethyl, or O-aminopropyl. B is a modified or unmodified nucleobase.

In some embodiments, the lipophilic monomer contains a lipophilic moiety conjugated to an internal position of a strand of the compound (a single strand of a single-stranded oligonucleotide; or sense strand and/or antisense strand of a double-stranded oligonucleotide) via a carrier of:

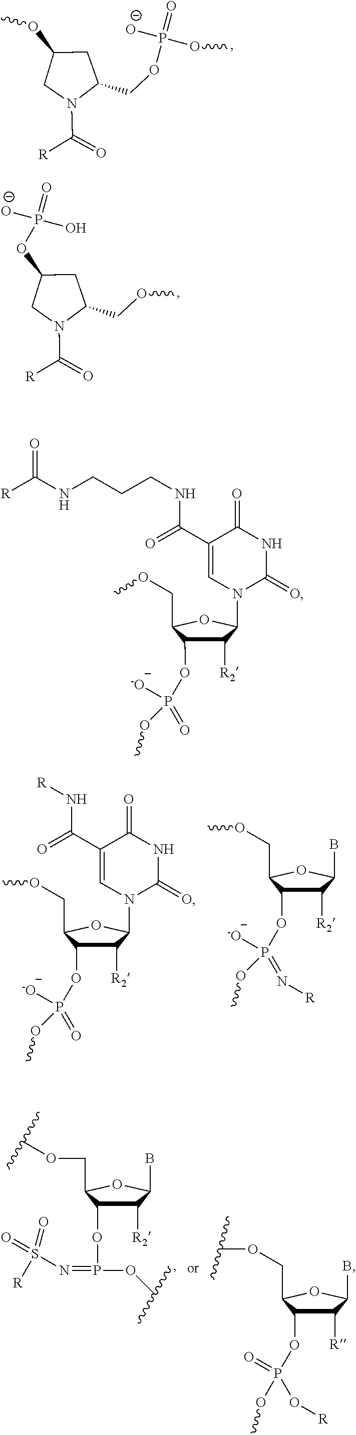

In these embodiments, R is the lipophilic moiety as defined herein. n is an integer of 1-21. R₂′ is H, OH, F, OMe, O-methoxyalkyl, O-allyl, O—N-methylacetamido, O-dimethylaminoethoxyethyl, or O-aminopropyl. B is a modified or unmodified nucleobase.

Additional examples of the lipophilic monomers can be found in the Examples.

In some embodiments, the sense and antisense strands of the compound are each 15 to 30 nucleotides in length.

In one embodiment, the sense and antisense strands of a compound are each 19 to 25 nucleotides in length.

In one embodiment, the sense and antisense strands of the compound are each 21 to 23 nucleotides in length.

In some embodiments, the compound comprises a single-stranded overhang on at least one of the termini, e.g., 3′ and/or 5′ overhang(s) of 1-10 nucleotides in length, for instance, an overhang of 1, 2, 3, 4, 5, or 6 nucleotides. In some embodiments, both strands have at least one stretch of 1-5 (e.g., 1, 2, 3, 4, or 5) single-stranded nucleotides in the double stranded region. In one embodiment, the single-stranded overhang is 1, 2, or 3 nucleotides in length. In some embodiments, the compound may also have a blunt end, located at the 5′-end of the antisense strand (or the 3′-end of the sense strand), or vice versa. In one embodiment, the compound comprises a 3′ overhang at the 3′-end of the antisense strand, and optionally a blunt end at the 5′-end of the antisense strand. In one embodiment, the compound has a 5′ overhang at the 5′-end of the sense strand, and optionally a blunt end at the 5′-end of the antisense strand. In one embodiment, the compound has two blunt ends at both ends of the iRNA duplex.

In one embodiment, the sense strand of the compound is 21-nucleotide in length, and the antisense strand is 23-nucleotide in length, wherein the strands form a double-stranded region of 21 consecutive base pairs having a 2-nucleotide long single-stranded overhangs at the 3′-end.

In some embodiments, the sense strand further comprises at least one phosphorothioate linkage at the 3′-end. In some embodiments, the sense strand further comprises at least two phosphorothioate linkages at the 3′-end. In some embodiments, one or more lipophilic monomers are located on the 3′-end of the sense strand. In one embodiment, one of the phosphorothioate linkages is located between the lipophilic monomer and the first nucleotide from the 3′-end of the sense strand.

In some embodiments, the sense strand further comprises at least one phosphorothioate linkage at the 5′-end. In some embodiments, the sense strand further comprises at least two phosphorothioate linkages at the 5′-end. In some embodiments, one or more lipophilic monomers are located on the 5′-end of the sense strand. In one embodiment, one of the phosphorothioate linkages is located between the lipophilic monomer and the first nucleotide from the 5′-end of the sense strand.

In some embodiments, the antisense strand further comprises at least one phosphorothioate linkage at the 3′-end. In some embodiments, the antisense strand further comprises at least two phosphorothioate linkages at the 3′-end. In some embodiments, one or more lipophilic monomers are located on the 3′-end of the antisense strand. In one embodiment, one of the phosphorothioate linkages is located between the lipophilic monomer and the first nucleotide from the 3′-end of the antisense strand.

In some embodiments, the compound further comprises a phosphate or phosphate mimic at the 5′-end of the antisense strand. In one embodiment, the phosphate mimic is a 5′-vinyl phosphonate (VP).

In some embodiments, the 5′-end of the antisense strand of the compound does not contain a 5′-vinyl phosphonate (VP).

In some embodiments, the compound further comprises at least one terminal, chiral phosphorus atom.

A site specific, chiral modification to the internucleotide linkage may occur at the 5′ end, 3′ end, or both the 5′ end and 3′ end of a strand. This is being referred to herein as a “terminal” chiral modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a strand. A chiral modification may occur on the sense strand, antisense strand, or both the sense strand and antisense strand. Each of the chiral pure phosphorus atoms may be in either Rp configuration or Sp configuration, and combination thereof. More details regarding chiral modifications and chirally-modified dsRNA agents can be found in PCT/US18/67103, entitled “Chirally-Modified Double-Stranded RNA Agents,” filed Dec. 21, 2018, which is incorporated herein by reference in its entirety.

In some embodiments, the compound further comprises a terminal, chiral modification occurring at the first internucleotide linkage at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp configuration or Sp configuration.

In one embodiment, the compound further comprises a terminal, chiral modification occurring at the first and second internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the compound further comprises a terminal, chiral modification occurring at the first, second, and third internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the compound further comprises a terminal, chiral modification occurring at the first and second internucleotide linkages at the 3′ end of the antisense strand, having the linkage phosphorus atom in Sp configuration; a terminal, chiral modification occurring at the third internucleotide linkages at the 3′ end of the antisense strand, having the linkage phosphorus atom in Rp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the compound further comprises a terminal, chiral modification occurring at the first and second internucleotide linkages at the 3′ end of the antisense strand, having the linkage phosphorus atom in Sp configuration; a terminal, chiral modification occurring at the first, and second internucleotide linkages at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In some embodiments, the compound has at least two phosphorothioate internucleotide linkages at the first five nucleotides on the antisense strand (counting from the 5′ end).

In some embodiments, the antisense strand comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In some embodiments, the compound further comprises a targeting ligand that targets a receptor which mediates delivery to a specific CNS tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, and LDL receptor ligand.

In some embodiments, the compound further comprises a targeting ligand that targets a receptor which mediates delivery to an ocular tissue. In one embodiment, the targeting ligand is selected from the group consisting of trans-retinol, RGD peptide, LDL receptor ligand, and carbohydrate-based ligands. In one embodiment, the targeting ligand is a RGD peptide, such as H-Gly-Arg-Gly-Asp-Ser-Pro-Lys-Cys-OH or Cyclo(-Arg-Gly-Asp-D-Phe-Cys).

In some embodiments, the compound further comprises a targeting ligand that targets a liver tissue. In some embodiments, the targeting ligand is a carbohydrate-based ligand. In one embodiment, the targeting ligand is a GalNAc conjugate.

In some embodiments, 100%, 95%, 90%, 85%, 80%, 75%, 70%, 65%, 60%, 55%, 50%, 45%, 40%, 35% or 30% of the antisense and sense strand of the compound is modified. For example, when 50% of the compound is modified, 50% of all nucleotides present in the compound contain a modification as described herein.

In some embodiments, the antisense and sense strands of the compound comprise at least 30%, 35%, 40%, 45%, 50%, 55%, 60%, 65%, 70%, 75%, 80%, 85%, 90%, 95%, or virtually 100% 2′-O-methyl modified nucleotides.

In one embodiment, the compound is an oligonucleotide, e.g., a double-stranded dsRNA agent, and at least 50% of the nucleotides of the double-stranded dsRNA agent is independently modified with 2′-O-methyl, 2′-O-allyl, 2′-deoxy, or 2′-fluoro.

In one embodiment, the oligonucleotide is an antisense, and at least 50% of the nucleotides of the antisense is independently modified with LNA, CeNA, 2′-methoxyethyl, or 2′-deoxy.

In some embodiments, the sense and antisense strands of the compound comprise less than 12, less than 10, less than 8, less than 6, less than 4, less than 2, or no 2′-F modified nucleotides. In some embodiments, the compound has less than 12, less than 10, less than 8, less than 6, less than 4, less than 2, or no 2′-F modifications on the sense strand. In some embodiments, the compound has less than 12, less than 10, less than 8, less than 6, less than 4, less than 2, or no 2′-F modifications on the antisense strand.

In some embodiments, the compound has one or more 2′-F modifications on any position of the sense strand or antisense strand.

In some embodiments, the compound has less than 20%, less than 15%, less than 10%, less than 5% non-natural nucleotide, or substantially no non-natural nucleotide. Examples of non-natural nucleotide include acyclic nucleotides, LNA, HNA, CeNA, 2′-O-methoxyalkyl (e.g., 2′-O-methoxymethyl, 2′-O-methoxyethyl, or 2′-O-2-methoxypropanyl), 2′-O-allyl, 2′-C-allyl, 2′-fluoro, 2′-O—N-methylacetamido (2′-O-NMA), a 2′-O-dimethylaminoethoxyethyl (2′-O-DMAEOE), 2′-O-aminopropyl (2′-O-AP), 2′-ara-F, L-nucleoside modification (such as 2′-modified L-nucleoside, e.g., 2′-deoxy-L-nucleoside), BNA abasic sugar, abasic cyclic and open-chain alkyl.

In some embodiments, the compound has greater than 80%, greater than 85%, greater than 90%, greater than 95%, or virtually 100% natural nucleotides. For the purpose of these embodiments, natural nucleotides can include those having 2′-OH, 2′-deoxy, and 2′-OMe.

In some embodiments, the antisense strand contains at least one unlocked nucleic acids (UNA) or glycerol nucleic acid (GNA) modification, e.g., at the seed region of the antisense strand. In one embodiment, the seed region is at positions 2-8 (or positions 5-7) of the 5′-end of the antisense strand.

In one embodiment, the compound comprises a sense strand and antisense strand each having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the compound has less than 20%, less than 15%, less than 10%, less than 5% non-natural nucleotide, or substantially no non-natural nucleotide.

In one embodiment, the compound comprises a sense strand and antisense strand each having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the compound has greater than 80%, greater than 85%, greater than 95%, or virtually 100% natural nucleotides, such as those having 2′-OH, 2′-deoxy, or 2′-OMe.

One aspect of the invention provides a compound comprising a sense strand and an antisense strand, each strand independently having a length of 15 to 35 nucleotides; at least two phosphorothioate internucleotide linkages between the first five nucleotides counting from the 5′ end of the antisense strand; at least three, four, five, or six 2′-deoxy modifications on the sense and/or antisense strands; wherein the compound has a double stranded (duplex) region of between 19 to 25 base pairs; wherein the compound comprises a ligand; and wherein the sense strand does not comprise a glycol nucleic acid (GNA).

It is understood that the antisense strand has sufficient complementarity to a target sequence to mediate RNA interference. In other words, the compound is capable of inhibiting the expression of a target gene.

In one embodiment, the compound comprises at least three 2′-deoxy modifications. The 2′-deoxy modifications are at positions 2 and 14 of the antisense strand, counting from 5′-end of the antisense strand, and at position 11 of the sense strand, counting from 5′-end of the sense strand.

In one embodiment, the compound comprises at least five 2′-deoxy modifications. The 2′-deoxy modifications are at positions 2, 12 and 14 of the antisense strand, counting from 5′-end of the antisense strand, and at positions 9 and 11 of the sense strand, counting from 5′-end of the sense strand.

In one embodiment, the compound comprises at least seven 2′-deoxy modifications. The 2′-deoxy modifications are at positions 2, 5, 7, 12 and 14 of the antisense strand, counting from 5′-end of the antisense strand, and at positions 9 and 11 of the sense strand, counting from 5′-end of the sense strand.

In one embodiment, the antisense strand comprises at least five 2′-deoxy modifications at positions 2, 5, 7, 12 and 14, counting from 5′-end of the antisense strand. The antisense strand has a length of 18-25 nucleotides, or a length of 18-23 nucleotides.

In one embodiment, the compound can comprise one or more non-natural nucleotides. For example, the compound can comprise less than 20%, e.g., less than 15%, less than 10%, or less than 5% non-natural nucleotides, or the compound comprises no non-natural nucleotides. For example, the compound comprises all natural nucleotides. Some exemplary non-natural nucleotides include, but are not limited to, acyclic nucleotides, locked nucleic acid (LNA), HNA, CeNA, 2′-methoxyethyl, 2′-O-allyl, 2′-C-allyl, 2′-fluoro, 2′-O—N-methylacetamido (2′-O-NMA), a 2′-O-dimethylaminoethoxyethyl (2′-O-DMAEOE), 2′-O-aminopropyl (2′-O-AP), and 2′-ara-F.

In one embodiment, the compound comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the compound has a duplex region of between 19 to 25 base pairs; wherein the compound comprises a ligand; wherein the sense strand does not comprise a glycol nucleic acid (GNA); and wherein the compound comprises less than 20%, e.g., less than 15%, less than 10%, or less than 5% non-natural nucleotides or the compound comprises all natural nucleotides.

In one embodiment, at least one the sense and antisense strands comprises at least one, e.g., at least two, at least three, at least four, at least five, at least six, or at least seven or more, 2′-deoxy modifications in a central region of the sense or antisense strand. Accordingly, in one embodiment, the compound comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the compound has a duplex region of between 19 to 25 base pairs; wherein the compound comprises a ligand; and wherein the sense strand and/or the antisense strand comprises at least one, e.g., at least two, at least three, at least four, at least five, at least six, or at least seven or more, 2′-deoxy modifications in a central region of the sense strand and/or the antisense strand.

In some embodiment, the sense strand has a length of 18 to 30 nucleotides and comprises at least two 2′-deoxy modifications in the central region of the sense strand. For example, the sense strand has a length of 18 to 30 nucleotides and comprises at least two 2′-deoxy modifications within positions 7, 8, 9, 10, 11, 12, and 13, counting from 5′-end of the sense strand.

In one embodiment, the antisense strand has a length of 18 to 30 nucleotides and comprises at least two 2′-deoxy modifications in the central region of the antisense strand. For example, the antisense strand has length of 18 to 30 nucleotides and comprises at least two 2′-deoxy modifications within positions 10, 11, 12, 13, 14, 15 and 16, counting from 5′-end of the antisense strand.

In one embodiment, the compound comprises a sense strand and an antisense strand; wherein the sense strand has a length of 17-30 nucleotide and comprises at least one 2′-deoxy modification in the central region of the sense strand; and wherein the antisense strand independently has a length of 17-30 nucleotides and comprises at least two 2′-deoxy modifications in the central region of the antisense strand.

In one embodiment, the compound comprises a sense strand and an antisense strand; wherein the sense strand has a length of 17-30 nucleotide and comprises at least two 2′-deoxy modifications in the central region of the sense strand; and wherein the antisense strand independently has a length of 17-30 nucleotides and comprises at least one 2′-deoxy modification in the central region of the antisense strand.

In one embodiment, the compound comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the compound has a duplex region of between 19 to 25 base pairs; wherein the compound comprises a ligand; and wherein the sense strand comprises at least one, e.g., at least two, at least three, at least four, at least five, at least six, at least seven or more, 2′-deoxy modifications in a central region of the sense strand.

In one embodiment, the compound comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the compound has a duplex region of between 19 to 25 base pairs; wherein the compound comprises a ligand; and wherein the antisense strand comprises at least one, e.g., at least two, at least three, at least four, at least five, at least six, at least seven or more, 2′-deoxy modifications in a central region of the antisense strand.

In one embodiment, the compound comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the compound has a duplex region of between 19 to 25 base pairs; wherein the compound comprises a ligand; wherein the compound comprises less than 20%, e.g., less than 15%, less than 10%, or less than 5% non-natural nucleotides or the compound comprises all natural nucleotides; and wherein the sense strand and/or the antisense strand comprises at least one, e.g., at least two, at least three, at least four, at least five, at least six, at least seven or more, 2′-deoxy modifications in a central region of the sense strand and/or the antisense strand.

In one embodiment, the compound comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the compound has a duplex region of between 19 to 25 base pairs; wherein the compound comprises a ligand; wherein the compound comprises less than 20%, e.g., less than 15%, less than 10%, or less than 5% non-natural nucleotides or the compound comprises all natural nucleotides; and wherein the sense strand comprises at least one, e.g., at least two, at least three, at least four, at least five, at least six, at least seven or more, 2′-deoxy modifications in a central region of the sense strand.

In one embodiment, the compound comprises a sense strand and an antisense strand, each strand independently having a length of 15 to 35 nucleotides; at least two phosphorothioate internucleotide linkages between the first five nucleotides counting from the 5′ end of the antisense strand; at least three, four, five or six 2′-deoxy nucleotides on the sense and/or antisense strands; and wherein the compound has a duplex region of between 19 to 25 base pairs; wherein the compound comprises a ligand; wherein the compound comprises less than 20%, e.g., less than 15%, less than 10%, or less than 5% non-natural nucleotides or the compound comprises all natural nucleotides; and wherein the antisense strand comprises at least one, e.g., at least two, at least three, at least four, at least five, at least six, at least seven or more, 2′-deoxy modifications in a central region of the antisense strand.

In one embodiment, when the compound comprises less than 8 non-2′OMe nucleotides, the antisense stand comprises at least one DNA. For example, in any one of the embodiments of the invention when the compound comprises less than 8 non-2′OMe nucleotides, the antisense stand comprises at least one DNA.

In one embodiment, when the antisense comprises two deoxy nucleotides and said nucleotides are at positions 2 and 14, counting from the 5′-end of the antisense strand, the compound comprises 8 or less (e.g., 8, 7, 6, 5, 4, 3, 2, 1 or 0) non-2′OMe nucleotides. For example, in any one of the embodiments of the invention when the antisense comprises two deoxy nucleotides and said nucleotides are at positions 2 and 14, counting from the 5′-end of the antisense strand, the compound comprises 0, 1, 2, 3, 4, 5, 6, 7 or 8 non 2′-OMe nucleotides.

In another aspect, the invention further provides a method for delivering the compound of the invention to a specific target in a subject by subcutaneous or intravenous administration. The invention further provides the compound of the invention for use in a method for delivering said agents to a specific target in a subject by subcutaneous or intravenous administration.

Another aspect of the invention relates to a method of reducing the expression of a target gene in a cell, comprising contacting said cell with a compound comprising an antisense strand which is complementary to a target gene; a sense strand which is complementary to said antisense strand; and one or more lipophilic monomers, containing one or more lipophilic moieties, conjugated to one or more positions on at least one strand, optionally via a linker or carrier.

All the above embodiments relating to the lipophilic monomers, lipophilic moieties, and their conjugation to the compound in the first aspect of the invention relating to the compound are suitable in this aspect of the invention relating to a method of reducing the expression of a target gene in a cell.

In one embodiment, the cell is an extrahepatic cell.

In one embodiment, the cell is not a hepatocyte.

Another aspect of the invention relates to a method of reducing the expression of a target gene in a subject, comprising administering to the subject a compound comprising contacting said cell with a compound comprising an antisense strand which is complementary to a target gene; a sense strand which is complementary to said antisense strand; and one or more lipophilic monomers, containing one or more lipophilic moieties, conjugated to one or more internal positions on at least one strand, optionally via a linker or carrier.

All the above embodiments relating to the lipophilic monomers, lipophilic moieties, and their conjugation to the compound in the first aspect of the invention relating to the compound are suitable in this aspect of the invention relating to a method of reducing the expression of a target gene in a subject.

In some embodiments, the compound is administered extrahepatically.

In one embodiment, the compound is administered intrathecally or intracerebroventricularly. By intrathecal or intracerebroventricular administration of the compound, the method can reduce the expression of a target gene in a brain or spine tissue, for instance, cortex, cerebellum, cervical spine, lumbar spine, and thoracic spine.

In some embodiments, exemplary target genes are APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, and TTR. To reduce the expression of these target genes in the subject, the compound can be administered directly to the eye(s), e.g., intravitreally. By intravitreal administration of the compound, the method can reduce the expression of the target gene in an ocular tissue.

Another aspect of the invention relates to a method of treating a subject having a CNS disorder, comprising administering to the subject a therapeutically effective amount of a double-stranded RNAi agent, thereby treating the subject. The double-stranded RNAi agent comprises an antisense strand which is complementary to a target gene; a sense strand which is complementary to said antisense strand; and one or more lipophilic monomers, containing one or more lipophilic moieties conjugated to one or more internal positions on at least one strand, optionally via a linker or carrier.

All the above embodiments relating to the lipophilic monomers, lipophilic moieties, and their conjugation to the compound in the first aspect of the invention relating to the compound are suitable in this aspect of the invention relating to a method of treating a subject having a CNS disorder. Exemplary CNS disorders that can be treated by the method of the invention include Alzheimer, amyotrophic lateral sclerosis (ALS), frontotemporal dementia, Huntington, Parkinson, spinocerebellar, prion, and lafora.

### BRIEF DESCRIPTION OF THE DRAWINGS

FIG. 1 is a scheme showing the general structure of ceramide.

FIG. 2 is a graph depicting the stability of the siRNA conjugates in rat CSF after incubating the siRNA duplexes with rat CSF for 24 hours.

FIG. 3 is a graph depicting the stability of the siRNA conjugates in the vitreous humor of rabbit and cyno (NHP) for 24 hours. The remaining amounts of ligand-conjugated duplexes were plotted.

FIG. 4 is a graph depicting the stability of the siRNA conjugates in the vitreous humor of rabbit and cyno (NHP) for 24 hours. The remaining amounts of ligand-conjugated duplexes were plotted.

FIGS. 5A and 5B are graphs depicting the stability of the siRNA conjugates in rat brain homogenate for 4 hours. The remaining amounts of ligand-conjugated duplexes were plotted in FIG. 5A and the stability of PS linkages were plotted in FIG. 5B.

FIG. 6 is a graph depicting the stability of the siRNA conjugates having esterase cleavable conjugates in the vitreous humor of rabbit and cyno (NHP) for 24 hours. The percentage of the ligand-conjugated duplexes hydrolyzed were plotted.

FIG. 7 is a graph depicting the stability of the siRNA conjugates having esterase cleavable conjugates in rat plasma, CSF and brain homogenate for 24 hours. The percentage of the hydrolyzed ligand-conjugated duplexes were plotted.

FIG. 8 is a graph depicting human serum albumin binding of siRNA conjugates at different concentrations of HSA. Fraction of bound siRNA was plotted against human serum albumin concentration.

FIG. 9 is a graph depicting human serum albumin binding of siRNA conjugates having exposed carboxylic acids at different concentrations of HSA. Fraction of bound siRNA was plotted against human serum albumin concentration.

FIG. 10 is a graph depicting the inhibition of ocular TTR expression by qPCR in mouse eyes following intravitreal administration of a single 7.5 μg dose of siRNA duplexes compared to PBS control.

FIG. 11 is a graph depicting the inhibition of ocular TTR expression by qPCR in rat eyes following intravitreal administration of a single 1 μg dose of siRNA duplexes compared to PBS control.

FIG. 12 is a graph depicting the inhibition of ocular TTR expression by qPCR in mouse eyes following intravitreal administration of a single 7.5 μg dose of siRNA duplexes compared to PBS control.

FIG. 13 is a graph depicting the inhibition of ocular TTR expression by qPCR in rat eyes following intravitreal administration of a single 1 μg dose of siRNA duplexes compared to PBS control.

FIG. 14 is a graph depicting the inhibition of ocular TTR expression by qPCR in mouse eyes following intravitreal administration of a single 7.5 μg dose of siRNA duplexes compared to PBS control.

FIG. 15 is a graph depicting the inhibition of ocular TTR expression by qPCR in rat eyes following intravitreal administration of a single 1 μg dose of siRNA duplexes compared to PBS control.

FIG. 16 is a graph depicting the inhibition of TTR gene expression in primary mouse hepatocytes 24 hours after transfection of cells with the siRNA duplexes modified by Q367, as compared to the control duplex AD-900954 at three different concentrations. Each of the nucleotides was modified across sense strand by Q367.

FIG. 17 is a graph depicting the inhibition of SOD1 gene expression in primary mouse hepatocytes 24 hour after transfection of cells with the siRNA duplexes modified by Q367, as compared to the control duplex AD-900954 at three different concentrations. Each of the nucleotides was modified across sense strand by Q367.

FIGS. 18A-18D are graphs depicting the inhibition of SOD1 expression by qPCR in rat spinal cord (FIG. 18A), cerebellum (FIG. 18B), frontal cortex (FIG. 18C) and heart (FIG. 18D) following IT administration of a single 0.9 mg of the siRNA duplexes/rat, as compared to artificial CSF dosed control group after 14 days.

FIGS. 19A-19E are graphs depicting the inhibition of SOD1 expression by qPCR in rat spinal cord (FIG. 19A), brain stem (FIG. 19B), cerebellum (FIG. 19C), frontal cortex (FIG. 19D) and heart (FIG. 19E) following IT administration of a single 0.9 mg of the siRNA duplexes/rat, as compared to artificial CSF dosed control group after 14 days.

FIG. 20 is a graph depicting the inhibition of SOD1 expression by qPCR in rat brain (cerebellum and frontal cortex) and spinal cord (thoracic spinal cord) following IT administration of a single 0.9 mg of siRNA duplexes/rat, as compared to artificial CSF dosed control group after 14 days.

FIGS. 21A and 21B are graphs depicting the inhibition of SOD1 expression by qPCR in mouse brain (right hemisphere) and heart following ICV administration of a single 50 μg (FIG. 21A) and 110 μg (FIG. 21B) of siRNA duplexes/mice, as compared to artificial CSF dosed control group after 14 days (FIG. 21A) and 7 days (FIG. 21B).

### DETAILED DESCRIPTION

The inventors have found, inter alia, that conjugating a lipophilic monomer containing a lipophilic moiety to one or more positions on at least one strand of the compound provides surprisingly good results for in vivo ocular delivery (e.g., intravitreal delivery) and intrathecal or intracerebroventricular delivery of the double-stranded iRNAs, resulting in efficient entry of CNS tissues and ocular tissues and are efficiently internalized into cells of the CNS system and ocular system.

One aspect of the invention provides a compound comprising: an antisense strand which is complementary to a target gene; a sense strand which is complementary to said antisense strand; and one or more lipophilic monomers, containing one or more lipophilic moieties, conjugated to one or more positions on at least one strand, optionally via a linker or carrier.

The term “lipophile” or “lipophilic moiety” broadly refers to any compound or chemical moiety having an affinity for lipids. One way to characterize the lipophilicity of the lipophilic moiety is by the octanol-water partition coefficient, logK_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf. Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its logK_{ow} exceeds 0. Typically, the lipophilic moiety possesses a logK_{ow} exceeding 1, exceeding 1.5, exceeding 2, exceeding 3, exceeding 4, exceeding 5, or exceeding 10. For instance, the logK_{ow} of 6-amino hexanol, for instance, is predicted to be approximately 0.7. Using the same method, the logK_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., logK_{ow}) value of the lipophilic moiety.

Alternatively, the hydrophobicity of the compound (e.g., the double-stranded iRNA agent), conjugated to one or more lipophilic monomers, containing one or more lipophilic moieties, can be measured by its protein binding characteristics. For instance, the unbound fraction in the plasma protein binding assay of the compound can be determined to positively correlate to the relative hydrophobicity of the double-stranded iRNA agent, which can positively correlate to the silencing activity of the double-stranded iRNA agent.

In one embodiment, the plasma protein binding assay determined is an electrophoretic mobility shift assay (EMSA) using human serum albumin protein. The hydrophobicity of the double-stranded iRNA agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

Accordingly, conjugating the lipophilic monomers, containing lipophilic moieties, to the compound provides optimal hydrophobicity for the enhanced in vivo delivery of siRNA.

In certain embodiments, the lipophilic moiety is an aliphatic, cyclic such as alicyclic, or polycyclic such as polyalicyclic compound, such as a steroid (e.g., sterol) or a linear or branched aliphatic hydrocarbon. The lipophilic moiety may generally comprises a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₁₈ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, the lipophilic moiety may contain a C₄-C₃₀ hydrocarbon chain (e.g., C₄-C₃₀ alkyl or alkenyl). In some embodiment the lipophilic moiety contains a saturated or unsaturated C₆-C₁₈ hydrocarbon chain (e.g., a linear C₆-C₁₈ alkyl or alkenyl). In one embodiment, the lipophilic moiety contains a saturated or unsaturated C₁₆ hydrocarbon chain (e.g., a linear C₁₆ alkyl or alkenyl).

The lipophilic monomer containing the lipophilic moiety may be attached to the iRNA agent by any method known in the art, including via a functional grouping already present in the lipophilic monomer or introduced into the iRNA agent, such as a hydroxy group (e.g., —CO—CH₂—OH). The functional groups already present in the lipophilic monomer or introduced into the iRNA agent include, but are not limited to, hydroxyl, amine, carboxylic acid, sulfonate, phosphate, thiol, azide, and alkyne.

Conjugation of the iRNA agent and the lipophilic monomer may occur, for example, through formation of an ether or a carboxylic or carbamoyl ester linkage between the hydroxy and an alkyl group R—, an alkanoyl group RCO— or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl or octadecyl group, or the like.

In some embodiments, the lipophilic monomer comprising the lipophilic moiety is conjugated to the compound via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In another embodiment, the lipophilic moiety is a steroid, such as sterol. Steroids are polycyclic compounds containing a perhydro-1,2-cyclopentanophenanthrene ring system. Steroids include, without limitation, bile acids (e.g., cholic acid, deoxycholic acid and dehydrocholic acid), cortisone, digoxigenin, testosterone, cholesterol, and cationic steroids, such as cortisone. A “cholesterol derivative” refers to a compound derived from cholesterol, for example by substitution, addition or removal of substituents.

In another embodiment, the lipophilic moiety is an aromatic moiety. In this context, the term “aromatic” refers broadly to mono- and polyaromatic hydrocarbons. Aromatic groups include, without limitation, C₆-C₁₄ aryl moieties comprising one to three aromatic rings, which may be optionally substituted; “aralkyl” or “arylalkyl” groups comprising an aryl group covalently linked to an alkyl group, either of which may independently be optionally substituted or unsubstituted; and “heteroaryl” groups. As used herein, the term “heteroaryl” refers to groups having 5 to 14 ring atoms, preferably 5, 6, 9, or 10 ring atoms; having 6, 10, or 14π electrons shared in a cyclic array, and having, in addition to carbon atoms, between one and about three heteroatoms selected from the group consisting of nitrogen (N), oxygen (O), and sulfur (S).

As employed herein, a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

In some embodiments, the lipophilic moiety is an aralkyl group, e.g., a 2-arylpropanoyl moiety. The structural features of the aralkyl group are selected so that the lipophilic moiety will bind to at least one protein in vivo. In certain embodiments, the structural features of the aralkyl group are selected so that the lipophilic moiety binds to serum, vascular, or cellular proteins. In certain embodiments, the structural features of the aralkyl group promote binding to albumin, an immunoglobulin, a lipoprotein, α-2-macroglubulin, or α-1-glycoprotein.

In certain embodiments, the ligand is naproxen or a structural derivative of naproxen. Procedures for the synthesis of naproxen can be found in U.S. Pat. Nos. 3,904,682 and 4,009,197, which are hereby incorporated by reference in their entirety. Naproxen has the chemical name (S)-6-Methoxy-α-methyl-2-naphthaleneacetic acid and the structure is

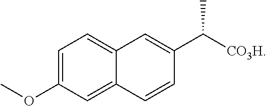

In certain embodiments, the ligand is ibuprofen or a structural derivative of ibuprofen. Procedures for the synthesis of ibuprofen can be found in U.S. Pat. No. 3,228,831, which are hereby incorporated by reference in their entirety. The structure of ibuprofen is

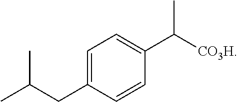

Additional exemplary aralkyl groups are illustrated in U.S. Pat. No. 7,626,014, which is incorporated herein by reference in its entirety.

In another embodiment, suitable lipophilic moieties include lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

In some embodiments, the lipophilic moiety is a C₆-C₃₀ acid (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, cis-4,7,10,13,16,19-docosahexaenoic acid, vitamin A, vitamin E, cholesterol etc.) or a C₆-C₃₀ alcohol (e.g., hexanol, heptanol, octanol, nonanol, decanol, undecanol, dodcanol, tridecanol, tetradecanol, pentadecanol, hexadecanol, heptadecanol, octadecanol, oleyl alcohol, linoleyl alcohol, arachidonic alcohol, cis-4,7,10,13,16,19-docosahexanol, retinol, vitamin E, cholesterol etc.).

In certain embodiments, lipophilic monomers containing more than one lipophilic moieties can be incorporated into the double-strand iRNA agent, particularly when the lipophilic moiety has a low lipophilicity or hydrophobicity. In one embodiment, lipophilic monomers containing two or more lipophilic moieties are incorporated into the same strand of the double-strand iRNA agent. In one embodiment, each strand of the double-strand iRNA agent has a lipophilic monomer containing one or more lipophilic moieties incorporated. In one embodiment, a lipophilic monomer containing two or more lipophilic moieties are incorporated into the same position (i.e., the same nucleobase, same sugar moiety, or same internucleosidic linkage) of the double-strand iRNA agent. This can be achieved by, e.g., a using a lipophilic monomer containing a carrier, and/or a branched linker, and/or one or more linkers that can link the two or more lipophilic moieties.

When the lipophilic moiety is conjugated to the iRNA agent via a direct attachment to the nucleobase, ribosugar, or internucleosidic linkage of the iRNA agent, the lipophilic monomer then comprises the nucleobase, ribosugar, or internucleosidic linkage, and the lipophilic moiety. Alternatively, the lipophilic monomer may comprise a lipophilic moiety conjugated to a non-ribose replacement unit, such as a linker or carrier. When the lipophilic moiety is conjugated to the double-strand iRNA agent via a non-ribose replacement unit, such as a linker or a carrier, the lipophilic monomer then comprises the non-ribose replacement unit, such as the linker or carrier, and the lipophilic moiety.

In certain embodiments, the lipophilic monomer comprises the lipophilic moiety conjugated to the iRNA agent via one or more linkers (tethers).

In one embodiment, the lipophilic monomer comprises the lipophilic moiety conjugated to the compound via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

### Linkers Tethers

Linkers/Tethers are connected to the lipophilic moiety at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for the lipophilic moiety. Non-limited examples of linkers/tethers (underlined) include TAP-(CH₂)ₙNH-; TAP-C(O)(CH₂)ₙNH-; TAP-NR″″(CH₂)ₙNH-, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂)ₙ-; TAP-C(O)—NH—; TAP-C(O)—; TAP-(CH₂)ₙ—C(O)—; TAP-(CH₂)ₙ—C(O)O—; TAP-(CH₂)ₙ-; or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND); TAP-NR″″(CH₂)ₙNH(LIGAND); TAP-(CH₂)ₙONH(LIGAND); TAP-C(O)(CH₂)ₙONH(LIGAND); TAP-NR″″(CH₂)ₙONH(LIGAND); TAP-(CH₂)ₙNHNH₂(LIGAND), TAP-C(O)(CH₂)ₙNHNH₂(LIGAND); TAP-NR″″(CH₂)ₙNHNH₂(LIGAND); TAP-C(O)—(CH₂)ₙ—C(O)(LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O)(LIGAND); TAP-(CH₂)ₙ—C(O)O(LIGAND); TAP-(CH₂)ₙ(LIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP—(CH₂)ₙ—SH, TAP-C(O)(CH₂)ₙSH, TAP—(CH₂)ₙ—(CH═CH₂), or TAP-C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP—(CH₂)ₙCHO; TAP-C(O)(CH₂)ₙCHO; or TAP-NR″″(CH₂)ₙCHO, in which n is 1-6 and R″″ is C₁-C₆ alkyl; or TAP—(CH₂)ₙC(O)ONHS; TAP-C(O)(CH₂)ₙC(O)ONHS; or TAP-NR″″(CH₂)ₙC(O)ONHS, in which n is 1-6 and R″″ is C₁-C₆ alkyl; TAP-(CH₂)ₙC(O)OC₆F₅; TAP-C(O)(CH₂)ₙC(O) OC₆F₅; or TAP-NR″″(CH₂)ₙC(O) OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the linker/tether.

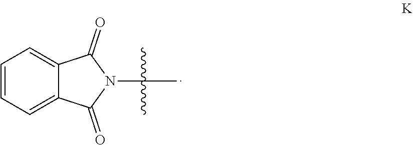 [SMILES: *N1C(=O)c2ccccc2C1=O]

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### Cleavable Linkers Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the iRNA agent.

A tether can include a linking group that is cleavable by a particular enzyme. The type of linking group incorporated into a tether can depend on the cell to be targeted by the iRNA agent. For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, S—P(O)(ORk)-S—, —O—P(S)(ORk)-S—, S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S, —O—P(S)(Rk)-S—. Preferred embodiments are O—P(O)(OH)—O—, —O—P(S)(OH)O—, —O—P(S)(SH)O—, —S—P(O)(OH)O—, —O—P(O)(OH)—S—, —S—P(O)(OH)S—, —O—P(S)(OH)S—, —S—P(S)(OH)O—, —O—P(O)(H)O—, —O—P(S)(H)O—, —S—P(O)(H)O—, —S—P(S)(H)O—, —S—P(O)(H)—S—, —O—P(S)(H)—S—. A preferred embodiment is —O—P(O)(OH)—O—. These candidates can be evaluated using methods analogous to those described above.

### Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula NHCHR¹C(O)NHCHR²C(O)—, where R¹ and R² are the R groups of the two adjacent amino acids. These candidates can be evaluated using methods analogous to those described above.

### Biocleavable Linkers Tethers

The linkers can also include biocleavable linkers that are nucleotide and non-nucleotide linkers or combinations thereof that connect two parts of a molecule, for example, one or both strands of two individual siRNA molecule to generate a bis(siRNA). In some embodiments, mere electrostatic or stacking interaction between two individual siRNAs can represent a linker. The non-nucleotide linkers include tethers or linkers derived from monosaccharides, disaccharides, oligosaccharides, and derivatives thereof, aliphatic, alicyclic, heterocyclic, and combinations thereof.

In some embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

### Exemplary Bio-Cleavable Linkers Include:

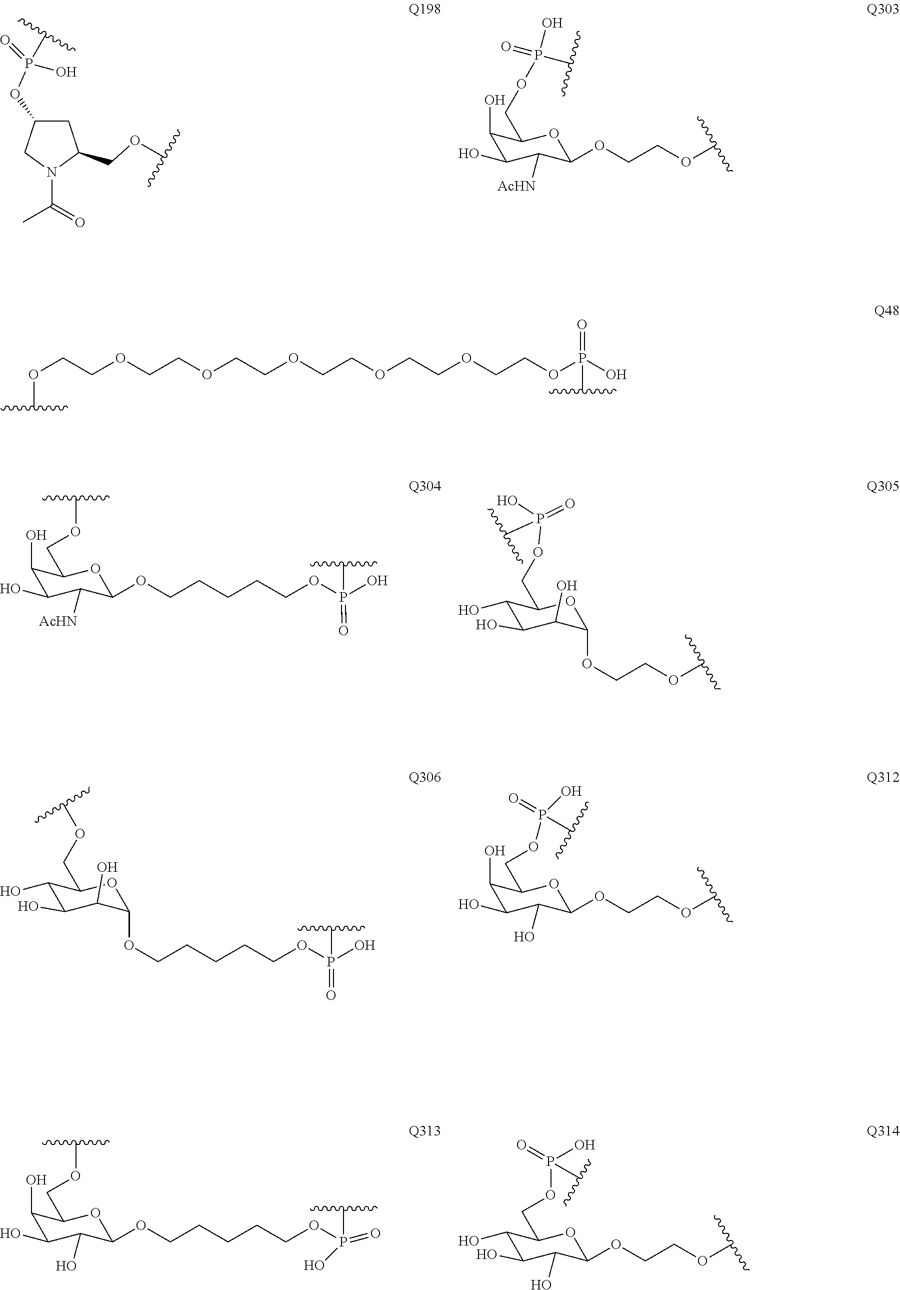

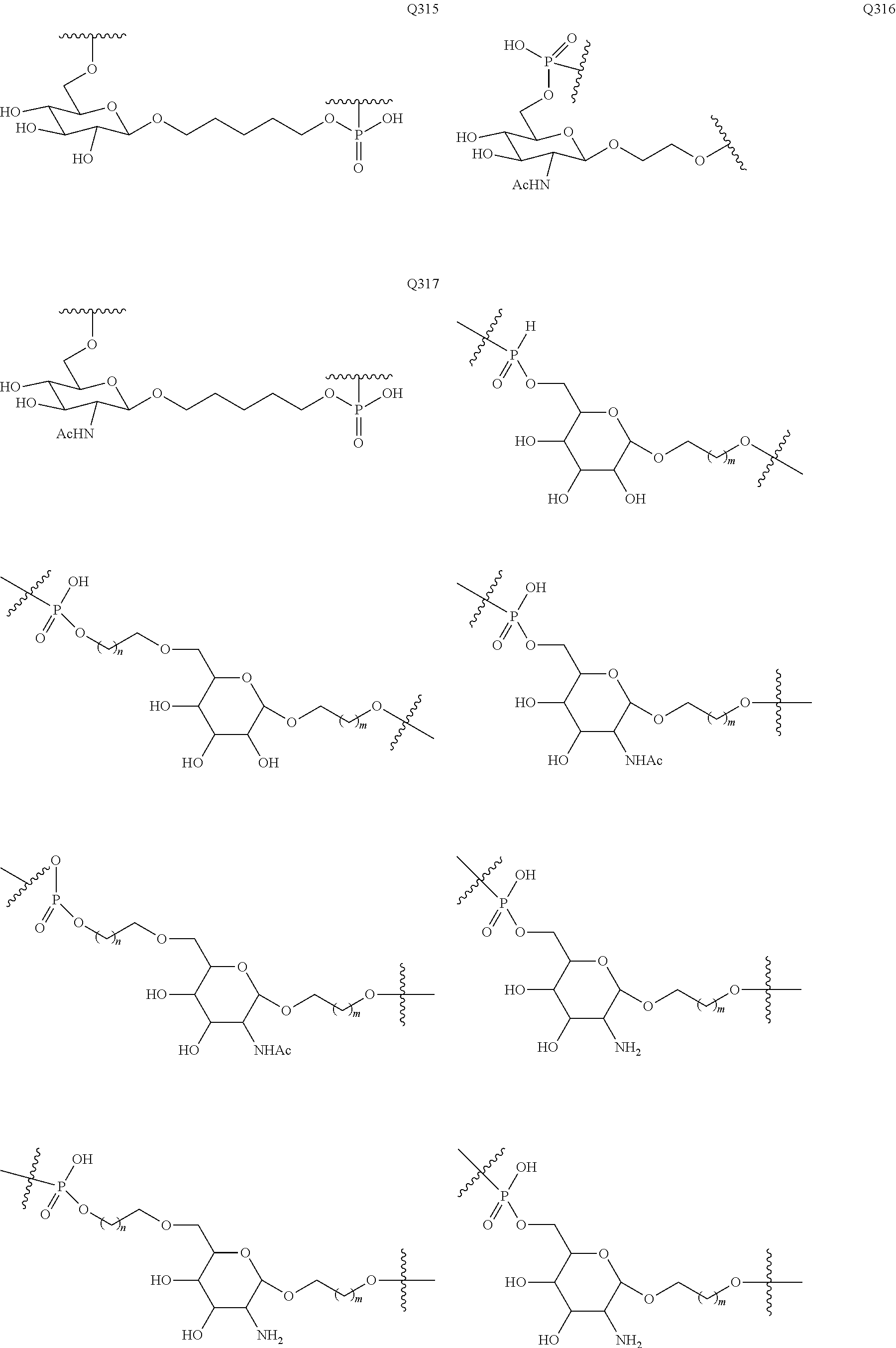

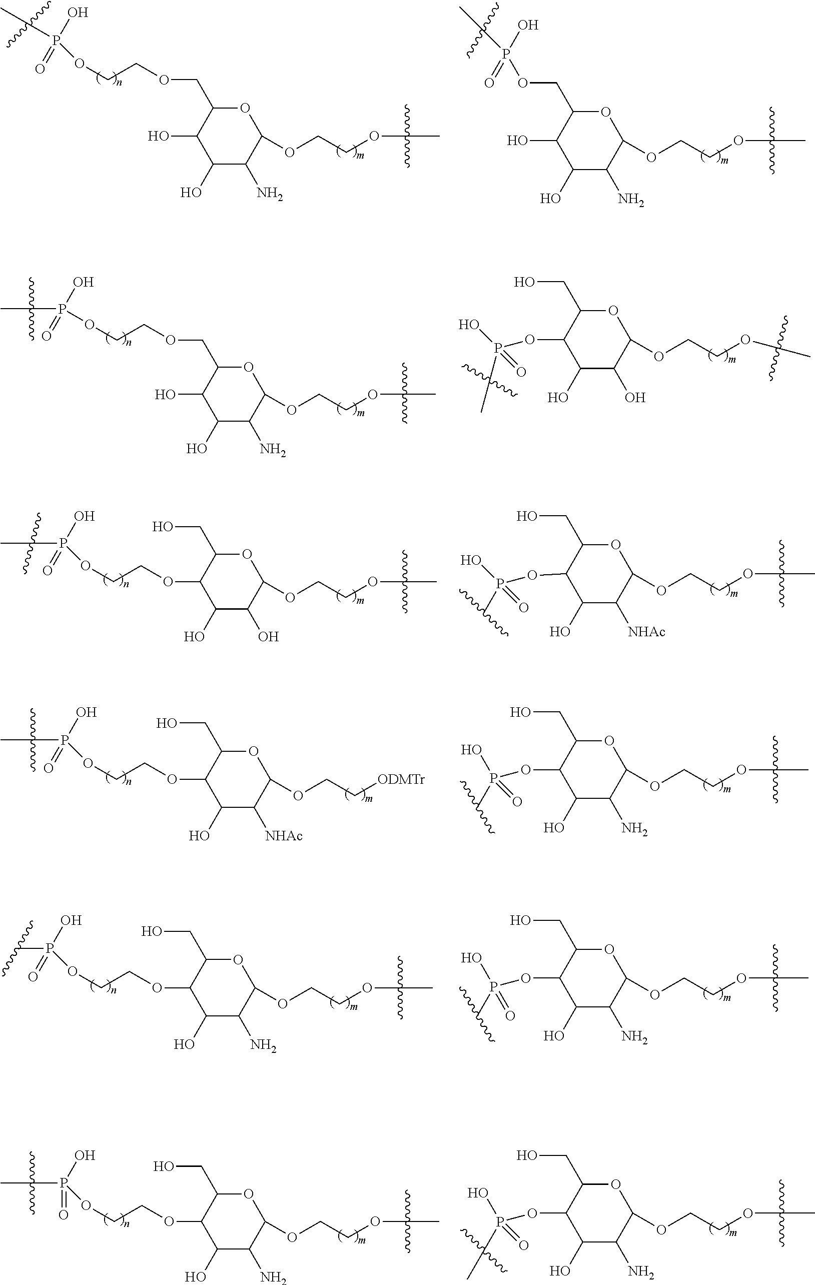

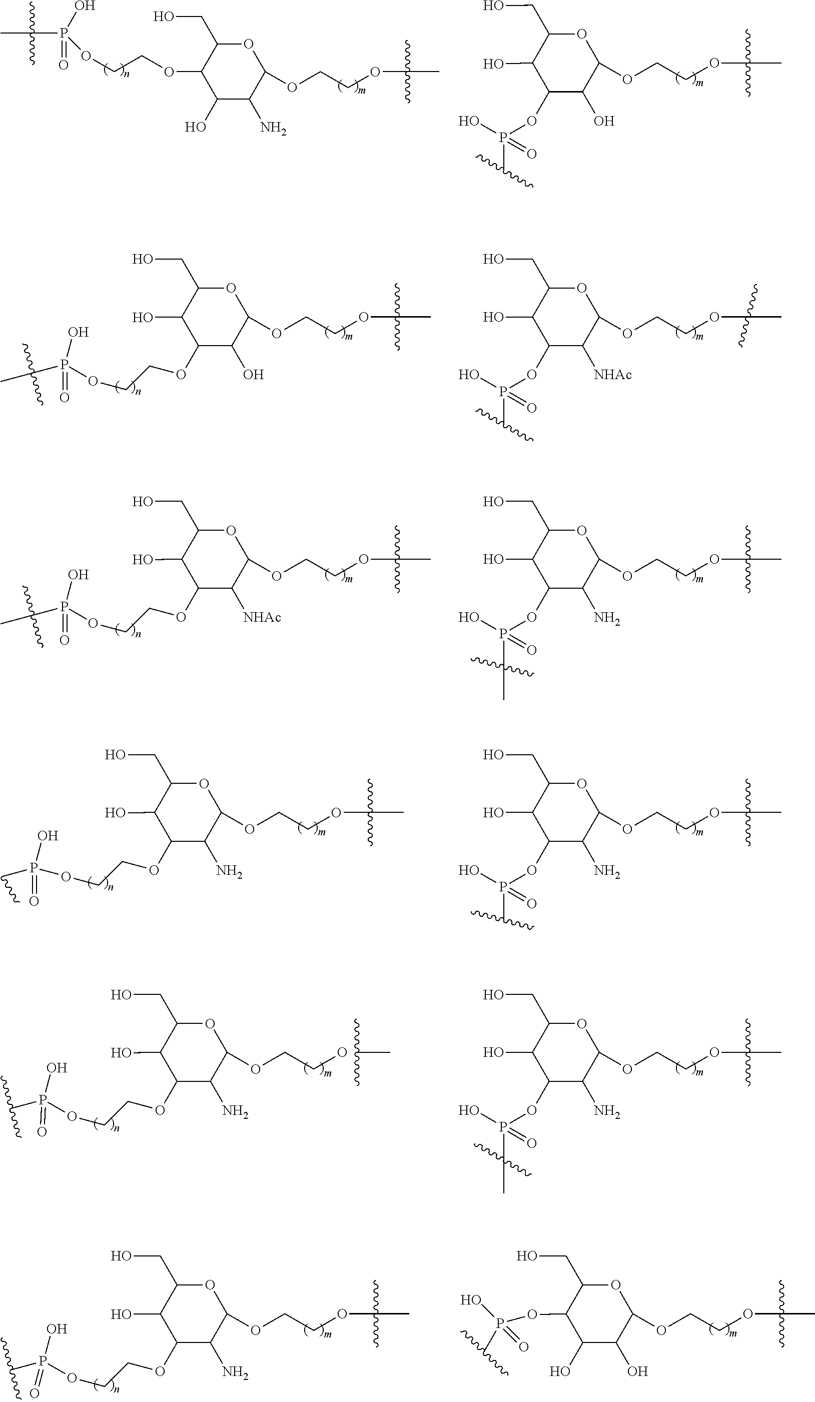

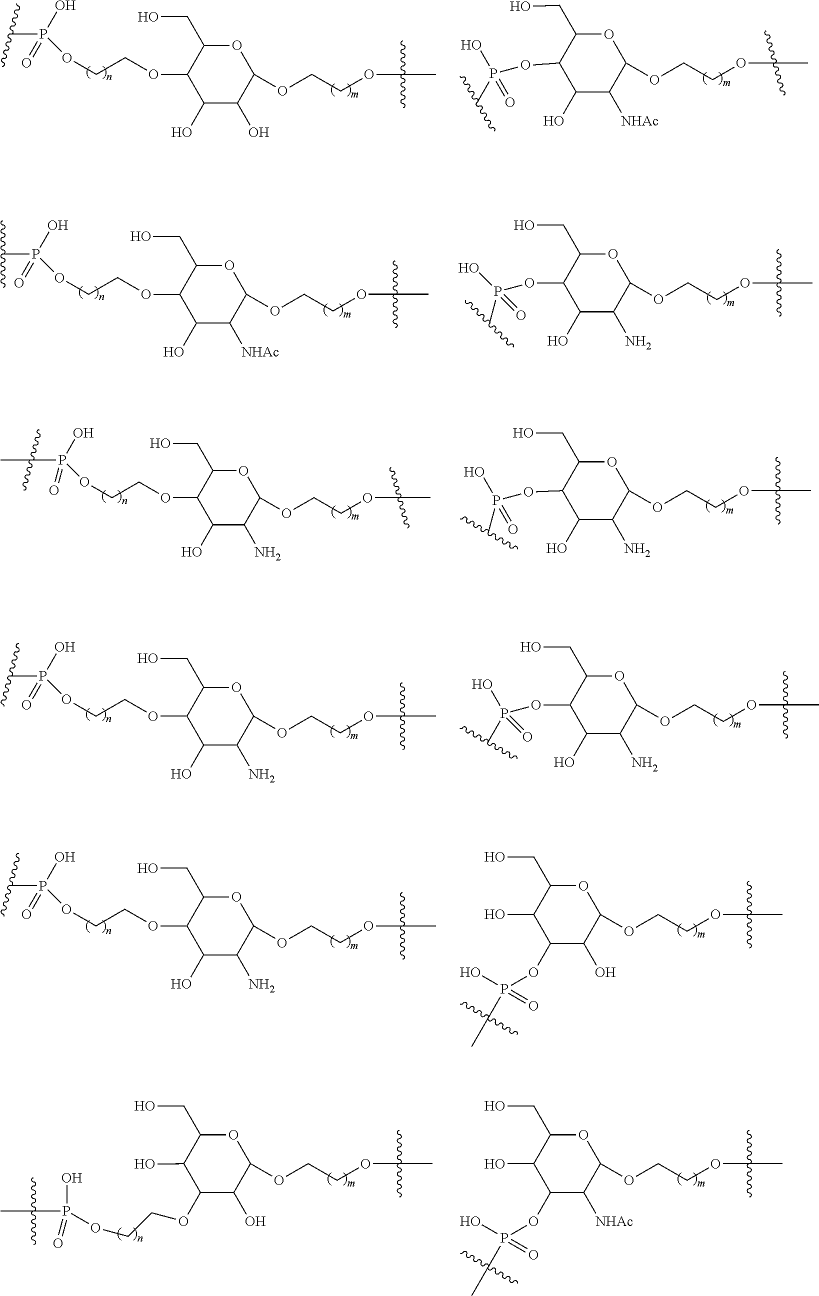

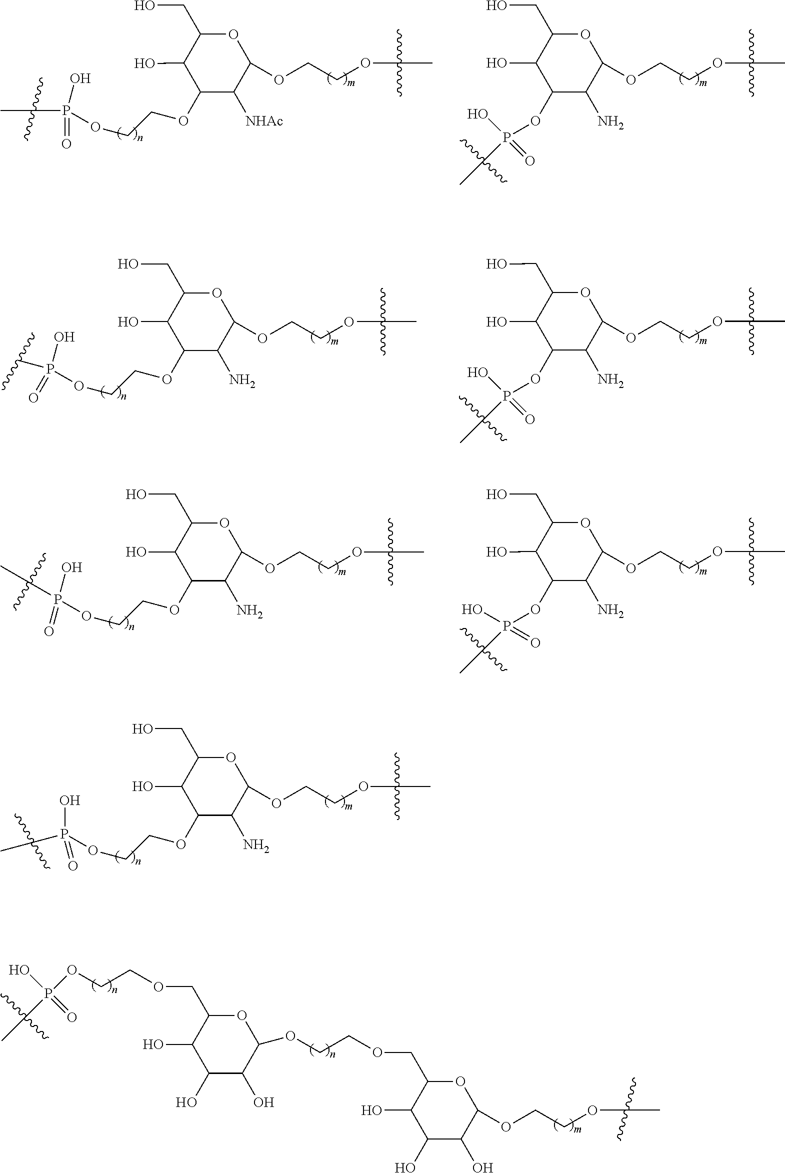

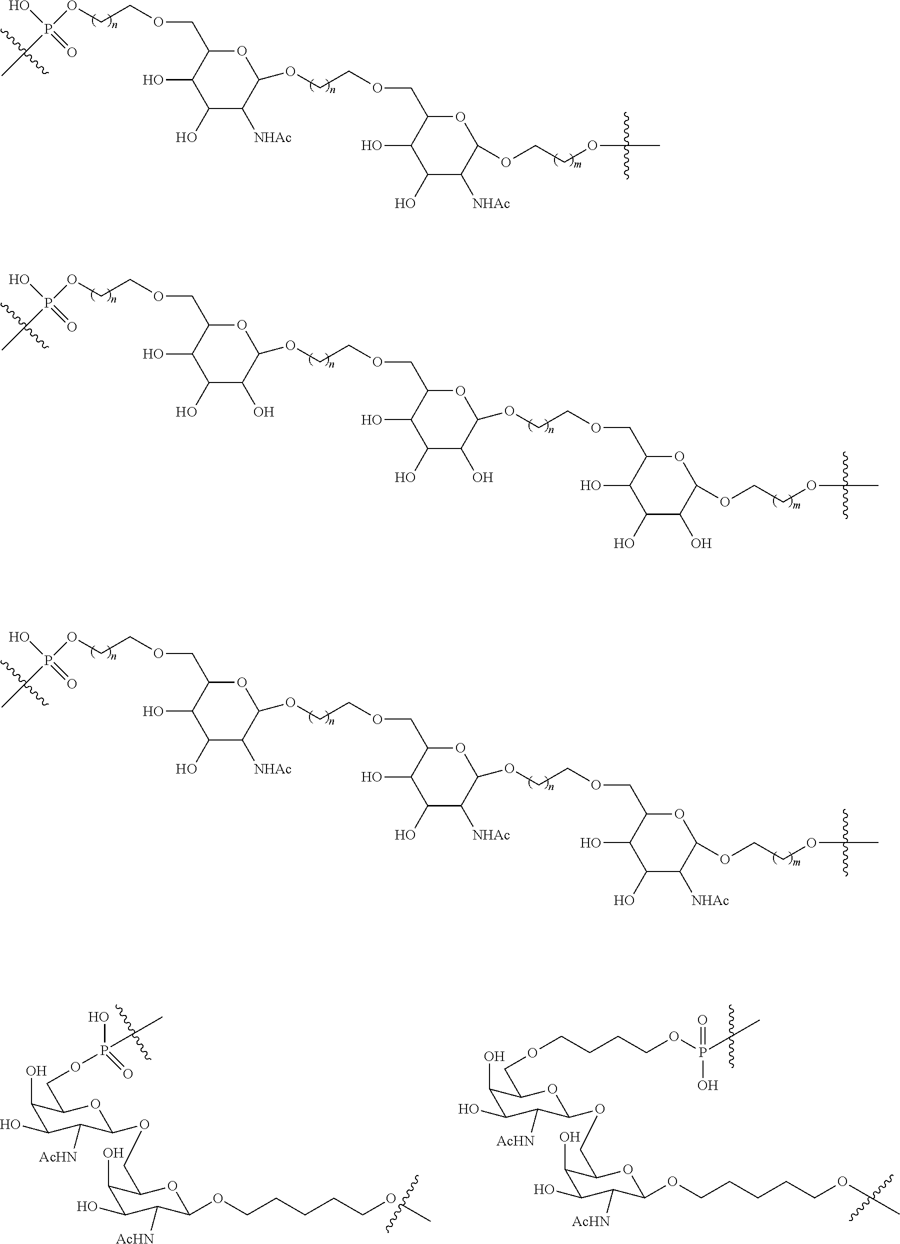

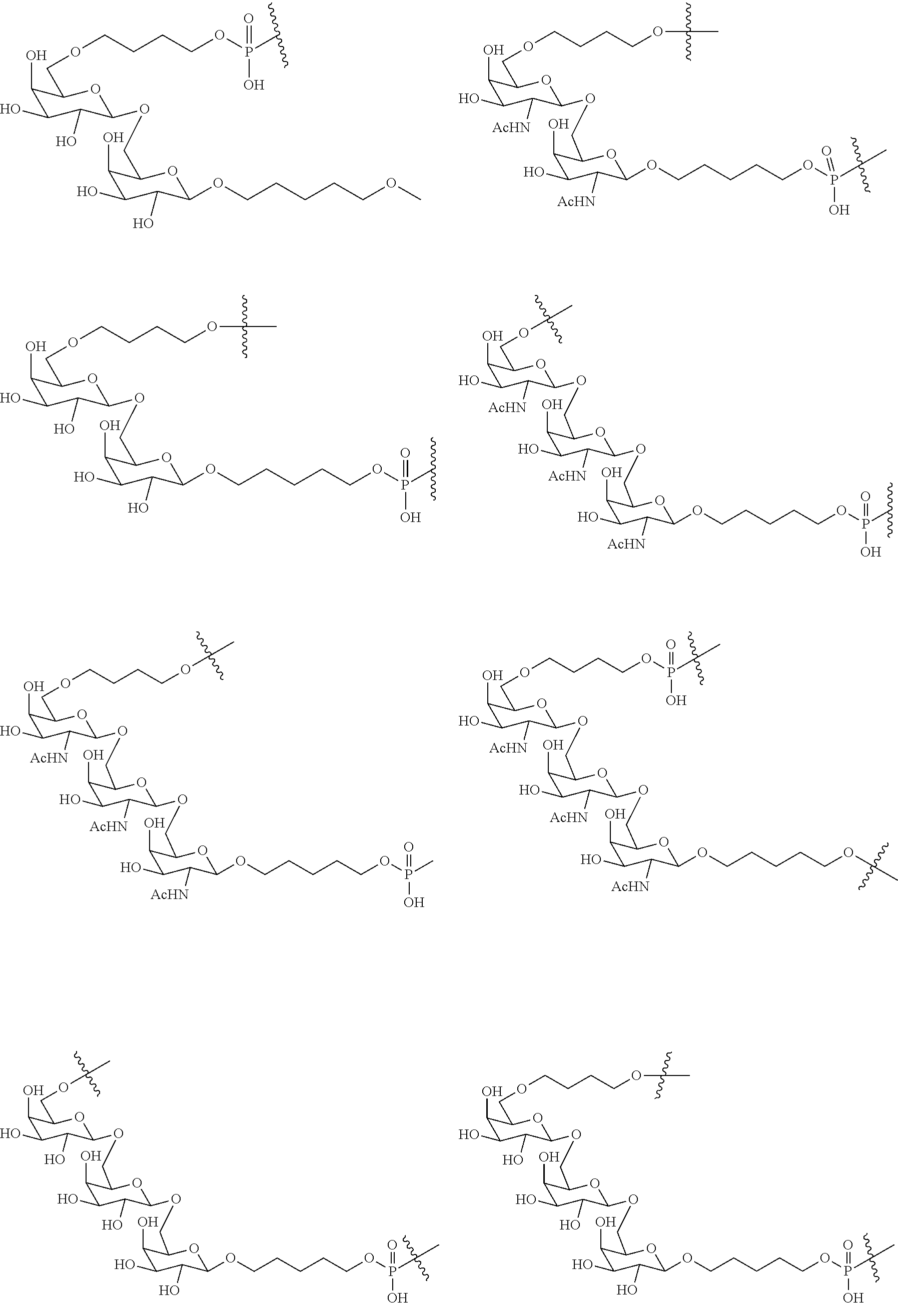

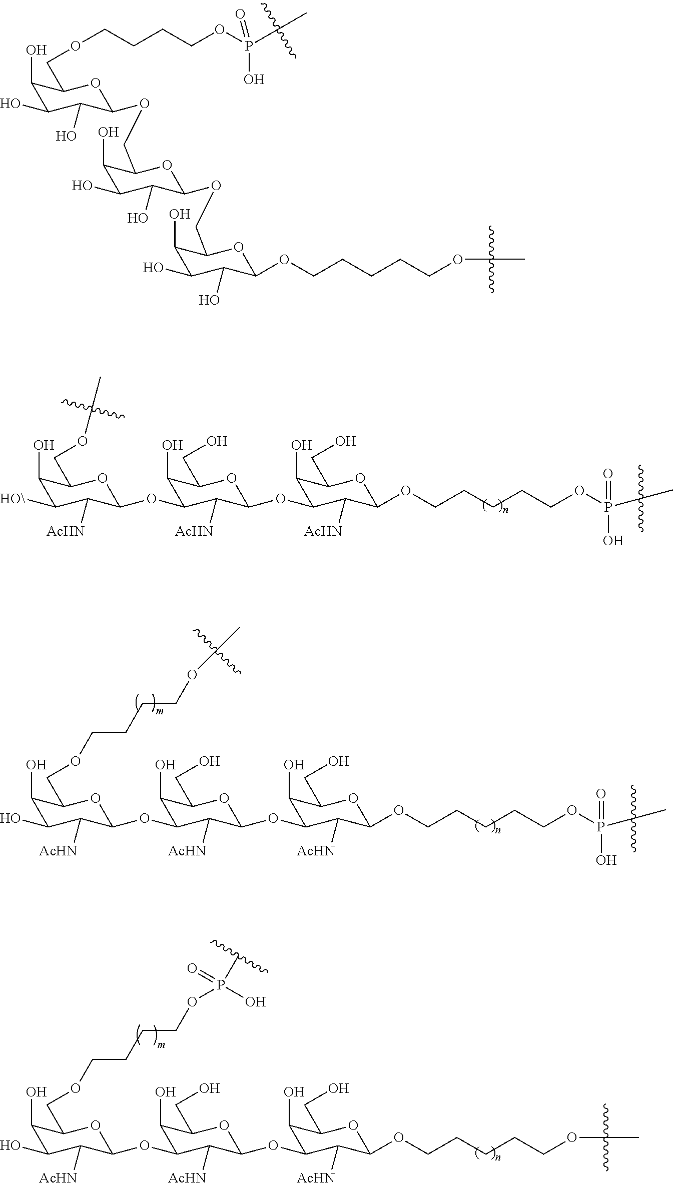

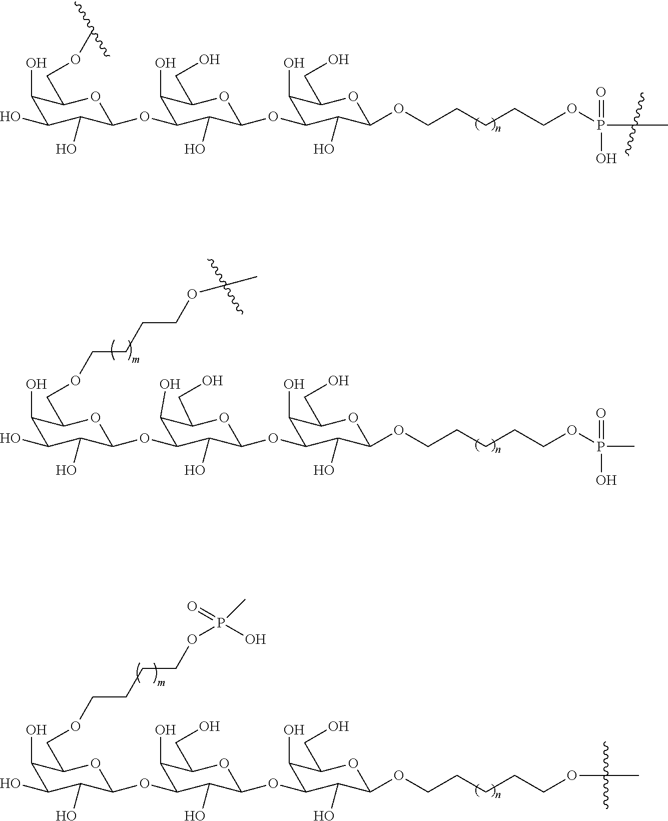

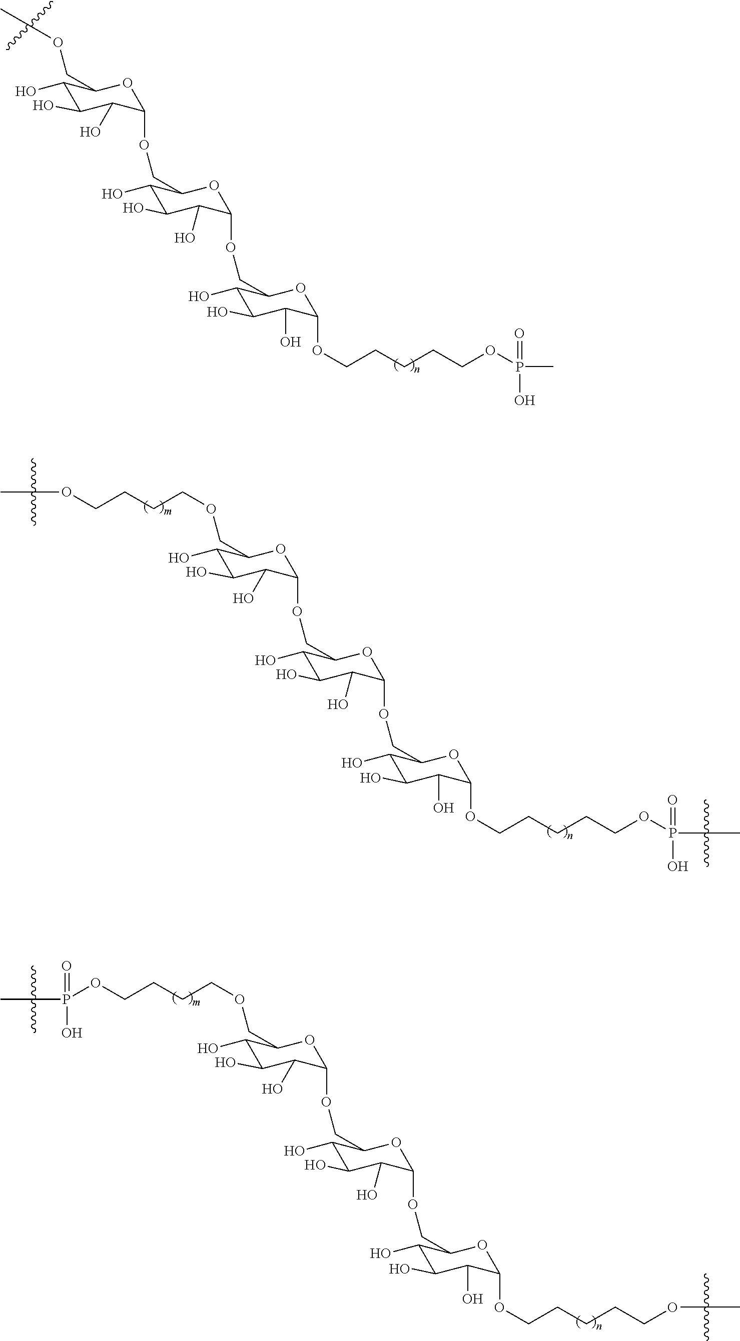

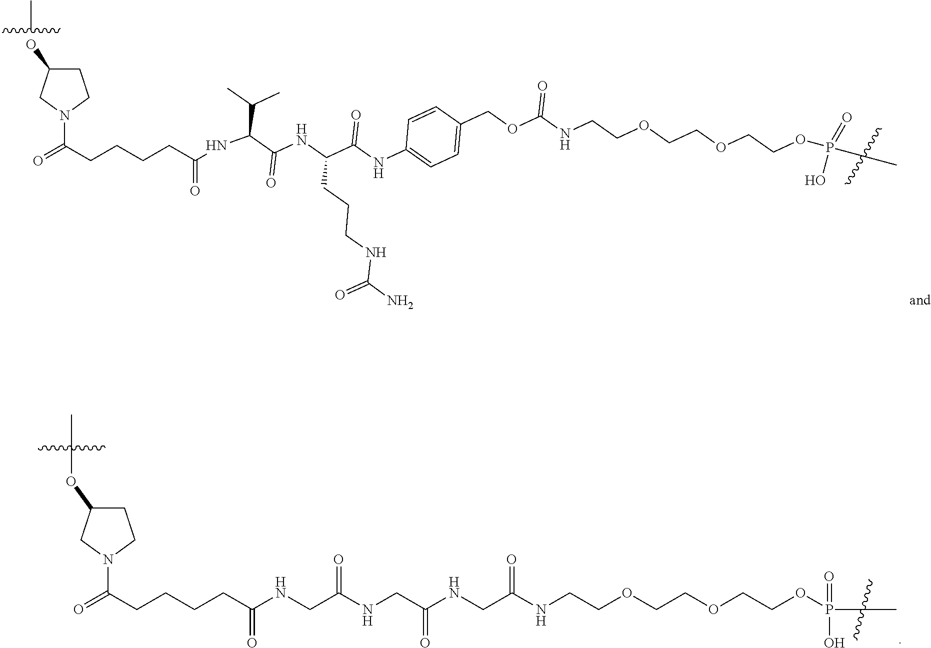

More discussion about the biocleavable linkers may be found in PCT application No. PCT/US18/14213, entitled “Endosomal Cleavable Linkers,” filed on Jan. 18, 2018, the content of which is incorporated herein by reference in its entirety.

### Carriers

In certain embodiments, the lipophilic monomer comprises the lipophilic moiety conjugated to the iRNA agent via a non-ribose replacement unit, i.e., a carrier that replaces one or more nucleotide(s).

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

The carrier can replace one or more nucleotide(s) of the double-stranded iRNA agent.

In some embodiments, the carrier replaces one or more nucleotide(s) in the internal position(s) of the double-stranded iRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand (e.g., the lipophilic moiety). The lipophilic moiety can be directly attached to the carrier or indirectly attached to the carrier by an intervening linker/tether, as described above.

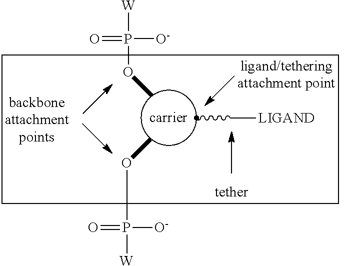

The ligand-conjugated monomer subunit may be the 5′ or 3′ terminal subunit of the iRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in an iRNA agent.

### Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

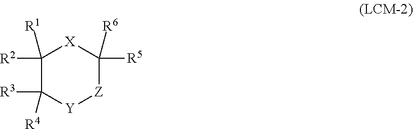

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR^{a} and/or (CH₂)ₙOR;
Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or Si(X^{5′})(X⁵″)(X^{5′″}) in which (X^{5′}), (X⁵″), and (X⁵′″) are as described elsewhere.
R^{b} is P(O)(O⁻)H, P(OR¹⁵)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy;
n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

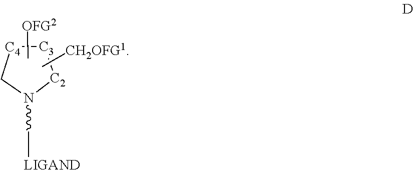

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

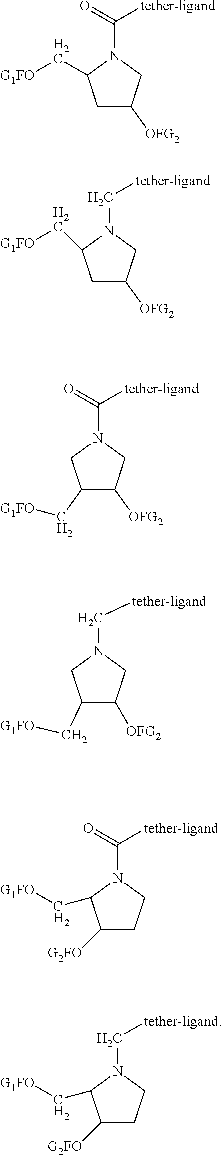

In certain embodiments, the carrier may be based on the piperidine ring system (E), e.g., X is N(CO)R⁷ or NR⁷, Y is CR⁹R¹⁰, and Z is CR¹¹R¹².

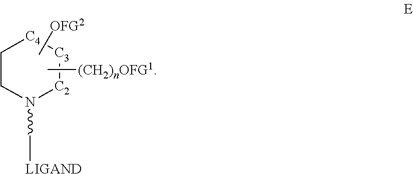

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹².

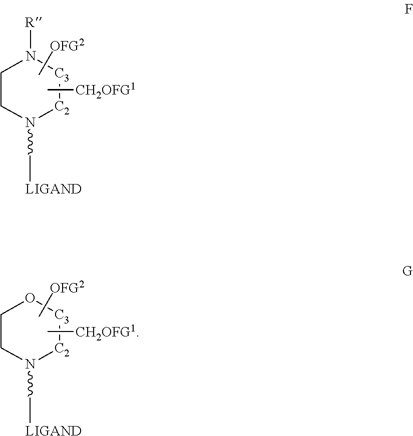

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). R′″ can be, e.g., C₁-C₆ alkyl, preferably CH₃. The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

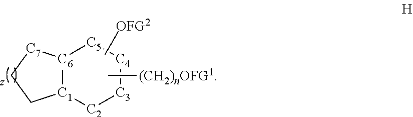

OFG¹ is preferably attached to a primary carbon, e.g., an exocyclic methylene group (n=1) or ethylene group (n=2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

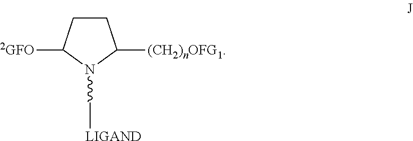

Thus, —(CH₂)ₙOFG¹ and OFG² may be cis or trans with respect to one another. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

### Sugar Replacement-Based Monomers (Acyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

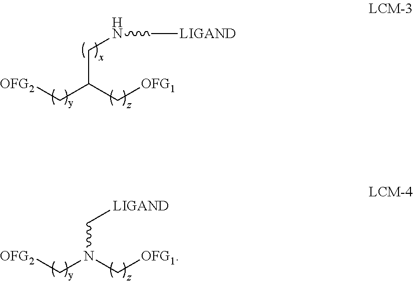

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

In some embodiments, the compound comprises one or more lipophilic monomers containing lipophilic moieties conjugated to the 5′ end of the sense strand or the 5′ end of the antisense strand.

In certain embodiments, the lipophilic monomer contains a lipophilic moiety conjugated to the 5′-end of a strand via a carrier and/or linker. In one embodiment, the lipophilic monomer containing a lipophilic moiety conjugated to the 5′-end of a strand via a carrier of a formula:

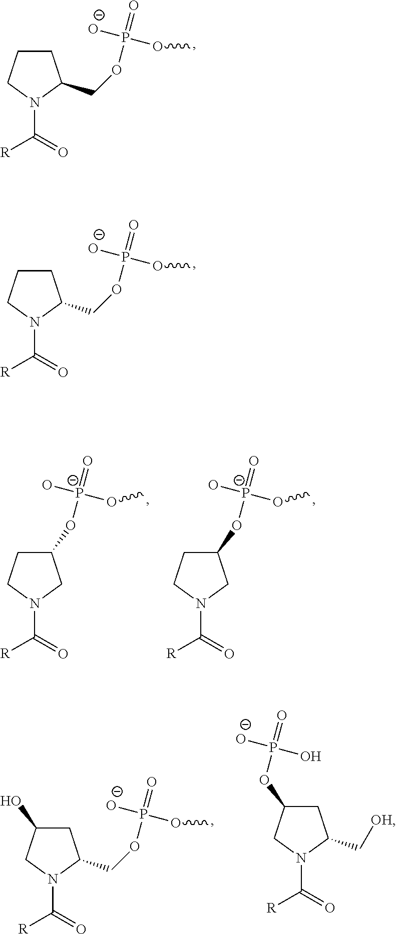

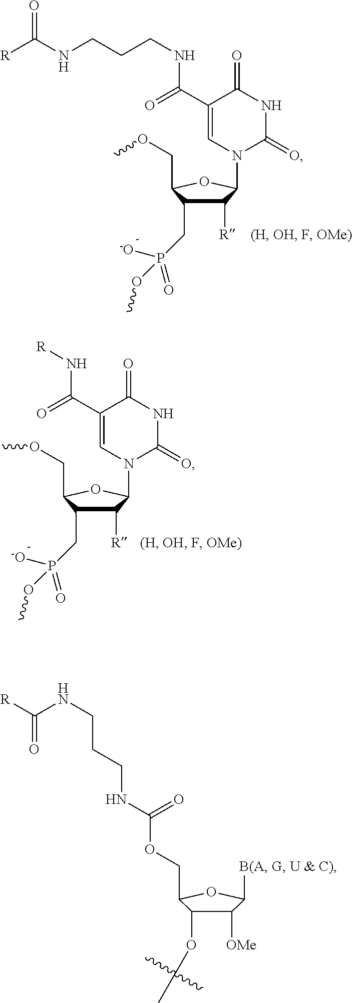

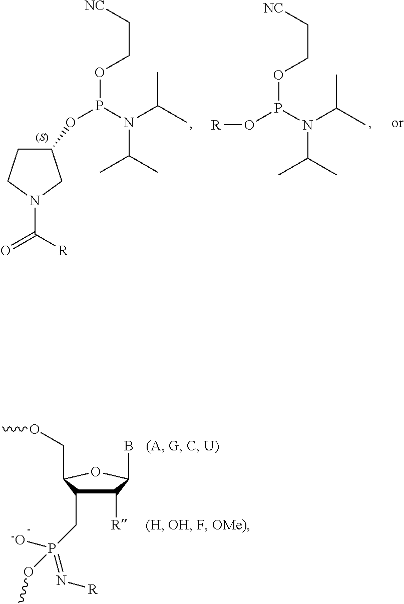

wherein R is a ligand such as the lipophilic moiety.

In some embodiments, the compound comprises one or more lipophilic monomers containing one or more lipophilic moieties conjugated to the 3′ end of the sense strand or the 3′ end of the antisense strand.

In certain embodiments, the lipophilic monomer contains a lipophilic moiety conjugated to the 3′-end of a strand via a carrier and/or linker. In one embodiment, the lipophilic monomer contains a lipophilic moiety conjugated to the 3′-end of a strand via a carrier of a formula:

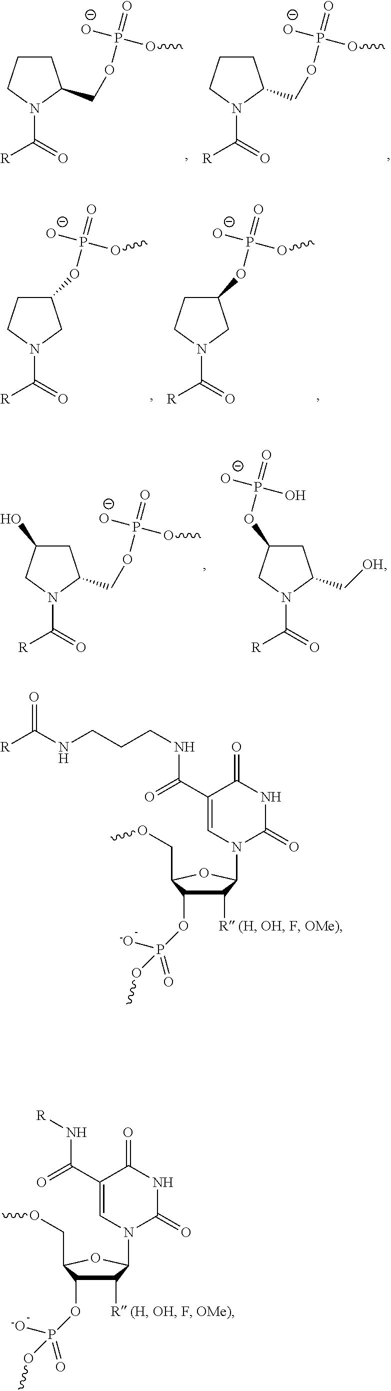

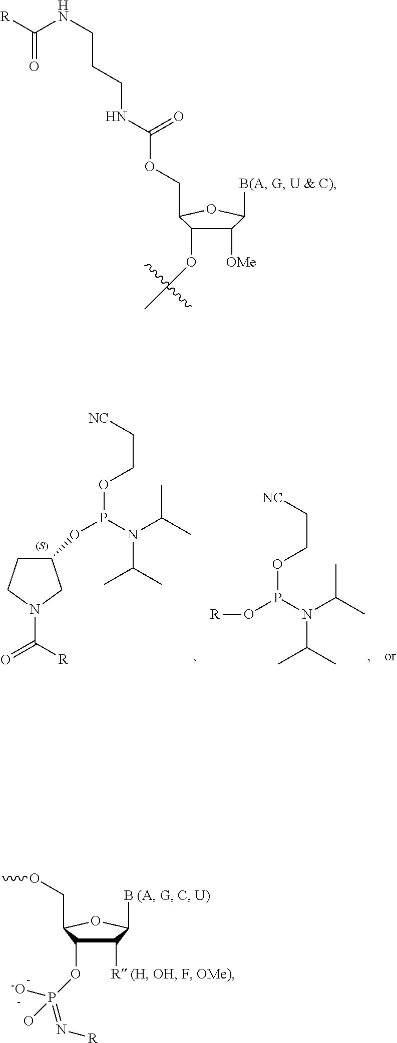

wherein R is a ligand such as the lipophilic moiety.

In certain embodiments, the lipophilic monomer contains a lipophilic moiety conjugated to the internal position of a strand via a carrier and/or linker. In one embodiment, the lipophilic monomer contains a lipophilic moiety conjugated to the internal position of a strand via a carrier of a formula:

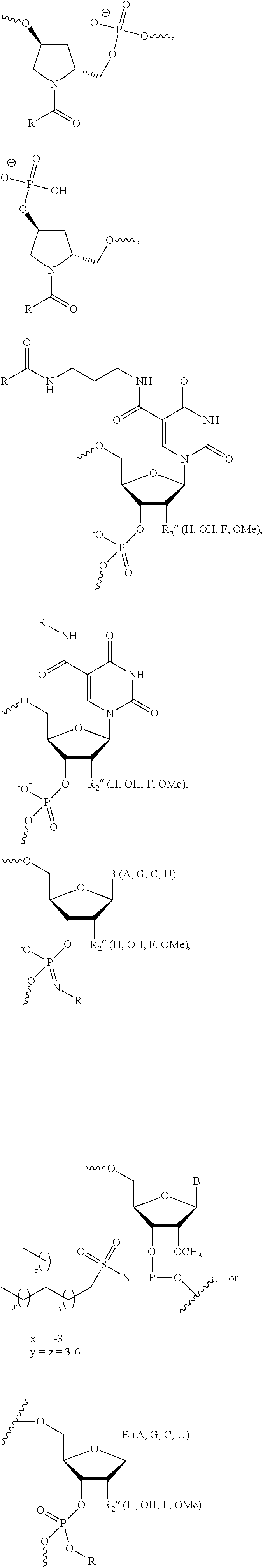

wherein R is a ligand such as the lipophilic moiety.

In some embodiments, the compound comprises one or more lipophilic monomers containing one or more lipophilic moieties conjugated to both ends of the sense strand.

In some embodiments, the compound comprises one or more lipophilic monomers containing one or more lipophilic moieties conjugated to both ends of the antisense strand.

In some embodiments, the compound comprises one or more lipophilic monomers containing one or more lipophilic moieties conjugated to internal position of the sense or antisense strand. In some embodiments, one or more lipophilic moieties are conjugated to the ribose, nucleobase, and/or at the internucleotide linkages. In some embodiments, one or more lipophilic moieties are conjugated to the ribose at the 2′ position, 3′ position, 4′ position, and/or 5′ position of the ribose. In some embodiments, one or more lipophilic moieties are conjugated at the nucleobase of natural (such as A, T, G, C, or U) or modified as defined herein. In some embodiments, one or more lipophilic moieties are conjugated at the phosphate or modified phosphate groups as defined herein.

In some embodiments, the compound comprises one or more lipophilic monomers containing one or more lipophilic moieties conjugated to the 5′ end or 3′ end of the sense strand, and one or more lipophilic monomers containing one or more lipophilic moieties conjugated to the 5′ end or 3′ end of the antisense strand,

In some embodiments, the lipophilic monomer containing a lipophilic moiety conjugated to the terminal end of a strand via one or more linkers (tethers) and/or a carrier.

In one embodiment, the lipophilic monomer containing a lipophilic moiety conjugated to the terminal end of a strand via one or more linkers (tethers).

In one embodiment, the lipophilic monomer containing lipophilic moiety conjugated to the 5′ end of the sense strand or antisense strand via a cyclic carrier, optionally via one or more intervening linkers (tethers).

In some embodiments, at least one lipophilic monomer is located on one or more terminal positions of the sense strand or antisense strand. In one embodiment, at least one lipophilic monomer is located on the 3′ end or 5′ end of the sense strand. In one embodiment, at least one lipophilic monomer is located on the 3′ end or 5′ end of the antisense strand.

In some embodiments, the lipophilic monomer containing a lipophilic moiety conjugated to one or more internal positions on at least one strand. Internal positions of a strand refers to the nucleotide on any position of the strand, except the terminal position from the 3′ end and 5′ end of the strand (e.g., excluding 2 positions: position 1 counting from the 3′ end and position 1 counting from the 5′ end).

In one embodiment, at least one lipophilic monomer is located on one or more internal positions on at least one strand, which include all positions except the terminal two positions from each end of the strand (e.g., excluding 4 positions: positions 1 and 2 counting from the 3′ end and positions 1 and 2 counting from the 5′ end). In one embodiment, the lipophilic monomer is located on one or more internal positions on at least one strand, which include all positions except the terminal three positions from each end of the strand (e.g., excluding 6 positions: positions 1, 2, and 3 counting from the 3′ end and positions 1, 2, and 3 counting from the 5′ end).

In one embodiment, at least one lipophilic monomer is located on one or more positions of at least one end of the duplex region, which include all positions within the duplex region, but not include the overhang region or the carrier that replaces the terminal nucleotide on the 3′ end of the sense strand.

In one embodiment, at least one lipophilic monomer is located on the sense strand within the first five, four, three, two, or first base pairs at the 5′-end of the antisense strand of the duplex region.

In one embodiment, at least one lipophilic monomer is located on one or more internal positions on at least one strand, except the cleavage site region of the sense strand, for instance, the lipophilic monomer is not located on positions 9-12 counting from the 5′-end of the sense strand, for example, the lipophilic monomer is not located on positions 9-11 counting from the 5′-end of the sense strand. Alternatively, the internal positions exclude positions 11-13 counting from the 3′-end of the sense strand.

In one embodiment, at least one lipophilic monomer is located on one or more internal positions on at least one strand, which exclude the cleavage site region of the antisense strand. For instance, the internal positions exclude positions 12-14 counting from the 5′-end of the antisense strand.

In one embodiment, at least one lipophilic monomer is located on one or more internal positions on at least one strand, which exclude positions 11-13 on the sense strand, counting from the 3′-end, and positions 12-14 on the antisense strand, counting from the 5′-end.

In one embodiment, one or more lipophilic monomers are located on one or more of the following internal positions: positions 4-8 and 13-18 on the sense strand, and positions 6-10 and 15-18 on the antisense strand, counting from the 5′end of each strand.

In one embodiment, one or more lipophilic monomers are located on one or more of the following internal positions: positions 5, 6, 7, 15, and 17 on the sense strand, and positions 15 and 17 on the antisense strand, counting from the 5′end of each strand.

### Definitions

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2d Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

As used herein, the term “target nucleic acid” refers to any nucleic acid molecule the expression or activity of which is capable of being modulated by an siRNA compound. Target nucleic acids include, but are not limited to, RNA (including, but not limited to pre-mRNA and mRNA or portions thereof) transcribed from DNA encoding a target protein, and also cDNA derived from such RNA, and miRNA. For example, the target nucleic acid can be a cellular gene (or mRNA transcribed from the gene) whose expression is associated with a particular disorder or disease state. In some embodiments, a target nucleic acid can be a nucleic acid molecule from an infectious agent.

As used herein, the term “iRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. Thus, these terms can be used interchangeably herein. As used herein, the term iRNA includes microRNAs and pre-microRNAs. Moreover, the “compound” or “compounds” of the invention as used herein, also refers to the iRNA agent, and can be used interchangeably with the iRNA agent.

The iRNA agent should include a region of sufficient homology to the target gene, and be of sufficient length in terms of nucleotides, such that the iRNA agent, or a fragment thereof, can mediate downregulation of the target gene. (For ease of exposition the term nucleotide or ribonucleotide is sometimes used herein in reference to one or more monomeric subunits of an iRNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more, or for example, 6, 5, 4, 3, 2, or fewer mismatches (with respect to the target RNA). The sense strand need only be sufficiently complementary with the antisense strand to maintain the overall double stranded character of the molecule.

iRNA agents include: molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60, 50, 40, or 30 nucleotide pairs. The siRNA agent, or a cleavage product thereof, can down regulate a target gene, e.g., by inducing RNAi with respect to a target RNA, wherein the target may comprise an endogenous or pathogen target RNA.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents may be antisense with regard to the target molecule. A single strand iRNA agent may be sufficiently long that it can enter the RISC and participate in RISC mediated cleavage of a target mRNA. A single strand iRNA agent is at least 14, and in other embodiments at least 15, 20, 25, 29, 35, 40, or 50 nucleotides in length. In certain embodiments, it is less than 200, 100, or 60 nucleotides in length.

A loop refers to a region of an iRNA strand that is unpaired with the opposing nucleotide in the duplex when a section of the iRNA strand forms base pairs with another strand or with another section of the same strand.

Hairpin iRNA agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

A “double stranded (ds) iRNA agent” as used herein, is an iRNA agent which includes more than one, and in some cases two, strands in which interchain hybridization can form a region of duplex structure.

As used herein, the terms “siRNA activity” and “RNAi activity” refer to gene silencing by an siRNA.

As used herein, “gene silencing” by a RNA interference molecule refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, gene expression modulation happens when the expression of the gene, or level of RNA molecule or equivalent RNA molecules encoding one or more proteins or protein subunits is at least 5%, 10%, 20%, 30%, 40%, 50%, 60%, 70%, 80%, 90%, 100%, 2-fold, 3-fold, 4-fold, 5-fold or more different from that observed in the absence of the siRNA, e.g., RNAi agent. The % and/or fold difference can be calculated relative to the control or the non-control, for example,

$$\% difference = \frac{[ expression with si RNA - expression without siRNA ]}{expression without siRNA}$$

$$or$$

$$\% difference = \frac{[ expression with siRNA - expression without siRNA ]}{expression without siRNA}$$

As used herein, the term “inhibit”, “down-regulate”, or “reduce” in relation to gene expression, means that the expression of the gene, or level of RNA molecules or equivalent RNA molecules encoding one or more proteins or protein subunits, or activity of one or more proteins or protein subunits, is reduced below that observed in the absence of modulator. The gene expression is down-regulated when expression of the gene, or level of RNA molecules or equivalent RNA molecules encoding one or more proteins or protein subunits, or activity of one or more proteins or protein subunits, is reduced at least 10% lower relative to a corresponding non-modulated control, and preferably at least 10%, 20%, 30%, 40%, 50%, 60%, 70%, 80%, 90%, 95%, 98%, 99% or most preferably, 100% (i.e., no gene expression).

As used herein, the term “increase” or “up-regulate” in relation to gene expression means that the expression of the gene, or level of RNA molecules or equivalent RNA molecules encoding one or more proteins or protein subunits, or activity of one or more proteins or protein subunits, is increased above that observed in the absence of modulator. The gene expression is up-regulated when expression of the gene, or level of RNA molecules or equivalent RNA molecules encoding one or more proteins or protein subunits, or activity of one or more proteins or protein subunits, is increased at least 10% relative to a corresponding non-modulated control, and preferably at least 10%, 20%, 30%, 40%, 50%, 60%, 70%, 80%, 90%, 95%, 98%, 100%, 1.1-fold, 1.25-fold, 1.5-fold, 1.75-fold, 2-fold, 3-fold, 4-fold, 5-fold, 10-fold, 50-fold, 100-fold or more.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The double-stranded iRNAs comprise two oligonucleotide strands that are sufficiently complementary to hybridize to form a duplex structure. Generally, the duplex structure is between 15 and 30, more generally between 18 and 25, yet more generally between 19 and 24, and most generally between 19 and 21 base pairs in length. In some embodiments, longer double-stranded iRNAs of between 25 and 30 base pairs in length are preferred. In some embodiments, shorter double-stranded iRNAs of between 10 and 15 base pairs in length are preferred. In another embodiment, the double-stranded iRNA is at least 21 nucleotides long.

In some embodiments, the double-stranded iRNA comprises a sense strand and an antisense strand, wherein the antisense RNA strand has a region of complementarity which is complementary to at least a part of a target sequence, and the duplex region is 14-30 nucleotides in length. Similarly, the region of complementarity to the target sequence is between 14 and 30, more generally between 18 and 25, yet more generally between 19 and 24, and most generally between 19 and 21 nucleotides in length.

The term “compound” as used herein, refers to an oligomeric compound that can be an oligonucleotide, an antisense, or an iRNA agent such as an siRNA.

The phrase “antisense strand” as used herein, refers to an oligomeric compound that is substantially or 100% complementary to a target sequence of interest. The phrase “antisense strand” includes the antisense region of both oligomeric compounds that are formed from two separate strands, as well as unimolecular oligomeric compounds that are capable of forming hairpin or dumbbell type structures. The terms “antisense strand” and “guide strand” are used interchangeably herein.

The phrase “sense strand” refers to an oligomeric compound that has the same nucleoside sequence, in whole or in part, as a target sequence such as a messenger RNA or a sequence of DNA. The terms “sense strand” and “passenger strand” are used interchangeably herein.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, /. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In some embodiments, the double-stranded region of a compound is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a compound is equal to or at least 14, 15, 16, 17, 18, 19, 20, 21, 22, 23, 23, 24, 25, 26, 27, 28, 29, or 30 nucleotides in length.

In some embodiments, the sense strand of a compound is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the compound are each 15 to 30 nucleotides in length.

In one embodiment, the sense and antisense strands of the compound are each 19 to 25 nucleotides in length.

In one embodiment, the sense and antisense strands of the compound are each 21 to 23 nucleotides in length.

In some embodiments, one strand has at least one stretch of 1-5 single-stranded nucleotides in the double-stranded region. By “stretch of single-stranded nucleotides in the double-stranded region” is meant that there is present at least one nucleotide base pair at both ends of the single-stranded stretch. In some embodiments, both strands have at least one stretch of 1-5 (e.g., 1, 2, 3, 4, or 5) single-stranded nucleotides in the double stranded region. When both strands have a stretch of 1-5 (e.g., 1, 2, 3, 4, or 5) single-stranded nucleotides in the double stranded region, such single-stranded nucleotides can be opposite to each other (e.g., a stretch of mismatches) or they can be located such that the second strand has no single-stranded nucleotides opposite to the single-stranded iRNAs of the first strand and vice versa (e.g., a single-stranded loop). In some embodiments, the single-stranded nucleotides are present within 8 nucleotides from either end, for example 8, 7, 6, 5, 4, 3, or 2 nucleotide from either the 5′ or 3′ end of the region of complementarity between the two strands.

In one embodiment, the compound comprises a single-stranded overhang on at least one of the termini. In one embodiment, the single-stranded overhang is 1, 2, or 3 nucleotides in length.

In one embodiment, the sense strand of the iRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, each strand of the double-stranded iRNA has a ZXY structure, such as is described in PCT Publication No. 2004080406, which is hereby incorporated by reference in its entirety.

In certain embodiment, the two strands of double-stranded oligomeric compound can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)ₙ; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)₄, (U)₄, and (dT)₄, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

Hairpin and dumbbell type oligomeric compounds will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligomeric compounds can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

In certain embodiments, two oligomeric strands specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

siRNA Design

In one embodiment, the iRNA agent is a double ended bluntmer of 19 nt in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In one embodiment, the iRNA agent is a double ended bluntmer of 20 nt in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In one embodiment, the iRNA agent is a double ended bluntmer of 21 nt in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the iRNA agent comprises a 21 nucleotides (nt) sense strand and a 23 nucleotides (nt) antisense, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the iRNA is blunt, while the other end is comprises a 2 nt overhang. Preferably, the 2 nt overhang is at the 3′-end of the antisense. Optionally, the iRNA agent further comprises a ligand (e.g., GalNAc₃).

In one embodiment, the iRNA agent comprises a sense and antisense strands, wherein: the sense strand is 25-30 nucleotide residues in length, wherein starting from the 5′ terminal nucleotide (position 1) positions 1 to 23 of said first strand comprise at least 8 ribonucleotides; antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the iRNA agent comprises a sense and antisense strands, wherein said iRNA agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein said 3′ end of said first strand and said 5′ end of said second strand form a blunt end and said second strand is 1-4 nucleotides longer at its 3′ end than the first strand, wherein the duplex region which is at least 25 nucleotides in length, and said second strand is sufficiently complementary to a target mRNA along at least 19 nt of said second strand length to reduce target gene expression when said iRNA agent is introduced into a mammalian cell, and wherein dicer cleavage of said iRNA preferentially results in an siRNA comprising said 3′ end of said second strand, thereby reducing expression of the target gene in the mammal. Optionally, the iRNA agent further comprises a ligand (e.g., GalNAc₃).

In one embodiment, the sense strand of the iRNA agent contains at least one motif of three identical modifications on three consecutive nucleotides, where one of the motifs occurs at the cleavage site in the sense strand. For instance, the sense strand can contain at least one motif of three 2′-F modifications on three consecutive nucleotides within 7-15 positions from the 5′end.

In one embodiment, the antisense strand of the iRNA agent can also contain at least one motif of three identical modifications on three consecutive nucleotides, where one of the motifs occurs at or near the cleavage site in the antisense strand. For instance, the antisense strand can contain at least one motif of three 2′-O-methyl modifications on three consecutive nucleotides within 9-15 positions from the 5′end.

For iRNA agent having a duplex region of 17-23 nt in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the iRNA from the 5′-end.

In some embodiments, the iRNA agent comprises a sense strand and antisense strand each having 14 to 30 nucleotides, wherein the sense strand contains at least two motifs of three identical modifications on three consecutive nucleotides, where at least one of the motifs occurs at or near the cleavage site within the strand and at least one of the motifs occurs at another portion of the strand that is separated from the motif at the cleavage site by at least one nucleotide. In one embodiment, the antisense strand also contains at least one motif of three identical modifications on three consecutive nucleotides, where at least one of the motifs occurs at or near the cleavage site within the strand. The modification in the motif occurring at or near the cleavage site in the sense strand is different than the modification in the motif occurring at or near the cleavage site in the antisense strand.

In some embodiments, the iRNA agent comprises a sense strand and antisense strand each having 14 to 30 nucleotides, wherein the sense strand contains at least one motif of three 2′-F modifications on three consecutive nucleotides, where at least one of the motifs occurs at or near the cleavage site in the strand. In one embodiment, the antisense strand also contains at least one motif of three 2′-O-methyl modifications on three consecutive nucleotides at or near the cleavage site.

In some embodiments, the iRNA agent comprises a sense strand and antisense strand each having 14 to 30 nucleotides, wherein the sense strand contains at least one motif of three 2′-F modifications on three consecutive nucleotides at positions 9, 10, 11 from the 5′end, and wherein the antisense strand contains at least one motif of three 2′-O-methyl modifications on three consecutive nucleotides at positions 11, 12, 13 from the 5′end.

In one embodiment, the iRNA agent comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the iRNA agent comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In one embodiment, 100%, 95%, 90%, 85%, 80%, 75%, 70%, 65%, 60%, 55%, 50%, 45%, 40%, 35% or 30% of the dsRNA agent is modified. For example, when 50% of the dsRNA agent is modified, 50% of all nucleotides present in the dsRNA agent contain a modification as described herein.

In one embodiment, each of the sense and antisense strands is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In one embodiment, each of the sense and antisense strands of the dsRNA agent contains at least two different modifications.

In one embodiment, the dsRNA agent does not contain any 2′-F modification.

In one embodiment, the sense strand and/or antisense strand of the dsRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, each of the sense and antisense strands of the dsRNA agent has 15-30 nucleotides. In one example, the sense strand has 19-22 nucleotides, and the antisense strand has 19-25 nucleotides. In another example, the sense strand has 21 nucleotides, and the antisense strand has 23 nucleotides.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the dsRNA agent is 100% complementary to a target RNA to hybridize thereto and inhibits its expression through RNA interference. In another embodiment, the antisense strand of the dsRNA agent is at least 95%, at least 90%, at least 85%, at least 80%, at least 75%, at least 70%, at least 65%, at least 60%, at least 55%, or at least 50% complementary to a target RNA.

In one aspect, the invention relates to a dsRNA agent as defined herein capable of inhibiting the expression of a target gene. The dsRNA agent comprises a sense strand and an antisense strand, each strand having 14 to 40 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand).

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′end of the antisense strand.

In one embodiment, the dsRNA agents of comprise:

(a) a sense strand having:
(i) a length of 18-23 nucleotides;
(ii) three consecutive 2′-F modifications at positions 7-15; and
   (b) an antisense strand having:
(i) a length of 18-23 nucleotides;
(ii) at least 2′-F modifications anywhere on the strand; and
(iii) at least two phosphorothioate internucleotide linkages at the first five nucleotides (counting from the 5′ end);
   wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents comprise:

(a) a sense strand having:
(i) a length of 18-23 nucleotides;
(ii) less than four 2′-F modifications;
   (b) an antisense strand having:
(i) a length of 18-23 nucleotides;
(ii) at less than twelve 2′-F modification; and
(iii) at least two phosphorothioate internucleotide linkages at the first five nucleotides (counting from the 5′ end);
   wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents comprise:

(a) a sense strand having:
(i) a length of 19-35 nucleotides;
(ii) less than four 2′-F modifications;
   (b) an antisense strand having:
(i) a length of 19-35 nucleotides;
(ii) at less than twelve 2′-F modification; and
(iii) at least two phosphorothioate internucleotide linkages at the first five nucleotides (counting from the 5′ end);
   wherein the duplex region is between 19 to 25 base pairs (preferably 19, 20, 21 or 22); and wherein the dsRNA agents have one or more lipophilic monomers containing one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have less than 20%, less than 15% and less than 10% non-natural nucleotide.

Examples of non-natural nucleotide includes acyclic nucleotides, LNA, HNA, CeNA, 2′-methoxyethyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F, and others.

In one embodiment, the dsRNA agents comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have greater than 80%, greater than 85% and greater than 90% natural nucleotide, such as 2′-OH, 2′-deoxy and 2′-OMe are natural nucleotides.

In one embodiment, the dsRNA agents comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic monomers containing one or more lipophilic moieties conjugated to one or more positions on at least one strand; and wherein the dsRNA agents have 100% natural nucleotide, such as 2′-OH, 2′-deoxy and 2′-OMe are natural nucleotides.

In one embodiment, the dsRNA agents a sense strand and an antisense strand, each strand having 14 to 30 nucleotides, wherein the sense strand sequence is represented by formula (I):

5′nₚ-Nₐ—(X X X)ᵢ—N_{b}—Y Y Y—N_{b}—(Z Z Z)ⱼ—Nₐ-n_{q}3′ (I)

wherein:

i and j are each independently 0 or 1;

p and q are each independently 0-6;

each Nₐ independently represents an oligonucleotide sequence comprising 0-25 modified nucleotides, each sequence comprising at least two differently modified nucleotides;

each N_{b} independently represents an oligonucleotide sequence comprising 1, 2, 3, 4, 5, or 6 modified nucleotides;

each nₚ and n_{q} independently represent an overhang nucleotide;

wherein N_{b} and Y do not have the same modification;

wherein XXX, YYY and ZZZ each independently represent one motif of three identical modifications on three consecutive nucleotides;

wherein the dsRNA agents have one or more lipophilic monomers containing one or more lipophilic moieties conjugated to one or more positions on at least one strand; and

wherein the antisense strand of the dsRNA comprises two blocks of one, two or three phosphorothioate internucleotide linkages separated by 1, 2, 3, 4, 5, 6, 7, 8, 9, 10, 11, 12, 13, 14, 15, 16, 17, or 18 phosphate internucleotide linkages.

Various publications described multimeric siRNA and can all be used with the iRNA of the invention. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, 100%, 95%, 90%, 85%, 80%, 75%, 70%, 65%, 60%, 55%, 50%, 45%, 40%, 35% or 30% of the iRNA agent of the invention is modified with 2′-OMe.

In some embodiments, each of the sense and antisense strands of the iRNA agent is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In some embodiments, each of the sense and antisense strands of the iRNA agent contains at least two different modifications.

In some embodiments, the compound of the invention of the invention does not contain any 2′-F modification.

In some embodiments, the compound of the invention contains one, two, three, four, five, six, seven, eight, nine, ten, eleven or twelve 2′-F modification(s). In one example, compound of the invention contains nine or ten 2′-F modifications.

The iRNA agent of the invention may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the iRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand and/or antisense strand of the iRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In some embodiments, the antisense strand of the iRNA agent is 100% complementary to a target RNA to hybridize thereto and inhibits its expression through RNA interference. In another embodiment, the antisense strand of the iRNA agent is at least 95%, at least 90%, at least 85%, at least 80%, at least 75%, at least 70%, at least 65%, at least 60%, at least 55%, or at least 50% complementary to a target RNA.

Nucleic Acid Modifications

In some embodiments, the compound comprises at least one nucleic acid modification described herein. For example, at least one modification selected from the group consisting of modified internucleoside linkage, modified nucleobase, modified sugar, and any combinations thereof. Without limitations, such a modification can be present anywhere in the compound. For example, the modification can be present in one of the RNA molecules.

### Nucleic Acid Modifications (Nucleobases)

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligomeric compound described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl,para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic includes more complicated structures, such as for example a tricyclic phenoxazine nucleobase mimetic. Methods for preparation of the above noted modified nucleobases are well known to those skilled in the art.

### Nucleic Acid Modifications (Sugar)

Compound of the inventions provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligomeric compounds comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furnaosyl is connected to the 4′ position by a linker selected independently from —[C(R1)(R2)]ₙ—, —[C(R1)(R2)]ₙ—O—, —[C(R1)(R2)]ₙ—N(R1)-, —[C(R1)(R2)]ₙ—N(R1)-O—, —[C(R1R2)]ₙ—O—N(R1)-, —C(R1)=C(R2)-O—, —C(R1)=N—, —C(R1)=N—O—, —C(═NR1)-, —C(═NR1)-O—, —C(═O)—, —C(═O)O—, —C(═S)—, —C(═S)O—, —C(═S)S—, —O—, —Si(R1)2-, —S(═O)ₓ— and —N(R1)-;

wherein:

x is 0, 1, or 2;

n is 1, 2, 3, or 4;

each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)₂-J1), or sulfoxyl (S(═O)-J1); and

each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, —[C(R1)(R2)]ₙ—, —[C(R1)(R2)]n-O—, C(R1R2)-N(R1)-O— or —C(R1R2)-O—N(R1)-. In another embodiment, each of said linkers is, independently, 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R1)-2′ and 4′-CH₂—N(R1)-O-2′-wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)₂—O-2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)CH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligomeric compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

Compound of the inventions disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. Compound of the inventions can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in a configuration.

Sugar modifications can also include a “acyclic nucleotide,” which refers to any nucleotide having an acyclic ribose sugar, e.g., wherein a C—C bonds between ribose carbons (e.g., C1′-C2′, C2′-C3′, C3′-C4′, C4′-04′, C1′-04′) is absent and/or at least one of ribose carbons or oxygen (e.g., C1′, C2′, C3′, C4′ or 04′) are independently or in combination absent from the nucleotide. In some embodiments, acyclic nucleotide is

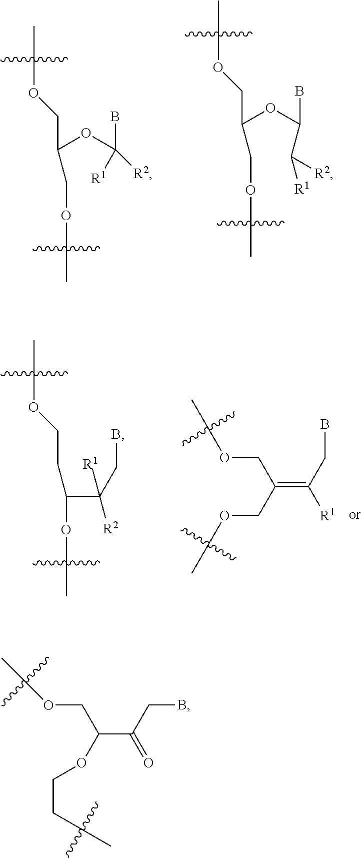

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

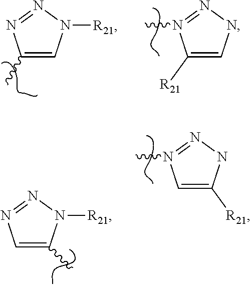

NR₂₁R₃₁, CONR₂₁R₃₁, CON(H)NR₂₁R₃₁, ONR₂₁R₃₁, CON(H)N═CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alkali metal or transition metal with an overall charge of +1; and Y is O, S, or NR′, where R′ is hydrogen, optionally substituted aliphatic. Preferably this modification is at the 5′ terminal of the iRNA.

In certain embodiments, the compound of the invention comprises at least two regions of at least two contiguous monomers of the above formula. In certain embodiments, the compound of the invention comprises a gapped motif. In certain embodiments, the compound of the invention comprises at least one region of from about 8 to about 14 contiguous β-D-2′-deoxyribofuranosyl nucleosides. In certain embodiments, the Compound of the invention comprises at least one region of from about 9 to about 12 contiguous β-D-2′-deoxyribofuranosyl nucleosides.

In certain embodiments, the compound of the invention comprises at least one (e.g., 1, 2, 3, 4, 5, 6, 7, 8, 9, 10, 11, 12, 13, 14, 15 or more) comprises at least one (S)-cEt monomer of the formula:

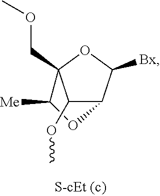

wherein Bx is heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### Nucleic Acid Modifications (Intersugar Linkage)

Described herein are linking groups that link monomers (including, but not limited to, modified and unmodified nucleosides and nucleotides) together, thereby forming an oligomeric compound, e.g., an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotides. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′—O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phosphotriesters, aminoalkylphosphotriesters, alkyl-phosphonaters (e.g., methyl-phosphonate), selenophosphates, phosphoramidates (e.g., N-alkylphosphoramidate), and boranophosphonates.

In some embodiments, the compound of the invention comprises at least one (e.g., 1, 2, 3, 4, 5, 6, 7, 8, 9, 10, 11, 12, 13, 14, 15 or more and up to including all) modified or nonphosphodiester linkages. In some embodiments, the compound of the invention comprises at least one (e.g., 1, 2, 3, 4, 5, 6, 7, 8, 9, 10, 11, 12, 13, 14, 15 or more and up to including all) phosphorothioate linkages.

The compound of the inventions can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backbone-extended pyrrolidine PNA (bepPNA) nucleoside surrogates. A preferred surrogate is a PNA surrogate.

The compound of the inventions described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the compound of the inventions provided herein are all such possible isomers, as well as their racemic and optically pure forms.

### Nucleic Acid Modifications (Terminal Modifications

In some embodiments, the compound further comprises a phosphate or phosphate mimic at the 5′-end of the antisense strand. In one embodiment, the phosphate mimic is a 5′-vinyl phosphonate (VP).

In some embodiments, the 5′-end of the antisense strand of the compound does not contain a 5′-vinyl phosphonate (VP).

Ends of the iRNA agent of the invention can be modified. Such modifications can be at one end or both ends. For example, the 3′ and/or 5′ ends of an iRNA can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligomeric compound, this array can substitute for a hairpin loop in a hairpin-type oligomeric compound.

Terminal modifications useful for modulating activity include modification of the 5′ end of iRNAs with phosphate or phosphate analogs. In certain embodiments, the 5′end of an iRNA is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligomeric compound comprises the modification

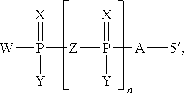

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)₂(X)P—O[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### Thermally Destabilizing Modifications

The compounds of the invention, such as iRNAs or dsRNA agents, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nucleic acid (GNA).

Exemplified abasic modifications are:

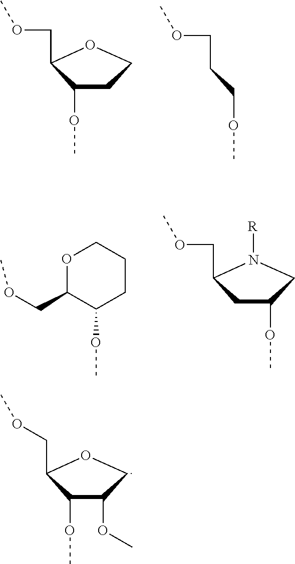

Exemplified sugar modifications are:

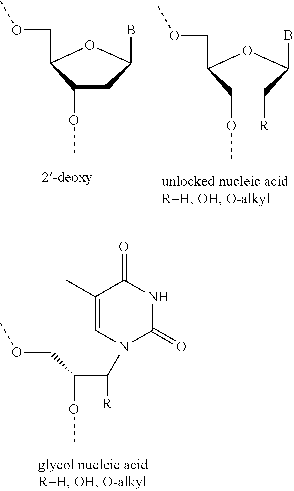

The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

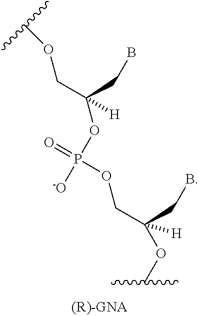

The thermally destabilizing modification can be mismatches (i.e., noncomplementary base pairs) between the thermally destabilizing nucleotide and the opposing nucleotide in the opposite strand within the dsRNA duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the compounds of the invention, such as siRNA or iRNA agent, contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

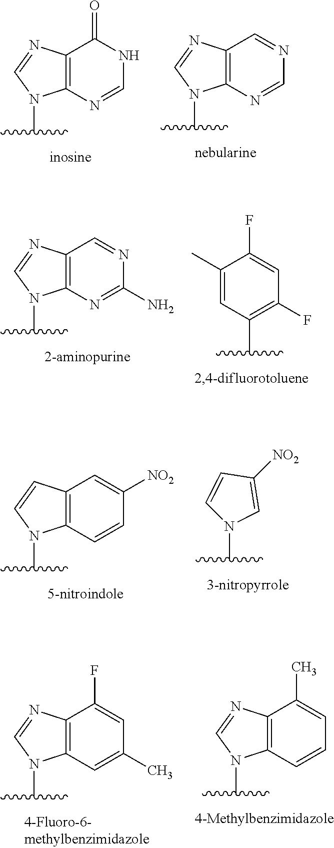

Exemplary phosphate modifications known to decrease the thermal stability of dsRNA duplexes compared to natural phosphodiester linkages are:

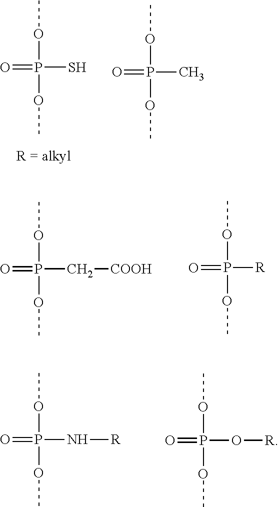

In some embodiments, compounds of the invention can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, compounds of the invention can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In one embodiment, the iRNA agent of the invention is conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and and decalin; preferably, the acyclic group is selected from serinol backbone or diethanolamine backbone.

In some embodiments, at least one strand of the iRNA agent disclosed herein is 5′ phosphorylated or includes a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH2)(O)P—O-5′), 5′-alkylphosphonates (R=alkyl=methyl, ethyl, isopropyl, propyl, etc., e.g. R^{P}(OH)(O)—O-5′-, 5′-alkenylphosphonates (i.e. vinyl, substituted vinyl), (OH)₂(O)P-5′-CH₂—), 5′-alkyletherphosphonates (R=alkylether=methoxymethyl (MeOCH2-), ethoxymethyl, etc., e.g. RP(OH)(O)—O-5′-).

### Target Genes

Without limitations, target genes for siRNAs include, but are not limited to genes promoting unwanted cell proliferation, growth factor gene, growth factor receptor gene, genes expressing kinases, an adaptor protein gene, a gene encoding a G protein super family molecule, a gene encoding a transcription factor, a gene which mediates angiogenesis, a viral gene, a gene required for viral replication, a cellular gene which mediates viral function, a gene of a bacterial pathogen, a gene of an amoebic pathogen, a gene of a parasitic pathogen, a gene of a fungal pathogen, a gene which mediates an unwanted immune response, a gene which mediates the processing of pain, a gene which mediates a neurological disease, an allene gene found in cells characterized by loss of heterozygosity, or one allege gene of a polymorphic gene.

Specific exemplary target genes for the siRNAs include, but are not limited to, PCSK-9, ApoC3, AT3, AGT, ALAS1, TMPR, HAO1, AGT, C5, CCR-5, PDGF beta gene; Erb-B gene, Src gene; CRK gene; GRB2 gene; RAS gene; MEKK gene; INK gene; RAF gene; Erk1/2 gene; PCNA(p21) gene; MYB gene; c-MYC gene; JUN gene; FOS gene; BCL-2 gene; Cyclin D gene; VEGF gene; EGFR gene; Cyclin A gene; Cyclin E gene; WNT-1 gene; beta-catenin gene; c-MET gene; PKC gene; NFKB gene; STAT3 gene; survivin gene; Her2/Neu gene; topoisomerase I gene; topoisomerase II alpha gene; p73 gene; p21(WAF1/CIP1) gene, p27(KIP1) gene; PPM1D gene; caveolin I gene; MIB I gene; MTAI gene; M68 gene; tumor suppressor genes; p53 gene; DN-p63 gene; pRb tumor suppressor gene; APC1 tumor suppressor gene; BRCA1 tumor suppressor gene; PTEN tumor suppressor gene; MLL fusion genes, e.g., MLL-AF9, BCR/ABL fusion gene; TEL/AML1 fusion gene; EWS/FLIl fusion gene; TLS/FUS1 fusion gene; PAX3/FKHR fusion gene; AML1/ETO fusion gene; alpha v-integrin gene; Flt-1 receptor gene; tubulin gene; Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, I-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNL1A4 gene, SCA7 gene, SCA8 gene, allele gene found in loss of heterozygosity (LOH) cells, one allele gene of a polymorphic gene and combinations thereof.

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in duploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific modulation of one allele of an essential gene with a composition of the invention.

In certain embodiments, the invention provides a compound of the invention that modulates a micro-RNA.

### Targeting CNS

In some embodiments, the invention provides a compound that targets APP for Early Onset Familial Alzheimer Disease, ATXN2 for Spinocerebellar Ataxia 2 and ALS, and C9orf72 for Amyotrophic Lateral Sclerosis and Frontotemporal Dementia.

In some embodiments, the invention provides a compound that targets TARDBP for ALS, MAPT (Tau) for Frontotemporal Dementia, and HTT for Huntington Disease.

In some embodiments, the invention provides a compound that targets SNCA for Parkinson Disease, FUS for ALS, ATXN3 for Spinocerebellar Ataxia 3, ATXN1 for SCA1, genes for SCA7 and SCA8, ATN1 for DRPLA, MeCP2 for XLMR, PRNP for Prion Diseases, recessive CNS disorders: Lafora Disease, DMPK for DM1 (CNS and Skeletal Muscle), and TTR for hATTR (CNS, ocular and systemic).

Spinocerebellar ataxia is an inherited brain-function disorder. Dominantly inherited forms of spinocerebellar ataxias, such as SCA1-8, are devastating disorders with no disease-modifying therapy. Exemplary targets include SCA2, SCA3, and SCA1.

### Targeting ATXN2 for SCA2

Spinocerebellar Ataxia 2 (SCA2), a progressive ataxia, is the second most common SCA. Another disease associated with this target is amyotrophic lateral sclerosis (ALS). These diseases are debilitating and ultimately lethal diseases with no disease-modifying therapy. The prevalence of SCA is 2-6 per 100,000 people; ATXN2 causes 15% of SCA population worldwide and much more SCA populations in some countries, especially in Cuba (40 per 100,000 people). Targeting ATXN2 can be excellent via human molecular genetics, e.g., coding CAG repeat expansion in ATXN2 was discovered in familial and sporadic SCA and ALS, in tissues such as spinal cord, brainstem, or cerebellum. The mechanism of this targeting may be because autosomal dominant coding CAG expansion of ATXN2 causes expression of toxic, misfolded protein and Purkinje cell and neuronal death. The efficacy has been shown by 70% knockdown (KD) of ATXN2 mRNA; and mATXN2 mice KD POC has been demonstrated. With respect to safety, mATXN2 knockout (KO) mice have been reported healthy. Possible diagnosis includes family history; genetic testing; or early symptoms. Biomarkers that can be used include, e.g., CSF CAG mRNA and peptide repeat proteins

### Targeting ATXA3 for SCA3

Spinocerebellar Ataxia 3 (SCA3), a progressive ataxia, is the most common SCA worldwide. This disease is debilitating and ultimately lethal disease with no disease-modifying therapy. It is the most common cause of SCA and the prevalence of SCA is 2-6 per 100,000 people; ATXN3 causes 21% of SCA population in US and much more in Europe, especially in Portugal. Targeting ATXN3 can be excellent via human molecular genetics, e.g., coding CAG repeat expansion in ATXN3 was discovered in familial and sporadic SCA, in tissues such as spinal cord, brainstem, or cerebellum. The mechanism of this targeting may be because autosomal dominant coding CAG expansion of ATXN3 causes expression of toxic, misfolded protein, Purkinje cell and neuron death. The efficacy has been shown by 70% KD of ATXN3 mRNA; and mATXN3 KD mice POC has been demonstrated. With respect to safety, mATXN3 KO mice have been reported healthy. Possible diagnosis includes family history; genetic testing; or early symptoms. Biomarkers that can be used include, e.g., CSF CAG mRNA and peptide repeat proteins.

### Targeting ATXN1 for SCA1

Spinocerebellar Ataxia 1 (SCA1), a progressive ataxia, is the first SCA gene discovered in 1993. This disease is debilitating and ultimately lethal disease with no disease-modifying therapy. The prevalence of SCA is 2-6 per 100,000 people; ATXN1 causes 6% of SCA population in US and worldwide, and much more in some countries (25% in Japan), especially in Poland (64%) and Siberia (100%). Targeting ATXN1 can be excellent via human molecular genetics, e.g., coding CAG repeat expansion in ATXN1 was discovered in familial and sporadic SCA, in tissues such as spinal cord, brainstem, or cerebellum. The mechanism of this targeting may be because autosomal dominant coding CAG expansion of ATXN1 causes expression of toxic, misfolded protein, Purkinje cell and neuronal death. The efficacy has been shown by 70% KD of ATXN1 mRNA; and mATXN1 mice POC has been demonstrated. With respect to safety, mATXN1 KO mice have been reported healthy. Possible diagnosis includes family history; genetic testing; or early symptoms. Biomarkers that can be used include, e.g., CSF CAG mRNA and peptide repeat proteins.

### Targeting ATXA7 for SCA7

Spinocerebellar Ataxia 7 (SCA7) causes progressive ataxia and retinal degeneration. This disease is debilitating and ultimately lethal retinal and cerebellar disorder with no disease-modifying therapy. The prevalence of SCA is 2-6 per 100,000 people; ATXN7 causes 5% of SCA population worldwide, and much more in some countries, especially in South Africa. Targeting ATXN7 can be excellent via human molecular genetics, e.g., coding CAG repeat expansion in ATXN7 discovered in familial and sporadic SCA, in tissues such as spinal cord, brainstem, cerebellum, or retina. The mechanism of this targeting may be because autosomal dominant coding CAG expansion of ATXN1 causes expression of toxic, misfolded protein, inciting cone and rod dystrophy, Purkinje cell and neuronal lethality. The efficacy has been shown by 70% KD of ATXN1 mRNA, via intrathecal (IT) and intravitreal (IVT) administrations. Possible diagnosis includes family history; genetic testing; or early symptoms. Biomarkers that can be used include, e.g., CSF CAG mRNA and peptide repeat proteins.

### Targeting ATXN8 for SCA8

Spinocerebellar Ataxia 8 (SCA8), a progressive neurodegenerative ataxia is caused by CTG repeat expansion in ATXN8. This disease is debilitating and ultimately lethal disease with no disease-modifying therapy. The prevalence: SCA is 2-6 per 100,000 people; ATXN8 causes 3% of SCA population worldwide, and much more in some countries, especially in Finland. Targeting ATXN8 can be excellent via human molecular genetics, e.g., coding CTG repeat expansion in ATXN8 was discovered in familial and sporadic SCA, in tissues such as spinal cord, brainstem, or cerebellum. The mechanism of this targeting may be because autosomal dominant coding CTG expansion of ATXN8 causes expression of toxic, misfolded protein, inciting Purkinje cell and neuronal lethality. The efficacy has been shown by 70% KD of ATXN8 mRNA. Possible diagnosis includes family history; genetic testing; or early symptoms. Biomarkers that can be used include, e.g., CSF CTG mRNA and peptide repeat proteins.

### Targeting CACNA1A for SCA6

Spinocerebellar ataxia 6 (SCA6) is a progressive ataxia. This disease is debilitating and ultimately lethal disease with no disease-modifying therapy. The prevalence of SCA is 2-6 per 100,000 people; and CACNA1A causes 15% of SCA population worldwide. Targeting CACNA1A can be excellent via human molecular genetics, e.g., coding CAG repeat expansion in CACNA1A was discovered in familial and sporadic SCA, in tissues such as spinal cord, brainstem, or cerebellum. The mechanism of this targeting may be because autosomal dominant coding CAG expansion of CACNA1A causes expression of toxic, misfolded protein and Purkinje cell and neuronal death. The efficacy has been shown by 70% KD of CACNA1A CAG expansion. Possible diagnosis includes family history; genetic testing; or early symptoms. Biomarkers that can be used include, e.g., CSF CAG mRNA and peptide repeat proteins.

Exemplary target for inherited polyglutamine disorders includes Huntington disease (HD).

### Targeting HTT for Huntington Disease

Huntington mutations causes HD, a progressive CNS degenerative disease. This disease is debilitating and ultimately lethal disease with no disease-modifying therapy. The prevalence of HD is 5-10 per 100,000 people worldwide, and much more common in certain countries, especially in Venezuela. Targeting HTT can be excellent via human molecular genetics, e.g., coding CAG repeat expansion in HTT discovered in familial and sporadic HD, in tissues such as striatum, or cortex. The mechanism of this targeting may be because autosomal dominant coding CAG expansion of HTT causes expression of toxic, misfolded protein and neuronal death. The efficacy has been shown by 70% KD of HTT CAG expansion only; and murine POC has been demonstrated. With respect to safety, KO of HTT in mice can be lethal; KD in humans has been demonstrated. Possible diagnosis includes family history; genetic testing; early symptoms. Biomarkers that can be used include, e.g., CSF mRNA and peptide repeat proteins.

### Targeting ATN1 for DRPLA

Atrophin 1 mutations causes dentatorubral-pallidoluysian atrophy (DRPLA), which is a progressive spinocerebellar disorder similar to HD. This disease is debilitating and ultimately lethal disease with no disease-modifying therapy. The prevalence of DRPLA is 2-7 per 1,000,000 people in Japan. Targeting ATN1 can be excellent via human molecular genetics, e.g., coding CAG repeat expansion in ATN1 was discovered in familial and sporadic SCA, in tissues such as spinal cord, brainstem, cerebellum, or cortex. The mechanism of this targeting may be because autosomal dominant coding CAG expansion of ATN1 causes expression of toxic, misfolded protein and neuronal death. The efficacy has been shown by 70% KD of ATN1. With respect to safety, ATN1 KO mice have been reported healthy. Possible diagnosis includes family history; genetic testing; or early symptoms. Biomarkers that can be used include, e.g., CSF CAG mRNA and peptide repeat proteins.

### Targeting AR for Spinal and Bulbar Muscular Atrophy

Androgen receptor mutations causes spinal and bulbar muscular atrophy (SBMA, Kennedy disease), a progressive muscle wasting disease, and other diseases. This disease is debilitating and ultimately lethal disease with no disease-modifying therapy. The prevalence of SBMA is 2 per 100,000 males; females have a mild phenotype. Targeting AR can be excellent via human molecular genetics, e.g., coding CAG repeat expansion in AR discovered in familial SBMA, in tissues such as spinal cord, or brainstem. The mechanism of this targeting may be because X-linked coding CAG expansion of AR causes toxic gain-or-function and motor neuron lethality. The efficacy has been shown by 70% KD of AR. Possible diagnosis includes family history; genetic testing; or early symptoms. Biomarkers that can be used include, e.g., CSF CAG mRNA and peptide repeat proteins.

### Targeting FXN for Friedrich Ataxia

Recessive loss of function GAA expansion of FXN causes friedrich ataxia (FA), a progressive degenerative ataxia. This disease is debilitating and ultimately lethal disease with no disease-modifying therapy. The prevalence of FA is 2 per 100,000 people worldwide. Targeting FXN can be excellent via human molecular genetics, e.g., intron GAA repeat expansion in FXN was discovered in familial FA, in tissues such as spinal cord, cerebellum, or perhaps retina and heart. The mechanism of this targeting may be because autosomal recessive non-coding FAA expansion of FXN causes deceased expression of FXN, an important mitochondrial protein. The efficacy has been shown by 70% KD of FXN intron GAS expansion. With respect to safety, KD of intron GAA is safe and effective in mice. Possible diagnosis includes family history; genetic testing; or early symptoms. Biomarkers that can be used include, e.g., CSF mRNA and peptide repeat proteins.

### Targeting FMR1 for FXTAS

Fragile X-associated tremor/ataxia syndrome (FXTAS), a progressive disorder of ataxia and cognitive loss in adults caused by FMR1 overexpression. This disease is debilitating disease with no disease-modifying therapy. The prevalence of FMR1 permutation is 1 in 500 males. Targeting FMR1 can be excellent via human molecular genetics, e.g., coding CCG repeat expansion pre-mutations in FMR1 was discovered in FXTAS, in tissues such as spinal cord, cerebellum, or cortex. The mechanism of this targeting may be because X-linked coding CCG expansion of FMR1 causes toxic mRNA. The efficacy has been shown by 70% KD of toxic mRNA. Possible diagnosis includes family history; genetic testing; or early symptoms. Biomarkers that can be used include, e.g., CSF mRNA and peptide repeat proteins.

Targeting upstream of FMR1 for Fragile X Syndrome

Fragile X syndrome (FRAXA), a progressive disorder of mental retardation, may be treated by targeting upstream mRNA of FMR1. This disease is debilitating disease with no disease-modifying therapy. The prevalence of FRAXA is 1 per 4,000 males and 1 per 8,000 females. Targeting FMR1 can be excellent via human molecular genetics, e.g., coding CCG repeat expansion in FMR1 was discovered in FRAXA, in tissues such as CNS. The mechanism of this targeting may be because X-linked coding CCG expansion of FMR1 causes LOF; and normal FMR1 functions to transport specific mRNAs from nucleus. The efficacy has been shown by 70% KD of toxic mRNA. Possible diagnosis includes family history; genetic testing; or early symptoms. Biomarkers that can be used include, e.g., CSF mRNA and peptide repeat proteins.

Dominant Inherited Amyotrophic Lateral Sclerosis is a devastating disorders with no disease-modifying therapy. Exemplary targets include C9orf72, ATXN2 (also causes SCA2), and MAPT.

### Targeting C9orf72 for ALS

C9orf72 is the most common cause of Amyotrophic Lateral Sclerosis (ALS) and Frontotemporal Dementia (FTD). These diseases are lethal disorders of motor neurons with no disease-modifying therapy. The prevalence of ALS is 2-5 per 100,000 people (10% is familial); C9orf72 causes 39% of familial ALS in US and Europe and 7% of sporadic ALS. Targeting C9orf72 can be excellent via human molecular genetics, e.g., hexa-nucleotide expansion was discovered in familial and sporadic ALS, in tissues such as upper and lower motor neurons (for ALS); or cortex (for FTD). The mechanism of this targeting may be because autosomal dominant hexa-nucleotide expansion causes repeat-associated non-AUG-dependent translation of toxic dipeptide repeat proteins and neuron lethality. The efficacy has been shown by 70% KD of C9orf72. With respect to safety, heterozygous LOF mutations of C9orf72 appear to be safe in humans and mice. Possible diagnosis includes family history; genetic testing; or early symptoms. Biomarkers that can be used include, e.g., CSF hexa-nucleotide repeat mRNAs and dipeptide repeat proteins.

### Targeting TARDBP for ALS

TARDBP mutations causes ALS and Frontotemporal Dementia (FTD). These diseases are lethal disorders of motor neurons with no disease-modifying therapy. The prevalence of ALS is 2-5 per 100,000 people (10% is familial); TARDBP causes 5% of familial ALS and 1.5% of sporadic ALS. Targeting TARDBP can be excellent via human molecular genetics, e.g., mutations were discovered in familial and sporadic ALS, in tissues such as upper and lower motor neurons (for ALS); or cortex (for FTD). The mechanism of this targeting may be because autosomal dominant TRDBP mutations cause toxic TRDBP protein and neuron lethality. The efficacy has been shown by 70% KD of TARDBP mutant alleles. Possible diagnosis includes family history; genetic testing; or early symptoms. Biomarkers that can be used include, e.g., CSF proteins.

### Targeting FUS for ALS

FUS mutations causes ALS and FTD. These diseases are lethal disorder of motor neurons with no disease-modifying therapy. The prevalence of ALS is 2-5 per 100,000 people (10% is familial); FUS causes 5% of familial ALS; FUS inclusions are often found in sporadic ALS. Targeting FUS can be excellent via human molecular genetics, e.g., mutations were discovered in familial ALS, in tissues such as upper and lower motor neurons for ALS. The mechanism of this targeting may be because autosomal dominant FUS mutations cause abnormal protein folding and neuron lethality. The efficacy has been shown by 70% KD of FUS mutant alleles. With respect to safety, KO mice struggle but survive and have an ADHD phenotype. Possible diagnosis includes family history; genetic testing; or early symptoms. Biomarkers that can be used include, e.g., CSF proteins.

### Targeting SOD1 for ALS

Dominant and recessive mutations of SOD1 cause ALS. This disease is lethal disorder of motor neurons with no disease-modifying therapy. The prevalence of ALS is 2-5 per 100,000 people (10% is familial); SOD1 causes 5-20% of familial ALS. Target SOD1 can be excellent via human molecular genetics, e.g., many SOD1 mutations associate with AD and AR ALS in families, in tissues such as upper and lower motor neurons for ALS. The efficacy of this targeting may need mutation-specific KD. Possible diagnosis includes family history; genetic testing; or early symptoms. Biomarkers may be mutation-specific.

Dominant Inherited Frontotemporal Dementia and Progressive Supra-nuclear Palsy. The targets include MAPT because it may be important for AD, or C9orf72.

### Targeting Microtubule-Associated Protein Tau for FTD-17 and PSP

Familial Frontotemporal Dementia 17 (FTD-17), a familial form of FTD lined to chromosome 17, and Familial Progressive Supra-nuclear Palsy may be caused by MAPT mutations, which may also cause rare forms of Progressive Supra-nuclear Palsy, Corticobasal Degeneration, Tauopathy with Respiratory Failure, Dementia with Seizures. These diseases are lethal neurodegenerative disorders with no disease-modifying therapy. The prevalence of FTD is 15-22 per 100,000 people; the prevalence of FTD-17 in Netherlands is 1 in 1,000,000 population. Targeting MAPT can be excellent via human molecular genetics, e.g., GOF point and splice site mutations of MAPT were discovered in familial and sporadic FTD, in tissues such as frontal or temporal cortex. The mechanism of this targeting may be because autosomal dominant GOF mutations of MAPT lead to toxic Tau peptides and neuronal death. The efficacy has been shown by 70% KD of MAPT. With respect to safety, MAPT KO mice have been reported healthy. Possible diagnosis includes family history; genetic testing; early symptoms. Biomarkers that can be used include, e.g., CSF Tau mRNAs and proteins.

### Targeting Sequestosome 1 for FTD and ALS

Sporadic FTD/ALS associate with dominant SQSTM1 mutations. This disease is lethal neurodegenerative disorder with no disease-modifying therapy. This is a very rare disease. Targeting Sequestosome 1 is reasonable via human molecular genetic association in sporadic cases, in tissues such as frontal and temporal cortex, or cerebellum and spinal cord. Possible diagnosis includes genetic testing; early symptoms.

Dominant Inherited Parkinson Disease is a devastating disorders with no disease-modifying therapy. The targets include SNCA.

### Targeting SNCA for Parkinson Disease

Alpha Synuclein mutations causes familial Parkinson disease (PD) and Lewy body dementia. These diseases are lethal neurodegenerative disorders with no disease-modifying therapy. The prevalence of PD is 4 million worldwide; ⅓ of PD is familial; 1% of fPD is caused by SNCA. Targeting SNCA can be excellent via human molecular genetics, e.g., SNCA point mutations and duplications cause familial PD, in tissues such as medulla oblongata; or substantia nigra of the midbrain. The mechanism of this targeting may be because overexpression or expression of abnormal SNCA protein leads to toxic peptides and neuronal death. The efficacy has been shown by 70% KD of SNCA. With respect to safety, SNCA KO mice are healthy. Possible diagnosis includes family history; genetic testing; or early symptoms. Biomarkers that can be used include, e.g., CSF SNCA mRNAs and proteins.

### Targeting LRRK2 for Parkinson Disease

Leucine-rich repeat kinase 2 mutations cause familial Parkinson disease. This disease is lethal neurodegenerative disorder with no disease-modifying therapy. The prevalence of PD is 4 million worldwide; ⅓ of PD is familial; 3-7% of fPD is caused by LRRK2. Targeting LRRK2 can be excellent via human molecular genetics, e.g., LRRK2 point mutations cause familial PD, in tissues such as medulla oblongata; or substantia nigra of the midbrain. Possible diagnosis includes family history; genetic testing; early symptoms. Biomarkers that can be used include, e.g., CSF mRNAs and proteins.

### Targeting GARS for Spinal Muscular Atrophy V

Autosomal dominant Glycyl-tRNA Synthetase mutations cause spinal muscular atrophy V (SMAV) or distal hereditary motor neuropathy Va. These diseases are neurodegenerative disorders with no disease-modifying therapy. These are very rare diseases. Targeting GARs can be good via human molecular genetics, e.g., GARS point mutations cause familial SMA, in tissues such as spinal cord. Possible diagnosis includes family history; genetic testing; early symptoms.

Targeting Seipin for spinal Muscular Atrophy

Autosomal dominant Seipin mutations causes spinal muscular atrophy (SMA) or distal hereditary motor neuropathy. These diseases are neurodegenerative disorders with no disease-modifying therapy. These are very rare diseases. Targeting Seipin can be good via human molecular genetics, e.g., Seipin point mutations cause familial SMA, in tissues such as spinal cord. The mechanism of this targeting is probably GOF and toxic peptides. The efficacy has been shown by 50% KD. With respect to safety, recessive LOF mutations cause progressive encephalopathy with or without lipodystrophy. Possible diagnosis includes family history; genetic testing; or early symptoms.

Dominant Inherited Alzheimer Disease is a devastating disorders with no disease-modifying therapy. The targets include APP because of central mechanistic role in familial disease and possible role in common AD.

### Targeting APP for Alzheimer Disease

Amyloid precursor protein mutations causes early onset familial Alzheimer disease (EOFAD); AD in down syndrome; or AD. These diseases are lethal neurodegenerative disorders with no disease-modifying therapy. The prevalence of EOFAD-APP is 1% AD; the prevalence of Trisomy 21 is 1% AD; and the prevalence of AD is about 2.5-5 million in US. Targeting APP can be excellent via human molecular genetics, e.g., APP duplications and point mutations cause EOFAD, in tissues such as cerebral cortex or hippocampus. The mechanism of this targeting may be because APP overexpression or expression of toxic metabolites cause progressive neuronal death. The efficacy has been shown by 70% KD of APP. With respect to safety, KD mice have been reported healthy with some behavioral abnormalities; KD mice have been reported healthy with some spatial memory effects. Possible diagnosis includes family history; genetic testing; early symptoms; or MRI. Biomarkers that can be used include, e.g., CSF APP mRNA and peptides.

### Targeting PSEN1 for Alzheimer Disease

Presenilin 1 mutations causes early onset familial Alzheimer disease (EOFAD); or AD. These diseases are lethal neurodegenerative disorder with no disease-modifying therapy. Targeting PSEN1 can be excellent via human molecular genetics, e.g., PSEN1 point mutations cause EOFAD, in tissues such as cerebral cortex; or hippocampus. The mechanism of this targeting may be because autosomal dominant mutations of PSEN1 cause abnormal APP metabolism and toxic peptides cause progressive neuronal death. The efficacy has been shown by APP KD may obviate need for PSEN1-specific therapy. Possible diagnosis includes family history; genetic testing; early symptoms; or MRI. Biomarkers that can be used include, e.g., CSF PSEN1 and APP peptides.

### Targeting PSEN2 for Alzheimer Disease

Presenilin 2 mutations causes early onset familial Alzheimer disease (EOFAD); or AD. These diseases are lethal neurodegenerative disorder with no disease-modifying therapy. Targeting PSEN2 can be excellent via human molecular genetics, e.g., PSEN2 point mutations cause EOFAD, in tissues such as cerebral cortex or hippocampus. The mechanism of this targeting may be because autosomal dominant mutations of PSEN2 cause abnormal APP metabolism and toxic peptides cause progressive neuronal death. Possible diagnosis includes family history; genetic testing; early symptoms; or MRI. Biomarkers that can be used include, e.g., CSF PSEN2 and APP peptides.

### Targeting Apo E for Alzheimer Disease

Apolipoprotein E4 is associated with sporadic AD in the elderly. This disease is lethal neurodegenerative disorder with no disease-modifying therapy. The prevalence of AD is 2.5-5 million in US. Targeting Apo E may be effective because genomic evidence supporting the association between ApoE4 and AD is excellent in many populations. The target tissue may be cerebral cortex. It is not yet clear if Apo E4 contributes to the pathogenesis of AD despite the strong association in many populations. Thus far, data indicate that Apo E4 homozygosity indicates increased risk of AD in the elderly but is not sufficient for causing AD, even in the elderly. With respect to safety, KD of Apo E in CNS may be safe as human LOF mutations in Apo E are not associated with obvious neurologic defects, although systemic exposure may cause hyperlipoproteinemia type III. Possible diagnosis includes clinical diagnosis of AD; exclusion of EOFAD mutation; genetic testing for the Apo E4 genotype. Biomarkers that can be used include, e.g., CSF APP, Tau mRNA and peptides.

CNS Gene Duplication Disorders. Consistent KD by half may ameliorate these disorders. The targets include MeCP2.

### Targeting MeCP2 for X-Linked Mental Retardation

Methyl CpG Binding Protein 2 gene duplication causes X-linked Mental Retardation (XLMR). This disease is lethal cognitive disorder with no disease-modifying therapy. 1-15% of X-linked MR is caused by MeCP2 duplication; 2-3% of population has MR. Targeting MeCP2 can be excellent via human molecular genetics, e.g., MeCP2 duplication causes XLMR, in tissues such as cerebral cortex. The mechanism of this targeting may be because MeCP2 over-expression cause dysregulation of other gene and neurodegeneration. The efficacy has been shown by 50% KD of MeCP2; and ASO KD in mouse models reverse phenotype. With respect to safety, MeCP2 LOF mutations may cause Rett syndrome. Possible diagnosis includes family history; genetic testing; or early symptoms. Biomarkers that can be used include, e.g., CSF MeCP2 mRNA and peptides.

Dominant Inherited Cerebral Amyloid Angiopathy is a devastating disorder with no disease-modifying therapy. The targets include TTR.

Targeting TTR for hATTR CAA

This targeting may be a low risk introduction to CNS siRNA. Cerebral Amyloid Angiopathy (CAA) and Meningeal Amyloid are lethal disorders with no disease-modifying therapy. Targeting TTR can be excellent via human genetics and pharmacology. The target tissues can be CNS vascular system, or CNS. The mechanism of this targeting may be because Mutant protein accumulates in vascular adventitia, causing CNS bleeds. The efficacy has been shown by 70% KD of TTR. Possible diagnosis includes family history; genetic testing; or early symptoms. Biomarkers that can be used include, e.g., CSF mRNA and protein.

### Targeting ITM2B for CAA

Integral Membrane Protein 2B mutations causes Cerebral Amyloid Angiopathy (CAA), British Type or Familial British Dementia (FBD). Specific mutation may also cause dominant retinal degeneration. This disease is lethal disorder with no disease-modifying therapy. This is a rare disease. Targeting ITM2B can be excellent via human molecular genetics. The target tissues can be CNS vascular system, or CNS. The mechanism of this targeting probably involves GOF mutations. The efficacy has been shown by 70% KD of ITM2B mutant allele. Possible diagnosis includes family history; genetic testing; or early symptoms. Biomarkers that can be used include, e.g., CSF mRNA and protein possible.

### Targeting CST3 for CAA

Cystatin C mutations causes familial cerebral amyloid angiopathy, Icelandic type. This disease is lethal disorder with no disease-modifying therapy. This is a rare disease, except in Iceland and Denmark. Targeting CST3 can be excellent via human genetics. The target tissue can be CNS vascular system. The mechanism of this targeting may be because mutant protein accumulates in vascular adventitia, causing CNS bleeds. The efficacy has been shown by possibly 70% KD of mutant allele. With respect to safety, CST3 KO mice may have risk of arthritis. Possible diagnosis includes family history; genetic testing; or early symptoms. Biomarkers that can be used include, e.g., CSF mRNA and protein possible.

### Targeting SPAST for Spastic Paraplegia

SPASTIN mutations causes Spastic Paraplegia (SP) 4 with cognitive loss. This disease is lower motor neurodegenerative disorder with no disease-modifying therapy. The prevalence of SP is 5 per 100,000 population; SP4 is 45% of dominant SP. Targeting SPAST can be excellent via human molecular genetics, e.g., SPAST trinucleotide mutations causes familial SP, in tissues such as spinal cord; or CNS. The mechanism of this targeting may be because nonsense and probable dominant-negative mutations cause abnormal microtubule metabolism and neurodegeneration. Possible diagnosis includes family history; genetic testing; or early symptoms. Biomarkers that can be used include, e.g., CSF SPAST mRNAs and proteins possible.

### Targeting KIF5A for Spastic Paraplegia

Kinesin Family Member 5A mutations causes Spastic Paraplegia (SP) 10 with peripheral neuropathy and other disorders. This disease is lower motor neurodegenerative disorder with no disease-modifying therapy. The prevalence of SP is 5 per 100,000 people; SP10 is 1 per 1,000,000 people. Targeting KIF5A can be excellent via human molecular genetics, e.g., KIF5A amino terminal missense mutations cause SP10; and KIF5A is expressed in the CNS and encodes a microtubule motor protein. The target tissue may be spinal cord. The mechanism of this targeting may be because autosomal dominant missense mutations cause SP10 possibly affect microtubule binding to the motor. The efficacy may be provided by possibly KD of mutant alleles. With respect to safety, KIF5A frameshift mutations cause Neonatal intractable myoclonus and splice site mutations are associated with familial ALS, possibly through LOF mechanisms. Possible diagnosis includes family history; genetic testing; or early symptoms. Biomarkers that can be used include, e.g., CSF mRNAs and proteins possible.

### Targeting ATL1 for Spastic Paraplegia

Atlastin mutations causes Spastic Paraplegia 3A and Sensory Neuropathy 1D, Hereditary Sensory Neuropathy (HSN). This disease is a lower motor neurodegenerative disorder with no disease-modifying therapy. The prevalence of SP is 5 per 100,000 people; SP3A is a rare dominant form. Targeting ATL1 can be excellent via human molecular genetics, e.g., ATL1 point mutations cause familial SP. The target tissue may be spinal cord. The mechanism of this targeting may be because autosomal dominant expression of dominant-negative ATL1 protein causes SP3A; however, LOF mutations causes Sensory Neuropathy ID. The efficacy has been shown by 70% KD of specific ATL1 allele. With respect to safety, ATL1 heterozygous LOF mutations causes HSN1D. Possible diagnosis includes family history; genetic testing; or early symptoms. Biomarkers that can be used include, e.g., CSF ATL1 mRNAs and proteins.

### Targeting NIPA1 for Spastic Paraplegia

LOF NIPA1 mutations cause Spastic Paraplegia 6 with epilepsy and seizures. This disease is lower motor neurodegenerative disorder with no disease-modifying therapy. The prevalence of SP is 5 per 100,000 people; SP6 is a rare dominant form. Targeting NIPA1 can be excellent via human molecular genetics, e.g., NIPA1 point mutations cause familial SP. The target tissues can be spinal cord; or CNS. The mechanism of this targeting may be because autosomal dominant expression of defective membrane protein causes SP3A; and possibly LOF. Possible diagnosis includes family history; genetic testing; or early symptoms. Biomarkers that can be used include, e.g., CSF mRNAs and proteins possible.

Dominant Inherited Myotonic Dystrophy is a disorder of CNS, Skeletal Muscle and Cardiac Muscle Requiring CNS and Systemic Therapy. The targets include MPK for DM1.

### Targeting DMPK for Myotonic Dystrophy 1

CNS and systemic therapy needed for effective therapy targeting dystrophia Myotonica Protein Kinase. Myotonic dystrophy 1 (DM1) is a degenerative disorder of muscle and CNS. It is a lethal disorder with no disease-modifying therapy. The prevalence of DM1 is 1 per 8,000 people worldwide. Targeting DMPK can be excellent via human molecular genetics, e.g., DMPK CTG repeat expansion causes familial DM1. The target tissues may be skeletal muscle, cardiac muscle, or CNS. The mechanism of this targeting may be because autosomal dominant non-coding CTG repeat causes abnormal RNA processing and dominant negative effect; anticipation from extreme expansion causes early onset disease. The efficacy has been shown by 70% of DMPK; and ASO efficacy have been demonstrated in mice. The safety has been demonstrated in mice with KO and ASO KD. Possible diagnosis includes family history; genetic testing; or early symptoms. Biomarkers that can be used include, e.g., Blood and CSF mRNAs and proteins.

### Targeting ZNF9 for Myotonic Dystrophy 2

Zinc Finger Protein 9 mutations causes Myotonic dystrophy 2 (DM2), a degenerative disorder of skeletal muscle. This is a serious disorder with no disease-modifying therapy. The prevalence of DM2 is 1 per 8,000 people worldwide; it is the most common muscular dystrophy in adults. Targeting ZNF9 can be excellent via human molecular genetics, e.g., ZNF9 CTTG repeat expansion in intron 1 causes familial DM2. The target tissues can be skeletal muscle, or cardiac muscle. The mechanism of this targeting may be because autosomal dominant CTTG repeat expansion in intron 1 causes abnormal RNA metabolism and dominant negative effects. The efficacy has been shown by 70% of ZNF9. Safe KD in mice has been demonstrated. Possible diagnosis includes family history; genetic testing; or early symptoms. Biomarkers that can be used include, e.g., Blood mRNAs and proteins.

Dominant Inherited Prion Diseases are inherited, sporadic and transmissible PRNP disorders. The targets include PRNP.

### Targeting PRNP for Myotonic Prion Diseases

Myotonic prion diseases are dominant inherited Prion diseases, including PRNP-Related Cerebral Amyloid Angiopathy, Gerstmann-Straussler Disease (GSD), Creutzfeldt-Jakob Disease (CJD), Fatal Familial Insomnia (FFI), Huntington Disease-Like 1 (HDL1), and Kuru susceptibility. These diseases are lethal neurodegenerative disorders with no disease-modifying therapy. The prevalence of this type of diseases is 1 per 1,000,000 people. Targeting PRNP can be excellent via human molecular genetics, e.g., PRNP mutations cause familial and sporadic Prion disease. The target tissue can be CNS. The mechanism of this targeting may be because autosomal dominant protein mid-folding causes neurotoxicity. The efficacy has been shown by 70% of PRNP KD; and PRNP polymorphisms appear protective for Kuru. With respect to safety, PRNP KO mice have been reported healthy. Possible diagnosis includes family history; genetic testing; or early symptoms. Biomarkers that can be used include, e.g., CSF mRNAs and proteins.

### Targeting Glycogen Synthase for Myoclonic Epilepsy of Lafora

Laforin (EPM2A) gene mutations causes AR Myoclonic Epilepsy, an inherited progressive seizure disorder. This disease is a lethal disorder of seizures and cognitive decline with no disease-modifying therapy. The prevalence of this disease is 4 per 1,000,000 people. Targeting Glycogen Synthase can be excellent via human molecular genetics, e.g., mutations causes AR familial Myoclonic Epilepsy of Lafora. The target tissue may be CNS. The mechanism of this targeting may be because autosomal recessive dysfunction of Laforin causes misfolding of glycogen and foci for seizures. The efficacy has been shown by 70% KD of Glycogen synthase GYS1. With respect to safety, GYS1 deficiency causes skeletal and cardiac muscle glycogen deficiency; GYS1 mice that survive have muscle defects. Possible diagnosis includes family history; genetic testing; or early symptoms. Biomarkers that can be used include, e.g., CSF mRNAs and protein.

In some embodiments, the invention provides a compound that target genes for diseases including, but are not limited to, age-related macular degeneration (AMD) (dry and wet), birdshot chorioretinopathy, dominant retinitis pigmentosa 4, Fuch's dystrophy, hATTR amyloidosis, hereditary and sporadic glaucoma, and stargardt's disease.

In some embodiments, the invention provides a compound that targets VEGF for wet (or exudative) AMD.

In some embodiments, the invention provides a compound that targets C3 for dry (or nonexudative) AMD.

In some embodiments, the invention provides a compound that targets CFB for dry (or nonexudative) AMD.

In some embodiments, the invention provides a compound that targets MYOC for glaucoma.

In some embodiments, the invention provides a compound that targets ROCK2 for glaucoma.

In some embodiments, the invention provides a compound that targets ADRB2 for glaucoma.

In some embodiments, the invention provides a compound that targets CA2 for glaucoma.

In some embodiments, the invention provides a compound that targets CRYGC for cataract.

In some embodiments, the invention provides a compound that targets PPP3CB for dry eye syndrome.

### Ligands

In certain embodiments, the compound of the invention is further modified by covalent attachment of one or more conjugate groups. In general, conjugate groups modify one or more properties of the attached compound of the invention including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the compound further comprises a targeting ligand that targets a receptor which mediates delivery to a specific CNS tissue. These targeting ligands can be conjugated in combination with the lipophilic moiety to enable specific intrathecal and systemic delivery.

Exemplary targeting ligands that targets the receptor mediated delivery to a CNS tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells, glial cells), glucose transporter protein, and LDL receptor ligand.

In some embodiments, the compound further comprises a targeting ligand that targets a receptor which mediates delivery to a specific ocular tissue. These targeting ligands can be conjugated in combination with the lipophilic moiety to enable specific ocular delivery (e.g., intravitreal delivery) and systemic delivery. Exemplary targeting ligands that targets the receptor mediated delivery to a ocular tissue are lipophilic ligands such as all-trans retinol (which targets the retinoic acid receptor); RGD peptide (which targets retinal pigment epithelial cells), such as H-Gly-Arg-Gly-Asp-Ser-Pro-Lys-Cys-OH or Cyclo(-Arg-Gly-Asp-D-Phe-Cys; LDL receptor ligands; and carbohydrate based ligands (which targets endothelial cells in posterior eye).

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g., steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-1B, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

AALEALAEALEALAEALEALAEAAAAGGC (GALA);
AALAEALAEALAEALAEALAEALAAAAGGC (EALA);
ALEALAEALEALAEA;
GLFEAIEGFIENGWEGMIWDYG (INF-7);
GLFGAIAGFIENGWEGMIDGWYG (Inf HA-2);
GLFEAIEGFIENGWEGMIDGWYGCGLFEAIEGFIENGWEGMID
GWYGC (diINF-7);
GLFEAIEGFIENGWEGMIDGGCGLFEAIEGFIENGWEGMIDGGC
(diINF-3);
GLFGALAEALAEALAEHLAEALAEALEALAAGGSC (GLF);
GLFEAIEGFIENGWEGLAEALAEALEALAAGGSC (GALA-INF3);
GLF EAIEGFI ENGW EGnI DG K GLF EAI EGFI ENGW EGnI
DG (INF-5, n is norleucine);
LFEALLELLESLWELLLEA (JTS-1);
GLFKALLKLLKSLWKLLLKA (ppTG1);
GLFRALLRLLRSLWRLLLRA (ppTG20);
WEAKLAKALAKALAKHLAKALAKALKACEA (KALA);
GLFFEAIAEFIEGGWEGLIEGC (HA);
GIGAVLKVLTTGLPALISWIKRKRQQ (Melittin);
H₅WYG;
and
CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to,

RQIKIWFQNRRMKWKK (penetratin);
GRKKRRQRRRPPQC (Tat fragment 48-60);
GALFLGWLGAAGSTMGAWSQPKKKRKV (signal sequence
based peptide);
LLIILRRRIRKQAHAHSK (PVEC);
GWTLNSAGYLLKINLKALAALAKKIL (transportan);
KLALKLALKALKAALKLA (amphiphilic model peptide);
RRRRRRRRR (Arg9);
KFFKFFKFFK (Bacterial cell wall permeating
peptide);
LLGDFFRKSKEKIGKEFKRIVQRIKDFLRNLVPRTES (LL-37);
SWLSKTAKKLENSAKKRISEGIAIAIQGGPR (cecropin P1);
ACYCRIPACIAGERRYGTCIYQGRLWAFCC (α-defensin);
DHYNCVS SGGQCLYSACPIFTKIQGTCYRGKAKCCK (β-defensin);
RRRPRPPYLPRPRPPPFFPPRLPPRIPPGFPPRFPPRFPGKR-NH2
(PR-39);
ILPWKWPWWPWRR-NH2 (indolicidin);
AAVALLPAVLLALLAP (RFGF);
AALLPVLLAAP (RFGF analogue);
and
RKCRIVVIRVCR (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃ (GalNAc and multivalent GalNAc are collectively referred to herein as GalNAc conjugates); D-mannose, multivalent mannose, multivalent lactose, N-acetyl-glucosamine, Glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the compound of the invention (e.g., a compound of the invention or linker). In some embodiments, the ligand can be incorporated via coupling to a “precursor” monomer after said “precursor” monomer has been incorporated into a component of the compound of the invention (e.g., a compound of the invention or linker). For example, a monomer having, e.g., an amino-terminated tether (i.e., having no associated ligand), e.g., monomer-linker-NH₂ can be incorporated into a component of the compounds of the invention (e.g., a compound of the invention or linker). In a subsequent operation, i.e., after incorporation of the precursor monomer into a component of the compounds of the invention (e.g., a compound of the invention or linker), a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the compound of the invention. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Exemplary carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

The ligand can be attached to the compound of the inventions via a linker or a carrier monomer, e.g., a ligand carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier monomer into the backbone, e.g., the phosphate, or modified phosphate, e.g., sulfur containing, backbone, of an oligonucleotide. A “tethering attachment point” (TAP) in refers to an atom of the carrier monomer, e.g., a carbon atom or a heteroatom (distinct from an atom which provides a backbone attachment point), that connects a selected moiety. The selected moiety can be, e.g., a carbohydrate, e.g. monosaccharide, disaccharide, trisaccharide, tetrasaccharide, oligosaccharide and polysaccharide. Optionally, the selected moiety is connected by an intervening tether to the carrier monomer. Thus, the carrier will often include a functional group, e.g., an amino group, or generally, provide a bond, that is suitable for incorporation or tethering of another chemical entity, e.g., a ligand to the constituent atom.

Representative U.S. patents that teach the preparation of conjugates of nucleic acids include, but are not limited to, U.S. Pat. Nos. 4,828,979; 4,948,882; 5,218, 105; 5,525,465; 5,541,313; 5,545,730; 5,552,538; 5,578, 717, 5,580,731; 5,580,731; 5,591,584; 5,109,124; 5,118, 802; 5,138,045; 5,414,077; 5,486,603; 5,512,439; 5,578, 718; 5,608,046; 4,587,044; 4,605,735; 4,667,025; 4,762, 779; 4,789,737; 4,824,941; 4,835,263; 4,876,335; 4,904, 582; 4,958,013; 5,082,830; 5,112,963; 5,214,136; 5,082, 830; 5,112,963; 5,149,782;

5,214,136; 5,245,022; 5,254, 469; 5,258,506; 5,262,536; 5,272,250; 5,292,873; 5,317, 098; 5,371,241, 5,391,723; 5,416,203, 5,451,463; 5,510, 475; 5,512,667; 5,514,785; 5,565,552; 5,567,810; 5,574, 142; 5,585,481; 5,587,371; 5,595,726; 5,597,696; 5,599, 923; 5,599,928; 5,672,662; 5,688,941; 5,714,166; 6,153, 737; 6,172,208; 6,300,319; 6,335,434; 6,335,437; 6,395, 437; 6,444,806; 6,486,308; 6,525,031; 6,528,631; 6,559, 279; contents of which are herein incorporated in their entireties by reference.

In some embodiments, the compound further comprises a targeting ligand that targets a liver tissue. In some embodiments, the targeting ligand is a carbohydrate-based ligand. In one embodiment, the targeting ligand is a GalNAc conjugate.

Because the ligand can be conjugated to the iRNA agent via a linker or carrier, and because the linker or carrier can contain a branched linker, the iRNA agent can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valences. In certain embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

Evaluation of Candidate iRNAs

One can evaluate a candidate iRNA agent, e.g., a modified RNA, for a selected property by exposing the agent or modified molecule and a control molecule to the appropriate conditions and evaluating for the presence of the selected property. For example, resistance to a degradant can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dssiRNA compounds.

In an alternative functional assay, a candidate dssiRNA compound homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dssiRNA compound would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### Physiological Effects

The siRNA compounds described herein can be designed such that determining therapeutic toxicity is made easier by the complementarity of the siRNA with both a human and a non-human animal sequence. By these methods, an siRNA can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the siRNA compound could be complementary to sequences within homologous genes, e.g., oncogenes or tumor suppressor genes, of the non-human mammal and the human. By determining the toxicity of the siRNA compound in the non-human mammal, one can extrapolate the toxicity of the siRNA compound in a human. For a more strenuous toxicity test, the siRNA can be complementary to a human and more than one, e.g., two or three or more, non-human animals.

The methods described herein can be used to correlate any physiological effect of an siRNA compound on a human, e.g., any unwanted effect, such as a toxic effect, or any positive, or desired effect.

Increasing Cellular Uptake of siRNAs

Described herein are various siRNA compositions that contain covalently attached conjugates that increase cellular uptake and/or intracellular targeting of the siRNAs.

Additionally provided are methods of the invention that include administering an siRNA compound and a drug that affects the uptake of the siRNA into the cell. The drug can be administered before, after, or at the same time that the siRNA compound is administered. The drug can be covalently or non-covalently linked to the siRNA compound. The drug can be, for example, a lipopolysaccharide, an activator of p38 MAP kinase, or an activator of NF-κB. The drug can have a transient effect on the cell. The drug can increase the uptake of the siRNA compound into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The drug can also increase the uptake of the siRNA compound into a given cell or tissue by activating an inflammatory response, for example. Exemplary drugs that would have such an effect include tumor necrosis factor alpha (TNFalpha), interleukin-1 beta, a CpG motif, gamma interferon or more generally an agent that activates a toll-like receptor.

siRNA Production

An siRNA can be produced, e.g., in bulk, by a variety of methods. Exemplary methods include: organic synthesis and RNA cleavage, e.g., in vitro cleavage.

Organic Synthesis. An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilot II reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

dsiRNA Cleavage. siRNAs can also be made by cleaving a larger siRNA. The cleavage can be mediated in vitro or in vivo. For example, to produce iRNAs by cleavage in vitro, the following method can be used:

In vitro transcription. dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be dotoxins that may contaminate preparations of the recombinant enzymes.

In Vitro Cleavage. In one embodiment, RNA generated by this method is carefully purified to remove endsiRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9; and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

Making Double-Stranded iRNA Agents Conjugated to a Lipophilic Moiety

In some embodiments, the lipophilic monomer containing a lipophilic moiety conjugated to the compound via a nucleobase, sugar moiety, or internucleosidic linkage.

Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a lipophilic moiety is conjugated to a nucleobase, the preferred position is one that does not interfere with hybridization, i.e., does not interfere with the hydrogen bonding interactions needed for base pairing. In one embodiment, the lipophilic monomer containing a lipophilic moieties may be conjugated to a nucleobase via a linker containing an alkyl, alkenyl or amide linkage.

Conjugation to sugar moieties of nucleosides can occur at any carbon atom. Exemplary carbon atoms of a sugar moiety that a lipophilic moiety can be attached to include the 2′, 3′, and 5′ carbon atoms. A lipophilic moiety can also be attached to the 1′ position, such as in an abasic residue. In one embodiment, the lipophilic moieties may be conjugated to a sugar moiety, via a 2′-O modification, with or without a linker.

Internucleosidic linkages can also bear lipophilic moieties. For phosphorus-containing linkages (e.g., phosphodiester, phosphorothioate, phosphorodithioate, phosphoroamidate, and the like), the lipophilic moiety can be attached directly to the phosphorus atom or to an O, N, or S atom bound to the phosphorus atom. For amine- or amide-containing internucleosidic linkages (e.g., PNA), the lipophilic moiety can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In one embodiment, a first (complementary) RNA strand and a second (sense) RNA strand can be synthesized separately, wherein one of the RNA strands comprises a pendant lipophilic moiety, and the first and second RNA strands can be mixed to form a dsRNA. The step of synthesizing the RNA strand preferably involves solid-phase synthesis, wherein individual nucleotides are joined end to end through the formation of internucleotide 3′-5′ phosphodiester bonds in consecutive synthesis cycles.

In one embodiment, a lipophilic molecule having a phosphoramidite group is coupled to the 3′-end or 5′-end of either the first (complementary) or second (sense) RNA strand in the last synthesis cycle. In the solid-phase synthesis of an RNA, the nucleotides are initially in the form of nucleoside phosphoramidites. In each synthesis cycle, a further nucleoside phosphoramidite is linked to the —OH group of the previously incorporated nucleotide. If the lipophilic molecule has a phosphoramidite group, it can be coupled in a manner similar to a nucleoside phosphoramidite to the free OH end of the RNA synthesized previously in the solid-phase synthesis. The synthesis can take place in an automated and standardized manner using a conventional RNA synthesizer. Synthesis of the lipophilic molecule having the phosphoramidite group may include phosphitylation of a free hydroxyl to generate the phosphoramidite group.

In general, the oligonucleotides can be synthesized using protocols known in the art, for example, as described in Caruthers et al., Methods in Enzymology (1992) 211:3-19; WO 99/54459; Wincott et al., Nucl. Acids Res. (1995) 23:2677-2684; Wincott et al., Methods Mol. Bio., (1997) 74:59; Brennan et al., Biotechnol. Bioeng. (1998) 61:33-45; and U.S. Pat. No. 6,001,311; each of which is hereby incorporated by reference in its entirety. In general, the synthesis of oligonucleotides involves conventional nucleic acid protecting and coupling groups, such as dimethoxytrityl at the 5′-end, and phosphoramidites at the 3′-end. In a non-limiting example, small scale syntheses are conducted on a Expedite 8909 RNA synthesizer sold by Applied Biosystems, Inc. (Weiterstadt, Germany), using ribonucleoside phosphoramidites sold by ChemGenes Corporation (Ashland, Mass.). Alternatively, syntheses can be performed on a 96-well plate synthesizer, such as the instrument produced by Protogene (Palo Alto, Calif.), or by methods such as those described in Usman et al., J. Am. Chem. Soc. (1987) 109:7845; Scaringe, et al., Nucl. Acids Res. (1990) 18:5433; Wincott, et al., Nucl. Acids Res. (1990) 23:2677-2684; and Wincott, et al., Methods Mol. Bio. (1997) 74:59, each of which is hereby incorporated by reference in its entirety.

The nucleic acid molecules of the present invention may be synthesized separately and joined together post-synthetically, for example, by ligation (Moore et al., Science (1992) 256:9923; WO 93/23569; Shabarova et al., Nucl. Acids Res. (1991) 19:4247; Bellon et al., Nucleosides & Nucleotides (1997) 16:951; Bellon et al., Bioconjugate Chem. (1997) 8:204; or by hybridization following synthesis and/or deprotection. The nucleic acid molecules can be purified by gel electrophoresis using conventional methods or can be purified by high pressure liquid chromatography (HPLC; see Wincott et al., supra, the totality of which is hereby incorporated herein by reference) and re-suspended in water.

### Pharmaceutical Compositions

In one aspect, the invention features a pharmaceutical composition that includes an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) including a nucleotide sequence complementary to a target RNA, e.g., substantially and/or exactly complementary. The target RNA can be a transcript of an endogenous human gene. In one embodiment, the siRNA compound (a) is 19-25 nucleotides long, for example, 21-23 nucleotides, (b) is complementary to an endogenous target RNA, and, optionally, (c) includes at least one 3′ overhang 1-5 nt long. In one embodiment, the pharmaceutical composition can be an emulsion, microemulsion, cream, jelly, or liposome.

In one example the pharmaceutical composition includes an siRNA compound mixed with a topical delivery agent. The topical delivery agent can be a plurality of microscopic vesicles. The microscopic vesicles can be liposomes. In some embodiments the liposomes are cationic liposomes.

In another aspect, the pharmaceutical composition includes an siRNA compound, e.g., a double-stranded siRNA compound, or ssiRNA compound (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) admixed with a topical penetration enhancer. In one embodiment, the topical penetration enhancer is a fatty acid. The fatty acid can be arachidonic acid, oleic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C₁₋₁₀ alkyl ester, monoglyceride, diglyceride or pharmaceutically acceptable salt thereof.

In another embodiment, the topical penetration enhancer is a bile salt. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether or a pharmaceutically acceptable salt thereof.

In another embodiment, the penetration enhancer is a chelating agent. The chelating agent can be EDTA, citric acid, a salicyclate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorchemical emulsion or mixture thereof.

In another embodiment, the penetration enhancer can be selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpenes.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a form suitable for oral delivery. In one embodiment, oral delivery can be used to deliver an siRNA compound composition to a cell or a region of the gastro-intestinal tract, e.g., small intestine, colon (e.g., to treat a colon cancer), and so forth. The oral delivery form can be tablets, capsules or gel capsules. In one embodiment, the siRNA compound of the pharmaceutical composition modulates expression of a cellular adhesion protein, modulates a rate of cellular proliferation, or has biological activity against eukaryotic pathogens or retroviruses. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methylcellulose phthalate or cellulose acetate phthalate.

In another embodiment, the oral dosage form of the pharmaceutical composition includes a penetration enhancer. The penetration enhancer can be a bile salt or a fatty acid. The bile salt can be ursodeoxycholic acid, chenodeoxycholic acid, and salts thereof. The fatty acid can be capric acid, lauric acid, and salts thereof.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an siRNA compound and a delivery vehicle. In one embodiment, the siRNA compound is (a) is 19-25 nucleotides long, for example, 21-23 nucleotides, (b) is complementary to an endogenous target RNA, and, optionally, (c) includes at least one 3′ overhang 1-5 nucleotides long.

In one embodiment, the delivery vehicle can deliver an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) to a cell by a topical route of administration. The delivery vehicle can be microscopic vesicles. In one example the microscopic vesicles are liposomes. In some embodiments the liposomes are cationic liposomes. In another example the microscopic vesicles are micelles. In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in oral dosage form. In one embodiment, the oral dosage form is selected from the group consisting of tablets, capsules and gel capsules. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the pharmaceutical composition includes a penetration enhancer, e.g., a penetration enhancer described herein.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a rectal dosage form. In one embodiment, the rectal dosage form is an enema. In another embodiment, the rectal dosage form is a suppository.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a vaginal dosage form. In one embodiment, the vaginal dosage form is a suppository. In another embodiment, the vaginal dosage form is a foam, cream, or gel.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a pulmonary or nasal dosage form. In one embodiment, the siRNA compound is incorporated into a particle, e.g., a macroparticle, e.g., a microsphere. The particle can be produced by spray drying, lyophilization, evaporation, fluid bed drying, vacuum drying, or a combination thereof. The microsphere can be formulated as a suspension, a powder, or an implantable solid.

### Treatment Methods and Routes of Delivery

Another aspect of the invention relates to a method of reducing the expression of a target gene in a cell, comprising contacting said cell with the compound of the invention. In one embodiment, the cell is an extrahepatic cell.

Another aspect of the invention relates to a method of reducing the expression of a target gene in a subject, comprising administering to the subject the compound of the invention.

Another aspect of the invention relates to a method of treating a subject having a CNS disorder, comprising administering to the subject a therapeutically effective amount of the double-stranded RNAi agent of the invention, thereby treating the subject. Exemplary CNS disorders that can be treated by the method of the invention include Alzheimer, amyotrophic lateral sclerosis (ALS), frontotemporal dementia, Huntington, Parkinson, spinocerebellar, prion, and lafora.

The compound of the invention can be delivered to a subject by a variety of routes, depending on the type of genes targeted and the type of disorders to be treated. In some embodiments, the compound is administered extrahepatically, such as an ocular administration (e.g., intravitreal administration) or an intrathecal or intracerebroventricular administration.

In one embodiment, the compound is administered intrathecally or intracerebroventricularly. By intrathecal or intracerebroventricular administration of the double-stranded iRNA agent, the method can reduce the expression of a target gene in a brain or spine tissue, for instance, cortex, cerebellum, cervical spine, lumbar spine, and thoracic spine.

In some embodiments, exemplary target genes are APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, and TTR. To reduce the expression of these target genes in the subject, the compound can be administered to the eye(s) directly (e.g., intravitreally). By intravitreal administration of the double-stranded iRNA agent, the method can reduce the expression of the target gene in an ocular tissue.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. A composition that includes a iRNA can be delivered to a subject by a variety of routes. Exemplary routes include: intravenous, topical, rectal, anal, vaginal, nasal, pulmonary, ocular.

The iRNA molecules of the invention can be incorporated into pharmaceutical compositions suitable for administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular or intracerebroventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular or intracerebroventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes may be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

In one embodiment, the administration of the siRNA compound, e.g., a double-stranded siRNA compound, or ssiRNA compound, composition is parenteral, e.g., intravenous (e.g., as a bolus or as a diffusible infusion), intradermal, intraperitoneal, intramuscular, intrathecal, intraventricular, intracerebroventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

IntrathecalAdministration. In one embodiment, the compound is delivered by intrathecal injection (i.e. injection into the spinal fluid which bathes the brain and spinal cord tissue). Intrathecal injection of iRNA agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal cord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in PCT/US2015/013253, filed on Jan. 28, 2015, which is incorporated by reference in its entirety.

The amount of intrathecally or intracerebroventricularly injected iRNA agents may vary from one target gene to another target gene and the appropriate amount that has to be applied may have to be determined individually for each target gene. Typically, this amount ranges between 10 μg to 2 mg, preferably 50 μg to 1500 μg, more preferably 100 μg to 1000 μg.

Rectal Administration. The invention also provides methods, compositions, and kits, for rectal administration or delivery of siRNA compounds described herein.

Accordingly, an siRNA compound, e.g., a double-stranded siRNA compound, or ssiRNA compound, (e.g., a precursor, e.g., a larger siRNA compound which can be processed into a ssiRNA compound, or a DNA which encodes a an siRNA compound, e.g., a double-stranded siRNA compound, or ssiRNA compound, or precursor thereof) described herein, e.g., a therapeutically effective amount of a siRNA compound described herein, e.g., a siRNA compound having a double stranded region of less than 40, and, for example, less than 30 nucleotides and having one or two 1-3 nucleotide single strand 3′ overhangs can be administered rectally, e.g., introduced through the rectum into the lower or upper colon. This approach is particularly useful in the treatment of, inflammatory disorders, disorders characterized by unwanted cell proliferation, e.g., polyps, or colon cancer.

The medication can be delivered to a site in the colon by introducing a dispensing device, e.g., a flexible, camera-guided device similar to that used for inspection of the colon or removal of polyps, which includes means for delivery of the medication.

The rectal administration of the siRNA compound is by means of an enema. The siRNA compound of the enema can be dissolved in a saline or buffered solution. The rectal administration can also by means of a suppository, which can include other ingredients, e.g., an excipient, e.g., cocoa butter or hydropropylmethylcellulose.

Ocular Delivery. The iRNA agents described herein can be administered to an ocular tissue. For example, the medications can be applied to the surface of the eye or nearby tissue, e.g., the inside of the eyelid. They can be applied topically, e.g., by spraying, in drops, as an eyewash, or an ointment. Administration can be provided by the subject or by another person, e.g., a health care provider. The medication can be provided in measured doses or in a dispenser which delivers a metered dose. The medication can also be administered to the interior of the eye, and can be introduced by a needle or other delivery device which can introduce it to a selected area or structure. Ocular treatment is particularly desirable for treating inflammation of the eye or nearby tissue.

In certain embodiments, the double-stranded iRNA agents may be delivered directly to the eye by ocular tissue injection such as periocular, conjunctival, subtenon, intracameral, intravitreal, intraocular, anterior or posterior juxtascleral, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or within the eye. Intracameral injection may be through the cornea into the anterior chamber to allow the agent to reach the trabecular meshwork. Intracanalicular injection may be into the venous collector channels draining Schlemm's canal or into Schlemm's canal.

In one embodiment, the double-stranded iRNA agents may be administered into the eye, for example the vitreous chamber of the eye, by intravitreal injection, such as with pre-filled syringes in ready-to-inject form for use by medical personnel.

For ophthalmic delivery, the double-stranded iRNA agents may be combined with ophthalmologically acceptable preservatives, co-solvents, surfactants, viscosity enhancers, penetration enhancers, buffers, sodium chloride, or water to form an aqueous, sterile ophthalmic suspension or solution. Solution formulations may be prepared by dissolving the conjugate in a physiologically acceptable isotonic aqueous buffer. Further, the solution may include an acceptable surfactant to assist in dissolving the double-stranded iRNA agents. Viscosity building agents, such as hydroxymethyl cellulose, hydroxyethyl cellulose, methylcellulose, polyvinylpyrrolidone, or the like may be added to the pharmaceutical compositions to improve the retention of the double-stranded iRNA agents.

To prepare a sterile ophthalmic ointment formulation, the double-stranded iRNA agents is combined with a preservative in an appropriate vehicle, such as mineral oil, liquid lanolin, or white petrolatum. Sterile ophthalmic gel formulations may be prepared by suspending the double-stranded iRNA agents in a hydrophilic base prepared from the combination of, for example, CARBOPOL®-940 (BF Goodrich, Charlotte, N.C.), or the like, according to methods known in the art.

Topical Delivery. Any of the siRNA compounds described herein can be administered directly to the skin. For example, the medication can be applied topically or delivered in a layer of the skin, e.g., by the use of a microneedle or a battery of microneedles which penetrate into the skin, but, for example, not into the underlying muscle tissue. Administration of the siRNA compound composition can be topical. Topical applications can, for example, deliver the composition to the dermis or epidermis of a subject. Topical administration can be in the form of transdermal patches, ointments, lotions, creams, gels, drops, suppositories, sprays, liquids or powders. A composition for topical administration can be formulated as a liposome, micelle, emulsion, or other lipophilic molecular assembly. The transdermal administration can be applied with at least one penetration enhancer, such as iontophoresis, phonophoresis, and sonophoresis.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. In some embodiments, an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) is delivered to a subject via topical administration. “Topical administration” refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

The term “skin,” as used herein, refers to the epidermis and/or dermis of an animal. Mammalian skin consists of two major, distinct layers. The outer layer of the skin is called the epidermis. The epidermis is comprised of the stratum corneum, the stratum granulosum, the stratum spinosum, and the stratum basale, with the stratum corneum being at the surface of the skin and the stratum basale being the deepest portion of the epidermis. The epidermis is between 50 μm and 0.2 mm thick, depending on its location on the body.

Beneath the epidermis is the dermis, which is significantly thicker than the epidermis. The dermis is primarily composed of collagen in the form of fibrous bundles. The collagenous bundles provide support for, inter alia, blood vessels, lymph capillaries, glands, nerve endings and immunologically active cells.

One of the major functions of the skin as an organ is to regulate the entry of substances into the body. The principal permeability barrier of the skin is provided by the stratum corneum, which is formed from many layers of cells in various states of differentiation. The spaces between cells in the stratum corneum is filled with different lipids arranged in lattice-like formations that provide seals to further enhance the skins permeability barrier.

The permeability barrier provided by the skin is such that it is largely impermeable to molecules having molecular weight greater than about 750 Da. For larger molecules to cross the skin's permeability barrier, mechanisms other than normal osmosis must be used.

Several factors determine the permeability of the skin to administered agents. These factors include the characteristics of the treated skin, the characteristics of the delivery agent, interactions between both the drug and delivery agent and the drug and skin, the dosage of the drug applied, the form of treatment, and the post treatment regimen. To selectively target the epidermis and dermis, it is sometimes possible to formulate a composition that comprises one or more penetration enhancers that will enable penetration of the drug to a preselected stratum.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose position and retention at the site of administration (Lee et al., Critical Reviews in Therapeutic Drug Carrier Systems, 1991, p. 168) may be useful methods for enhancing the transport of topically applied compositions across skin and mucosal sites.

The compositions and methods provided may also be used to examine the function of various proteins and genes in vitro in cultured or preserved dermal tissues and in animals. The invention can be thus applied to examine the function of any gene. The methods of the invention can also be used therapeutically or prophylactically. For example, for the treatment of animals that are known or suspected to suffer from diseases such as psoriasis, lichen planus, toxic epidermal necrolysis, ertythema multiforme, basal cell carcinoma, squamous cell carcinoma, malignant melanoma, Paget's disease, Kaposi's sarcoma, pulmonary fibrosis, Lyme disease and viral, fungal and bacterial infections of the skin.

Pulmonary Delivery. Any of the siRNA compounds described herein can be administered to the pulmonary system. Pulmonary administration can be achieved by inhalation or by the introduction of a delivery device into the pulmonary system, e.g., by introducing a delivery device which can dispense the medication. Certain embodiments may use a method of pulmonary delivery by inhalation. The medication can be provided in a dispenser which delivers the medication, e.g., wet or dry, in a form sufficiently small such that it can be inhaled. The device can deliver a metered dose of medication. The subject, or another person, can administer the medication. Pulmonary delivery is effective not only for disorders which directly affect pulmonary tissue, but also for disorders which affect other tissue. siRNA compounds can be formulated as a liquid or nonliquid, e.g., a powder, crystal, or aerosol for pulmonary delivery.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. A composition that includes an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) can be administered to a subject by pulmonary delivery. Pulmonary delivery compositions can be delivered by inhalation by the patient of a dispersion so that the composition, for example, iRNA, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are may be used. One of the benefits of using an atomizer or inhaler is that the potential for contamination is minimized because the devices are self contained. Dry powder dispersion devices, for example, deliver drugs that may be readily formulated as dry powders. A iRNA composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” For example, the average particle size is less than about 10 μm in diameter with a relatively uniform spheroidal shape distribution. In some embodiments, the diameter is less than about 7.5 μm and in some embodiments less than about 5.0 μm. Usually the particle size distribution is between about 0.1 μm and about 5 μm in diameter, sometimes about 0.3 μm to about 5 m.

The term “dry” means that the composition has a moisture content below about 10% by weight (% w) water, usually below about 5% w and in some cases less it than about 3% w. A dry composition can be such that the particles are readily dispersible in an inhalation device to form an aerosol.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A group of carbohydrates may include lactose, threhalose, raffinose maltodextrins, and mannitol. Suitable polypeptides include aspartame. Amino acids include alanine and glycine, with glycine being used in some embodiments.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate may be used in some embodiments.

Pulmonary administration of a micellar iRNA formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Oral or Nasal Delivery. Any of the siRNA compounds described herein can be administered orally, e.g., in the form of tablets, capsules, gel capsules, lozenges, troches or liquid syrups. Further, the composition can be applied topically to a surface of the oral cavity.

Any of the siRNA compounds described herein can be administered nasally. Nasal administration can be achieved by introduction of a delivery device into the nose, e.g., by introducing a delivery device which can dispense the medication. Methods of nasal delivery include spray, aerosol, liquid, e.g., by drops, or by topical administration to a surface of the nasal cavity. The medication can be provided in a dispenser with delivery of the medication, e.g., wet or dry, in a form sufficiently small such that it can be inhaled. The device can deliver a metered dose of medication. The subject, or another person, can administer the medication.

Nasal delivery is effective not only for disorders which directly affect nasal tissue, but also for disorders which affect other tissue siRNA compounds can be formulated as a liquid or nonliquid, e.g., a powder, crystal, or for nasal delivery. As used herein, the term “crystalline” describes a solid having the structure or characteristics of a crystal, i.e., particles of three-dimensional structure in which the plane faces intersect at definite angles and in which there is a regular internal structure. The compositions of the invention may have different crystalline forms. Crystalline forms can be prepared by a variety of methods, including, for example, spray drying.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. Both the oral and nasal membranes offer advantages over other routes of administration. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many drugs. Further, the sublingual mucosa is convenient, acceptable and easily accessible.

The ability of molecules to permeate through the oral mucosa appears to be related to molecular size, lipid solubility and peptide protein ionization. Small molecules, less than 1000 daltons appear to cross mucosa rapidly. As molecular size increases, the permeability decreases rapidly. Lipid soluble compounds are more permeable than non-lipid soluble molecules. Maximum absorption occurs when molecules are un-ionized or neutral in electrical charges. Therefore charged molecules present the biggest challenges to absorption through the oral mucosae.

A pharmaceutical composition of iRNA may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity. For example, the medication can be sprayed into the buccal cavity or applied directly, e.g., in a liquid, solid, or gel form to a surface in the buccal cavity. This administration is particularly desirable for the treatment of inflammations of the buccal cavity, e.g., the gums or tongue, e.g., in one embodiment, the buccal administration is by spraying into the cavity, e.g., without inhalation, from a dispenser, e.g., a metered dose spray dispenser that dispenses the pharmaceutical composition and a propellant.

An aspect of the invention also relates to a method of delivering an oligonucleotide into the CNS by intrathecal or intracerebroventricular delivery, or into an ocular tissue by ocular delivery, e.g., an intravitreal delivery.

Some embodiments relates to a method of reducing the expression of a target gene in a cell, comprising contacting said cell with an oligonucleotide having one or more lipophilic monomers containing lipophilic moieties conjugated to oligonucleotide, optionally via a linker or carrier. In one embodiment, the cell is a cell in the CNS system. In one embodiment, the cell is an ocular cell.

Some embodiments relates to a method of reducing the expression of a target gene in a subject, comprising administering to the subject an oligonucleotide having one or more lipophilic monomer containing lipophilic moieties conjugated to oligonucleotide, optionally via a linker or carrier. In one embodiment, the oligonucleotide conjugate is administered intrathecally or intracerebroventricularly (to reduce the expression of a target gene in a brain or spine tissue). In one embodiment, the oligonucleotide conjugate is administered ocularly, e.g., intravitreally, (to reduce the expression of a target gene in an ocular tissue).

In some embodiments, the oligonucleotide is double-stranded. In one embodiment, the oligonucleotide is a compound comprising an antisense strand which is complementary to a target gene and a sense strand which is complementary to said antisense strand.

In some embodiments, the oligonucleotide is single-stranded. In one embodiment, the oligonucleotide is an antisense.

In some embodiments, the lipophilic monomer containing a lipophilic moiety is located on one or more internal positions on at least one strand of the oligonucleotide. In some embodiments, the lipophilic monomer containing a lipophilic moiety is located on one or more terminal positions on at least one strand of the oligonucleotide.

The invention is further illustrated by the following examples, which should not be construed as further limiting. The contents of all references, pending patent applications and published patents, cited throughout this application are hereby expressly incorporated by reference.

### EXAMPLES

The invention now being generally described, it will be more readily understood by reference to the following examples which are included merely for purposes of illustration of certain aspects and embodiments of the present invention and are not intended to limit the invention.

### Example 1. Synthesis of Lipophilic Monomers

Lipophilic monomers were synthesized to introduce lipophilic ligands at various locations of siRNAs (terminal and/or internal positions) as solid support or phosphoramidites. A variety of lipids can be conjugated via hydroxyprolinol derivatives using methods as shown in the schemes below (e.g., Schemes 1-3 for general procedures), and the resulting building block phosphoramidites can be incorporated into siRNAs.

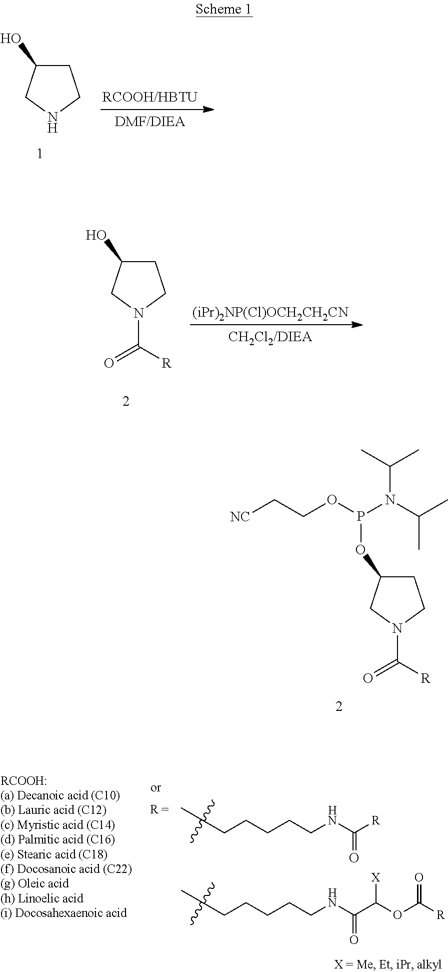

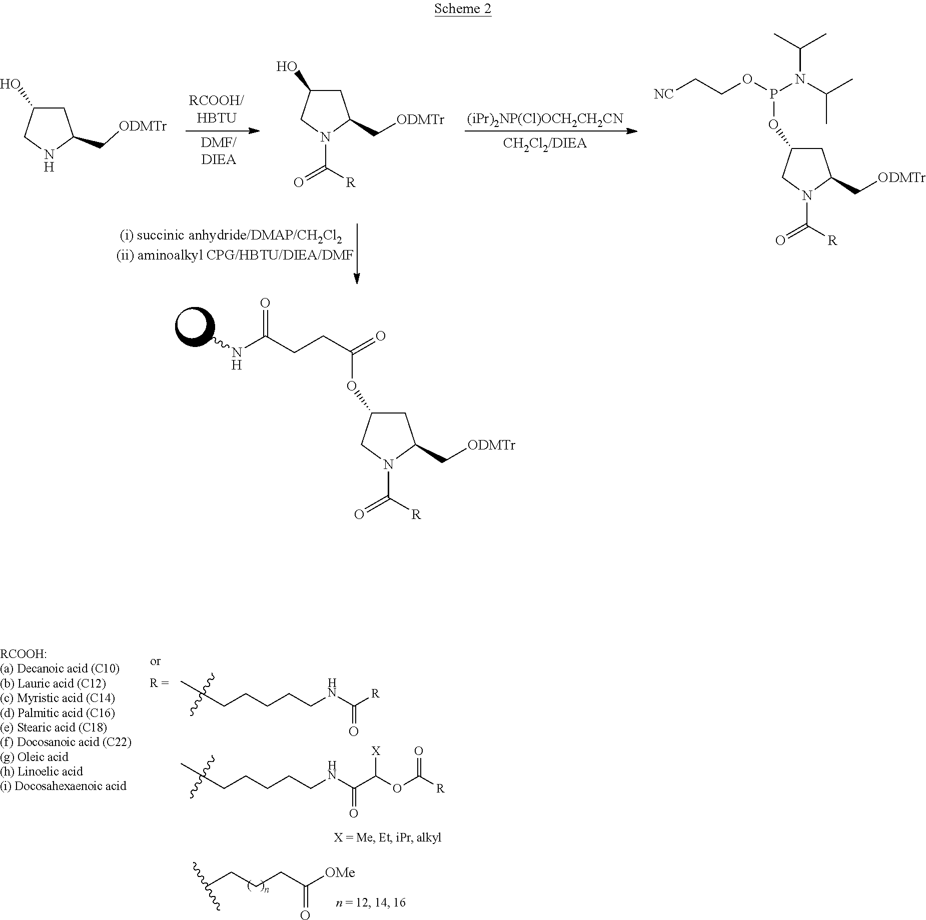

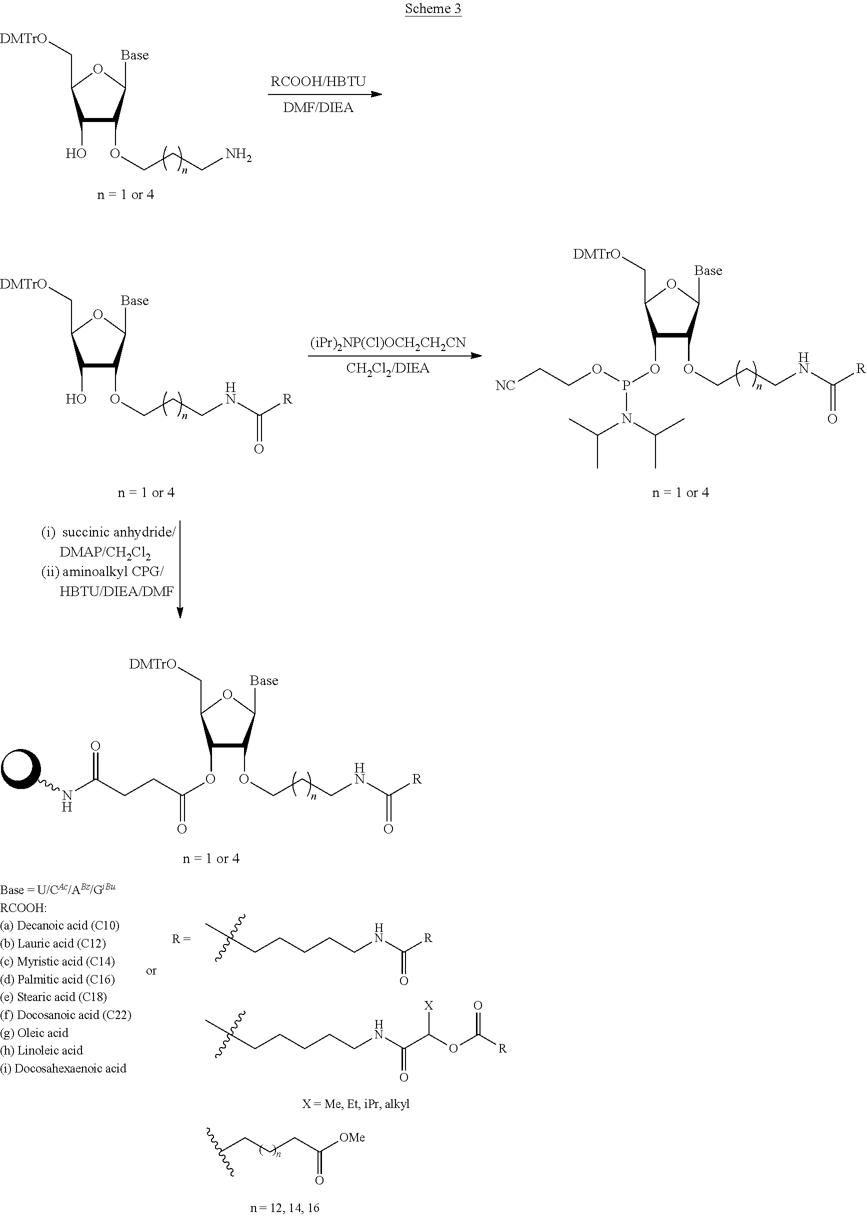

### Synthesis of Lipophilic Conjugate on Prolinol at 5′ End

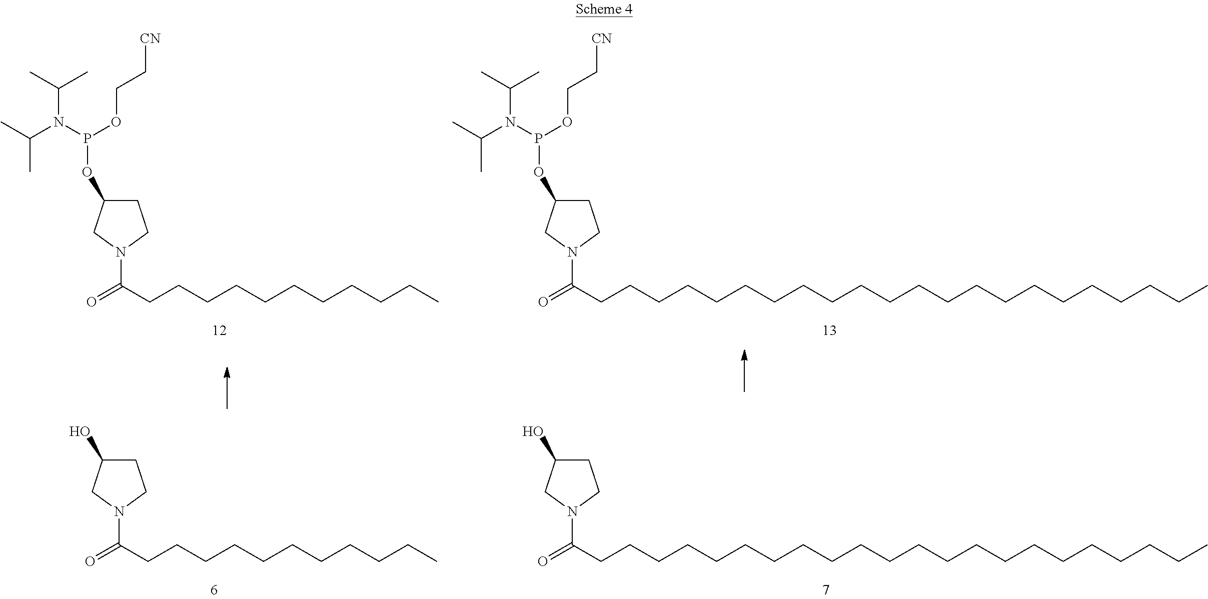

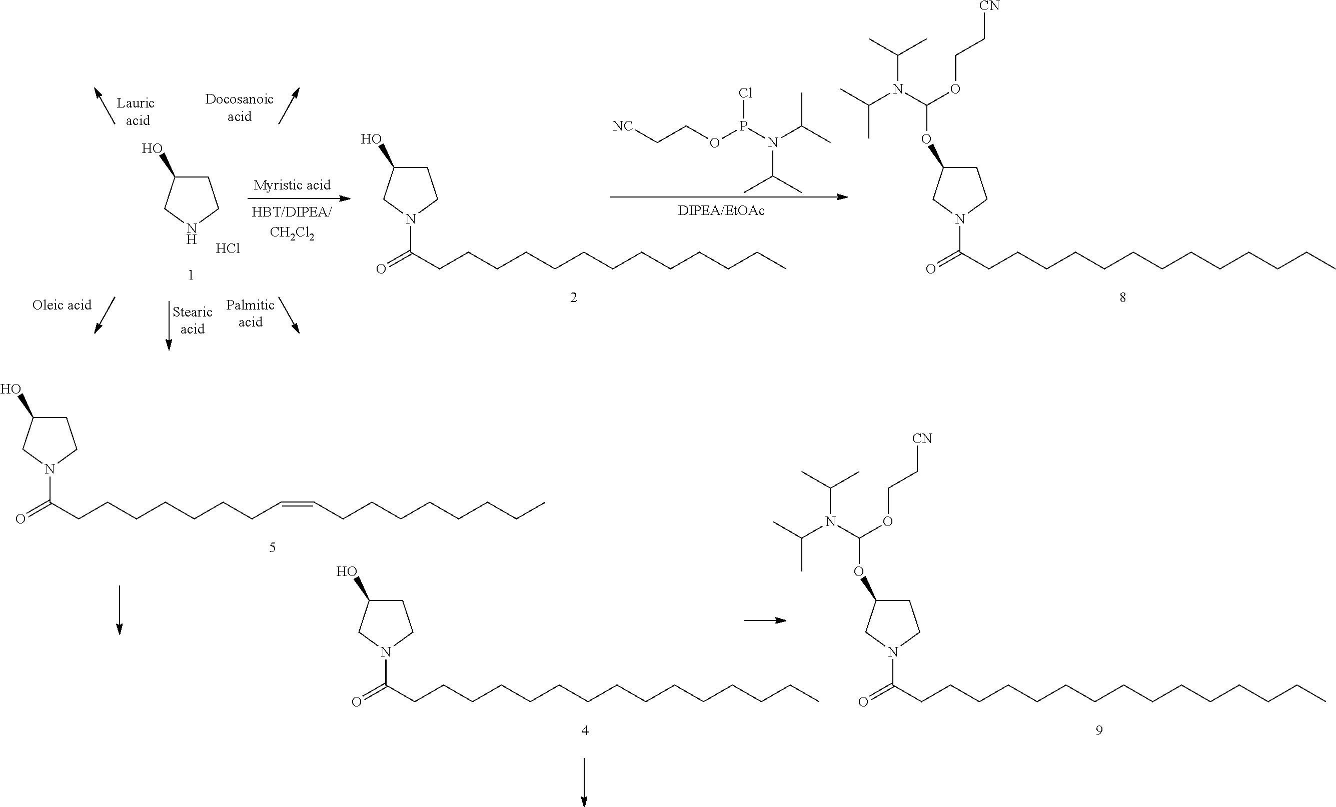

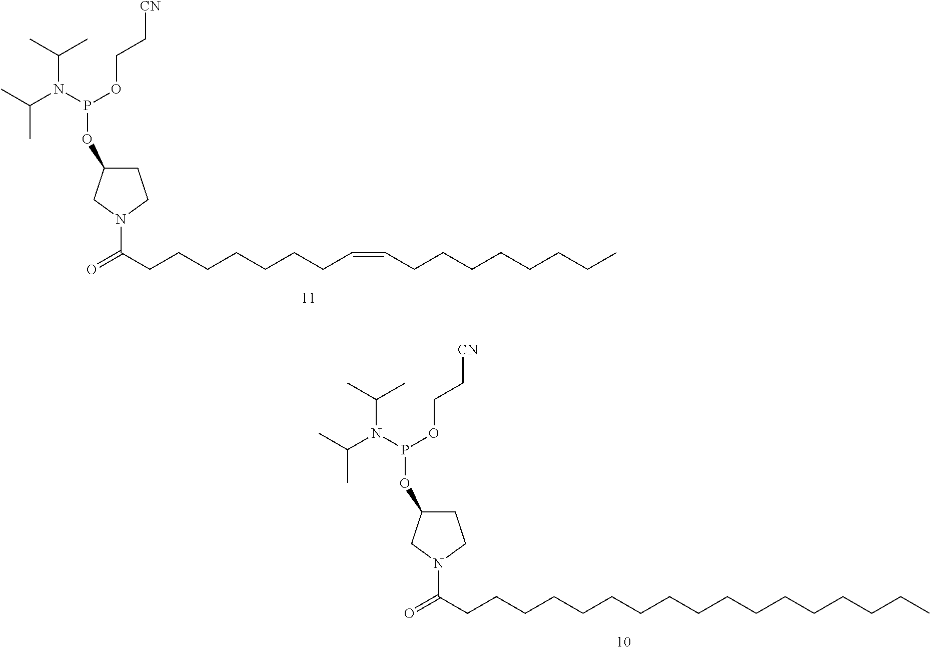

Compound 2: To a heat-oven dried 100 mL round bottle flask, added a solution of Compound 1, (3 g, 24.28 mmol, 1.0 equiv.) in anhydrous DCM (50 mL). Tetradecanoic acid 2a (6.10 g, 26.70 mmol, 1.1 eq.) was added to the solution, followed by HBTU (10.13 g, 26.70 mmol, 1.1 eq.) and DIPEA (12.68 mL, 72.53 mmol, 3 eq.). The resultant solution was stirred at room temperature under argon overnight. TLC with 80% EtOAc/hexane showed the formation of the product. The reaction mixture was quenched with brine solution, and extracted with DCM. The combined organic solution was dried over anhydrous Na₂SO₄, filtered and concentrated to an oil form residue. Purification through ISCO column chromatography with 80 g silica gel column eluted compound 2 with 0-70% EtOAc/hexane. A white oily compound was yielded (7.2 g). ¹H NMR (400 MHz, chloroform-d) δ 4.58-4.45 (m, 1H), 3.70-3.37 (m, 4H), 2.31-2.18 (m, 2H), 2.09-1.87 (m, 3H), 1.63 (t, J=7.4 Hz, 2H), 1.36-1.27 (m, 6H), 1.25 (s, 14H), 0.87 (t, J=6.8 Hz, 3H). M+1=298.3.

Compound 3: Compound 3 was obtained by using Compound 1 and palmitic acid in a procedure similar to the procedure above for synthesizing Compound 2. ¹H NMR (500 MHz, chloroform-d) δ 8.00 (s, 1H), 3.67-3.47 (m, 2H), 2.95 (s, 3H), 2.87 (s, 3H), 2.79 (s, 6H), 2.30-2.18 (m, 1H), 2.04 (h, J=3.5 Hz, 1H), 1.62 (p, J=7.2, 6.8 Hz, 2H), 1.32-1.26 (m, 4H), 1.24 (s, 11H), 0.87 (t, J=6.8 Hz, 2H). M+1=326.4.

Compound 4: Compound 4 was obtained by using Compound 1 and stearic acid in a procedure similar to the procedure above for synthesizing Compound 2. ¹H NMR (400 MHz, chloroform-d) δ 4.57-4.45 (m, 1H), 3.56 (dddd, J=31.4, 13.1, 10.0, 6.5 Hz, 4H), 2.80 (s, 3H), 2.31-2.18 (m, 3H), 2.04 (td, J=5.8, 2.9 Hz, 1H)), 1.28 (d, J=8.1 Hz, 28H), 0.87 (t, J=6.7 Hz, 3H). M+1=354.4.

Compound 5: Compound 5 was obtained by using compound 1 and oleic acid in a procedure similar to the procedure above for synthesizing Compound 2. ¹H NMR (400 MHz, chloroform-d) δ 5.40-5.27 (m, 2H), 3.67-3.46 (m, 4H), 2.80 (s, 9H), 2.36-2.16 (m, 3H), 1.36-1.21 (m, 20H), 0.91-0.83 (m, 3H). M+1=352.3.

Compound 6: Compound 6 was obtained by using compound 1 and dodecanoic acid in a procedure similar to the procedure above for synthesizing Compound 2. M+1=270.3.

Compound 7: Compound 7 was obtained by using compound 1 and docosanoic acid in a procedure similar to the procedure above for synthesizing Compound 2. ¹H NMR (400 MHz, chloroform-d) δ 4.52 (d, J=18.9 Hz, 2H), 3.69-3.15 (m, 5H), 2.32-2.18 (m, 2H), 2.03 (ddp, J=13.4, 9.0, 4.4 Hz, 2H), 1.73-1.60 (m, 3H), 1.32 (t, J=9.6 Hz, 8H), 1.25 (s, 25H), 0.88 (t, J=6.6 Hz, 3H). M+1=410.4.

Compound 8: Compound 2 (7.2 g, 24.2 mmol, 1 eq.) was dissolved in anhydrous EtOAc (120 mL). In an ice bath and under argon, DIPEA (12.65 mL, 72.61 mmol, 3 eq.) was added to the solution, followed by N,N-diisopropylaminocyanoethyl phosphonamidic-Cl (6.30 g, 26.61 mmol, 1.1 eq.). The resultant reaction mixture was stirred at room temperature overnight. TLC at 50% EtOAc/hexane showed the completion of the reaction. The reaction mixture was quenched with brine, and extracted with EtOAc. The organic layer was separated, dried over Na₂SO₄ and concentrated to a white oil. ISCO purification eluted Compound 8 with 0-50% EtOAc/hexane, with a yield of 65% (7.71 g). ¹H NMR (400 MHz, acetonitrile-d₃) δ 4.54 (dddt, J=17.4, 10.1, 5.8, 2.8 Hz, 1H), 3.88-3.34 (m, 7H), 2.66 (q, J=5.7 Hz, 2H), 2.33-2.15 (m, 3H), 2.09 (ddt, J=11.9, 7.8, 3.9 Hz, 1H), 1.62-1.51 (m, 2H), 1.38-1.25 (m, 20H), 1.25-1.13 (m, 13H), 0.95-0.87 (m, 3H). ³¹P NMR (162 MHz, CD₃CN) δ 147.33, 147.15, 146.97, 146.88.

Compound 9: Compound 9 was obtained using Compound 3 and N,N-diisopropylamino-cyanoethyl phosphonamidic-Cl in a procedure similar to the procedure above for synthesizing Compound 8. ¹H NMR (400 MHz, Acetonitrile-d₃) δ 4.61-4.43 (m, 1H), 3.87-3.70 (m, 2H), 3.70-3.34 (m, 6H), 2.67 (t, J=5.8 Hz, 2H), 2.33-2.14 (m, 3H), 2.09 (ddt, J=12.1, 7.9, 3.9 Hz, 1H), 1.30 (s, 25H), 1.25-1.14 (m, 13H), 0.97-0.87 (m, 3H). ³¹P NMR (162 MHz, CD₃CN) 6147.33, 147.15, 146.97, 146.88.

Compound 10: Compound 10 was obtained using Compound 4 and N, N-diisopropylamino-cyanoethyl phosphonamidic-Cl in a procedure similar to the procedure above for synthesizing Compound 8. ¹H NMR (400 MHz, acetonitrile-d₃) δ 4.66-4.40 (m, 1H), 3.87-3.34 (m, 8H), 2.67 (t, J=5.8 Hz, 2H), 2.30-2.16 (m, 3H), 2.15-2.02 (m, 1H), 1.30 (s, 27H), 1.29-1.16 (m, 15H), 0.95-0.87 (m, 3H). ³¹P NMR (162 MHz, CD₃CN) δ 147.32, 147.15, 146.97, 146.88.

Compound 11: Compound 11 was obtained using Compound 5 and N, N-diisopropylamino-cyanoethyl phosphonamidic-Cl in a procedure similar to the procedure above for synthesizing Compound 8. ¹H NMR (400 MHz, acetonitrile-d₃) δ 5.43-5.33 (m, 2H), 4.54 (dddd, J=20.3, 9.7, 4.8, 2.1 Hz, 1H), 3.88-3.72 (m, 2H), 3.72-3.34 (m, 6H), 2.66 (q, J=5.7 Hz, 2H), 2.33-2.16 (m, 4H), 1.42-1.28 (m, 21H), 1.28-1.14 (m, 14H), 0.95-0.87 (m, 3H). ³¹P NMR (162 MHz, CD₃CN) δ 147.34, 147.17, 147.00, 146.90.

Compound 12: Compound 12 was obtained using Compound 6 and N, N-diisopropylamino-cyanoethyl phosphonamidic-Cl in a procedure similar to the procedure above for synthesizing Compound 8. ¹H NMR (400 MHz, acetonitrile-d₃) δ 4.63-4.43 (m, 1H), 3.88-3.70 (m, 2H), 3.70-3.34 (m, 6H), 2.67 (t, J=5.8 Hz, 2H), 2.33-2.15 (m, 5H), 2.09 (ddt, J=12.3, 8.1, 3.9 Hz, 1H), 1.40-1.13 (m, 29H), 0.95-0.87 (m, 3H). ³¹P NMR (162 MHz, CD₃CN) δ 147.33, 147.15, 146.97, 146.86.

Compound 13: Compound 13 was obtained using Compound 7 and N, N-diisopropylamino-cyanoethyl phosphonamidic-Cl in a procedure similarly to the procedure above for synthesizing Compound 8. ¹H NMR (400 MHz, acetonitrile-d₃) δ 4.64-4.38 (m, 1H), 3.86-3.70 (m, 2H), 3.70-3.34 (m, 6H), 2.66 (q, J=5.7 Hz, 2H), 2.32-2.15 (m, 3H), 1.30 (s, 37H), 1.25-1.12 (m, 13H), 0.95-0.87 (m, 3H). ³¹P NMR (162 MHz, CD₃CN) δ 148.29, 147.33, 147.19, 147.01, 146.94.

### Synthesis of Terminal Acid-Containing Lipophilic Conjugate on Prolinol at 5′ End

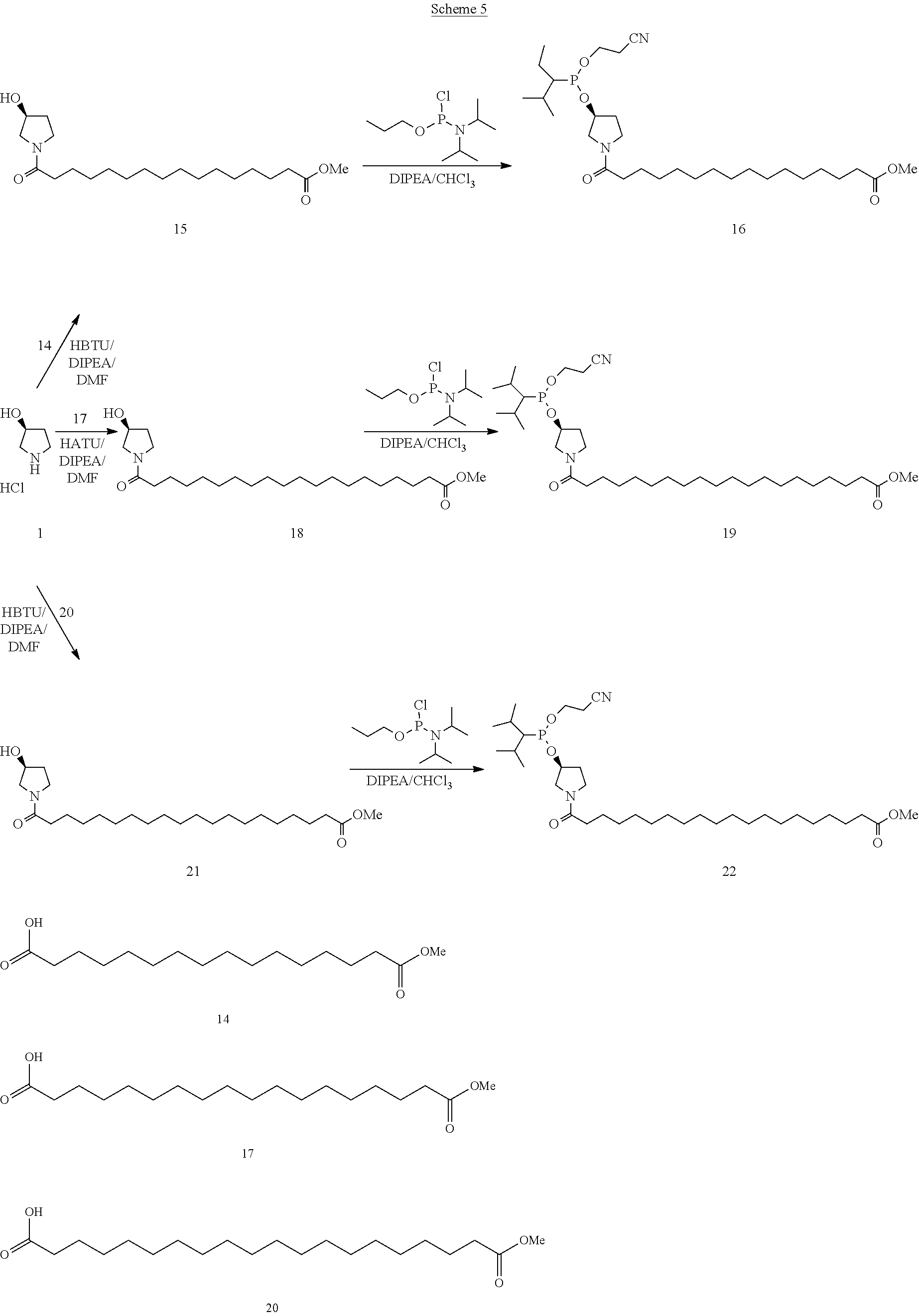

Compound 15: A 3-L, three-neck round bottle flask equipped with a mechanical stirrer was charged with Compound 14 (15 g, 49.9 mmol, 1 eq.), HBTU (20.8 g, 54.9 mmol) and anhydrous DMF (350 mL). The mixture was stirred for 30 minutes to dissolve the starting materials and then DIPEA (17.3 mL, 99.8 mmol) was added dropwise while vigorously stirring at room temperature. The mixture was stirred at room temperature for 1.5 hours and then cooled to 0° C. A mixture of (S)-3-pyrrolidinol 1 (6.78 g, 54.9 mmol) and DIPEA (17.3 mL, 99.8 mmol) in DMF (110 mL) was added dropwise to the reaction mixture at 0° C. over 30 minutes, and then warmed to room temperature. The reaction mixture was stirred at room temperature for 12 hours. The reaction progress was monitored by TLC (5% MeOH/ethylacetate or 50% ethylacetate/hexanes). The reaction mixture was cooled to 0-5° C. and diluted with water (1.5 L), stirred for 30 minutes, and then filtered to collect brown solid Compound 15, which was purified by column chromatography to afford Compound 15 as light brown solid (17 g, 92% yield). ¹H NMR (600 MHz, CDCl₃): δ 4.52 (d, 1H, J=30 Hz); 3.66 (s, 3H); 3.60-3.51 (m, 2H); 3.41 (d, 1H, 12 Hz); 2.34-2.20 (m, 4H); 2.07-2.01 (m, 4H); 1.68-1.56 (m, 4H); 1.36-1.20 (m, 20H).

Compound 16: An oven dried 500 mL single-neck round bottle flask was charged with Compound 15 (8 g, 21.6 mmol, 1 eq.) and chloroform (100 mL) under argon. The reaction mixture was cooled to 0° C., and then DIPEA was added followed by dropwise addition of 2-cyanoethyl-N,N-diisopropyl-chlorophosphoramidite (5.31 mL, 23.8 mmol) at 0° C. The reaction mixture was slowly warmed to room temperature and stirred for 3 hours. The reaction progress was monitored by TLC. The reaction mixture was cooled to 0° C., quenched with MeOH (3 ml), stirred for 30 minutes, and then concentrated to afford crude product 16, which was purified by silica gel column chromatography. Pure fractions were combined, and concentrated to afford Compound 16 as thick syrup (4.38 g, 36% yield). ¹H NMR (600 MHz, CD₃CN): δ 4.58-4.45 (m, 1H); 4.08-3.93 (m, 2H); 3.82-3.68 (m, 2H); 3.65 (s, 3H); 3.27-3.20 (m, 1H); 2.72-2.59 (m, 4H); 2.27 (t, J=6 Hz, 2H); 1.94-1.93 (m, 4H); 1.58-1.48 (m, 6H); 1.33-1.21 (m, 20H); 1.19-1.14 (m, 12H). ³¹P NMR (243 MHz, CD₃CN): 147.34, 147.16, 146.99, 146.89.

Compound 18: A 3-L, three neck round bottle flask equipped with a mechanical stirrer was charged with Compound 17 (14 g, 42.6 mmol, 1 eq.), HBTU (17.8 g, 46.9 mmol), and anhydrous DMF (330 mL). The mixture was stirred for 30 minutes to dissolve solids, and then DIPEA (14.8 mL, 85.2 mmol) was added dropwise while vigorously stirring at room temperature. The reaction mixture was stirred at room temperature for 1.5 hours, and then cooled to 0° C. A mixture of (S)-3-pyrrolidinol 1 (5.79 g, 46.9 mmol) and DIPEA (14.8 mL, 85.2 mmol) in anhydrous DMF (125 mL) was added dropwise to the reaction mixture at 0° C. over 30 minutes. The mixture was warmed to room temperature and stirred for 18 hours. The reaction progress was monitored by TLC (5% MeOH/ethyl acetate). The mixture was cooled to 0-5° C., quenched with water (1.5 L) slowly, stirred for 30 minutes, and then filtered to collect brown solid Compound 18. The crude product was purified by column chromatography to afford Compound 18 as light brown solid (16.1 g, 95% yield). ¹H NMR (600 MHz, CDCl₃): δ 4.53 (d, 1H, J=30 Hz); 3.66 (s, 3H); 3.60-3.49 (m, 2H); 3.41 (d, 1H, 12 Hz); 2.33-2.21 (m, 4H); 2.04-2.03 (m, 4H); 1.64-1.58 (m, 4H); 1.33-1.22 (m, 24H).

Compound 19: An oven dried 500 mL, single-neck round bottle flask was charged with Compound 18 (13 g, 32.6 mmol, 1 eq.) and chloroform (130 mL) under argon. The mixture was cooled to 0° C. and catalytic amounts of DMAP and DIPEA (17.1 mL, 98.0 mmol, 3 eq.) were added, followed by dropwise addition of 2-cyanoethyl-N,N-diisopropylchlorophosphoramidite (8.02 mL, 35.9 mmol) over a period of 15 minutes. The reaction mixture was warmed to room temperature and stirred for 5 hours. The reaction progress was monitored by TLC (5% MeOH/ethyl acetate). The mixture was cooled to 0° C., quenched with MeOH (7 ml), stirred for 1 hour, and then concentrated to afford crude product 19. The crude product was purified by silica gel column chromatography. Pure fractions were combined, concentrated, and dried under high vacuum to afford Compound 19 as thick syrup (10.17 g, 52% yield). ¹H NMR (600 MHz, CD₃CN): δ 4.58-4.45 (m, 1H); 4.08-3.93 (m, 2H); 3.82-3.68 (m, 2H); 3.65 (s, 3H); 3.27-3.20 (m, 1H); 2.72-2.59 (m, 4H); 2.27 (t, J=6 Hz, 2H); 1.94-1.93 (m, 4H); 1.58-1.48 (m, 6H); 1.33-1.21 (m, 20H); 1.19-1.14 (m, 12H). ³¹P NMR (243 MHz, CD₃CN): 147.4, 147.3, 147.2, 147.0, 146.9.

Compound 21: A 3-L, three-neck round bottle flask equipped with a mechanical stirrer was charged with Compound 20 (15 g, 35.2 mmol, 1 eq.), HBTU (14.7 g, 38.7 mmol) and DMF (600 mL). The mixture was stirred for 30 minutes to dissolve solids, and DIPEA (12.3 mL, 70.5 mmol) was added dropwise while vigorously stirring at room temperature. The reaction mixture was stirred at room temperature for 1.5 hours, and then cooled to 0° C. A mixture of (S)-3-pyrrolidinol 1 (4.79 g, 38.7 mmol) and DIPEA (12.3 mL, 70.5 mmol) in anhydrous DMF (110 mL) was added dropwise to the reaction mixture at 0° C. over 30 minutes, and then warmed to room temperature. The reaction mixture was stirred at room temperature for 15 hours. The reaction progress was monitored by TLC (5% MeOH/ethyl acetate). The mixture was cooled to 0-5° C., quenched with water (1.5 L) slowly, stirred for 1.5 hours, and then filtered to collect brown solid Compound 21, which was purified by column chromatography to afford Compound 21 as light brown solid (16.1 g, 90% yield). ¹H NMR (600 MHz, CDCl₃): δ 4.52 (d, 1H, J=30 Hz); 3.66 (s, 3H); 3.62-3.51 (m, 2H); 3.39 (d, 1H, 12 Hz); 2.31-2.19 (m, 4H); 2.06-2.02 (m, 4H); 1.62-1.55 (m, 4H); 1.31-1.26 (m, 28H).

Compound 22: An oven dried 500 mL single-neck round bottle flask was charged with Compound 21 (16 g, 37.6 mmol, 1 eq.) and chloroform (200 mL) under argon. The mixture was cooled to 0° C., and catalytic amounts of DMAP and DIPEA (14.4 mL, 83.0 mmol, 3 eq.) were added, followed by dropwise addition of 2-cyanoethyl-N,N-diisopropylchlorophosphoramidite (6.78 mL, 30.4 mmol) over a period of 15 minutes. The reaction mixture was warmed to room temperature and stirred for 4 hours. The reaction progress was monitored by TLC (5% MeOH/ethyl acetate). The mixture was cooled to 0° C., quenched with MeOH (7 ml), stirred for 30 minutes, and then concentrated to afford crude product 6, which was purified by silica gel column chromatography. Pure fractions were combined, concentrated, and dried under high vacuum to afford Compound 22 as thick syrup (9.7 g, 41% yield). ¹H NMR (600 MHz, CDCN): δ 4.58-4.55 (m, 1H); 4.08-3.93 (m, 2H); 3.83-3.67 (m, 2H); 3.65 (m, 4H); 2.68-2.59 (m, 4H); 2.27 (t, J=6 Hz, 2H); 1.97-1.91 (m, 4H); 1.60-1.49 (m, 6H); 1.33-1.21 (m, 28H); 1.19-1.14 (m, 12H). ³¹P NMR (243 MHz, CD₃CN): 147.34, 147.16, 146.99, 146.90.

### Synthesis of Lipophilic Conjugate on Prolinol at 3′ End

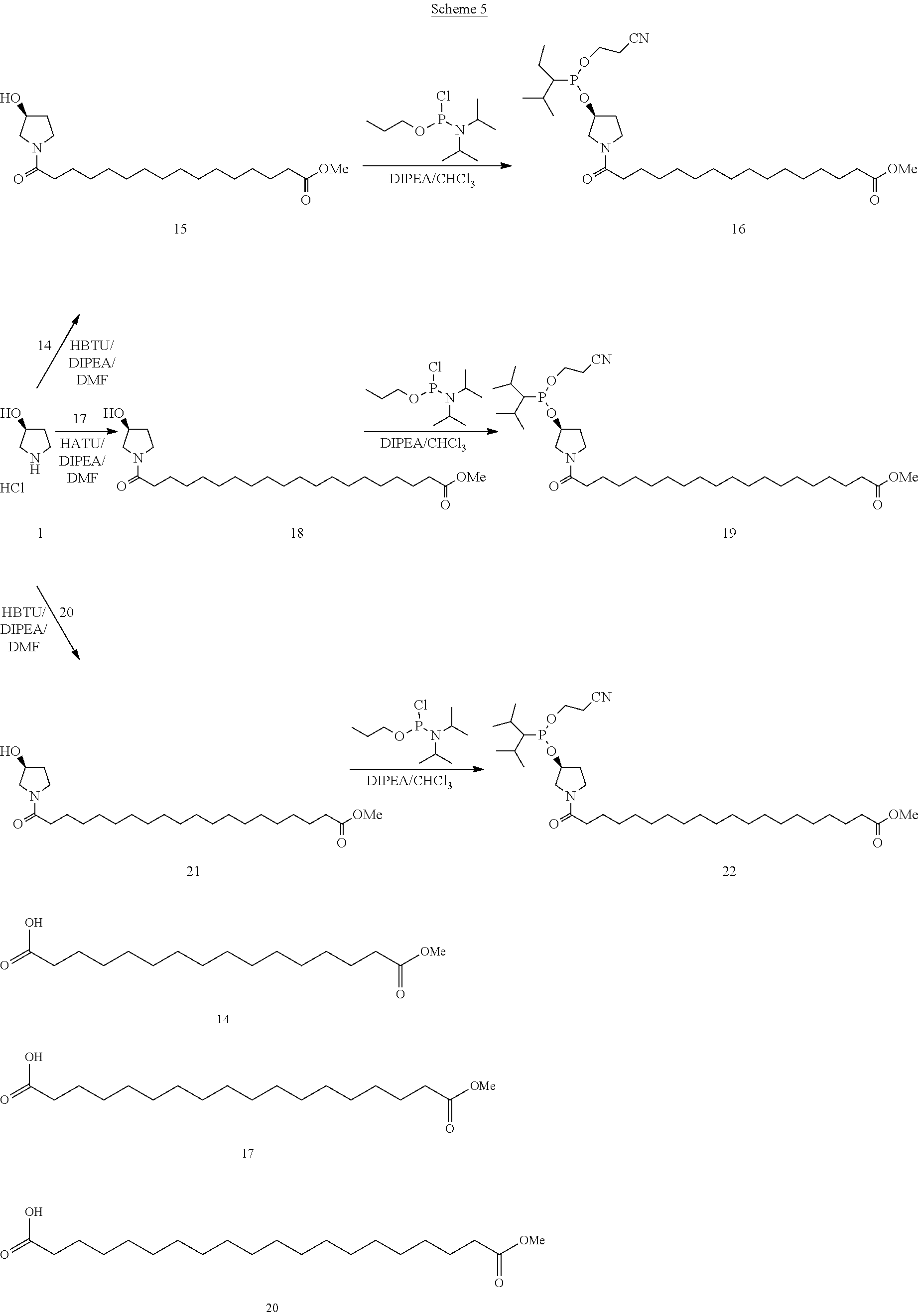

Compound 22: Compound 22 was synthesized using Compound 21 and myristic acid under standard peptide coupling conditions in CH₂Cl₂. ¹H NMR (400 MHz, DMSO) δ 7.35-7.26 (m, 6H), 7.25-7.15 (m, 7H), 6.90-6.83 (m, 6H), 4.97 (d, J=4.0 Hz, 1H), 4.39 (dd, J=8.8, 4.3 Hz, 1H), 4.28 (dd, J=9.6, 4.4 Hz, 1H), 4.18-4.08 (m, 1H), 3.73 (s, 9H), 3.57 (dt, J=10.2, 5.1 Hz, 1H), 3.35-3.30 (m, 4H), 3.28-3.20 (m, 1H), 3.17 (dd, J=8.8, 5.0 Hz, 1H), 3.01-2.94 (m, 2H), 2.69 (s, 9H), 2.25-2.16 (m, 2H), 2.10-2.05 (m, 2H), 1.83 (ddd, J=12.8, 8.4, 4.7 Hz, 1H), 1.51-1.40 (m, 2H), 1.20 (d, J=18.9 Hz, 30H), 0.90-0.81 (m, 5H).

Compound 23: Compound 23 was synthesized using Compound 21 and palmitic acid under standard peptide coupling conditions in CH₂Cl₂. ¹H NMR (400 MHz, DMSO) δ 7.36-7.24 (m, 7H), 7.24-7.15 (m, 8H), 6.91-6.81 (m, 7H), 4.97 (s, 1H), 4.39 (t, J=4.8 Hz, 1H), 4.20-4.07 (m, 2H), 3.71 (d, J=12.4 Hz, 10H), 3.57 (dt, J=10.5, 5.3 Hz, 1H), 3.38-3.28 (m, 4H), 3.18 (dd, J=8.8, 5.0 Hz, 1H), 3.02-2.94 (m, 2H), 2.71-2.64 (m, 14H), 2.20 (t, J=7.4 Hz, 2H), 2.02-1.96 (m, 4H), 1.46 (q, J=7.1 Hz, 2H), 1.30-1.20 (m, 33H), 0.84 (t, J=6.6 Hz, 5H).

Compound 24: Compound 24 was synthesized using Compound 21 and stearic acid under standard peptide coupling conditions in CH₂Cl₂. ¹H NMR (400 MHz, DMSO) δ 7.35-7.25 (m, 6H), 7.23-7.15 (m, 8H), 6.90-6.83 (m, 6H), 4.97 (d, J=4.0 Hz, 1H), 4.42-4.36 (m, 1H), 4.18-4.11 (m, 1H), 3.72 (s, 9H), 3.57 (dt, J=10.1, 5.1 Hz, 1H), 3.45 (dd, J=12.1, 3.9 Hz, 1H), 3.24 (dd, J=12.1, 5.6 Hz, 1H), 3.18 (dd, J=8.8, 5.0 Hz, 1H), 3.02-2.95 (m, 2H), 2.69 (s, 14H), 2.20 (t, J=7.4 Hz, 2H), 2.04-1.96 (m, 2H), 1.52-1.43 (m, 2H), 1.30-1.14 (m, 40H), 0.84 (t, J=6.7 Hz, 4H).

Compound 25: Compound 25 was synthesized using Compound 21 and oleic acid under standard peptide coupling conditions in CH₂Cl₂. ¹H NMR (400 MHz, DMSO) δ 7.36-7.24 (m, 6H), 7.24-7.15 (m, 7H), 6.90-6.83 (m, 6H), 5.35-5.26 (m, 3H), 4.97 (d, J=3.9 Hz, 1H), 4.39 (d, J=5.3 Hz, 1H), 4.20-4.07 (m, 2H), 3.71 (d, J=12.7 Hz, 9H), 3.57 (dt, J=8.8, 4.4 Hz, 1H), 3.17 (dd, J=8.9, 5.1 Hz, 1H), 3.02-2.94 (m, 2H), 2.67 (d, J=13.5 Hz, 13H), 2.22-2.16 (m, 2H), 2.02-1.92 (m, 7H), 1.47 (t, J=7.1 Hz, 2H), 1.25 (t, J=11.6 Hz, 26H), 0.83 (td, J=6.4, 2.1 Hz, 4H).

Compound 26: To a solution of Compound 22 (5.67 g, 9.00 mmol) in anhydrous dichloromethane (86.26 mL), DMAP (1.10 g, 9.00 mmol) and succinic anhydride (1.80 g, 18.00 mmol) were added. The mixture was cooled to 0° C., and triethylamine (3.76 mL, 27.01 mmol) was added dropwise. The reaction mixture was stirred at room temperature for 18 hours, at which point no presence of starting material was shown (5% Et₃N in 5% MeOH in DCM). The mixture was concentrated under reduced pressure. The residue was purified by flash chromatography on silica gel (pre-treated with Et₃N) with gradient 0-5% MeOH in DCM to afford 4.91 g (75% yield) of the succinate. To a solution of the succinate (4.91 g, 6.73 mmol) in anhydrous DMF (331.64 mL), DIPEA (4.69 mL, 26.91 mmol) was added and then stirred until fully dissolved. HBTU (2.68 g, 7.06 mmol) was added to the mixture and stirred for 5 minutes. Controlled pore glass (CPG) (152 μmol/g, 48.68 g, 7.40 mmol) was added to the mixture. The round bottle flask was capped with a rubber septum, securely parafilmed, and then shaken on a mechanical shaker overnight. The mixture was filtered through a glass fritted funnel under vacuum, and rinsed in parallel with acetonitrile, methanol, acetonitrile, and diethyl ether (300 mL each). The filtrate was discarded, and the filtered material was vacuum dried on frit for 20 minutes. The filtered material was returned to the original flask and dried on high vacuum overnight. The loading of material on solid support was checked by UV-Vis and Beer's law on a Beckman Coulter spectrophotometer. The solid support material was weighed (53.5 mg) and dissolved in 0.1 M p-toluenesulfonic acid in acetonitrile in a 250 mL volumetric flask. The mixture was sonicated and allowed to sit undisturbed for 1 hour. The machine was blanked with the same solvent and the UV absorbance at 411 nm of the solution was measured in triplicate. The rest of the solid support materials was capped using 30% acetic anhydride in pyridine with 1% Et₃N (325 mL). The flask was capped and parafilmed, and then shaken on mechanical shaker for 3 hours. The mixture was filtered on glass frit funnel under vacuum and washed in order: 10% H₂O in THF, MeOH, 10% H₂O in THF, MeOH, ACN, and diethyl ether (300 mL each). The filtrates were discarded, and the solid support material was dried on frit under vacuum. The solid support material was transferred to a round bottle flask, and then dried on high vacuum overnight to afford Compound 26 (48.96 g, 106.92 μmol/g loading).

Compound 27: To a solution of Compound 23 (5.10 g, 7.75 mmol) in anhydrous dichloromethane (74.28 mL), DMAP (947 mg, 7.75 mmol) and succinic anhydride (1.55 g, 15.50 mmol) were added. The mixture was cooled to 0° C., and triethylamine (3.24 mL, 23.26 mmol) was added dropwise. The reaction mixture was stirred at room temperature for 18 hours, at which point no presence of starting material was shown (5% Et₃N in 5% MeOH in DCM). The mixture was concentrated under reduced pressure. The residue was purified by flash chromatography on silica gel (pre-treated with Et₃N) with gradient 0-5% MeOH in DCM to afford 3.85 g (65% yield) of the succinate. ¹H NMR (400 MHz, DMSO-d₆) δ 7.36-7.24 (m, 6H), 7.20 (ddd, J=8.9, 6.0, 3.1 Hz, 7H), 6.87 (ddd, J=8.9, 5.2, 2.4 Hz, 6H), 5.36 (t, J=4.4 Hz, 1H), 4.20 (dq, J=9.2, 4.7, 4.2 Hz, 1H), 3.73 (s, 10H), 3.55 (dd, J=11.4, 3.0 Hz, 1H), 3.24 (dd, J=9.0, 4.6 Hz, 1H), 3.03 (ddd, J=20.0, 9.9, 3.9 Hz, 2H), 2.66 (q, J=7.2 Hz, 2H), 2.49-2.41 (m, 5H), 2.19 (ddp, J=22.3, 9.0, 5.1, 4.6 Hz, 4H), 2.06-1.91 (m, 1H), 1.50-1.41 (m, 2H), 1.30-1.14 (m, 32H), 1.01 (t, J=7.2 Hz, 2H), 0.84 (t, J=6.8 Hz, 4H). To a solution of the succinate (3.85 g, 5.08 mmol) in anhydrous DMF (250.42 mL), DIPEA (3.54 mL, 20.32 mmol) was added and then stirred until fully dissolved. HBTU (2.02 g, 5.33 mmol) was added to the mixture and stirred for 5 minutes. Controlled pore glass (CPG) (152 μmol/g, 36.77 g, 5.59 mmol) was added to the mixture. The round bottle flask was capped with a rubber septum, securely parafilmed, and then shaken on a mechanical shaker overnight. The mixture was filtered through a glass fritted funnel under vacuum, and rinsed in parallel with acetonitrile, methanol, acetonitrile, and diethyl ether (300 mL each). The filtrate was discarded, and the filtered material was vacuum dried on frit for 20 minutes. The filtered material was returned to the original flask and dried on high vacuum overnight. The loading of material on solid support was checked by UV-Vis and Beer's law on a Beckman Coulter spectrophotometer. The solid support material was weighed (59.7 mg) and dissolved in 0.1 M p-toluenesulfonic acid in acetonitrile in a 250 mL volumetric flask. The mixture was sonicated and allowed to sit undisturbed for 1 hour. The machine was blanked with the same solvent and the UV absorbance at 411 nm of the solution was measured in triplicate. The rest of the solid support materials was capped using 30% acetic anhydride in pyridine with 1% Et₃N (325 mL). The flask was capped and parafilmed, and then shaken on mechanical shaker for 3 hours. The mixture was filtered on glass frit funnel under vacuum and washed in order: 10% H₂O in THF, MeOH, 10% H₂O in THF, MeOH, ACN, and diethyl ether (300 mL each). The filtrates were discarded, and the solid support material was dried on frit under vacuum. The solid support material was transferred to a round bottle flask and dried on high vacuum overnight to afford Compound 27 (38.53 g, 112.87 μmol/g loading).

Compound 28: To a solution of Compound 24 (5.53 g, 8.06 mmol) in anhydrous dichloromethane (77.24 mL), DMAP (984 mg, 8.06 mmol) and succinic anhydride (1.61 g, 16.12 mmol) were added. The mixture was cooled to 0° C., and triethylamine (3.37 mL, 24.18 mmol) was added dropwise. The reaction mixture was stirred at room temperature for 18 hours, at which point no presence of starting material was shown (5% Et₃N in 5% MeOH in DCM). The mixture was concentrated under reduced pressure. The residue was purified by flash chromatography on silica gel (pre-treated with Et₃N) with gradient 0-5% MeOH in DCM to afford 5.18 g (81%) of the succinate. ¹H NMR (400 MHz, DMSO-d₆) δ 8.13-8.08 (m, 1H), 7.37-7.24 (m, 6H), 7.20 (ddd, J=9.1, 6.2, 3.3 Hz, 7H), 6.87 (ddd, J=8.7, 5.1, 2.4 Hz, 6H), 6.63-6.57 (m, 1H), 5.39-5.32 (m, 1H), 4.24-4.15 (m, 2H), 3.73 (s, 10H), 3.55 (dd, J=11.6, 3.0 Hz, 1H), 3.23 (dd, J=9.0, 4.6 Hz, 1H), 3.09-2.97 (m, 2H), 2.96 (s, 4H), 2.78 (q, J=7.2 Hz, 1H), 2.49-2.43 (m, 6H), 2.26-2.11 (m, 4H), 2.09-1.91 (m, 1H), 1.45 (q, J=7.1 Hz, 2H), 1.22 (d, J=4.9 Hz, 36H), 1.06 (t, J=7.2 Hz, 1H), 0.84 (t, J=6.8 Hz, 4H). To a solution of the succinate (5.18 g, 6.59 mmol) in anhydrous DMF (324.91 mL), DIPEA (4.59 mL, 26.36 mmol) was added and stirred until fully dissolved. HBTU (2.62 g, 6.92 mmol) was added to the mixture and stirred for 5 minutes. Controlled pore glass (CPG) (152 μmol/g, 47.69 g, 7.25 mmol) was added to the mixture. The round bottle flask was capped with a rubber septum, securely parafilmed, and then shaken on a mechanical shaker overnight. The mixture was filtered through a glass fritted funnel under vacuum and rinsed in parallel with acetonitrile, methanol, acetonitrile, and diethyl ether (300 mL each). The filtrate was discarded, and the filtered material was vacuum dried on frit for 20 minutes. The filtered material was returned to the original flask and dried on high vacuum overnight. The loading of material on solid support was checked by UV-Vis and Beer's law on a Beckman Coulter spectrophotometer. The solid support material was weighed (54.0 mg) and dissolved in 0.1 M p-toluenesulfonic acid in acetonitrile in a 250 mL volumetric flask. The mixture was sonicated and allowed to sit undisturbed for 1 hour. The machine was blanked with the same solvent and the UV absorbance at 411 nm of the solution was measured in triplicate. The rest of the solid support materials was capped using 30% acetic anhydride in pyridine with 1% Et₃N (325 mL). The flask was capped and parafilmed and then shaken on mechanical shaker for 3 hours. The mixture was filtered on glass frit funnel under vacuum and washed in order: 10% H₂O in THF, MeOH, 10% H₂O in THF, MeOH, ACN, and diethyl ether (300 mL each). The filtrates were discarded, and the solid support material was dried on frit under vacuum. The solid support material was transferred to a round bottle flask and dried on high vacuum overnight to afford Compound 28 (50.60 g, 108.88 μmol/g loading).

Compound 29: To a solution of Compound 25 (5.19 g, 7.59 mmol) in anhydrous dichloromethane (72.71 mL), DMAP (927 mg, 7.59 mmol) and succinic anhydride (1.52 g, 15.18 mmol) were added. The mixture was cooled to 0° C., and triethylamine (3.37 mL, 24.18 mmol) was added dropwise. The reaction mixture was stirred at room temperature for 18 hours, at which point no presence of starting material was shown (5% Et₃N in 5% MeOH in DCM). The mixture was concentrated under reduced pressure. The residue was purified by flash chromatography on silica gel (pre-treated with Et₃N) with gradient 0-5% MeOH in DCM to afford 5.47 g (92%) of compound 3d (R═C₁₈H₃₃). ¹H NMR (400 MHz, DMSO-d₆) δ 7.37-7.25 (m, 4H), 7.25-7.15 (m, 5H), 6.91-6.81 (m, 4H), 5.39-5.21 (m, 3H), 4.24-4.14 (m, 1H), 3.73 (s, 6H), 3.23 (dd, J=9.1, 4.6 Hz, 1H), 3.07-2.97 (m, 1H), 2.58 (q, J=7.2 Hz, 1H), 2.49-2.41 (m, 4H), 2.26-2.13 (m, 2H), 1.97 (q, J=6.9, 6.4 Hz, 4H), 1.45 (q, J=6.9 Hz, 1H), 1.24 (d, J=9.3 Hz, 19H), 0.99 (t, J=7.2 Hz, 2H), 0.83 (td, J=6.9, 1.9 Hz, 3H). To a solution of the succinate (5.47 g, 6.98 mmol) in anhydrous DMF (343.98 mL), DIPEA (4.86 mL, 27.91 mmol) was added then stirred until fully dissolved. HBTU (2.78 g, 7.33 mmol) was added to the mixture and stirred for 5 minutes. Controlled pore glass (CPG) (152 μmol/g, 50.46 g, 7.67 mmol) was added to the mixture. The round bottle flask was capped with a rubber septum, securely parafilmed, and then shaken on a mechanical shaker overnight. The mixture was filtered through a glass fritted funnel under vacuum and rinsed in parallel with acetonitrile, methanol, acetonitrile, and diethyl ether (300 mL each). The filtrate was discarded, and the filtered material was vacuum dried on frit for 20 minutes. The filtered material was returned to the original flask and dried on high vacuum overnight. The loading of material on solid support was checked by UV-Vis and Beer's law on a Beckman Coulter spectrophotometer. The solid support material was weighed out (52.7 mg) and dissolved in 0.1 M p-toluenesulfonic acid in acetonitrile in a 250 mL volumetric flask. The mixture was sonicated and allowed to sit undisturbed for 1 hour. The machine was blanked with the same solvent and the UV absorbance at 411 nm of the solution was measured in triplicate. The rest of the solid support materials was capped using 30% acetic anhydride in pyridine with 1% Et₃N (325 mL). The flask was capped and parafilmed and then shaken on mechanical shaker for 3 hours. The mixture was filtered on glass frit funnel under vacuum and washed in order: 10% H₂O in THF, MeOH, 10% H₂O in THF, MeOH, ACN, and diethyl ether (300 mL each). The filtrates were discarded, and the solid support material was dried on frit under vacuum. The solid support material was transferred to a round bottle flask and dried on high vacuum overnight to afford Compound 29 (51.63 g, 106.29 μmol/g loading).

Compound 31: Compound 31 was synthesized using Compound 30 and palmitic acid under standard peptide coupling conditions in CH₂Cl₂.

Compound 32: To a solution of Compound 31 (4.90 g, 6.35 mmol) in anhydrous dichloromethane (60.89 mL), DMAP (776 mg, 6.35 mmol) and succinic anhydride (1.27 g, 12.71 mmol) were added. The mixture was cooled to 0° C., and triethylamine (2.66 mL, 19.06 mmol) was added dropwise. The reaction mixture was stirred at room temperature for 18 hours, at which point no presence of starting material was shown (5% Et₃N in 5% MeOH in DCM). The mixture was concentrated under reduced pressure. The residue was purified by flash chromatography on silica gel (pre-treated with Et₃N) with gradient 0-10% MeOH in DCM to afford 4.34 g (78%) of the succinate. ¹H NMR (400 MHz, DMSO-d₆) δ 7.68 (q, J=5.5 Hz, 2H), 7.35-7.25 (m, 6H), 7.19 (ddt, J=8.9, 6.2, 2.9 Hz, 8H), 6.90-6.81 (m, 6H), 5.38-5.31 (m, 1H), 4.18 (d, J=4.5 Hz, 1H), 3.72 (s, 9H), 3.53 (dd, J=11.3, 3.2 Hz, 1H), 3.21 (dd, J=9.0, 4.7 Hz, 1H), 3.04-2.90 (m, 12H), 2.48-2.42 (m, 5H), 2.28-2.08 (m, 4H), 2.08-1.97 (m, 4H), 1.40 (dq, J=31.8, 7.0 Hz, 7H), 1.32-1.16 (m, 42H), 1.14 (t, J=7.2 Hz, 9H), 0.83 (t, J=6.6 Hz, 4H). To a solution of the succinate (4.34 g, 4.98 mmol) in anhydrous DMF (245.63 mL), DIPEA (3.74 mL, 19.93 mmol) was added then stirred until fully dissolved. HBTU (1.98 g, 5.23 mmol) was added to the mixture and stirred for 5 minutes. Controlled pore glass (CPG) (152 μmol/g, 36.05 g, 5.48 mmol) was added to the mixture. The round bottle flask was capped with a rubber septum, securely parafilmed, and then shaken on a mechanical shaker overnight. The mixture was filtered through a glass fritted funnel under vacuum and rinsed in parallel with acetonitrile, methanol, acetonitrile, and diethyl ether (300 mL each). The filtrate was discarded, and the filtered material was vacuum dried on frit for 20 minutes. The filtered material was returned to the original flask and dried on high vacuum overnight. The loading of material on solid support was checked by UV-Vis and Beer's law on a Beckman Coulter spectrophotometer. The solid support material was weighed (52.6 mg) and dissolved in 0.1 M p-toluenesulfonic acid in acetonitrile in a 250 mL volumetric flask. The mixture was sonicated and allowed to sit undisturbed for 1 hour. The machine was blanked with the same solvent and the UV absorbance at 411 nm of the solution was measured in triplicate. The rest of the solid support materials was capped using 30% acetic anhydride in pyridine with 1% Et₃N (325 mL). The flask was capped and parafilmed, and then shaken on mechanical shaker for 3 hours. The mixture was filtered on glass frit funnel under vacuum and washed in order: 10% H₂O in THF, MeOH, 10% H₂O in THF, MeOH, CAN, and diethyl ether (300 mL each). The filtrates were discarded, and the solid support material was dried on frit under vacuum. The solid support material was transferred to a round bottle flask and dried on high vacuum overnight to afford Compound 32 (37.59 g, 80.09 μmol/g loading).

### Synthesis of Terminal Acid-Containing Lipophilic Conjugate on Prolinol at 3′ End

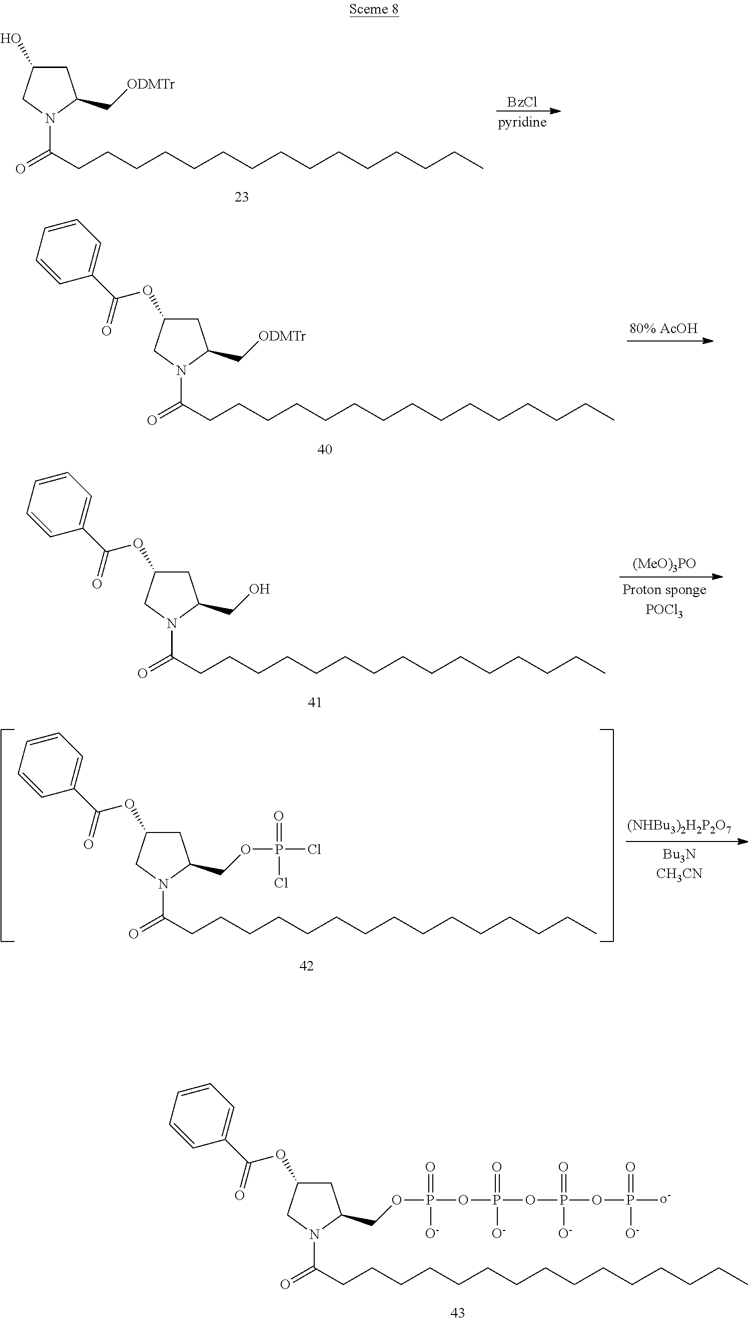

Compound 23: A solution of palmitic acid (12.22 g, 47.67 mmol) and HBTU (19.89 g, 52.44 mmol) in anhydrous dichloromethane was cooled to 0° C. DIPEA (24.91 mL, 143.02 mmol) was added to the solution dropwise. After stirring for 5 minutes, Compound 21 (20 g, 47.67 mmol) was added to the reaction. The mixture was stirred at room temperature for 24 hours, at which point no presence of starting material was shown (60% EtOAc in hexanes). The reaction mixture was diluted with DCM and performed standard aqueous workup with saturated aqueous NaHCO₃. The organic layers were combined, washed with saturated aqueous NaCl, dried over anhydrous sodium sulfate, and concentrated under reduced pressure. The residue was purified by flash chromatography on silica gel (pre-treated with Et₃N) with gradient 0-50% of EtOAc in hexanes to afford 28.01 g (89% yield) of Compound 23. ¹H NMR (500 MHz, DMSO-d₆) δ 7.36-7.26 (m, 5H), 7.23-7.16 (m, 6H), 6.90-6.83 (m, 5H), 4.96 (d, J=4.1 Hz, 1H), 4.39 (q, J=4.5 Hz, 1H), 4.18-4.07 (m, 2H), 3.73 (s, 8H), 3.58 (dd, J=10.6, 5.1 Hz, 1H), 3.17 (dd, J=8.9, 5.0 Hz, 1H), 3.02-2.94 (m, 2H), 2.69 (s, 12H), 2.20 (t, J=7.4 Hz, 2H), 2.06-1.90 (m, 2H), 1.83 (ddd, J=12.9, 8.5, 4.7 Hz, 1H), 1.46 (q, J=7.3 Hz, 2H), 1.30-1.16 (m, 28H), 0.87-0.81 (m, 4H).

Compound 33: Prior to reaction, Compound 23 (9.57 g, 14.55 mmol) was co-evaporated with acetonitrile twice, and then dried on high vacuum overnight. Compound 23 was dissolved in anhydrous dichloromethane (169.75 mL), and DIPEA (7.60 mL, 43.64 mmol) and 1-methylimidazole (579.7 uL, 7.27 mmol) were added dropwise. The mixture was cooled to 0° C. and chloro-2-cyanoethoxy-N,N-diisopropylaminophosphine (3.90 mL, 17.46 mmol) was added dropwise. The mixture was stirred at room temperature for 2 hours. The reaction mixture was checked by TLC (60% hexanes in EtOAc), and the solvent was removed under reduced pressure. The residue was resuspended in EtOAc and quickly performed aqueous work up with saturated aqueous NaHCO₃. The organic layers were combined, washed with saturated aqueous NaCl, dried over anhydrous sodium sulfate, and concentrated under reduced pressure. The residue was purified by flash chromatography on silica gel (pre-treated with Et₃N) with gradient 0-30% of EtOAc in hexanes to afford 10.11 g (81% yield) of Compound 33 (C₁₆H₃₁). ¹H NMR (400 MHz, Acetonitrile-d₃) δ 7.39 (ddd, J=8.1, 4.0, 1.4 Hz, 3H), 7.32-7.18 (m, 11H), 6.88-6.79 (m, 6H), 4.69 (td, J=9.1, 4.7 Hz, 1H), 4.20 (ddq, J=7.6, 4.9, 2.5 Hz, 1H), 3.76 (s, 12H), 3.59 (ddt, J=13.5, 11.3, 6.8 Hz, 4H), 3.33 (ddd, J=14.7, 9.1, 4.6 Hz, 1H), 3.02 (td, J=8.9, 3.0 Hz, 1H), 2.62 (tq, J=6.0, 4.1 Hz, 3H), 2.29-2.19 (m, 3H), 1.54 (t, J=7.3 Hz, 2H), 1.33-1.21 (m, 35H), 1.20-1.11 (m, 20H), 0.91-0.84 (m, 4H). ³¹P NMR (162 MHz, CD₃CN) δ 148.28, 147.41, 147.37, 147.23, 147.19, 146.85, 146.82.

Compound 35: A solution of methyl ester lipid carboxylic acid 34 (2.15 g, 7.15 mmol) and HBTU (2.98 g, 7.87 mmol) in anhydrous dichloromethane was cooled to 0° C. DIPEA (3.74 mL, 21.45 mmol) was added to the solution dropwise. After stirring for 5 minutes, Compound 21 (3 g, 7.15 mmol) was added to the reaction. The mixture was stirred at room temperature for 24 hours, at which point no presence of starting material was shown (60% EtOAc in hexanes). The reaction mixture was diluted with DCM and performed standard aqueous workup with saturated aqueous NaHCO₃. The organic layers were combined, washed with saturated aqueous NaCl, dried over anhydrous sodium sulfate, and concentrated under reduced pressure. The residue was purified by flash chromatography on silica gel (pre-treated with Et₃N) with gradient 0-62% of EtOAc in hexanes to afford 4.04 g (80% yield) of Compound 35. ¹H NMR (500 MHz, DMSO-d₆) δ 7.35-7.25 (m, 7H), 7.24-7.15 (m, 8H), 6.90-6.83 (m, 6H), 4.95 (d, J=4.0 Hz, 1H), 4.42-4.35 (m, 1H), 4.20-4.07 (m, 2H), 3.73 (s, 9H), 3.57 (s, 5H), 3.27-3.15 (m, 2H), 2.98 (dt, J=8.9, 4.5 Hz, 2H), 2.69 (s, 9H), 2.27 (t, J=7.4 Hz, 3H), 2.23-2.17 (m, 2H), 2.04-1.96 (m, 2H), 1.87-1.79 (m, 1H), 1.53-1.43 (m, 5H), 1.22 (d, J=5.9 Hz, 31H).

Compound 36: Prior to reaction, Compound 35 (4.04 g, 5.76 mmol) was co-evaporated with acetonitrile twice and then dried on high vacuum overnight. Compound 35 was dissolved in anhydrous dichloromethane (66.94 mL), and DIPEA (3.01 mL, 17.27 mmol) and 1-methylimidazole (458.7 uL, 5.76 mmol) were added dropwise. The mixture was cooled to 0° C. and chloro-2-cyanoethoxy-N,N-diisopropylaminophosphine (1.54 mL, 6.91 mmol) was added dropwise. The mixture was stirred at room temperature for 1.5 hours. The reaction mixture was checked by TLC (60% hexanes in EtOAc) and the solvent was removed under reduced pressure. The residue was resuspended in EtOAc and quickly performed aqueous work up with saturated aqueous NaHCO₃. The organic layers were combined, washed with saturated aqueous NaCl, dried over anhydrous sodium sulfate and concentrated under reduced pressure. The residue was purified by flash chromatography on silica gel (pre-treated with Et₃N) with gradient 0-30% of EtOAc in hexanes to afford 4.09 g (79%) of Compound 36. ¹H NMR (400 MHz, Acetonitrile-d₃) δ 7.39 (ddd, J=8.2, 4.0, 1.4 Hz, 6H), 7.33-7.15 (m, 20H), 6.88-6.79 (m, 11H), 4.69 (d, J=4.7 Hz, 1H), 4.21 (dp, J=7.8, 2.4 Hz, 2H), 3.85-3.67 (m, 24H), 3.59 (s, 16H), 3.38-3.27 (m, 2H), 3.02 (td, J=8.9, 3.0 Hz, 2H), 2.62 (tdd, J=7.5, 4.5, 2.9 Hz, 6H), 2.26 (q, J=7.5 Hz, 9H), 1.55 (h, J=7.5 Hz, 11H), 1.34-1.20 (m, 58H), 1.21-1.10 (m, 37H). ³¹P NMR (162 MHz, CD₃CN) δ 149.70, 148.82, 148.80, 148.63, 148.60, 148.26, 148.23.

Compound 38: A solution of methyl ester lipid carboxylic acid 37 (2.35 g, 7.15 mmol) and HBTU (2.98 g, 7.87 mmol) in anhydrous dichloromethane was cooled to 0° C. DIPEA (3.74 mL, 21.45 mmol) was added to the solution dropwise. After stirring for 5 minutes, Compound 21 (3 g, 7.15 mmol) was added to the reaction. The mixture was stirred at room temperature for 24 hours, at which point no presence of starting material was shown (60% EtOAc in hexanes). The reaction mixture was diluted with DCM and performed standard aqueous workup with saturated aqueous NaHCO₃. The organic layers were combined, washed with saturated aqueous NaCl, dried over anhydrous sodium sulfate, and concentrated under reduced pressure. The residue was purified by flash chromatography on silica gel (pre-treated with Et₃N) with gradient 0-68% of EtOAc in hexanes to afford 4.44 g (85% yield) of Compound 38. ¹H NMR (400 MHz, DMSO-d₆) δ 7.36-7.25 (m, 5H), 7.20 (td, J=8.9, 2.8 Hz, 6H), 6.90-6.83 (m, 5H), 4.97 (d, J=4.0 Hz, 1H), 4.39 (q, J=4.5 Hz, 1H), 3.73 (d, J=0.7 Hz, 8H), 3.57 (s, 4H), 3.17 (dd, J=8.9, 5.0 Hz, 1H), 3.01-2.94 (m, 2H), 2.69 (s, 15H), 2.27 (t, J=7.4 Hz, 3H), 2.20 (t, J=7.4 Hz, 2H), 2.04-1.96 (m, 1H), 1.83 (s, OH), 1.49 (q, J=5.6, 4.5 Hz, 2H), 1.22 (d, J=4.6 Hz, 30H).

Compound 39: Prior to reaction, Compound 38 (4.44 g, 6.08 mmol) was co-evaporated with acetonitrile twice and then dried on high vacuum overnight. Compound 38 was dissolved in anhydrous dichloromethane (70.74 mL), and DIPEA (3.18 mL, 18.25 mmol) and 1-methylimidazole (484.8 uL, 6.08 mmol) were added dropwise. The mixture was cooled to 0° C. and chloro-2-cyanoethoxy-N,N-diisopropylaminophosphine (1.63 mL, 7.30 mmol) was added dropwise. The mixture was stirred at room temperature for 1.5 hours. The reaction mixture was checked by TLC (30% Hexanes in EtOAc) and the solvent was removed under reduced pressure. The residue was resuspended in EtOAc and quickly performed aqueous work up with saturated aqueous NaHCO₃. The organic layers were combined, washed with saturated aqueous NaCl, dried over anhydrous sodium sulfate, and concentrated under reduced pressure. The residue was purified by flash chromatography on silica gel (pre-treated with Et₃N) with gradient 0-30% of EtOAc in hexanes to afford 4.43 g (78% yield) of Compound 39. ¹H NMR (400 MHz, Acetonitrile-d₃) δ 7.39 (ddd, J=8.1, 3.9, 1.4 Hz, 3H), 7.32-7.17 (m, 10H), 6.87-6.80 (m, 5H), 4.69 (ddq, J=13.6, 9.3, 4.3 Hz, 1H), 4.21 (ddt, J=7.7, 5.4, 2.6 Hz, 1H), 3.82-3.67 (m, 12H), 3.59 (s, 7H), 3.33 (ddd, J=14.7, 9.1, 4.6 Hz, 1H), 3.02 (td, J=8.9, 2.9 Hz, 1H), 2.62 (tq, J=6.0, 4.2 Hz, 3H), 2.25 (dt, J=14.0, 7.0 Hz, 4H), 2.19-2.13 (m, 3H), 1.55 (h, J=7.9, 7.2 Hz, 5H), 1.37-1.21 (m, 33H), 1.21-1.09 (m, 17H). ³¹P NMR (162 MHz, CD₃CN) δ 149.69, 148.81, 148.78, 148.62, 148.59, 148.55, 148.26, 148.22.

### Synthesis of Hexadecyl Hydroxyprolinol Triphosphate

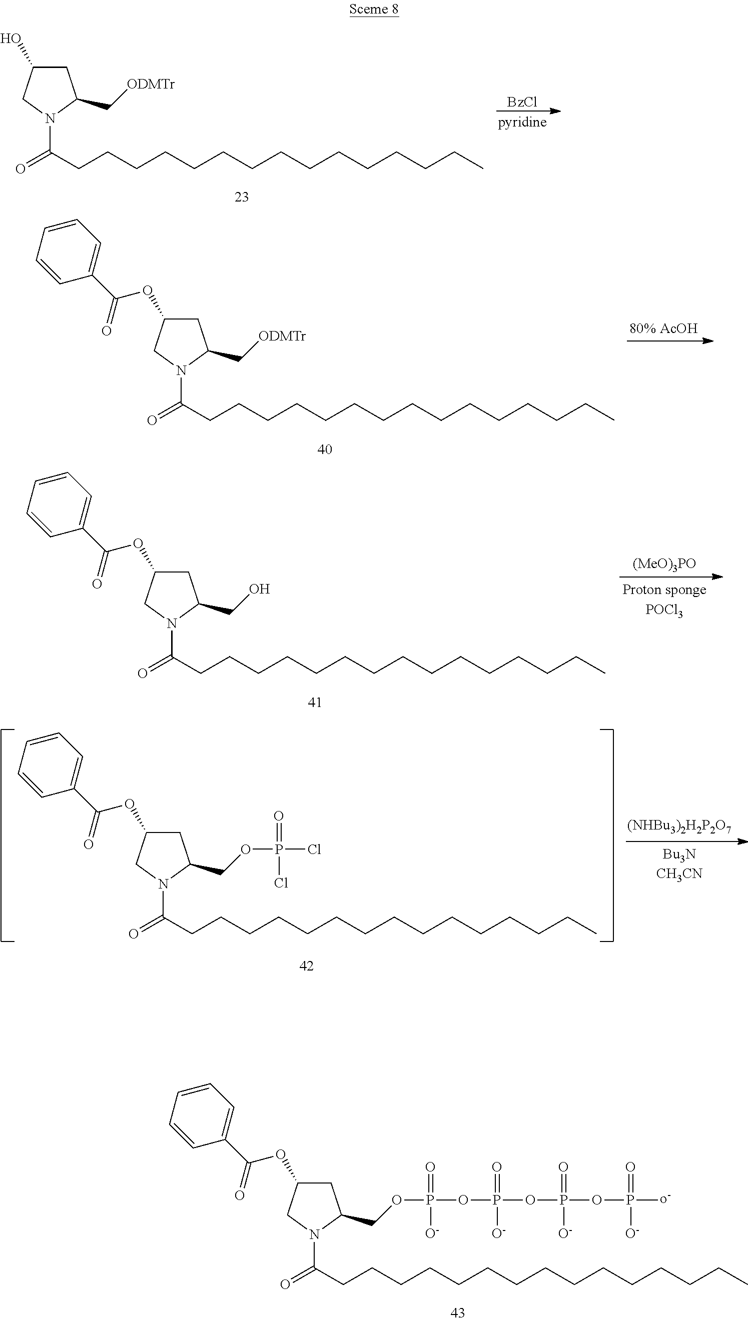

Compound 40: Prior to synthesis, the starting material, Compound 23, was co-evaporated with pyridine twice and dried on high vacuum overnight. The starting material (1.01 g, 1.54 mmol) was dissolved in anhydrous pyridine (7.46 mL) and cooled to 0° C., and benzoyl chloride (214 μL, 1.84 mmol) was added dropwise. The mixture was stirred for 1 hour at room temperature, and TLC was checked (80% hexanes in ethyl acetate). The solvent was stripped under reduced pressure, and the residue was resuspended in ethyl acetate. Standard aqueous workup was performed with saturated aqueous NaHCO₃. The organic layers were combined, washed with saturated aqueous NaCl, dried over anhydrous sodium sulfate, and concentrated under reduced pressure. The residue was purified by flash chromatography on silica gel (pre-treated with Et₃N) with gradient 0-20% of EtOAc in hexanes to afford 890 mg (76% yield) of Compound 40. ¹H NMR (500 MHz, DMSO-d₆) δ 7.93 (ddt, J=12.8, 7.0, 1.4 Hz, 3H), 7.68-7.62 (m, 1H), 7.56-7.46 (m, 3H), 7.35 (ddt, J=8.1, 3.2, 1.8 Hz, 3H), 7.30 (q, J=7.9, 7.5 Hz, 3H), 7.27-7.17 (m, 7H), 6.88 (ddd, J=9.0, 6.1, 2.9 Hz, 6H), 5.60 (p, J=4.5 Hz, 1H), 4.29 (q, J=5.5, 5.1 Hz, 2H), 3.90 (ddd, J=28.0, 12.4, 3.9 Hz, 1H), 3.80-3.75 (m, 1H), 3.73 (d, J=1.0 Hz, 9H), 3.36 (s, 1H), 3.27 (dd, J=9.0, 4.7 Hz, 1H), 3.15-3.04 (m, 2H), 2.36-2.16 (m, 5H), 1.44 (q, J=7.4 Hz, 2H), 1.29-1.20 (m, 29H), 0.87-0.81 (m, 4H).

Compound 41: In a round bottom flask charged with a stir bar, Compound 40 (890 mg, 1.17 mmol) was dissolved in 80% AcOH in water (13 mL). The mixture was stirred at room temperature for 48 hours, and the solvent was removed under reduced pressure. The residue was co-evaporated with toluene twice, and dried on high vacuum. The residue was purified by flash chromatography on silica gel (pre-treated with Et₃N) with gradient 0-60% of EtOAc in hexanes to afford 301 mg (56% yield) of Compound 41. ¹H NMR (500 MHz, DMSO-d₆) δ 7.97-7.89 (m, 3H), 7.66 (td, J=6.8, 6.1, 1.6 Hz, 1H), 7.51 (td, J=7.7, 6.0 Hz, 3H), 5.51-5.40 (m, 1H), 4.84 (t, J=5.5 Hz, 1H), 4.15 (dp, J=11.7, 3.8 Hz, 1H), 3.77 (dd, J=11.8, 5.0 Hz, 1H), 3.59 (dt, J=10.5, 5.2 Hz, 1H), 3.47 (ddd, J=14.8, 9.0, 4.7 Hz, 2H), 2.33-2.11 (m, 5H), 1.57-1.39 (m, 3H), 1.30-1.11 (m, 37H), 0.85 (t, J=6.8 Hz, 4H).

Compound 42: Prior to synthesis, the starting material, Compound 41 (200 mg, 0.435 mmol), was dried on high vacuum overnight. In a round bottle flask equipped with a stir bar, the starting material was charged with proton sponge (93 mg, 0.435 mmol) and trimethyl phosphate (1.81 mL, 15.64 mmol) at room temperature. The reaction flask was evacuated using a vacuum line then flushed with argon, repeated three times, and then kept under argon. The mixture was stirred at room temperature for 10 minutes, and cooled to between −5 to −10° C. on ice and NaCl bath for 30 minutes. After cooling, phosphoryl chloride (28.30 μL, 0.305 mmol) was added via sealed glass syringe, stirred for 4 minutes, and another portion of phosphoryl chloride (20.22 μL, 0.217 mmol) was added via sealed glass syringe. The mixture was stirred at −5 to −10° C. for 10 minutes. Pyrophosphate cocktail was prepared with tributylammonium pyrophosphate (255.50 mg, 0.348 mmol) dissolved in anhydrous acetonitrile (1.75 mL) and tributylamine (621.95 μL, 2.61 mmol), and kept at −20° C. in dry ice/acetone bath. After stirring for 10 minutes, the pyrophosphate cocktail was quickly but carefully added dropwise to the cold reaction mixture, and then stirred for additional 10 minutes. After removing the argon line from the flask, water (12 mL) was added via addition funnel. The mixture was transferred to a separatory funnel, and the aqueous layer was washed three time with dichloromethane (5 mL each). The aqueous layers were combined and the pH was adjusted to 6.5 using ammonium hydroxide (3 drops using syringe). The mixture was stored at 4° C. overnight. The solvent was stripped off under reduced pressure, and the remaining residue was frozen at −80° C. in acetone/dry ice bath. The residue was lyophilized overnight and submitted for ³¹P NMR analysis in D₂O. ³¹P NMR (202 MHz, D₂O) δ 3.72, −10.12, −20.99.

### Synthesis of 2′-O—C6-amino-TFA Uridine Amidite

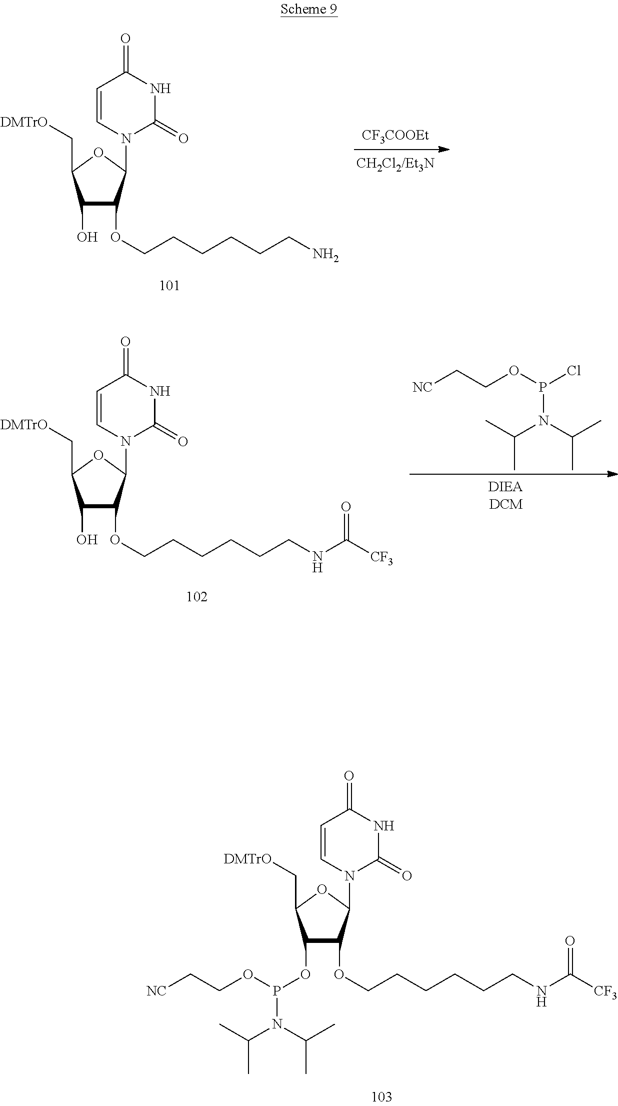

Compound 102: Compound 101 (5 g, 7.75 mmol) was added to a reaction flask. The starting material was dissolved in dichloromethane (50 ml), and triethylamine (4.23 ml, 31 mmol) was added via syringe. Ethyl trifluroacetate (2.75 g, 19.38 mmol) was added dropwise to the reaction. The reaction mixture was stirred at room temperature overnight and checked by TLC (5% MeOH/DCM), developed using phosphomolybdic acid, and concentrated under reduced pressure. The residue was dissolved in dichloromethane, added to separation funnel, and the organic layer was washed with saturated sodium bicarbonate. The organic layer was separated and washed with a brine solution. The organic layer was separated and dried with sodium sulfate. The solid was filtered off and the mother liquor was concentrated and put on high vacuum to yield (4.32 g, 75%) of Compound 102. ¹H NMR (500 MHz, DMSO-d6) δ 11.36 (d, J=2.6 Hz, 2H), 9.36 (s, 1H), 7.71 (d, J=8.1 Hz, 2H), 7.36 (d, J=8.4 Hz, 4H), 7.31 (t, J=7.6 Hz, 4H), 7.27-7.20 (m, 10H), 6.89 (d, J=8.5 Hz, 8H), 5.78 (d, J=3.6 Hz, 2H), 5.27 (dd, J=8.1, 2.1 Hz, 2H), 5.10 (dd, J=6.7, 2.7 Hz, 2H), 4.16 (m, 2H), 3.95 (m, 2H), 3.88 (m, 2H), 3.73 (s, 13H), 3.55 (m, 4H), 3.36 (m, 1H), 3.28 (d, J=4.4 Hz, 1H), 3.22 (dd, J=10.9, 2.8 Hz, 2H), 3.14 (m, 3H), 2.11 (s, 2H), 1.48 (m, 8H), 1.36-1.19 (m, 8H). Mass calc. for C₃₈H₄₂F₃N₃O₉: 741.76, found: 740.2 (M−H).

Compound 103: Compound 102 (4.3 g, 5.8 mmol) was added to a reaction flask, evacuated, and purged with argon. The starting material was dissolved in dichloromethane (40 ml), and diisopropylethylamine (2.02 ml, 11.6 mmol) was added via syringe. 2-cyanoethyl N,N-diisopropylchlorophosphoramidite (1.93 ml, 8.7 mmol) was added, and the reaction stirred at room temperature for 1-2 hours. The reaction mixture was checked by TLC (75% EtOAc/hexane), and concentrated under reduced pressure. The residue was dissolved in ethyl acetate, added to separation funnel, and the organic layer was washed with saturated sodium bicarbonate. The organic layer was separated and washed with a brine solution. The organic layer was separated and dried with sodium sulfate. The solid was filtered off and the mother liquor was concentrated. The residue was purified by flash chromatography on silica gel (10% to 100% EtOAc/hexane), and the product fractions were combined and concentrated on reduced pressure to yield (4.62 g, 85%) of Compound 103. ¹H NMR (400 MHz, acetonitrile-d3) δ 9.06 (s, 1H), 7.74 (d, J=8.1 Hz, 1H), 7.49-7.39 (m, 2H), 7.39-7.21 (m, 7H), 6.93-6.83 (m, 4H), 5.84 (dd, J=7.0, 3.2 Hz, 1H), 5.21 (m, 1H), 4.45 (m, 1H), 4.20-3.97 (m, 3H), 3.91-3.79 (m, 1H), 3.77 (d, J=2.4 Hz, 7H), 3.63 (m, 4H), 3.48-3.31 (m, 3H), 3.23 (m, 1H), 2.67 (m, 1H), 2.52 (t, J=6.0 Hz, 1H), 2.08 (d, J=1.9 Hz, 1H), 1.64-1.45 (m, 4H), 1.42-1.28 (m, 4H), 1.27-1.09 (m, 9H), 1.05 (d, J=6.7 Hz, 3H). ³¹P NMR (162 MHz, acetonitrile-d3) δ 149.53, 149.06. ¹⁹F NMR (376 MHz, acetonitrile-d3) δ −83.43, −83.89 (d, J=2.4 Hz).

### Synthesis of 2′-O—C3-amino-TFA Uridine Amidite

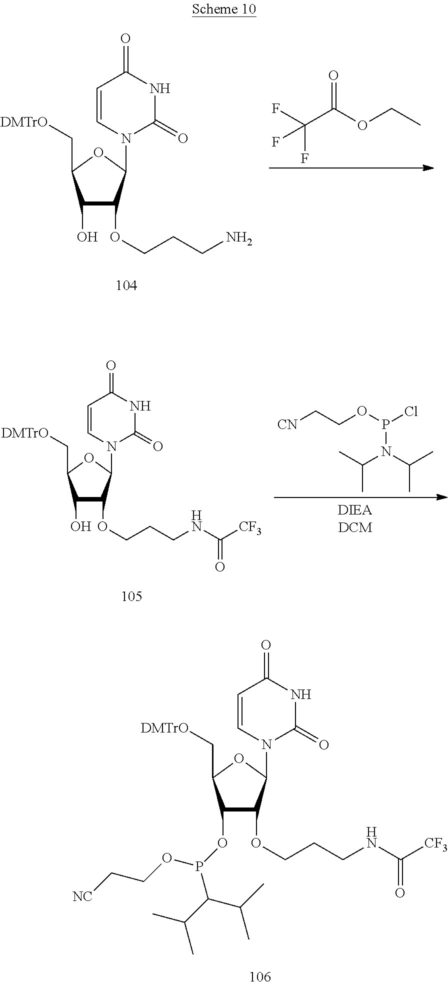

Compound 105: Compound 104 (2.5 g, 4.14 mmol) was added to a reaction flask. The starting material was dissolved in dichloromethane (20 ml), and triethylamine (2.26 ml, 16.56 mmol) was added via syringe. Ethyl trifluroacetate (1.47 g, 10.35 mmol) was added dropwise to the reaction. The reaction mixture was stirred at room temperature overnight and checked by TLC (3% MeOH/DCM), developed using phosphomolybdic acid, and concentrated under reduced pressure. The residue was dissolved in dichloromethane, added to separation funnel, and the organic layer was washed with saturated sodium bicarbonate. The organic layer was separated and washed with a brine solution. The organic layer was separated and dried with sodium sulfate. The solid was filtered off and the mother liquor was concentrated. The residue was purified by flash chromatography on silica gel (0% to 10% MeOH/DCM), and the product fractions were combined and concentrated on reduced pressure to yield (1.83 g, 63%) of Compound 105. ¹H NMR (400 MHz, DMSO-d6) δ 9.39 (m, 1H), 7.79 (d, J=8.1 Hz, 1H), 7.37 (d, J=7.3 Hz, 3H), 7.31 (t, J=7.5 Hz, 3H), 7.27-7.16 (m, 7H), 6.93-6.85 (m, 5H), 5.81-5.73 (m, 2H), 5.54 (d, J=4.9 Hz, 1H), 5.38 (d, J=8.1 Hz, 1H), 5.19 (dd, J=8.6, 6.4 Hz, 1H), 4.15-4.02 (m, 2H), 4.01-3.87 (m, 2H), 3.83-3.74 (m, 2H), 3.73 (s, 8H), 3.31-3.14 (m, 5H), 2.07 (s, 1H), 1.74 (dd, J=11.4, 4.6 Hz, 3H). ¹⁹F NMR (376 MHz, DMSO-d6) δ −81.24 (d, J=43.2 Hz). Mass calc. for C₃₅H₃₆F₃N₃O₉: 699.68, found: 698.2 (M−H).

Compound 106: Compound 105 (1.70 g, 2.43 mmol) was added to a reaction flask, evacuated, and purged with argon. The starting material was dissolved in dichloromethane (2 ml), and diisopropylethylamine (0.846 ml, 4.86 mmol) was added via syringe. 2-cyanoethyl N,N-diisopropylchlorophosphoramidite (0.649 ml, 2.92 mmol) was added, and the reaction stirred at room temperature for 1-2 hours. The reaction mixture was checked by TLC (50% EtOAc/hexane), and concentrated under reduced pressure. The residue was dissolved in ethyl acetate, added to separation funnel, and the organic layer was washed with saturated sodium bicarbonate. The organic layer was separated and washed with a brine solution. The organic layer was separated and dried with sodium sulfate. The solid was filtered off and the mother liquor was concentrated. The residue was purified by flash chromatography on silica gel (10% to 100% EtOAc/hexane), and the product fractions were combined and concentrated on reduced pressure to yield (0.787 g, 36%) of Compound 106. ¹H NMR (400 MHz, acetonitrile-d3) δ 7.89-7.63 (m, 2H), 7.49-7.39 (m, 2H), 7.38-7.20 (m, 7H), 6.88 (m, 4H), 6.13-5.97 (m, 1H), 5.53-5.34 (m, 1H), 4.52-4.32 (m, 2H), 4.24 (m, 1H), 3.94-3.80 (m, 4H), 3.80-3.74 (m, 7H), 3.71-3.53 (m, 5H), 3.52-3.29 (m, 3H), 3.25 (m, 2H), 2.64 (m, 3H), 1.86-1.75 (m, 2H), 1.36-0.96 (m, 25H). ¹⁹F NMR (376 MHz, Acetonitrile-d3) δ −77.26, −143.51. ³¹P NMR (202 MHz, acetonitrile-d3) δ 152.03 (d, J=6.2 Hz), 151.47-150.50 (m).

### Synthesis of 2′-O—C6-amide-C16 Conjugated Uridine Amidite

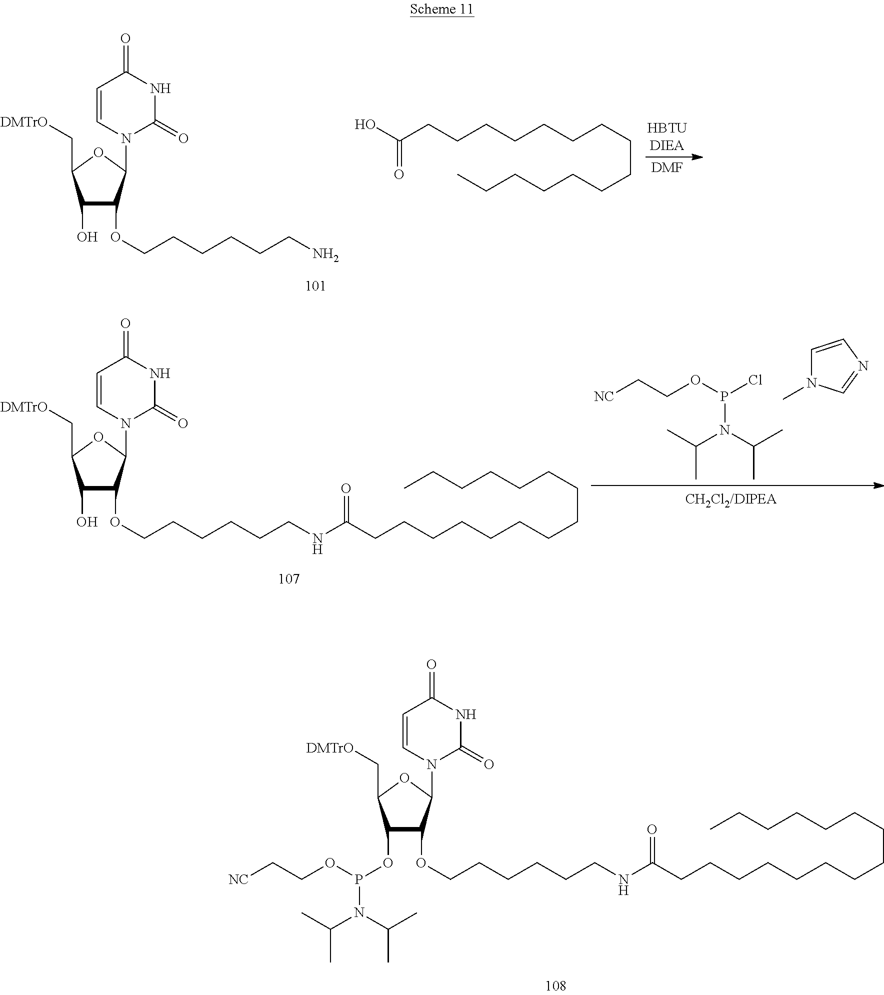

Compound 107: Compound 101 (5.7 g, 8.83 mmol) was added to a reaction flask, along with palmitic acid (2.51 g, 9.8 mmol) and HBTU (4.08 g, 10.77 mmol). The solids were dissolved in DMF (25 ml), and diisopropylethylamine (4.61 ml, 26.5 mmol) was added via syringe. The reaction mixture was stirred at room temperature overnight. The reaction mixture was checked by MS. The reaction mixture was diluted with diethyl ether and dilute sodium bicarbonate solution, and was added to separation funnel. The organic layer was washed with dilute sodium bicarbonate solution, saturated sodium bicarbonate, and then saturated brine solution. The organic layer was separated and dried with sodium sulfate. The solid was filtered off and the mother liquor was concentrated. The residue was purified by flash chromatography on silica gel (0% to 100% EtOAc/hexane), and the product fractions were combined and concentrated on reduced pressure to yield (6.33 g, 81%) of Compound 107. ¹H NMR (400 MHz, DMSO-d6) δ 11.40 (dd, J=27.8, 2.2 Hz, 1H), 7.76-7.63 (m, 2H), 7.33 (m, 4H), 7.23 (m, 5H), 6.89 (dd, J=9.3, 3.0 Hz, 4H), 5.78 (d, J=3.5 Hz, 1H), 5.27 (dd, J=8.1, 2.1 Hz, 1H), 5.21-5.07 (m, 1H), 4.26-4.06 (m, 1H), 3.91 (m, 2H), 3.73 (s, 6H), 3.63-3.43 (m, 2H), 3.29-3.18 (m, 2H), 2.98 (q, J=6.6 Hz, 2H), 2.00 (t, J=7.4 Hz, 2H), 1.47 (m, 4H), 1.34 (t, J=6.9 Hz, 2H), 1.21 (s, 23H), 0.83 (t, J=6.7 Hz, 3H). Mass calc. for C₅₂H₇₃N₃O₉: 884.17, found: 882.5 (M−H).

Compound 108: Compound 107 (5.83 g, 6.59 mmol) was added to a reaction flask, evacuated, and purged with argon. The starting material was dissolved in dichloromethane (60 ml), and diisopropylethylamine (3.45 ml, 19.78 mmol) was added via syringe. The reaction mixture was cooled to 0° C. via ice bath. 2-cyanoethyl N,N-diisopropylchlorophosphoramidite (1.91 ml, 8.57 mmol) and 1-methylimidazole (0.525 ml, 6.6 mmol) were added to the reaction mixture, and the reaction mixture was allowed to warm to room temperature and stirred for 1 hour. The reaction mixture was checked by TLC (80% EtOAc/hexane), and concentrated under reduced pressure. The residue was dissolved in dichloromethane, added to separation funnel, and the organic layer was washed with saturated sodium bicarbonate. The organic layer was separated and washed with a brine solution. The organic layer was separated and dried with sodium sulfate. The solid was filtered off and the mother liquor was concentrated. The residue was purified by flash chromatography on silica gel (10% to 80% EtOAc/hexane), and the product fractions were combined and concentrated on reduced pressure to yield (4.6 g, 64%) of Compound 108. ¹H NMR (500 MHz, acetonitrile-d3) δ 9.16 (s, 1H), 7.71 (d, J=8.1 Hz, 1H), 7.52-7.39 (m, 2H), 7.37-7.22 (m, 7H), 6.92-6.84 (m, 4H), 6.28 (d, J=7.2 Hz, 1H), 5.86 (dd, J=9.1, 3.7 Hz, 1H), 5.23 (t, J=8.2 Hz, 1H), 4.54-4.32 (m, 1H), 4.20-4.09 (m, 1H), 4.07-3.97 (m, 1H), 3.77 (d, J=2.8 Hz, 7H), 3.62 (m, 4H), 3.55-3.33 (m, 3H), 3.09 (m, 2H), 2.75 (s, 1H), 2.67 (m, 1H), 2.52 (s, 1H), 2.06 (m, 2H), 1.62-1.49 (m, 4H), 1.45-1.39 (m, 2H), 1.34 (m, 3H), 1.25 (d, J=16.3 Hz, 27H), 1.16 (dd, J=10.8, 6.8 Hz, 8H), 1.05 (d, J=6.8 Hz, 3H), 0.88 (t, J=6.9 Hz, 3H). ³¹P NMR (202 MHz, acetonitrile-d3) δ 151.06, 150.60.

### Synthesis of 2′-O—C3-amide-C16 Conjugated Uridine Amidite

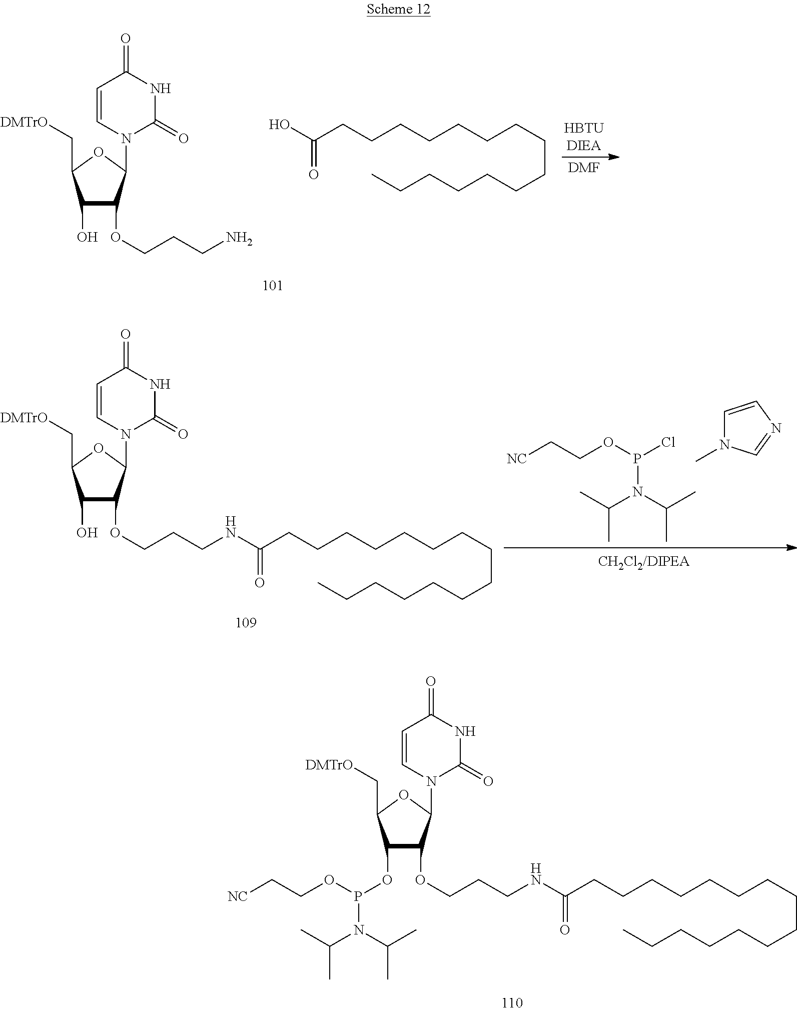

Compound 109: Compound 104 (5.3 g, 8.78 mmol) was added to a reaction flask, along with palmitic acid (2.50 g, 9.75 mmol) and HBTU (4.06 g, 10.71 mmol). The solids were dissolved in DMF (25 ml) and diisopropylethylamine (4.59 ml, 26.34 mmol) was added via syringe. The reaction mixture was stirred at room temperature overnight. The reaction mixture was checked by MS. The reaction mixture was diluted with diethyl ether and dilute sodium bicarbonate solution, and was added to separation funnel. The organic layer was washed with dilute sodium bicarbonate solution, saturated sodium bicarbonate, and then saturated brine solution. The organic layer was separated and dried with sodium sulfate. The solid was filtered off and the mother liquor was concentrated. The residue was purified by flash chromatography on silica gel (0% to 100% EtOAc/hexane), and the product fractions were combined and concentrated on reduced pressure to yield (4.66 g, 63%) of Compound 109. ¹H NMR (400 MHz, DMSO-d6) δ 11.37 (s, 1H), 7.75-7.67 (m, 2H), 7.34 (dd, J=19.6, 7.3 Hz, 4H), 7.29-7.14 (m, 6H), 6.89 (d, J=8.5 Hz, 4H), 5.78 (d, J=3.4 Hz, 1H), 5.27 (d, J=8.0 Hz, 1H), 5.19 (d, J=6.6 Hz, 1H), 4.18 (q, J=6.2 Hz, 1H), 3.92 (m, 2H), 3.73 (s, 6H), 3.57 (q, J=5.7, 5.0 Hz, 2H), 3.30-3.18 (m, 2H), 3.09 (m, 2H), 2.01 (t, J=7.4 Hz, 2H), 1.63 (m, 2H), 1.45 (t, J=7.2 Hz, 2H), 1.21 (d, J=5.1 Hz, 23H), 0.83 (t, J=6.7 Hz, 3H). Mass calc. for C₄₉H₆₇N₃O₉: 842.09, found: 840.5 (M−H).

Compound 110: Compound 109 (4.66 g, 5.53 mmol) was added to a reaction flask, evacuated, and purged with argon. The starting material was dissolved in dichloromethane (40 ml), and diisopropylethylamine (2.89 ml, 16.6 mmol) was added via syringe. The reaction mixture was cooled to 0° C. via ice bath. 2-cyanoethyl N,N-diisopropylchlorophosphoramidite (1.61 ml, 7.19 mmol) and 1-methylimidazole (0.441 ml, 5.53 mmol) were added to the reaction mixture, and the reaction mixture was allowed to warm to room temperature and stirred for 2 hours. The reaction mixture was checked by TLC (80% EtOAc/hexane) and concentrated under reduced pressure. The residue was dissolved in dichloromethane, added to separation funnel, and the organic layer was washed with saturated sodium bicarbonate. The organic layer was separated and washed with a brine solution. The organic layer was separated and dried with sodium sulfate. The solid was filtered off and the mother liquor was concentrated. The residue was purified by flash chromatography on silica gel (10% to 80% EtOAc/hexane), and the product fractions were combined and concentrated on reduced pressure to yield (3.86 g, 67%) of Compound 110. ¹H NMR (500 MHz, acetonitrile-d3) δ 9.01 (s, 1H), 7.74 (d, J=8.2 Hz, 1H), 7.52-7.40 (m, 3H), 7.36-7.21 (m, 7H), 6.92-6.85 (m, 4H), 6.40 (d, J=5.4 Hz, 1H), 5.85 (dd, J=7.6, 2.9 Hz, 1H), 5.21 (t, J=8.3 Hz, 1H), 4.46 (m, 1H), 4.22-4.09 (m, 2H), 4.09-3.98 (m, 2H), 3.91-3.80 (m, 1H), 3.80-3.69 (m, 9H), 3.68-3.55 (m, 3H), 3.55-3.34 (m, 3H), 3.22 (m, 2H), 2.75 (t, J=5.9 Hz, 1H), 2.68 (m, 1H), 2.52 (t, J=5.9 Hz, 1H), 2.06 (m, 2H), 1.71 (m, 2H), 1.54-1.49 (m, 2H), 1.25 (dd, J=9.5, 6.5 Hz, 28H), 1.22-1.10 (m, 10H), 1.05 (d, J=6.7 Hz, 3H), 0.88 (t, J=6.8 Hz, 3H). ³¹P NMR (202 MHz, acetonitrile-d3) δ 151.01, 150.56.

### Synthesis of 2′-O—C6-amide-C14 Conjugated Uridine Amidite

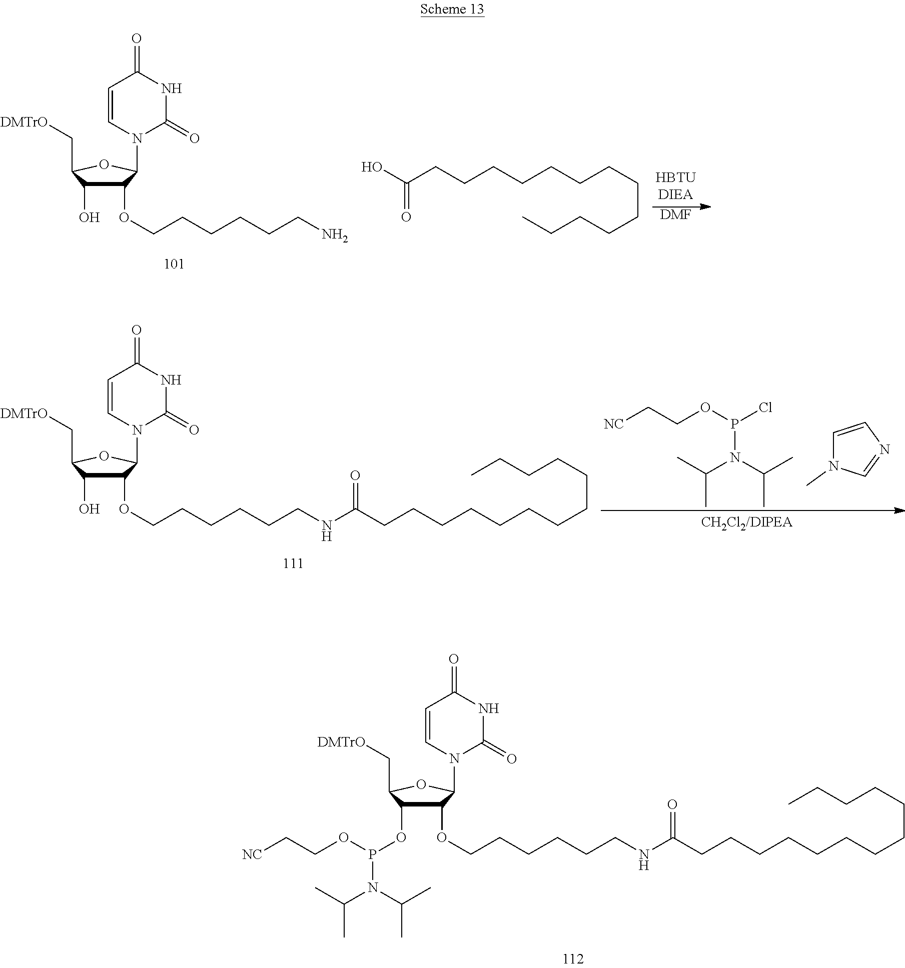

Compound 111: Compound 101 (5.0 g, 7.74 mmol) was added to a reaction flask, along with myristic acid (1.96 g, 8.6 mmol) and HBTU (3.58 g, 9.45 mmol). The solids were dissolved in DMF (25 ml), and diisopropylethylamine (4.05 ml, 23.23 mmol) was added via syringe. The reaction mixture was stirred at room temperature overnight. The reaction mixture was checked by TLC (80% EtOAc/hexane). The reaction mixture was diluted with diethyl ether and dilute sodium bicarbonate solution, and was added to separation funnel. The organic layer was washed with dilute sodium bicarbonate solution, saturated sodium bicarbonate, and then saturated brine solution. The organic layer was separated and dried with sodium sulfate. The solid was filtered off and the mother liquor was concentrated. The residue was purified by flash chromatography on silica gel (0% to 100% EtOAc/hexane), and the product fractions were combined and concentrated on reduced pressure to yield (3.78 g, 57%) of Compound 111. ¹H NMR (400 MHz, DMSO-d6) δ 11.37 (d, J=2.2 Hz, 1H), 7.72 (d, J=8.1 Hz, 1H), 7.67 (t, J=5.6 Hz, 1H), 7.41-7.28 (m, 4H), 7.23 (m, 5H), 6.89 (d, J=8.6 Hz, 4H), 5.78 (d, J=3.6 Hz, 1H), 5.27 (dd, J=8.0, 2.1 Hz, 1H), 5.11 (d, J=6.6 Hz, 1H), 4.16 (q, J=6.2 Hz, 1H), 3.95 (m, 1H), 3.73 (s, 6H), 3.63-3.47 (m, 2H), 3.31-3.18 (m, 3H), 2.98 (q, J=6.5 Hz, 2H), 2.00 (t, J=7.4 Hz, 2H), 1.47 (m, 4H), 1.34 (m, 3H), 1.21 (s, 23H), 0.83 (t, J=6.7 Hz, 3H).

Compound 112: Compound 111 (3.78 g, 4.42 mmol) was added to a reaction flask, evacuated, and purged with argon. The starting material was dissolved in dichloromethane (40 ml), and diisopropylethylamine (2.31 ml, 13.25 mmol) was added via syringe. The reaction mixture was cooled to 0° C. via ice bath. 2-cyanoethyl N,N-diisopropylchlorophosphoramidite (1.28 ml, 5.74 mmol) and 1-methylimidazole (0.352 ml, 4.42 mmol) were added, and the reaction mixture was allowed to warm to room temperature and stirred for 1 hour. The reaction mixture was checked by TLC (80% EtOAc/hexane), and concentrated under reduced pressure. The residue was dissolved in dichloromethane, added to separation funnel, and the organic layer was washed with saturated sodium bicarbonate. The organic layer was separated and washed with a brine solution. The organic layer was separated and dried with sodium sulfate. The solid was filtered off and the mother liquor was concentrated. The residue was purified by flash chromatography on silica gel (10% to 80% EtOAc/hexane), and the product fractions were combined and concentrated on reduced pressure to yield (4.04 g, 87%) of Compound 112. ¹H NMR (400 MHz, acetonitrile-d3) δ 9.18 (s, 1H), 7.44 (m, 2H), 7.38-7.21 (m, 7H), 6.93-6.83 (m, 4H), 6.29 (d, J=5.9 Hz, 1H), 5.86 (dd, J=7.4, 3.7 Hz, 1H), 5.23 (dd, J=8.1, 6.7 Hz, 1H), 4.53-4.33 (m, 1H), 4.15 (m, 1H), 4.08-3.97 (m, 1H), 3.86 (m, 1H), 3.77 (d, J=2.3 Hz, 6H), 3.62 (m, 4H), 3.48-3.32 (m, 2H), 3.09 (m, 2H), 2.67 (m, 1H), 2.52 (t, J=6.0 Hz, 1H), 2.06 (m, 2H), 1.54 (m, 4H), 1.41 (m, 2H), 1.26 (s, 25H), 1.16 (dd, J=8.7, 6.8 Hz, 10H), 1.05 (d, J=6.8 Hz, 3H), 0.92-0.83 (m, 3H). ³¹P NMR (202 MHz, acetonitrile-d3) δ 151.06, 150.60.

### Synthesis of 2′-O—C6-amide-C18 Conjugated Uridine Amidite

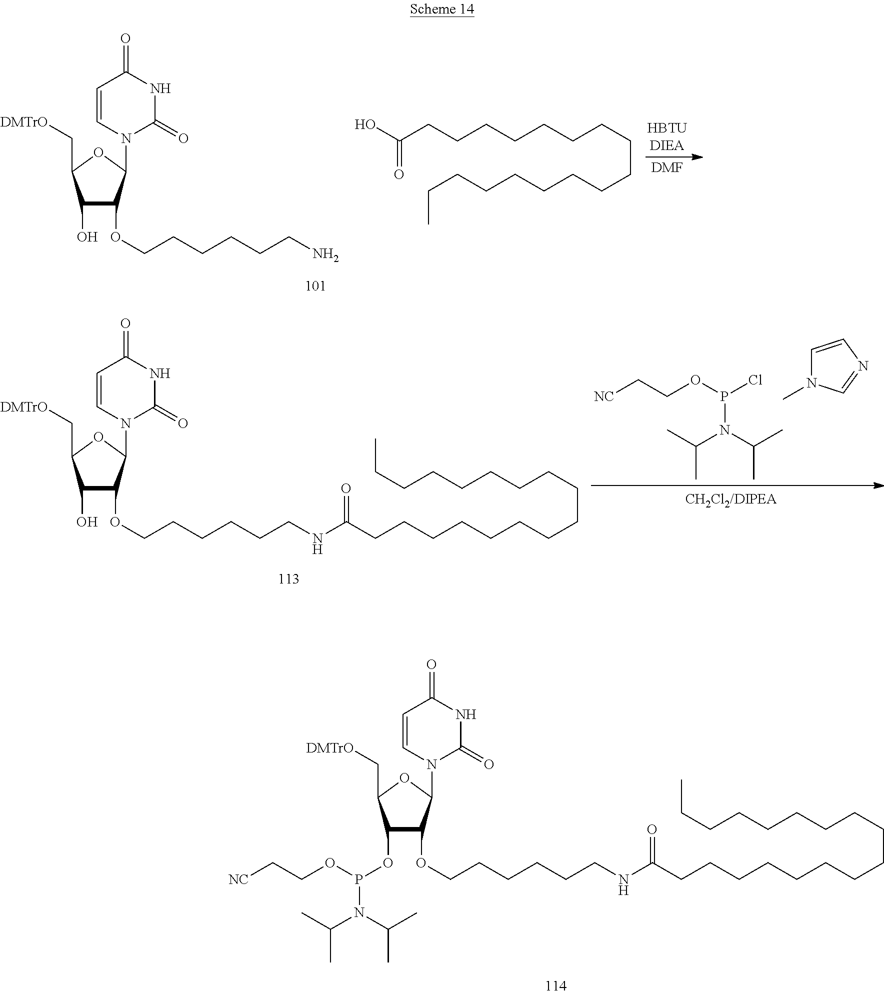

Compound 113: Compound 101 (5.0 g, 7.74 mmol) was added to a reaction flask, along with stearic acid (2.45 g, 8.6 mmol) and HBTU (3.58 g, 9.45 mmol). The solids were dissolved in DMF (25 ml), and diisopropylethylamine (4.05 ml, 23.23 mmol) was added via syringe. The reaction mixture was stirred at room temperature overnight. The reaction mixture was checked by TLC (80% EtOAc/hexane). The reaction mixture was diluted with diethyl ether and dilute sodium bicarbonate solution and was added to separation funnel. The organic layer was washed with dilute sodium bicarbonate solution, saturated sodium bicarbonate, and then saturated brine solution. The organic layer was separated and dried with sodium sulfate. The solid was filtered off and the mother liquor was concentrated. The residue was purified by flash chromatography on silica gel (0% to 100% EtOAc/hexane), and the product fractions were combined and concentrated on reduced pressure to yield (3.56 g, 50%) of Compound 113. ¹H NMR (400 MHz, DMSO-d6) δ 11.36 (d, J=2.0 Hz, 1H), 7.72 (d, J=8.1 Hz, 1H), 7.67 (t, J=5.6 Hz, 1H), 7.42-7.27 (m, 4H), 7.27-7.18 (m, 5H), 6.89 (d, J=8.6 Hz, 4H), 5.78 (d, J=3.6 Hz, 1H), 5.27 (m, 1H), 5.11 (d, J=6.6 Hz, 1H), 4.16 (q, J=6.1 Hz, 1H), 4.02 (q, J=7.1 Hz, 1H), 3.95 (m, 1H), 3.87 (m, 1H), 3.73 (s, 6H), 3.63-3.47 (m, 2H), 3.31-3.18 (m, 2H), 2.98 (q, J=6.5 Hz, 2H), 2.04-1.95 (m, 2H), 1.48 (m, 4H), 1.34 (m, 3H), 1.30-1.15 (m, 31H), 0.83 (t, J=6.7 Hz, 3H).

Compound 114: Compound 113 (5.86 g, 6.44 mmol) was added to a reaction flask, evacuated, and purged with argon. The starting material was dissolved in dichloromethane (60 ml), and diisopropylethylamine (3.36 ml, 19.31 mmol) was added via syringe. The reaction mixture was cooled to 0° C. via ice bath. 2-cyanoethyl N,N-diisopropylchlorophosphoramidite (1.87 ml, 1.98 mmol) and 1-methylimidazole (0.513 ml, 6.44 mmol) were added to the reaction mixture, and the reaction mixture was allowed to warm to room temperature and stirred for 1 hour. The reaction mixture was checked by TLC (80% EtOAc/hexane) and concentrated under reduced pressure. The residue was dissolved in dichloromethane, added to separation funnel, and the organic layer was washed with saturated sodium bicarbonate. The organic layer was separated and washed with a brine solution. The organic layer was separated and dried with sodium sulfate. The solid was filtered off and the mother liquor was concentrated. The residue was purified by flash chromatography on silica gel (0% to 50% EtOAc/hexane), and the product fractions were combined and concentrated on reduced pressure to yield (4.67 g, 65%) of Compound 114. ¹H NMR (400 MHz, acetonitrile-d3) δ 9.17 (s, 1H), 7.49-7.39 (m, 2H), 7.37-7.21 (m, 7H), 6.93-6.83 (m, 4H), 6.29 (d, J=6.0 Hz, 1H), 5.86 (dd, J=7.4, 3.7 Hz, 1H), 5.23 (dd, J=8.1, 6.6 Hz, 1H), 4.43 (m, 1H), 4.21-4.09 (m, 1H), 4.09-3.96 (m, 2H), 3.87 (m, 1H), 3.77 (d, J=2.3 Hz, 6H), 3.61 (m, 4H), 3.46-3.32 (m, 2H), 3.09 (m, 2H), 2.73 (s, 1H), 2.67 (m, 1H), 2.52 (t, J=6.0 Hz, 1H), 2.06 (m, 2H), 1.54 (m, 4H), 1.41 (m, 2H), 1.26 (s, 31H), 1.16 (dd, J=8.8, 6.8 Hz, 11H), 1.05 (d, J=6.8 Hz, 3H), 0.88 (t, J=6.7 Hz, 3H). ³¹P NMR (202 MHz, acetonitrile-d3) δ 151.06, 150.60.

### Synthesis of 2′-O—C6-amide-oleyl Conjugated Uridine Amidite

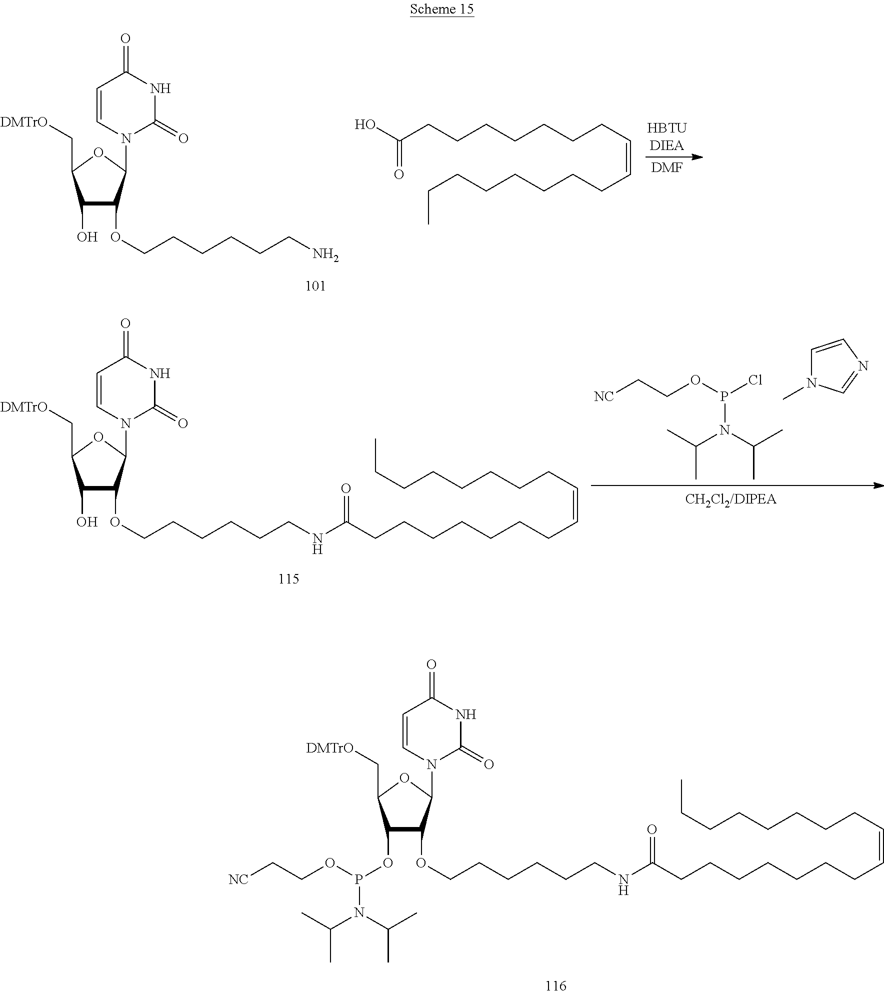

Compound 115: Compound 101 (5.0 g, 7.74 mmol) was added to a reaction flask, along with oleyl acid (2.43 g, 8.6 mmol) and HBTU (3.58 g, 9.45 mmol). The solids were dissolved in DMF (75 ml), and diisopropylethylamine (4.05 ml, 23.23 mmol) was added via syringe. The reaction mixture was stirred at room temperature overnight. The reaction mixture was checked by TLC (80% EtOAc/hexane). The reaction mixture was diluted with diethyl ether and dilute sodium bicarbonate solution, and was added to separation funnel. The organic layer was washed with dilute sodium bicarbonate solution, saturated sodium bicarbonate, and then saturated brine solution. The organic layer was separated and dried with sodium sulfate. The solid was filtered off and the mother liquor was concentrated. The residue was purified by flash chromatography on silica gel (0% to 100% EtOAc/hexane), and the product fractions were combined and concentrated on reduced pressure to yield (5.86 g, 84%) of Compound 115. ¹H NMR (400 MHz, DMSO-d6) δ 11.37 (d, J=2.0 Hz, 1H), 7.73 (d, J=8.1 Hz, 1H), 7.67 (t, J=5.6 Hz, 1H), 7.41-7.28 (m, 4H), 7.28-7.19 (m, 5H), 6.89 (d, J=8.7 Hz, 4H), 5.78 (d, J=3.6 Hz, 1H), 5.35-5.23 (m, 3H), 5.11 (d, J=6.7 Hz, 1H), 4.16 (q, J=6.2 Hz, 1H), 3.95 (m, 1H), 3.88 (m, 1H), 3.73 (s, 6H), 3.63-3.47 (m, 2H), 3.30-3.17 (m, 2H), 2.99 (q, J=6.5 Hz, 2H), 1.98 (m, 6H), 1.47 (m, 4H), 1.35 (q, J=7.0 Hz, 2H), 1.23 (d, J=12.7 Hz, 22H), 0.83 (t, J=6.7 Hz, 3H). Mass calc. for C₅₄H₇₅N₃O₉: 910.21, found: 908.5 (M−H)

Compound 116: Compound 115 (3.56 g, 3.90 mmol) was added to a reaction flask, evacuated, and purged with argon. The starting material was dissolved in dichloromethane (35 ml), and diisopropylethylamine (2.04 ml, 11.71 mmol) was added via syringe. The reaction mixture was cooled to 0° C. via ice bath. 2-cyanoethyl N,N-diisopropylchlorophosphoramidite (1.13 ml, 5.07 mmol) and 1-methylimidazole (0.311 ml, 3.9 mmol) were added to the reaction mixture, and the reaction mixture was allowed to warm to room temperature and stirred for 1 hour. The reaction mixture was checked by TLC (80% EtOAc/hexane) and concentrated under reduced pressure. The residue was dissolved in dichloromethane, added to separation funnel, and the organic layer was washed with saturated sodium bicarbonate. The organic layer was separated and washed with a brine solution. The organic layer was separated and dried with sodium sulfate. The solid was filtered off and the mother liquor was concentrated. The residue was purified by flash chromatography on silica gel (0% to 100% EtOAc/hexane), and the product fractions were combined and concentrated on reduced pressure to yield (3.5 g, 80%) of Compound 116. ¹H NMR (500 MHz, acetonitrile-d3) δ 9.16 (s, 1H), 7.48-7.40 (m, 2H), 7.38-7.22 (m, 7H), 6.92-6.84 (m, 4H), 6.28 (d, J=6.9 Hz, 1H), 5.86 (dd, J=9.2, 3.7 Hz, 1H), 5.34 (m, 2H), 5.23 (t, J=8.2 Hz, 1H), 4.51-4.36 (m, 1H), 4.15 (m, 1H), 4.07-3.97 (m, 1H), 3.93-3.81 (m, 1H), 3.77 (d, J=2.9 Hz, 7H), 3.61 (m, 4H), 3.45-3.33 (m, 2H), 3.09 (m, 2H), 2.81-2.69 (m, 1H), 2.69-2.58 (m, 1H), 2.52 (t, J=6.0 Hz, 1H), 2.10-1.97 (m, 6H), 1.54 (m, 4H), 1.47-1.39 (m, 2H), 1.39-1.19 (m, 25H), 1.16 (dd, J=10.8, 6.8 Hz, 9H), 1.05 (d, J=6.7 Hz, 3H), 0.88 (t, J=6.8 Hz, 3H). ³¹P NMR (202 MHz, acetonitrile-d3) δ 151.06, 150.60.

### Synthesis of 2′-O—C3-amide-oleyl Conjugated Uridine Amidite

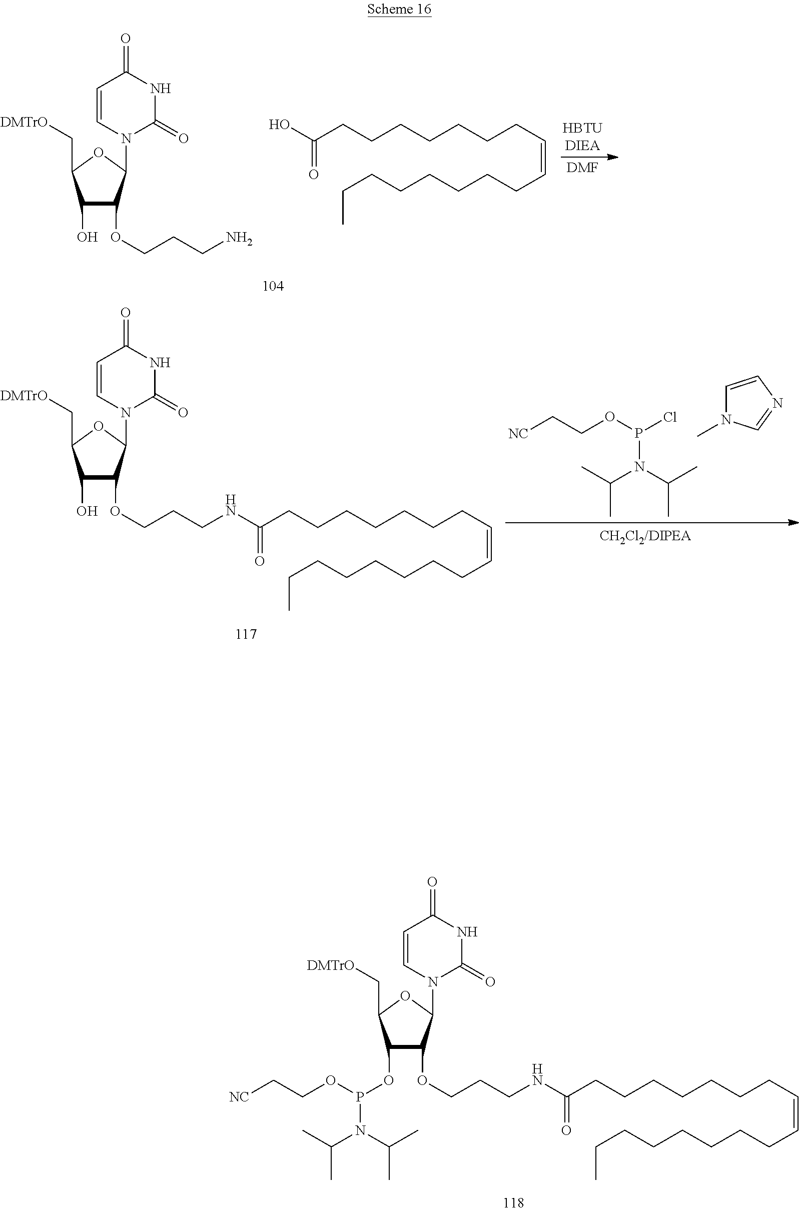

Compound 117: Compound 104 (5.0 g, 8.28 mmol) was added to a reaction flask, along with oleyl acid (2.6 g, 9.19 mmol) and HBTU (3.83 g, 10.11 mmol). The solids were dissolved in DMF (70 ml), and diisopropylethylamine (4.33 ml, 24.85 mmol) was added via syringe. The reaction mixture was stirred at room temperature overnight. The reaction mixture was checked by TLC (80% EtOAc/hexane). The reaction mixture was diluted with diethyl ether and dilute sodium bicarbonate solution, and was added to separation funnel. The organic layer was washed with dilute sodium bicarbonate solution, saturated sodium bicarbonate, and then saturated brine solution. The organic layer was separated and dried with sodium sulfate. The solid was filtered off and the mother liquor was concentrated. The residue was purified by flash chromatography on silica gel (0% to 100% EtOAc/hexane), and the product fractions were combined and concentrated on reduced pressure to yield (4.6 g, 64%) of Compound 117. ¹H NMR (400 MHz, DMSO-d6) δ 11.37 (d, J=2.2 Hz, 1H), 7.75-7.67 (m, 2H), 7.41-7.26 (m, 4H), 7.23 (m, 5H), 6.89 (d, J=8.5 Hz, 4H), 5.78 (d, J=3.4 Hz, 1H), 5.33-5.23 (m, 3H), 5.18 (d, J=6.6 Hz, 1H), 4.18 (q, J=6.3 Hz, 1H), 3.95 (m, 1H), 3.89 (dd, J=5.2, 3.5 Hz, 1H), 3.73 (s, 6H), 3.57 (q, J=5.6, 4.9 Hz, 2H), 3.31-3.18 (m, 2H), 3.09 (m, 2H), 2.05-1.90 (m, 6H), 1.63 (m, 2H), 1.45 (q, J=7.2 Hz, 2H), 1.23 (m, 20H), 0.83 (t, J=6.6 Hz, 3H). Mass calc. for C₅₁H₆₉N₃O₉: 868.13, found: 867.5 (M−H).

Compound 118: Compound 117 (4.6 g, 5.3 mmol) was added to a reaction flask, evacuated, and purged with argon. The starting material was dissolved in dichloromethane (45 ml), and diisopropylethylamine (2.77 ml, 15.9 mmol) was added via syringe. The reaction mixture was cooled to 0° C. via ice bath. 2-cyanoethyl N,N-diisopropylchlorophosphoramidite (1.54 ml, 6.89 mmol) and 1-methylimidazole (0.422 ml, 5.3 mmol) were added to the reaction mixture, and the reaction mixture was allowed to warm to room temperature and stirred for 1 hour. The reaction mixture was checked by TLC (80% EtOAc/hexane) and concentrated under reduced pressure. The residue was dissolved in dichloromethane, added to separation funnel, and the organic layer was washed with saturated sodium bicarbonate. The organic layer was separated and washed with a brine solution. The organic layer was separated and dried with sodium sulfate. The solid was filtered off and the mother liquor was concentrated. The residue was purified by flash chromatography on silica gel (0% to 60% EtOAc/hexane), and the product fractions were combined and concentrated on reduced pressure to yield (4.64 g, 82%) of Compound 118. ¹H NMR (400 MHz, acetonitrile-d3) δ 9.12 (s, 1H), 7.52-7.42 (m, 2H), 7.42-7.24 (m, 7H), 6.96-6.86 (m, 4H), 6.45 (d, J=4.9 Hz, 1H), 5.88 (dd, J=6.6, 2.8 Hz, 1H), 5.41-5.32 (m, 2H), 5.24 (dd, J=8.2, 7.2 Hz, 1H), 4.49 (m, 1H), 4.16 (m, 1H), 4.12-4.02 (m, 1H), 3.84-3.72 (m, 9H), 3.72-3.56 (m, 3H), 3.56-3.36 (m, 3H), 3.25 (m, 2H), 2.78 (t, J=5.9 Hz, 1H), 2.71 (m, 1H), 2.55 (t, J=6.0 Hz, 1H), 2.15-2.07 (m, 2H), 2.04 (m, 4H), 1.74 (m, F₂H), 1.55 (d, J=7.2 Hz, 2H), 1.40-1.23 (m, 26H), 1.23-1.12 (m, 9H), 1.07 (d, J=6.8 Hz, 3H), 0.94-0.86 (m, 3H). ³¹P NMR (162 MHz, acetonitrile-d3) δ 149.59 (d, J=2.2 Hz), 149.11 (d, J=2.6 Hz).

### Synthesis of 2′-O—C3 and 2′-O—C6 Phosphoramidite of A, G, C and U

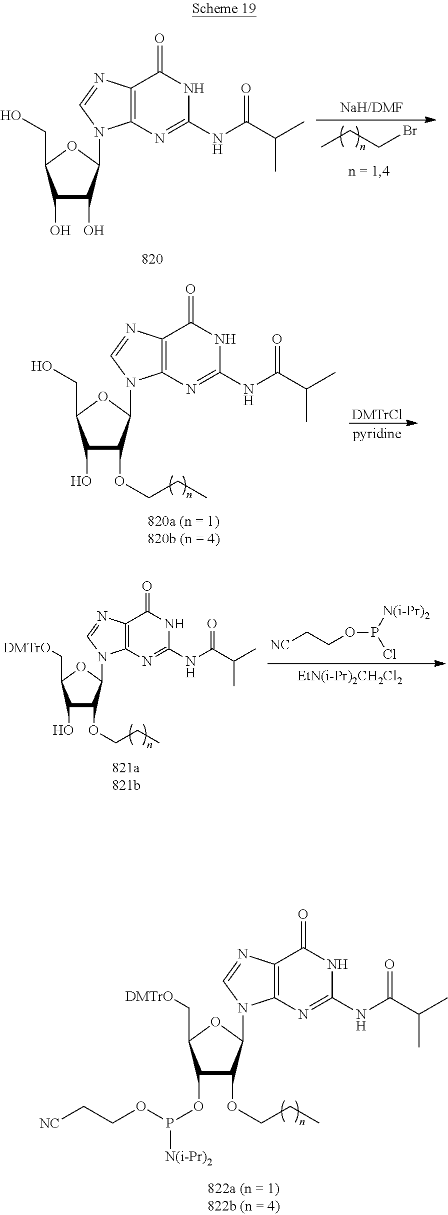

Synthesis of 2′-O—C3 Uridine Phosphoramidite 804a: Prior to synthesis, the starting material, Compound 803a (4.00 g, 6.80 mmol) was co-evaporated with acetonitrile twice and dried on high vacuum overnight. To a solution of Compound 803a in anhydrous dichloromethane (79.03 mL) and DIPEA (4.14 mL, 23.78 mmol) were added. The mixture was cooled to 0° C. on ice bath and 2-cyanoethyl N,N-diisopropylchlorophosphoramidite (2.73 mL, 12.23 mmol) was added dropwise. The mixture was warmed to room temperature and stirred for 4 hours, and TLC was checked (60% EtOAc in hexanes). The solvent was stripped under reduced pressure and the residue was dried on high vacuum for 1 hour. The residue was resuspended in EtOAc and quickly performed standard aqueous workup with saturated aqueous NaHCO₃. The organic layers were combined, washed with saturated aqueous NaCl, dried over anhydrous sodium sulfate, and concentrated under reduced pressure. The residue was purified by flash chromatography on silica gel (pre-treated with Et₃N) with gradient 0-60% of EtOAc in hexanes to afford 4.53 g (84% yield) of Compound 804a. ¹H NMR (400 MHz, acetonitrile-d₃) δ 9.09 (s, 1H), 7.79 (dd, J=35.3, 8.1 Hz, 1H), 7.45 (ddt, J=10.6, 8.2, 1.3 Hz, 2H), 7.38-7.21 (m, 7H), 6.92-6.83 (m, 4H), 5.85 (dd, J=6.0, 3.2 Hz, 1H), 5.22 (dd, J=8.2, 5.3 Hz, 1H), 4.46 (dddd, J=31.1, 10.0, 6.6, 4.9 Hz, 1H), 4.15 (ddt, J=13.4, 6.3, 2.9 Hz, 1H), 4.04 (ddd, J=13.8, 4.9, 3.2 Hz, 1H), 3.80-3.73 (m, 7H), 3.68-3.54 (m, 3H), 3.45-3.37 (m, 2H), 2.70-2.63 (m, 1H), 2.15 (s, 1H), 1.64-1.52 (m, 2H), 1.16 (dd, J=9.9, 6.8 Hz, 9H), 1.05 (d, J=6.8 Hz, 3H), 0.91 (td, J=7.4, 5.2 Hz, 3H). ³¹P NMR (162 MHz, CD₃CN) δ 150.15, 150.10, 149.74, 149.69, 14.24, 6.08.

Synthesis of 2′-O—C6 Uridine Phosphoramidite 804b: Compound 803b (4.0 g, 6.35 mmol) was added to a reaction flask, evacuated and purged with argon. The starting material was dissolved in dichloromethane, and diisopropylamine (2.21 ml, 12.7 mmol) was added via syringe. 2-Cyanoethyl N,N-diisopropylchlorophosphoramidite (2.12 ml, 9.53 mmol) was added and stirred at room temperature for 3 hours. The reaction was checked by TLC (70% EtOAc in hexane) and the reaction was concentrated under reduced pressure. The residue was dissolved in dichloromethane, added to separation funnel and organic layer was washed with saturated sodium bicarbonate solution. The organic layer was separated and washed with a brine solution. The organic layer was separated and dried with sodium sulfate. The solid was filtered off and the mother liquor was concentrated. The residue was purified by flash chromatography on silica gel (30% to 100% EtOAc in hexane) and the product fractions combined and concentrated on reduced pressure to yield (3.42 g, 65%) of 804b. ¹H NMR (400 MHz, acetonitrile-d₃) δ 8.98 (s, 1H), 7.86-7.66 (m, 1H), 7.49-7.39 (m, 2H), 7.39-7.21 (m, 7H), 6.93-6.83 (m, 4H), 5.85 (dd, J=6.2, 3.5 Hz, 1H), 5.22 (dd, J=8.2, 6.3 Hz, 1H), 4.44 (m, 1H), 4.20-3.98 (m, 2H), 3.93-3.82 (m, 1H), 3.77 (d, J=2.4 Hz, 7H), 3.71-3.55 (m, 5H), 3.47-3.32 (m, 2H), 2.72-2.61 (m, 1H), 2.52 (t, J=6.0 Hz, 1H), 1.62-1.49 (m, 2H), 1.41-1.23 (m, 6H), 1.17 (dd, J=8.8, 6.8 Hz, 9H), 1.05 (d, J=6.8 Hz, 3H), 0.88 (m, 3H). 31P NMR (202 MHz, acetonitrile-d₃) δ 149.63, 149.26.

### Synthesis of 2′-O—C6 and 2′-O—C3 Adenosine Phosphoramidite

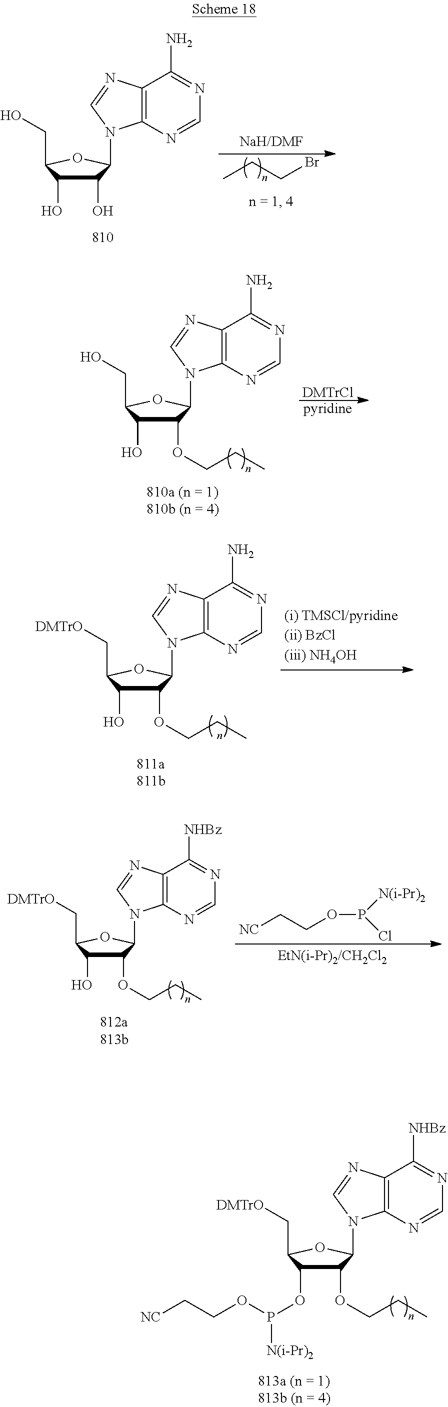

Synthesis of Compounds 813a and 813b: By utilizing a procedure shown in the above synthetic Scheme 18 and a procedure similar to the phosphitylation process described for the synthesis of Compound 804b, Compounds 813a and 813b were synthesized and characterized.

### Synthesis of 2′-O—C6 and 2′-O—C3 Guanosine Phosphoramidite

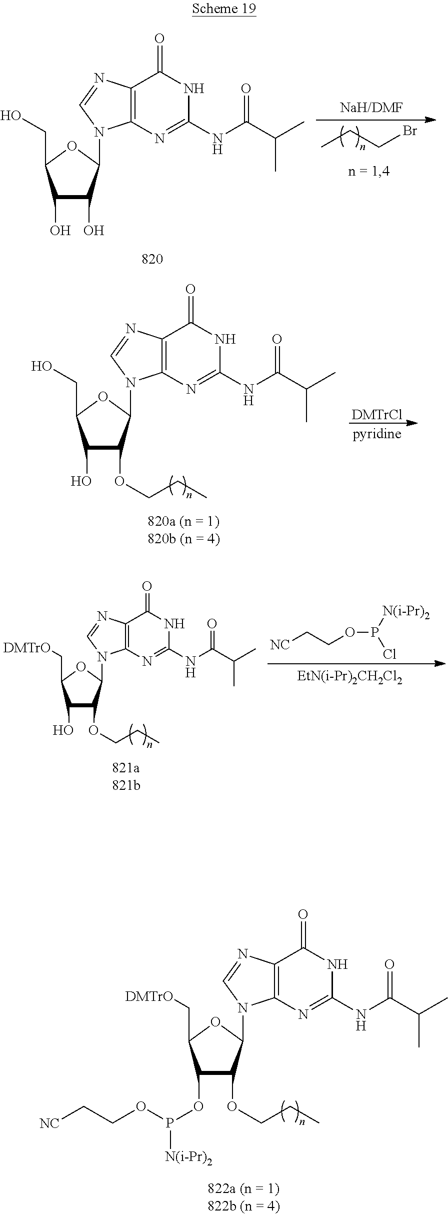

Synthesis of Compounds 822a and 822b: By utilizing a procedure shown in the above synthetic Scheme 19 and a procedure similar to the phosphitylation processes described for the synthesis of Compound 804b, Compounds 822a and 822b were synthesized and characterized.

### Synthesis of 2′-O—C6-amide-C16 Ester Conjugated Uridine Amidite

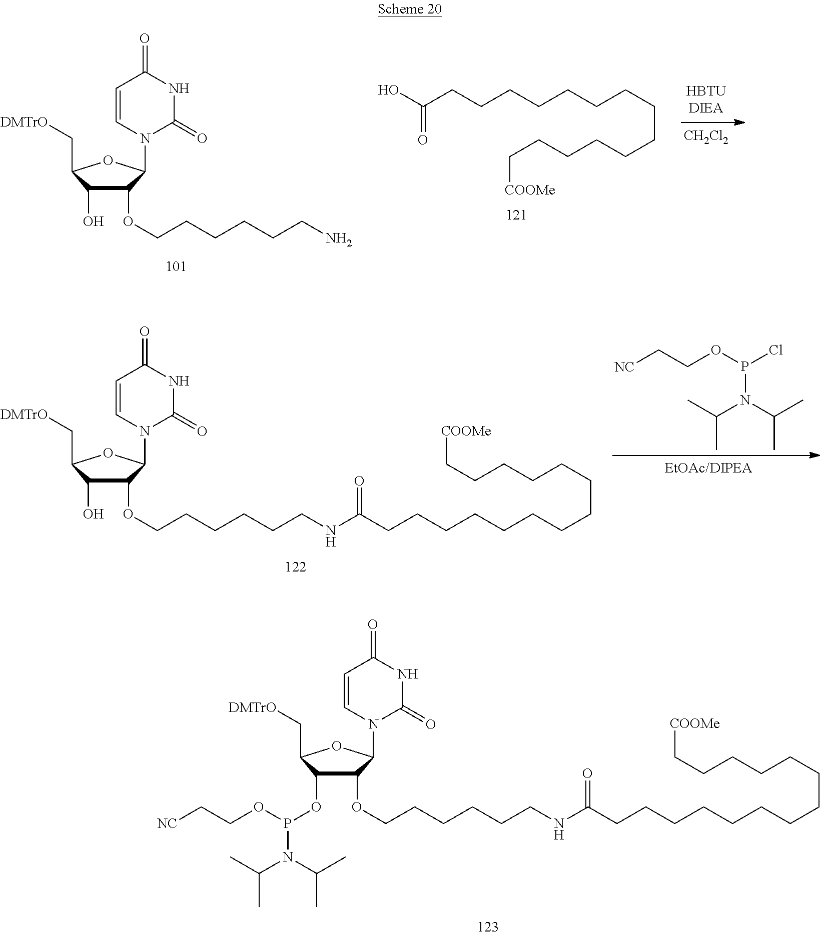

Compound 122: To a heat-oven dried 100 mL round bottle flask, added a solution of Compound 101, (4 g, 6.19 mmol, 1.0 eq.) in anhydrous DCM (120 mL). 16-methoxy-16-oxohexadecanoic acid, Compound 121 (2.05 g, 6.81 mmol, 1.1 eq.), was added to the solution, followed by HBTU (2.58 g, 6.81 mmol, 1.1 eq.) and DIPEA (3.24 mL, 18.58 mmol, 3 eq.). The resultant solution was stirred at room temperature under argon overnight. TLC with 100% EtOAc/hexane showed the formation of the product. The reaction mixture was quenched with brine solution, and extracted with DCM. The combined organic solution was dried over anhydrous Na₂SO₄, filtered, and concentrated to an oil. Purification through ISCO column chromatography with 80 g silica gel column eluted with 0-100% EtOAc/hexane gave Compound 122. A thick oil product was yielded (4.81 g, 84%). ¹H NMR (500 MHz, chloroform-d) δ 8.41 (s, 1H), 8.00 (d, J=8.2 Hz, 1H), 7.41-7.35 (m, 2H), 7.34-7.20 (m, 10H), 6.88-6.81 (m, 4H), 5.94 (d, J=1.9 Hz, 1H), 5.48 (t, J=5.6 Hz, 1H), 5.32-5.23 (m, 1H), 4.49-4.41 (m, 1H), 4.03 (dt, J=7.6, 2.4 Hz, 1H), 3.93-3.84 (m, 2H), 3.80 (d, J=1.1 Hz, 6H), 3.66 (s, 4H), 3.54 (qd, J=11.1, 2.4 Hz, 2H), 3.24 (td, J=7.2, 5.9 Hz, 2H), 2.80 (s, 10H), 2.75 (d, J=8.7 Hz, 1H), 2.30 (t, J=7.5 Hz, 2H), 2.18-2.11 (m, 2H), 1.49 (q, J=7.3 Hz, 2H), 1.29-1.23 (m, 17H).

Compound 123: Compound 122 (4.81 g, 5.18 mmol, 1 eq.) was dissolved in anhydrous EtOAc (120 mL). Under argon and cooled in an ice bath, added DIPEA (2.71 ml, 15.55 mmol, 3 eq.) followed by N,N-diisopropylaminocyanoethyl phosphonamidic-Cl (1.35 g, 5.70 mmol, 1.1 eq.). The reaction mixture was stirred at room temperature overnight. TLC at 100% EtOAc/hexane showed the completion of the reaction. The reaction mixture was quenched with brine, and extracted with EtOAc. The organic layer was separated, dried over Na₂SO₄, and concentrated to a white oil. ISCO purification eluted with 0-100% EtOAc/hexane gave Compound 123, with a yield 78.3% (4.58 g). ¹H NMR (500 MHz, acetonitrile-d3) δ 9.41 (s, 1H), 7.90 (s, 1H), 7.78 (dd, J=42.9, 8.1 Hz, 1H), 7.48-7.40 (m, 2H), 7.38-7.21 (m, 7H), 6.92-6.84 (m, 4H), 6.39-6.32 (m, 1H), 5.86 (dd, J=9.1, 3.6 Hz, 1H), 5.45 (s, 3H), 5.24 (t, J=7.9 Hz, 1H), 4.15 (ddt, J=17.6, 6.1, 2.9 Hz, 1H), 4.07-3.98 (m, 1H), 3.77 (d, J=3.1 Hz, 8H), 3.66-3.56 (m, 7H), 3.47-3.34 (m, 2H), 3.13-3.05 (m, 2H), 2.73 (s, 8H), 2.71-2.62 (m, 1H), 2.26 (t, J=7.5 Hz, 2H), 2.06 (td, J=7.5, 2.2 Hz, 2H), 1.54 (dtd, J=13.4, 6.3, 3.4 Hz, 6H), 1.47-1.38 (m, 2H), 1.34 (t, J=7.3 Hz, 2H), 1.26 (d, J=6.2 Hz, 22H), 1.05 (d, J=6.8 Hz, 3H). ³¹P NMR (202 MHz, acetonitrile-d₃) δ 151.59, 151.11.

### Synthesis of 2′-O—C6-amide-C18 Ester Conjugated Uridine Amidite

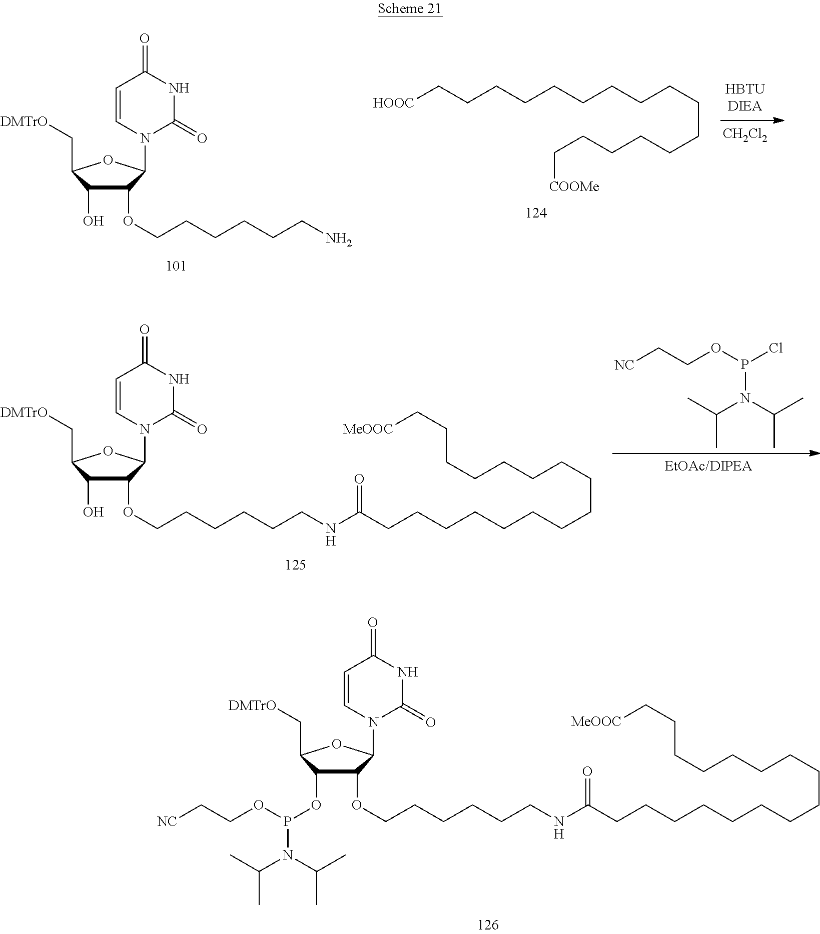

Compound 125: Compound 125 was obtained by using compound 101 and 18-methoxy-18-oxooctadecanoic acid 124 in a procedure similar to the procedure above for synthesizing Compound 122. ¹H NMR (500 MHz, chloroform-d) δ 8.57 (s, 1H), 8.00 (d, J=8.2 Hz, 1H), 7.41-7.35 (m, 2H), 7.33-7.20 (m, 9H), 6.88-6.81 (m, 4H), 5.51 (t, J=5.8 Hz, 1H), 5.31-5.24 (m, 1H), 4.45 (td, J=8.1, 5.2 Hz, 1H), 4.03 (dt, J=7.6, 2.4 Hz, 1H), 3.88 (td, J=6.6, 6.0, 4.5 Hz, 2H), 3.79 (d, J=1.1 Hz, 6H), 3.66 (s, 4H), 3.54 (qd, J=11.2, 2.4 Hz, 2H), 3.24 (td, J=7.2, 5.9 Hz, 2H), 2.80 (s, 11H), 2.76 (d, J=8.7 Hz, 2H), 2.30 (t, J=7.6 Hz, 2H), 2.18-2.07 (m, 2H), 1.48 (q, J=7.2 Hz, 2H), 1.29-1.23 (m, 21H).

Compound 126: Compound 126 was obtained by using compound 125 with N,N-diisopropylaminocyanoethyl phosphonamidic-Cl in a procedure similar to the procedure above for synthesizing Compound 123. ¹H NMR (500 MHz, acetonitrile-d₃) δ 9.44 (s, 1H), 7.78 (dd, J=42.6, 8.2 Hz, 1H), 7.48-7.40 (m, 2H), 7.38-7.21 (m, 7H), 6.92-6.83 (m, 4H), 6.37 (q, J=5.6 Hz, 1H), 5.86 (dd, J=9.1, 3.5 Hz, 1H), 5.24 (dd, J=8.1, 7.1 Hz, 1H), 4.15 (ddt, J=17.5, 6.2, 2.9 Hz, 1H), 4.10-3.98 (m, 2H), 3.82-3.54 (m, 15H), 3.46-3.34 (m, 2H), 3.09 (tdd, J=7.0, 5.8, 3.3 Hz, 2H), 2.71-2.62 (m, 1H), 2.55-2.49 (m, 1H), 2.26 (t, J=7.5 Hz, 2H), 2.06 (td, J=7.4, 2.2 Hz, 2H), 1.61-1.49 (m, 6H), 1.41 (dtd, J=12.2, 7.2, 6.3, 3.4 Hz, 2H), 1.37-1.20 (m, 30H), 1.17-1.13 (m, 7H), 1.05 (d, J=6.8 Hz, 3H). ³¹P NMR (202 MHz, acetonitrile-d₃) δ 151.36.

### Synthesis of 2′-O—C6-amide-C20 Ester Conjugated Uridine Amidite

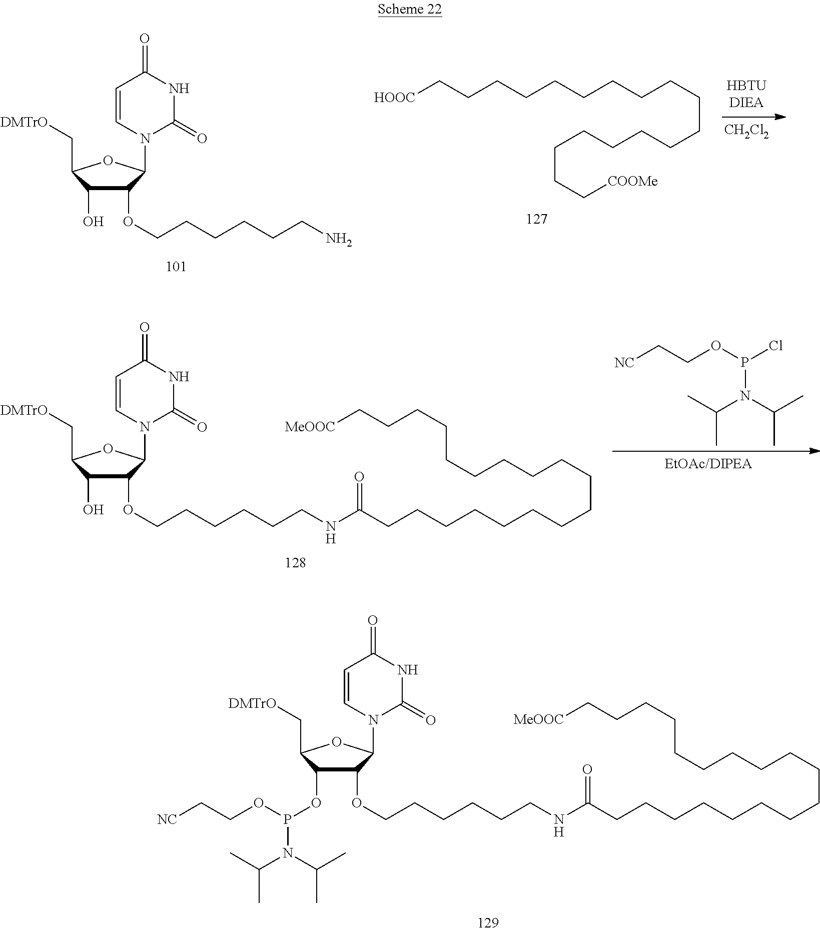

Compound 128: Compound 128 was obtained by using compound 101 and 20-methoxy-20-oxoicosanoic acid 127 in a procedure similar to the procedure above for synthesizing Compound 128. ¹H NMR (500 MHz, chloroform-d) δ 8.00 (d, J=8.2 Hz, 1H), 7.41-7.35 (m, 2H), 7.34-7.21 (m, 10H), 6.88-6.81 (m, 4H), 5.94 (d, J=1.8 Hz, 1H), 5.27 (d, J=8.2 Hz, 1H), 4.45 (td, J=8.1, 5.3 Hz, 1H), 4.03 (dt, J=7.6, 2.5 Hz, 1H), 3.93-3.85 (m, 2H), 3.80 (d, J=1.0 Hz, 6H), 3.66 (s, 4H), 3.59-3.49 (m, 2H), 3.24 (q, J=6.8 Hz, 2H), 2.80 (s, 11H), 2.75 (d, J=8.6 Hz, 1H), 2.30 (t, J=7.6 Hz, 2H), 2.18-2.11 (m, 2H), 1.49 (q, J=7.3 Hz, 2H), 1.25 (d, J=6.6 Hz, 25H).

Compound 129: Compound 129 was obtained by using compound 128 with N,N-diisopropylaminocyanoethyl phosphonamidic-Cl in a procedure similar to the procedure above for synthesizing Compound 123. ¹H NMR (400 MHz, acetonitrile-d₃) δ 9.27 (s, 1H), 7.76 (dd, J=34.6, 8.1 Hz, 1H), 7.49-7.39 (m, 2H), 7.38-7.21 (m, 7H), 6.93-6.83 (m, 4H), 6.33 (d, J=5.9 Hz, 1H), 5.86 (dd, J=7.4, 3.6 Hz, 1H), 5.23 (dd, J=8.1, 6.3 Hz, 1H), 4.15 (ddt, J=13.6, 6.1, 2.9 Hz, 1H), 4.08-3.97 (m, 1H), 3.77 (d, J=2.3 Hz, 7H), 3.71-3.54 (m, 7H), 3.46-3.33 (m, 2H), 3.09 (qd, J=7.1, 2.5 Hz, 2H), 2.27 (t, J=7.5 Hz, 2H), 2.17 (s, 6H), 2.06 (td, J=7.4, 1.9 Hz, 2H), 1.61-1.47 (m, 6H), 1.47-1.37 (m, 3H), 1.26 (s, 32H), 1.18-1.12 (m, 7H), 1.05 (d, J=6.7 Hz, 3H). ³¹P NMR (162 MHz, acetonitrile-d₃) S 151.08, 150.60 (d, J=7.1 Hz).

### Synthesis of 2′, 3′-O-pentadecyl ω Carboxymethyl Ester Uridine Phosphoramidites

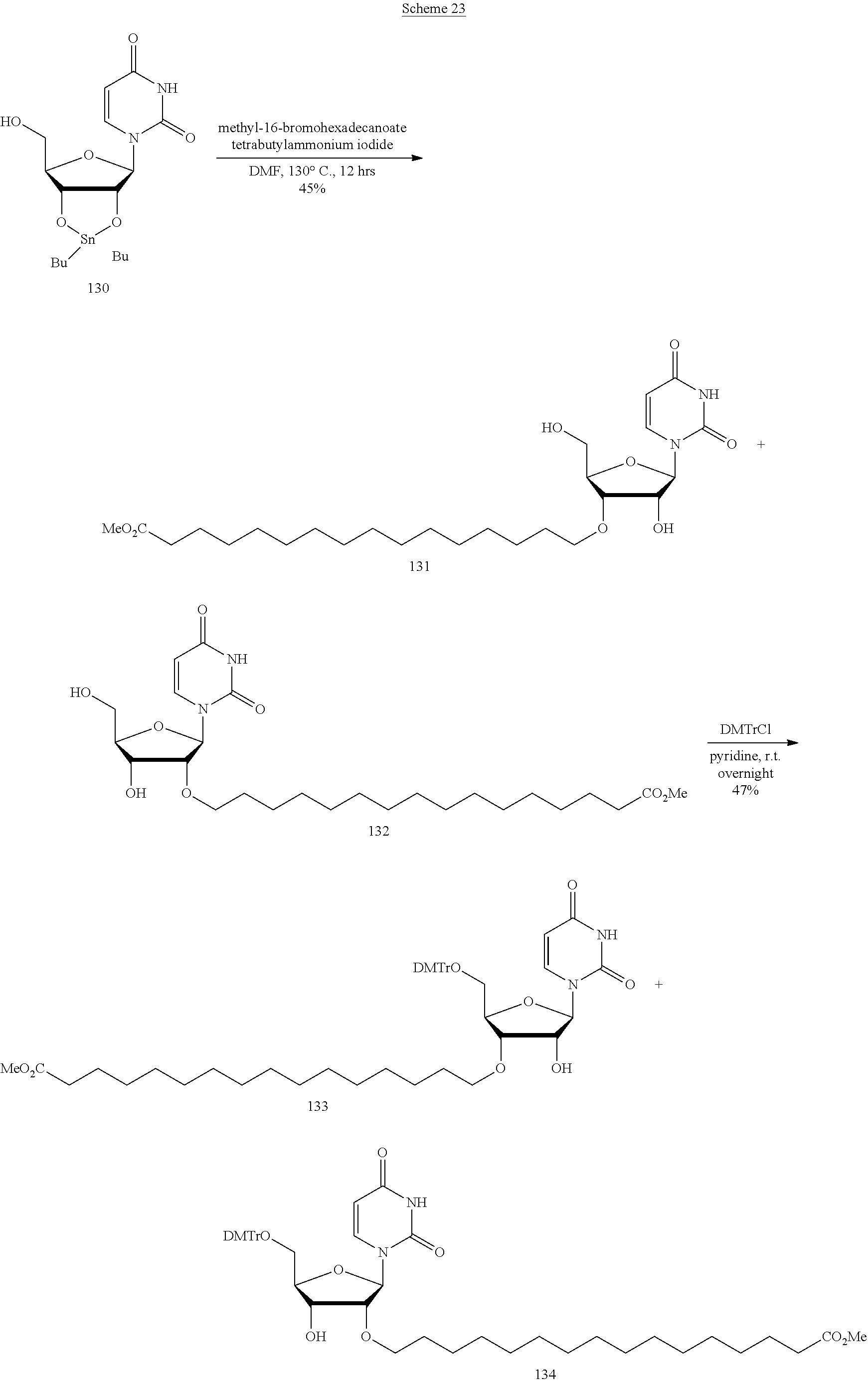

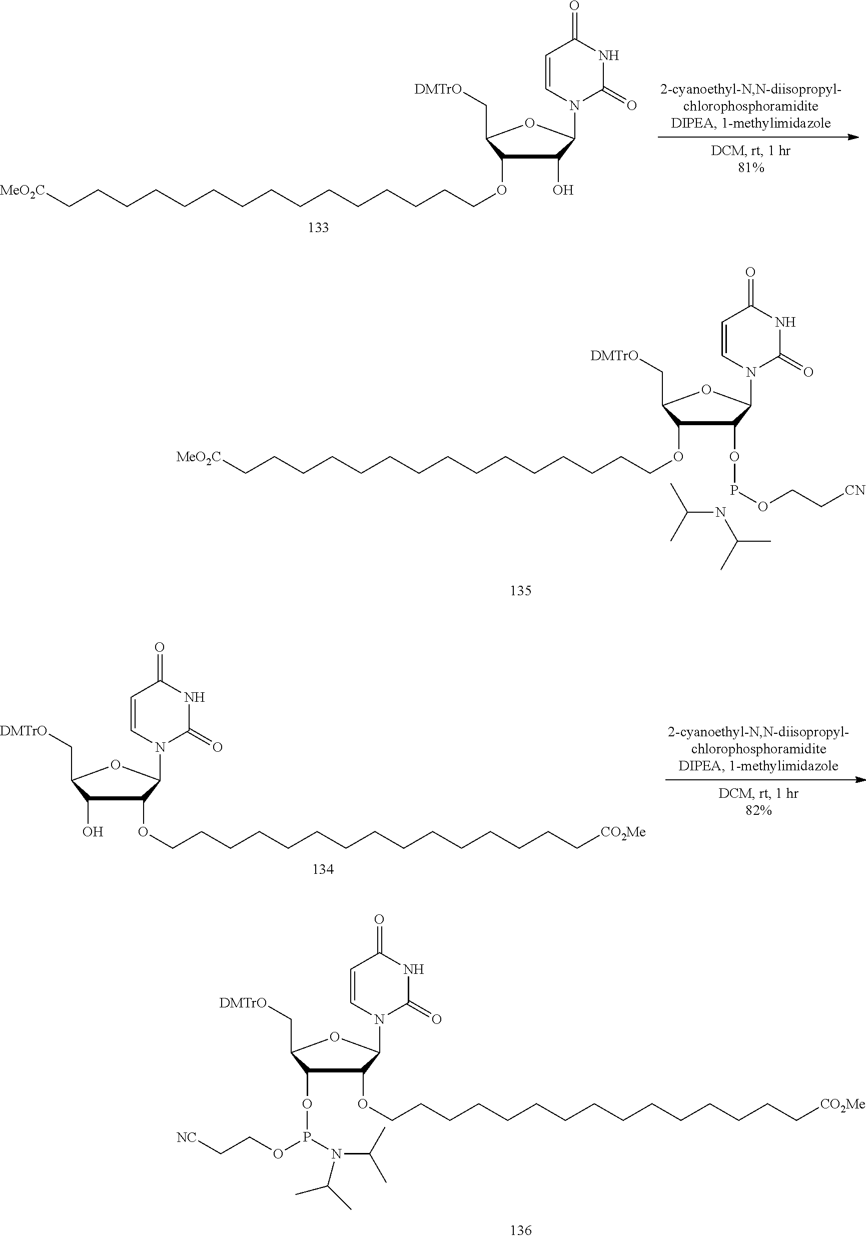

Compounds 131 and 132: To a solution of Compound 130 (3.3 g, 6.95 mmol) in dimethylformamide (DMF) (60 mL), was added methyl-16-bromohexadecanoate (5.00 g, 13.89 mmol) and tetrabutylammonium iodide (5.24 g, 13.89 mmol) in a single portion. The resulting mixture was heated to reflux at 130° C. for 12 hours. DMF of the resulting red colored solution was removed under high vacuum to obtain a gummy brown mass which was purified by combiflash chromatography (gradient: 0-10% MeOH in DCM) to afford a mixture of Compounds 131 and 132 (1.45 g, 41% yield) and as yellowish brown solid. ¹H NMR (400 MHz, DMSO-d₆) δ 11.32 (dd, J=5.1, 2.3 Hz, 2H), 7.93 (d, J=8.1 Hz, 1H), 7.88 (d, J=8.1 Hz, 1H), 5.83 (d, J=5.2 Hz, 1H), 5.74 (d, J=5.3 Hz, 1H), 5.64 (dt, J=8.1, 2.6 Hz, 2H), 5.31 (d, J=6.1 Hz, 1H), 5.13 (td, J=5.1, 1.9 Hz, 2H), 5.04 (d, J=5.8 Hz, 1H), 4.16 (q, J=5.5 Hz, 1H), 4.08 (q, J=5.0 Hz, 1H), 3.99 (t, J=6.4 Hz, 1H), 3.91 (q, J=3.5 Hz, 1H), 3.87-3.80 (m, 2H), 3.75 (t, J=4.6 Hz, 1H), 3.68-3.49 (m, 10H), 3.43 (tt, J=9.6, 6.7 Hz, 2H), 3.22-3.06 (m, 8H), 2.28 (dd, J=8.3, 6.6 Hz, 4H), 1.66-1.43 (m, 17H), 1.31-1.15 (m, 58H), 0.93 (t, J=7.3 Hz, 11H) ppm. ¹³C NMR (126 MHz, DMSO-d₆) δ 173.30, 172.84, 163.05, 163.00, 150.69, 150.49, 140.52, 140.34, 101.76, 101.65, 87.99, 86.06, 85.12, 82.77, 81.04, 79.16, 77.44, 72.61, 69.74, 69.60, 68.33, 63.54, 60.75, 60.50, 57.55, 57.53, 57.50, 51.11, 33.56, 33.24, 29.33, 29.05, 29.02, 29.00, 28.94, 28.91, 28.88, 28.84, 28.79, 28.63, 28.55, 28.42, 28.38, 28.09, 25.53, 25.36, 24.51, 24.40, 23.06, 19.20, 19.18, 13.46 ppm.

Compounds 133 and 134: To a clear solution of Compounds 131 and 132 (1.5 g, 2.92 mmol) in dry pyridine (30 mL) was added 4,4′-dimethoxytrityl chloride (1.25 g, 3.51 mmol) in three portions. Reaction mixture was stirred for 12 hours at 22° C. and then quenched with saturated NaHCO₃ solution (30 mL). The resulting mixture was extracted with DCM (2×40 mL). The combined organic layer was separated, washed with brine (40 mL), dried over anhydrous Na₂SO₄, filtered, and the filtrate was evaporated to dryness. Crude compound was purified by combiflash chromatography (gradient: 10-50% ethyl acetate in hexane) to afford Compound 133 as white foam (0.32 g, 13%) and Compound 134 as yellowish white foam (0.81 g, 34%). Spectral data for Compound 133: ¹H NMR (500 MHz, DMSO-d₆) δ 11.34 (d, J=2.3 Hz, 1H), 7.77 (d, J=8.1 Hz, 1H), 7.46-7.13 (m, 9H), 6.89 (dd, J=9.0, 1.8 Hz, 4H), 5.70 (d, J=3.6 Hz, 1H), 5.40 (s, 1H), 5.30 (dd, J=8.1, 2.3 Hz, 1H), 4.24 (t, J=4.3 Hz, 1H), 4.03-3.94 (m, 1H), 3.92 (dd, J=6.5, 4.9 Hz, 1H), 3.74 (s, 6H), 3.57 (s, 4H), 3.43-3.33 (m, 1H), 3.27 (ddd, J=31.4, 10.8, 3.6 Hz, 2H), 2.27 (t, J=7.4 Hz, 2H), 1.60-1.41 (m, 4H), 1.22 (d, J=6.6 Hz, 23H) ppm. ¹³C NMR (126 MHz, DMSO-d₆) δ 173.66, 163.25, 158.34, 150.52, 144.75, 140.73, 135.46, 135.35, 129.92, 129.89, 129.09, 128.08, 127.85, 127.79, 127.62, 127.02, 113.40, 112.96, 101.54, 89.62, 86.15, 80.58, 76.85, 72.08, 69.86, 62.49, 55.22, 51.35, 33.45, 29.32, 29.15, 29.13, 29.10, 29.00, 28.81, 28.60, 25.63, 24.59 ppm. Spectral data for Compound 134: ¹H NMR (500 MHz, DMSO-d₆) δ 11.36 (d, J=2.2 Hz, 1H), 7.72 (d, J=8.1 Hz, 1H), 7.47-7.14 (m, 9H), 6.90 (d, J=8.9 Hz, 4H), 5.80 (d, J=3.9 Hz, 1H), 5.29 (dd, J=8.0, 2.2 Hz, 1H), 5.10 (s, 1H), 4.17 (t, J=5.7 Hz, 1H), 3.96 (ddd, J=6.4, 4.4, 2.8 Hz, 1H), 3.90 (dd, J=5.2, 4.0 Hz, 1H), 3.74 (s, 6H), 3.57 (s, 4H), 3.55-3.50 (m, 1H), 3.34-3.28 (m, 2H), 3.23 (dd, J=10.7, 2.8 Hz, 1H), 2.27 (t, J=7.4 Hz, 2H), 1.50 (td, J=7.5, 7.0, 3.3 Hz, 4H), 1.22 (s, 24H) ppm. ¹³C NMR (126 MHz, DMSO-d₆) δ 173.32, 162.92, 158.13, 150.27, 144.60, 140.15, 135.33, 135.05, 129.75, 127.88, 127.69, 126.78, 113.25, 113.23, 101.48, 86.97, 85.90, 82.72, 80.80, 69.77, 68.49, 62.69, 55.03, 51.11, 33.24, 29.02, 29.00, 28.94, 28.84, 28.79, 28.63, 28.42, 25.37, 24.40 ppm.

Compound 135: To a clear solution of Compound 133 (1.0 g, 1.23 mmol) in dry dichloromethane (30 mL) at 22° C. was added diisopropylethylamine (800.89 mg, 6.13 mmol, 1.08 mL) and N-methylimidazole (152.63 mg, 1.84 mmol, 148.19 μL) slowly. The resulting solution was stirred for 5 minutes after which 2-cyanoethyl-N,N-diisopropylchlorophosphoramidite (611.38 mg, 2.45 mmol, 576.77 μL) was added in single portion. The reaction mixture was kept for 1 hr stirring at 22° C. and TLC was checked. The reaction mixture was diluted with dichloromethane (50 mL) and washed with 10% sodium bicarbonate solution (2×50 mL). The organic layer separated, dried over anhydrous Na₂SO₄, and filtered, and the filtrate was evaporated to dryness. The crude mass obtained was purified by combiflash chromatography (gradient: 20-50% ethyl acetate in hexane) to afford Compound 135 (1.01 g, 81% yield). ¹H NMR (400 MHz, Acetonitrile-d₃) δ 8.92 (s, 1H), 7.75 (dd, J=12.7, 8.2 Hz, 1H), 7.43 (dt, J=8.3, 1.2 Hz, 2H), 7.35-7.25 (m, 7H), 6.98-6.74 (m, 4H), 6.16-5.66 (m, 1H), 5.30 (dd, J=18.4, 8.1 Hz, 1H), 4.61-4.35 (m, 1H), 4.06 (ddt, J=15.5, 6.4, 3.6 Hz, 2H), 3.89-3.72 (m, 8H), 3.60 (s, 6H), 3.48-3.27 (m, 3H), 2.73-2.57 (m, 2H), 2.27 (t, J=7.5 Hz, 2H), 1.54 (q, J=6.8 Hz, 4H), 1.26 (q, J=3.7, 2.5 Hz, 19H), 1.16 (dd, J=10.7, 6.8 Hz, 12H) ppm. ³¹P NMR (162 MHz, CD₃CN) δ 150.91, 150.18 ppm.

Compound 136: To a clear solution of 135 (0.95 g, 1.17 mmol) in dry dichloromethane (40 mL) at 22° C. was added diisopropylethylamine (760.85 mg, 5.83 mmol, 1.03 mL) and N-methylimidazole (193.33 mg, 2.33 mmol, 187.70 μL) slowly. The resulting solution was stirred for 5 minutes after which 2-cyanoethyl-N,N-diisopropylchlorophosphoramidite (580.81 mg, 2.33 mmol, 547.93 μL) was added in single portion. The reaction mixture was kept for 1 hour stirring at 22° C. and TLC was checked. The reaction mixture was diluted with dichloromethane (50 mL) and washed with 10% sodium bicarbonate solution (2×50 mL). The organic layer separated, dried over anhydrous Na₂SO₄, filtered, and the filtrate was evaporated to dryness. The crude mass obtained was purified by combiflash chromatography (gradient: 20-50% ethyl acetate in hexane) to 136 (0.97 g, 82% yield). ¹H NMR (400 MHz, CD₃CN) δ 8.91 (s, 1H), 7.76 (dd, J=34.7, 8.2 Hz, 1H), 7.44 (ddt, J=9.9, 8.1, 1.3 Hz, 2H), 7.38-7.20 (m, 7H), 6.95-6.78 (m, 4H), 5.85 (dd, J=6.0, 3.5 Hz, 1H), 5.22 (dd, J=8.1, 5.9 Hz, 1H), 4.68-4.31 (m, 1H), 4.21-4.09 (m, 1H), 4.03 (ddd, J=11.5, 4.9, 3.5 Hz, 1H), 3.77 (d, J=2.4 Hz, 7H), 3.69-3.54 (m, 7H), 3.45-3.30 (m, 2H), 2.66 (ddd, J=6.5, 5.4, 3.7 Hz, 1H), 2.52 (t, J=6.0 Hz, 1H), 2.27 (t, J=7.5 Hz, 2H), 1.54 (d, J=10.2 Hz, 4H), 1.27 (d, J=5.4 Hz, 21H), 1.16 (dd, J=8.9, 6.8 Hz, 9H), 1.05 (d, J=6.8 Hz, 2H) ppm. ³¹P NMR (162 MHz, CD₃CN) δ 149.96, 149.58 ppm.

### Synthesis of 2′, 3′-O-pentadecyl ω Carboxymethyl Ester Adinosine Phosphoramidites

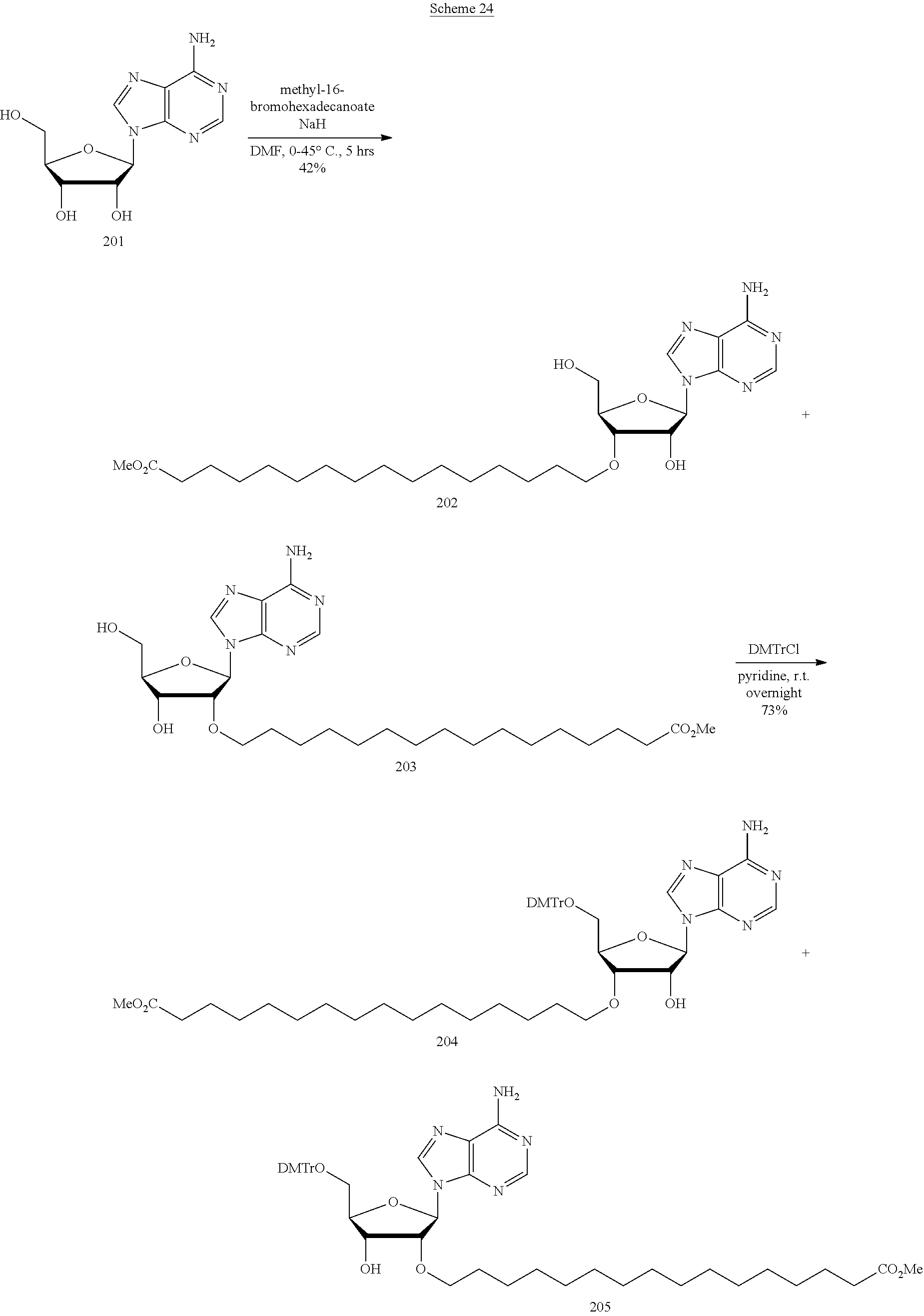

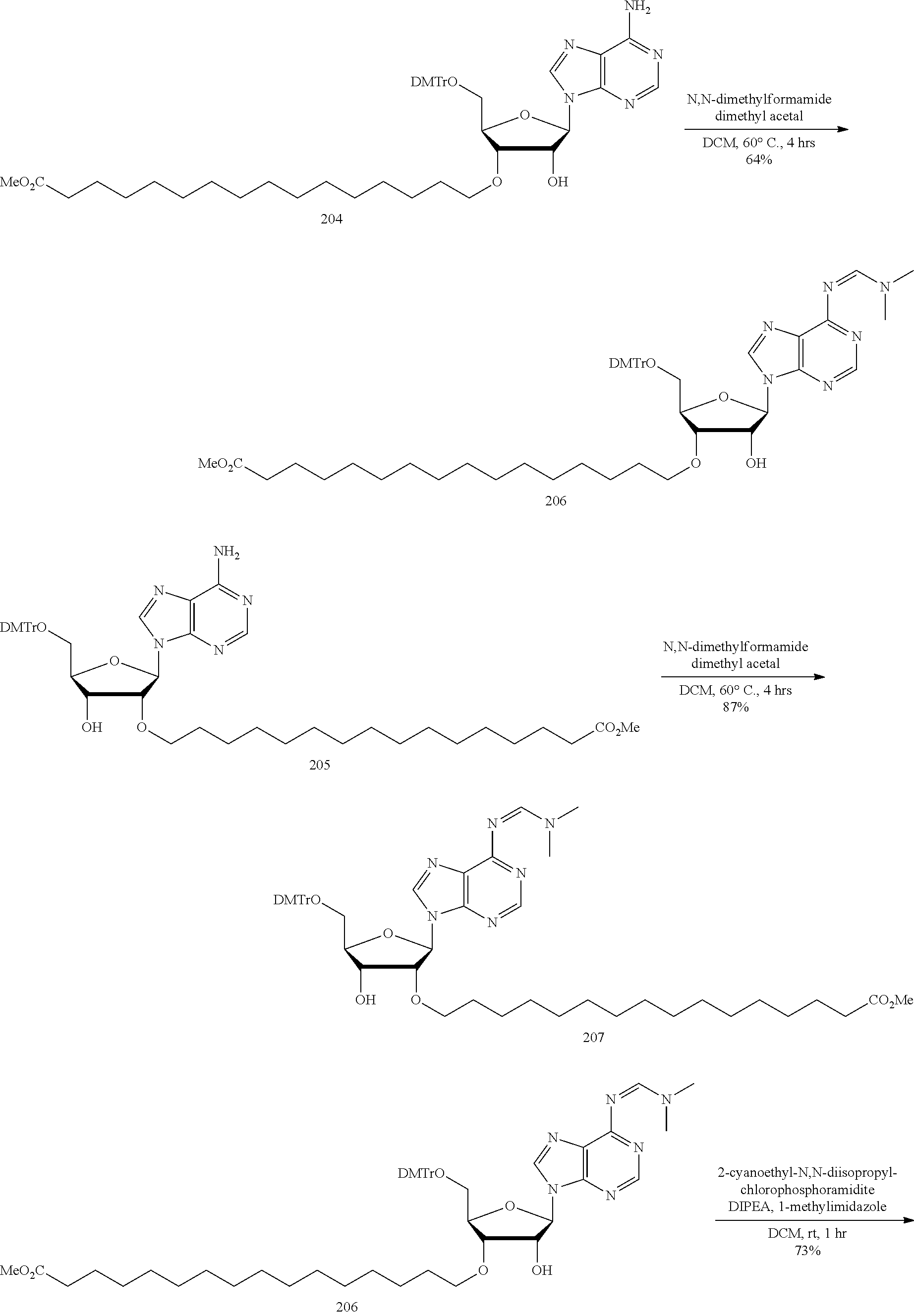

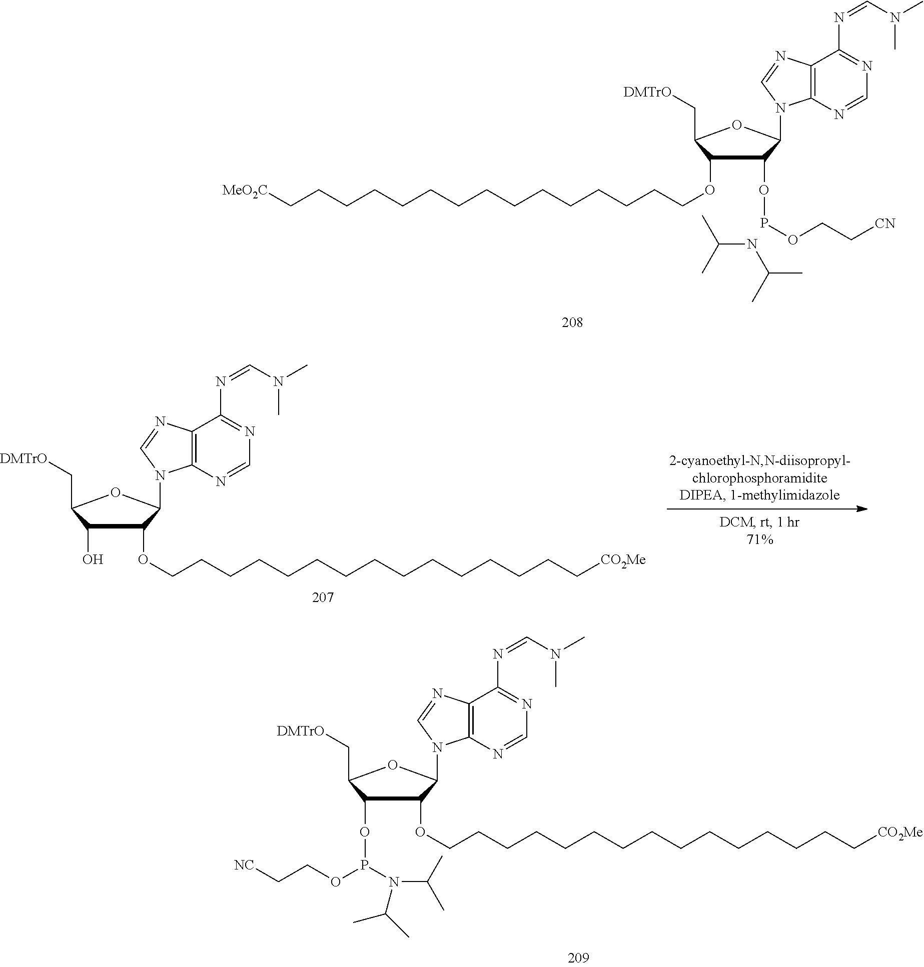

Compounds 202 and 203: To the suspension of Compound 201 (5.0 g, 18.71 mmol) in dry dimethylformamide (50 mL) was added sodium hydride (60% dispersion in mineral oil) (748.32 mg, 18.71 mmol) at 0° C. and stirred for 30 minutes. Ice bath was removed and the reaction mixture was warmed to 45° C. and stirred for 5 hours, after which the solvent was evaporated in high vacuum pump and solid mass was purified by combiflash chromatography (Gradient: 0-10% MeOH in DCM) to afford mixture of Compounds 202 and 203 (4.2 g, 42% yield) as white solid. ¹H NMR (500 MHz, DMSO-d₆) δ 8.37 (s, 1H), 8.35 (s, 0.2H), 8.13 (s, 1H), 7.33 (s, 3H), 5.98 (d, J=6.4 Hz, 0.8H), 5.88 (d, J=6.2 Hz, 0.2H), 5.51-5.40 (m, 1H), 5.38 (d, J=6.4 Hz, 0.2H), 5.14 (d, J=5.0 Hz, 1H), 4.47 (dd, J=6.4, 4.8 Hz, 1H), 4.29 (td, J=4.9, 2.9 Hz, 1H), 4.06-3.86 (m, 1H), 3.67 (tt, J=15.5, 5.0 Hz, 2H), 3.57 (s, 5H), 3.33 (dt, J=9.5, 6.5 Hz, 2H), 2.27 (t, J=7.4 Hz, 2H), 1.57-1.00 (m, 27H) ppm. 13C NMR (126 MHz, DMSO-d₆) δ 173.30, 156.16, 156.14, 152.38, 148.96, 139.73, 119.34, 86.48, 86.11, 80.76, 69.62, 69.02, 61.53, 51.11, 33.25, 29.07, 29.05, 29.02, 28.99, 28.97, 28.94, 28.86, 28.72, 28.66, 28.44, 25.25, 24.42 ppm.

Compounds 204 and 205: To a clear solution of a mixture of Compounds 2 and 3 (3.6 g, 6.72 mmol) in dry pyridine (25 mL) was added 4,4′-dimethoxytrityl chloride (2.88 g, 8.06 mmol) in three portions. The reaction mixture was stirred for 24 hour at 22° C. and then quenched with saturated NaHCO₃ solution (30 mL). The resulting mixture was extracted with DCM (2×40 mL). The combined organic layer was separated, washed with brine (40 mL), dried over anhydrous Na₂SO₄, filtered, and the filtrate was evaporated to dryness. The crude compound was purified by combiflash chromatography (gradient: 10-90% ethyl acetate in hexane) to afford Compounds 204 (0.36 g, 6%) and 205 (3.8 g, 67%) as white foam. Spectral data for Compound 204: ¹H NMR (400 MHz, DMSO-d₆) δ 8.27 (s, 1H), 8.10 (s, 1H), 7.42-7.06 (m, 10H), 6.82 (dd, J=9.1, 2.7 Hz, 4H), 5.90 (d, J=4.4 Hz, 1H), 5.46 (d, J=5.9 Hz, 1H), 4.86 (q, J=5.1 Hz, 1H), 4.18 (t, J=5.2 Hz, 1H), 4.07 (q, J=4.6 Hz, 1H), 3.72 (s, 6H), 3.62 (dt, J=9.5, 6.4 Hz, 1H), 3.43 (dt, J=9.5, 6.7 Hz, 1H), 3.32 (s, 5H), 3.17 (dd, J=10.5, 4.8 Hz, 1H), 2.27 (t, J=7.4 Hz, 2H), 1.50 (td, J=7.7, 7.3, 4.1 Hz, 4H), 1.22 (s, 24H) ppm. ¹³C NMR (126 MHz, DMSO-d₆) δ 173.32, 158.02, 156.04, 152.54, 149.23, 144.77, 139.57, 135.50, 135.48, 129.62, 129.59, 127.72, 127.63, 126.60, 119.16, 113.07, 88.21, 85.48, 80.84, 77.63, 71.69, 69.66, 63.07, 54.97, 51.11, 33.24, 29.19, 29.00, 28.99, 28.93, 28.83, 28.63, 28.42, 25.47, 24.40 ppm. Spectral data for Compound 205: ¹H NMR (500 MHz, DMSO-d₆) δ 8.25 (s, 1H), 8.08 (s, 1H), 7.48-7.04 (m, 10H), 6.83 (dd, J=8.8, 6.0 Hz, 4H), 6.00 (d, J=5.1 Hz, 1H), 5.16 (d, J=5.9 Hz, 1H), 4.58 (t, J=5.1 Hz, 1H), 4.37 (q, J=5.1 Hz, 1H), 4.06 (q, J=4.6 Hz, 1H), 3.72 (s, 6H), 3.57 (s, 4H), 3.47-3.40 (m, 1H), 3.24 (d, J=4.7 Hz, 2H), 2.26 (d, J=7.5 Hz, 2H), 1.46 (dt, J=31.8, 7.0 Hz, 4H), 1.27-1.08 (m, 23H) ppm. ¹³C NMR (126 MHz, DMSO-d₆) δ 173.32, 158.03, 158.01, 156.06, 152.57, 149.22, 144.81, 139.54, 135.54, 135.43, 129.68, 127.72, 127.67, 126.60, 119.19, 113.08, 85.89, 85.50, 83.56, 80.04, 69.79, 69.11, 63.55, 54.98, 51.11, 33.24, 29.00, 28.98, 28.94, 28.84, 28.71, 28.63, 28.42, 25.30, 24.40 ppm.

Compound 206: To a clear solution of Compound 204 (1.27 g, 1.52 mmol) in dimethylformamide (30 mL) was added N,N-dimethylformamide dimethyl acetal (288.16 mg, 2.27 mmol, 323.77 μL) in a single portion and the reaction mixture was stirred at 60° C. for 4 hour. TLC was checked, and volatile matters was removed under high vacuum pump. Residue was dissolved in DCM (100 mL) and the organic layer was washed with brine (3×50 mL). DCM layer was then dried over anhydrous Na₂SO₄, filtered and the filtrate was evaporated to dryness. Crude mass thus obtained, was purified by combiflash chromatography (gradient: 0-5% MeOH in DCM) to afford 206 (0.87 g, 64% yield) as white hygroscopic solid. ¹H NMR (500 MHz, CDCl₃) δ 8.95 (s, 1H), 8.50 (s, 1H), 8.09 (s, 1H), 7.47-7.36 (m, 2H), 7.31-7.15 (m, 8H), 6.83-6.74 (m, 4H), 6.02 (d, J=5.5 Hz, 1H), 4.82 (d, J=5.5 Hz, 1H), 4.27 (q, J=4.0 Hz, 1H), 4.19 (dd, J=5.5, 3.5 Hz, 1H), 3.78 (d, J=0.8 Hz, 6H), 3.69 (d, J=6.5 Hz, 1H), 3.66 (s, 3H), 3.56 (tdd, J=9.3, 6.7, 2.6 Hz, 2H), 3.46 (dd, J=10.4, 4.4 Hz, 1H), 3.31 (dd, J=10.5, 3.9 Hz, 1H), 3.26 (s, 3H), 3.20 (s, 3H), 2.30 (t, J=7.6 Hz, 2H), 1.60 (p, J=6.9 Hz, 5H), 1.27 (d, J=8.3 Hz, 25H) ppm. ¹³C NMR (101 MHz, CDCl₃) δ 174.49, 159.87, 158.66, 158.33, 152.76, 151.62, 144.62, 140.30, 135.82, 135.79, 130.13, 130.10, 128.22, 128.02, 127.03, 126.60, 113.31, 89.27, 86.66, 82.39, 78.68, 74.07, 71.14, 63.42, 55.35, 51.58, 41.42, 35.30, 34.26, 29.88, 29.80, 29.77, 29.74, 29.72, 29.60, 29.40, 29.29, 26.22, 25.10 ppm.

Compound 207: To a clear solution of 205 (2.0 g, 2.39 mmol) in dimethylformamide (30 mL) was added N,N-dimethylformamide dimethyl acetal (453.79 mg, 3.58 mmol, 505.90 μL) in single portion and the reaction mixture was stirred at 60° C. for 4 hr. TLC was checked, and volatile materials were removed under high vacuum pump. The residue was dissolved in DCM (100 mL) and the organic layer was washed with brine (3×50 mL). The DCM layer was then dried over anhydrous Na₂SO₄, filtered, and the filtrate was evaporated to dryness. The crude mass thus obtained was purified by combiflash chromatography (gradient: 0-5% MeOH in DCM) to afford Compound 207 (1.85 g, 87% yield) as white hygroscopic solid. ¹H NMR (500 MHz, CDCl₃) δ 8.95 (s, 1H), 8.49 (s, 1H), 8.10 (s, 1H), 7.47-7.41 (m, 2H), 7.36-7.30 (m, 4H), 7.28-7.17 (m, 3H), 6.89-6.67 (m, 4H), 6.17 (d, J=4.2 Hz, 1H), 4.52 (dd, J=5.3, 4.2 Hz, 1H), 4.45 (q, J=5.3 Hz, 1H), 4.21 (td, J=4.6, 3.1 Hz, 1H), 3.78 (d, J=1.0 Hz, 6H), 3.74-3.67 (m, 1H), 3.66 (s, 3H), 3.60-3.54 (m, 1H), 3.51 (dd, J=10.6, 3.2 Hz, 1H), 3.41 (dd, J=10.6, 4.4 Hz, 1H), 3.26 (s, 3H), 3.20 (s, 3H), 2.73 (d, J=5.9 Hz, 1H), 2.30 (t, J=7.6 Hz, 2H), 1.67-1.52 (m, 4H), 1.37-1.04 (m, 25H) ppm. ¹³C NMR (126 MHz, CDCl₃) δ 174.47, 159.73, 158.66, 158.24, 152.89, 151.46, 144.70, 140.20, 135.93, 135.83, 130.23, 130.20, 128.32, 128.00, 127.01, 126.60, 113.32, 86.89, 86.69, 84.04, 81.75, 71.60, 70.23, 63.39, 55.34, 51.56, 41.40, 35.30, 34.25, 29.77, 29.74, 29.72, 29.65, 29.58, 29.48, 29.39, 29.28, 26.04, 25.09 ppm.

Compound 208: To a clear solution of Compound 206 (0.68 g, 761.38 μmol) in dry dichloromethane (20 mL) at 22° C. was added, diisopropylethylamine (496.97 mg, 3.81 mmol, 669.77 μL) and N-methylimidazole (94.71 mg, 1.14 mmol, 91.95 μL) slowly. The resulting solution was stirred for 5 minutes after which 2-cyanoethyl-N,N-diisopropylchlorophosphoramidite (379.37 mg, 1.52 mmol, 357.90 μL) was added in single portion. The reaction mixture was kept for 1 hour stirring at 22° C. and TLC was checked. Reaction mixture was diluted with dichloromethane (50 mL) and washed with 10% sodium bicarbonate solution (2×50 mL). The organic layer was separated, dried over anhydrous Na₂SO₄, filtered, and the filtrate was evaporated to dryness. The crude mass obtained was purified by combiflash chromatography (gradient: 40-70% ethyl acetate in hexane) to afford Compound 208 (0.61 g, 73% yield) as white hygroscopic solid. ¹H NMR (500 MHz, CD₃CN) δ 8.89 (d, J=2.3 Hz, 1H), 8.34 (d, J=10.8 Hz, 1H), 8.08 (d, J=11.5 Hz, 1H), 7.46-7.38 (m, 2H), 7.34-7.15 (m, 7H), 6.88-6.73 (m, 4H), 6.04 (dd, J=5.2, 3.3 Hz, 1H), 4.95-4.60 (m, 2H), 4.28 (dq, J=21.0, 4.2 Hz, 1H), 4.14-3.84 (m, 1H), 3.77-3.70 (m, 7H), 3.67-3.58 (m, 5H), 3.30 (dd, J=15.2, 4.7 Hz, 1H), 3.19-3.10 (m, 7H), 2.50 (t, J=6.0 Hz, 1H), 2.27 (t, J=7.5 Hz, 2H), 1.51 (dt, J=45.3, 7.0 Hz, 4H), 1.32-1.07 (m, 40H) ppm. ³¹P NMR (202 MHz, CD₃CN) δ 151.07, 150.64 ppm.

Compound 209: To a clear solution of Compound 207 (0.2 g, 223.93 μmol) in dry dichloromethane (35 mL) at 22° C. was added diisopropylethylamine (146.17 mg, 1.12 mmol, 196.99 μL) and N-methylimidazole (27.86 mg, 335.90 μmol, 27.04 μL) slowly. The resulting solution was stirred for 5 minutes, after which 2-cyanoethyl-N,N-diisopropylchlorophosphoramidite (111.58 mg, 447.87 μmol, 105.26 μL) was added in a single portion. The reaction mixture was kept for 1 hour stirring at 22° C. and TLC was checked. The reaction mixture was diluted with dichloromethane (50 mL) and washed with 10% sodium bicarbonate solution (2×50 mL). The organic layer was separated, dried over anhydrous Na₂SO₄, filtered, and the filtrate was evaporated to dryness. The crude mass obtained was purified by combiflash chromatography (gradient: 20-50% ethyl acetate in hexane) to afford Compound 209 (0.173 g, 71% yield) as hygroscopic solid. ¹H NMR (400 MHz, CD₃CN) δ 8.89 (d, J=1.8 Hz, 1H), 8.34 (d, J=9.0 Hz, 1H), 8.08 (d, J=9.6 Hz, 1H), 7.50-7.37 (m, 2H), 7.35-7.14 (m, 6H), 6.81 (ddd, J=9.1, 6.0, 3.2 Hz, 4H), 6.03 (dd, J=5.2, 3.0 Hz, 1H), 4.79 (dt, J=15.9, 5.0 Hz, 1H), 4.68 (tt, J=9.4, 4.6 Hz, 1H), 4.34-4.20 (m, 1H), 3.95-3.78 (m, 1H), 3.76-3.73 (m, 5H), 3.59 (s, 6H), 3.51-3.37 (m, 2H), 3.29 (ddd, J=12.6, 10.7, 4.7 Hz, 1H), 3.16 (d, J=8.3 Hz, 5H), 2.71-2.62 (m, 1H), 2.50 (t, J=6.0 Hz, 1H), 2.27 (t, J=7.5 Hz, 2H), 1.50 (dt, J=36.2, 7.1 Hz, 4H), 1.35-1.04 (m, 31H). ³¹P NMR (162 MHz, CD₃CN) δ 149.86, 149.42 ppm.

### Synthesis of 2′, 3′-O-pentadecyl ω Carboxymethyl Ester Guanosine Phosphoramidites

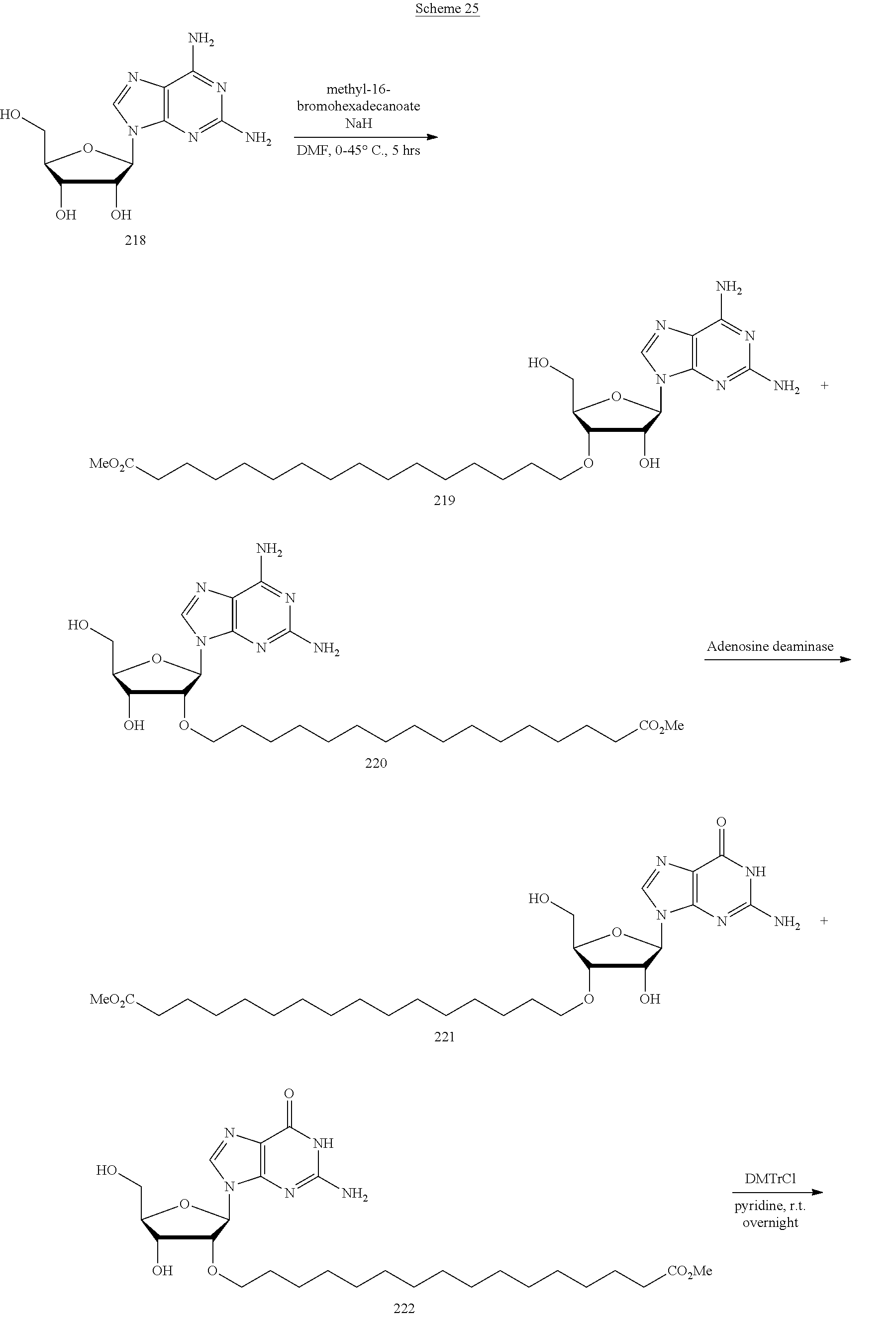

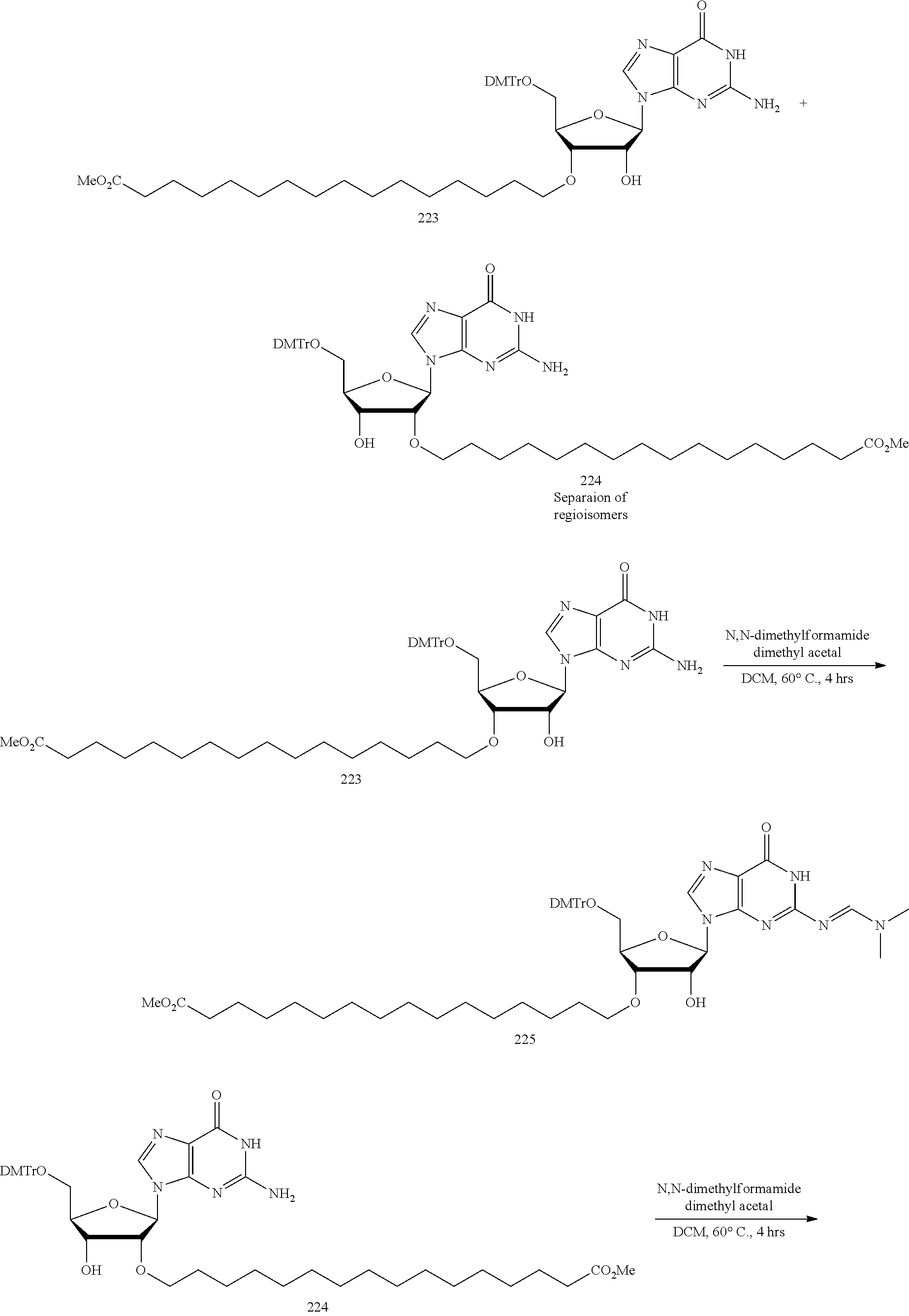

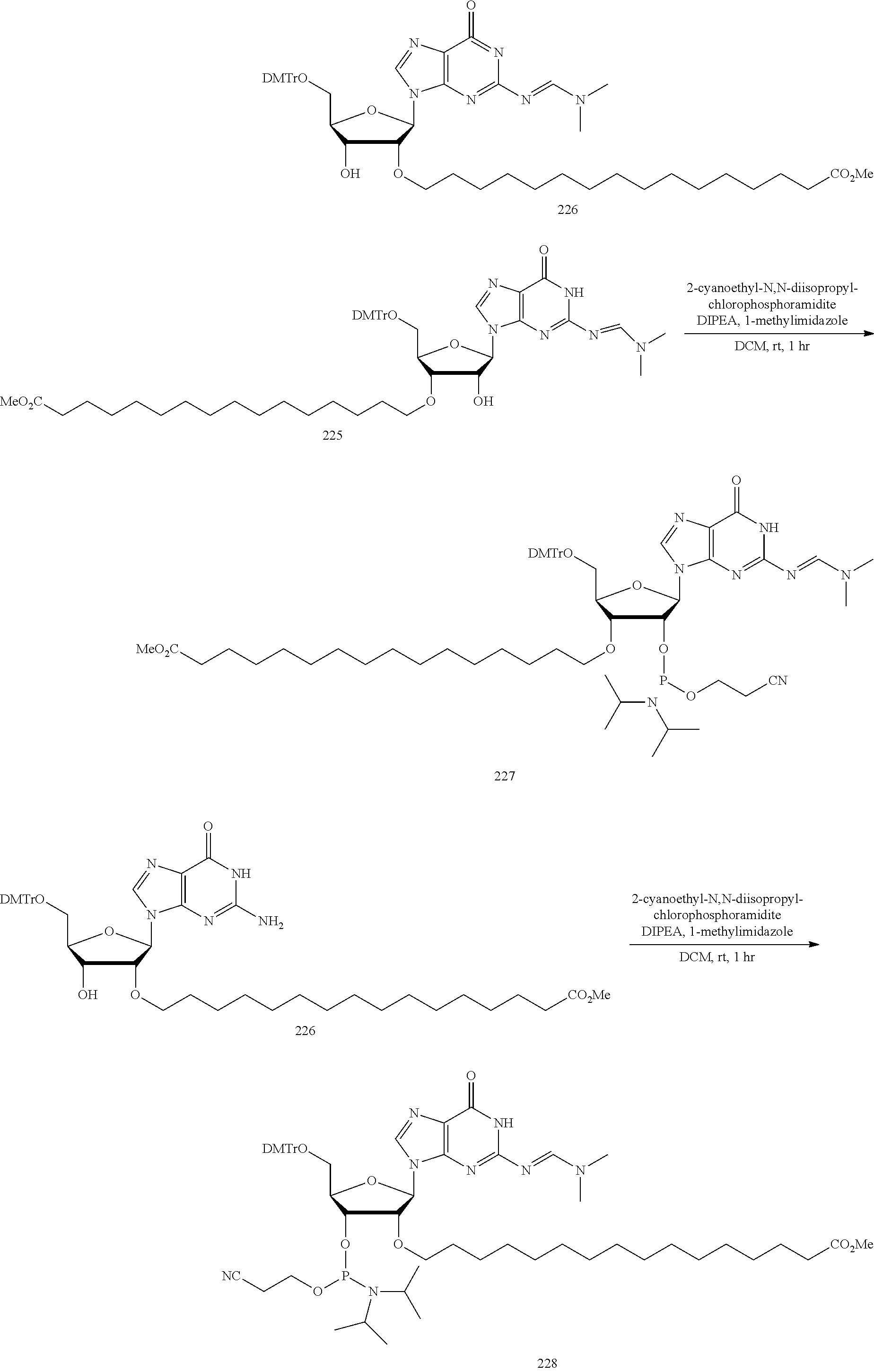

Compounds 219 and 220: To the suspension of Compound 218 in dry dimethylformamide, sodium hydride (60% dispersion in mineral oil) is added at 0° C. and stirred for 30 minutes. Ice bath is removed, and the reaction mixture is warmed to 45° C. and stirred for 5 hours, after which the solvent is evaporated in high vacuum pump and solid mass is purified by combiflash chromatography to afford a mixture of Compounds 219 and 220.

Compounds 221 and 222: Compounds 219 and 220 are converted to Compounds 221 and 222 respectively with adenosine deaminase (ADA), as described in Robins et. al. (Can. J. Chem. 1997, 75, 762-767).

Compounds 223 and 224: To a clear solution of a mixture of Compounds 221 and 222 in dry pyridine is added 4,4′-dimethoxytrityl chloride in three portions. The reaction mixture is stirred for 24 hour at 22° C. and then quenched with saturated NaHCO₃ solution. The resulting mixture is extracted with DCM. The combined organic layer is separated, washed with brine, dried over anhydrous Na₂SO₄, filtered, and the filtrate is evaporated to dryness. The crude compound is purified by combiflash chromatography to afford Compounds 223 and 224.

Compound 225: To a clear solution of Compound 223 in dimethylformamide is added N,N-dimethylformamide dimethyl acetal in single portion and the reaction mixture is stirred at 60° C. for 4 hr. TLC is checked, and volatile materials are removed under high vacuum pump. The residue is dissolved in DCM (100 mL) and the organic layer is washed with brine. DCM layer is then dried over anhydrous Na₂SO₄, filtered, and the filtrate is evaporated to dryness. The crude mass thus obtained, is purified by combiflash chromatography to afford Compound 225.

Compound 226: To a clear solution of Compound 224 in dimethylformamide is added N,N-dimethylformamide dimethyl acetal in a single portion and the reaction mixture is stirred at 60° C. for 4 hour. TLC is checked, and volatile materials are removed under high vacuum pump. The residue is dissolved in DCM and the organic layer is washed with brine. DCM layer is then dried over anhydrous Na₂SO₄, filtered, and the filtrate is evaporated to dryness. The crude mass thus obtained, is purified by combiflash chromatography to afford Compound 226.

Compound 227: To a clear solution of Compound 225 in dry dichloromethane at 22° C. is added diisopropylethylamine and N-methylimidazole slowly. The resulting solution is stirred for 5 minutes, after which 2-cyanoethyl-N,N-diisopropylchlorophosphoramidite is added in a single portion. The reaction mixture is kept for 1 hour stirring at 22° C. and TLC is checked. The reaction mixture is diluted with dichloromethane adding 10% sodium bicarbonate solution. The organic layer is separated, being dried over anhydrous Na₂SO₄, filtered, and the filtrate is evaporated to dryness. The crude mass obtained is purified by combiflash chromatography to afford Compound 227.

Compound 228: To a clear solution of Compound 226 in dry dichloromethane at 22° C. is added diisopropylethylamine and N-methylimidazole slowly. The resulting solution is stirred for 5 minutes, after which 2-cyanoethyl-N,N-diisopropylchlorophosphoramidite is added in a single portion. The reaction mixture is kept for 1 hour stirring at 22° C. and TLC was checked. The reaction mixture is diluted with dichloromethane adding 10% sodium bicarbonate solution. The organic layer is separated, being dried over anhydrous Na₂SO₄, filtered, and the filtrate is evaporated to dryness. The crude mass obtained is purified by combiflash chromatography to afford Compound 228.

### Synthesis of 2′, 3′-O-pentadecyl ω Carboxymethyl Ester Cytidine Phosphoramidites

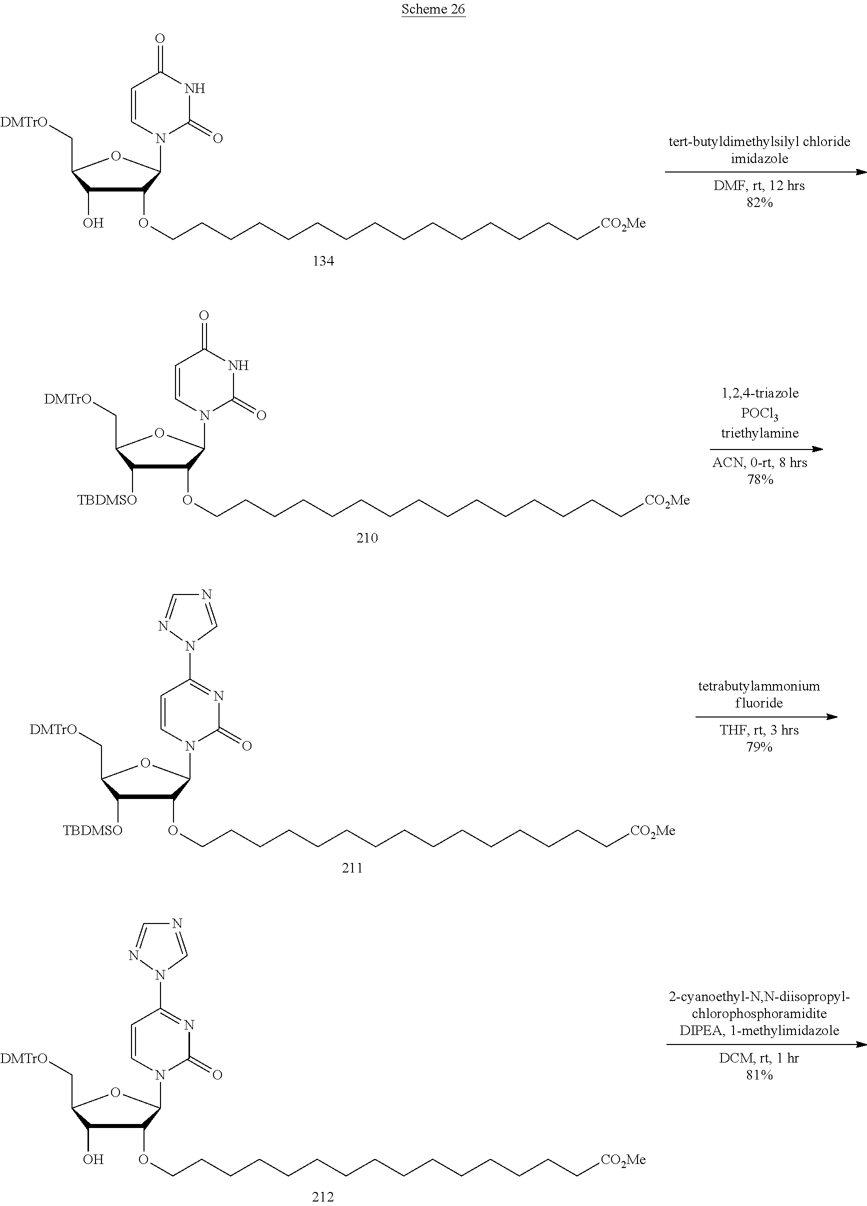

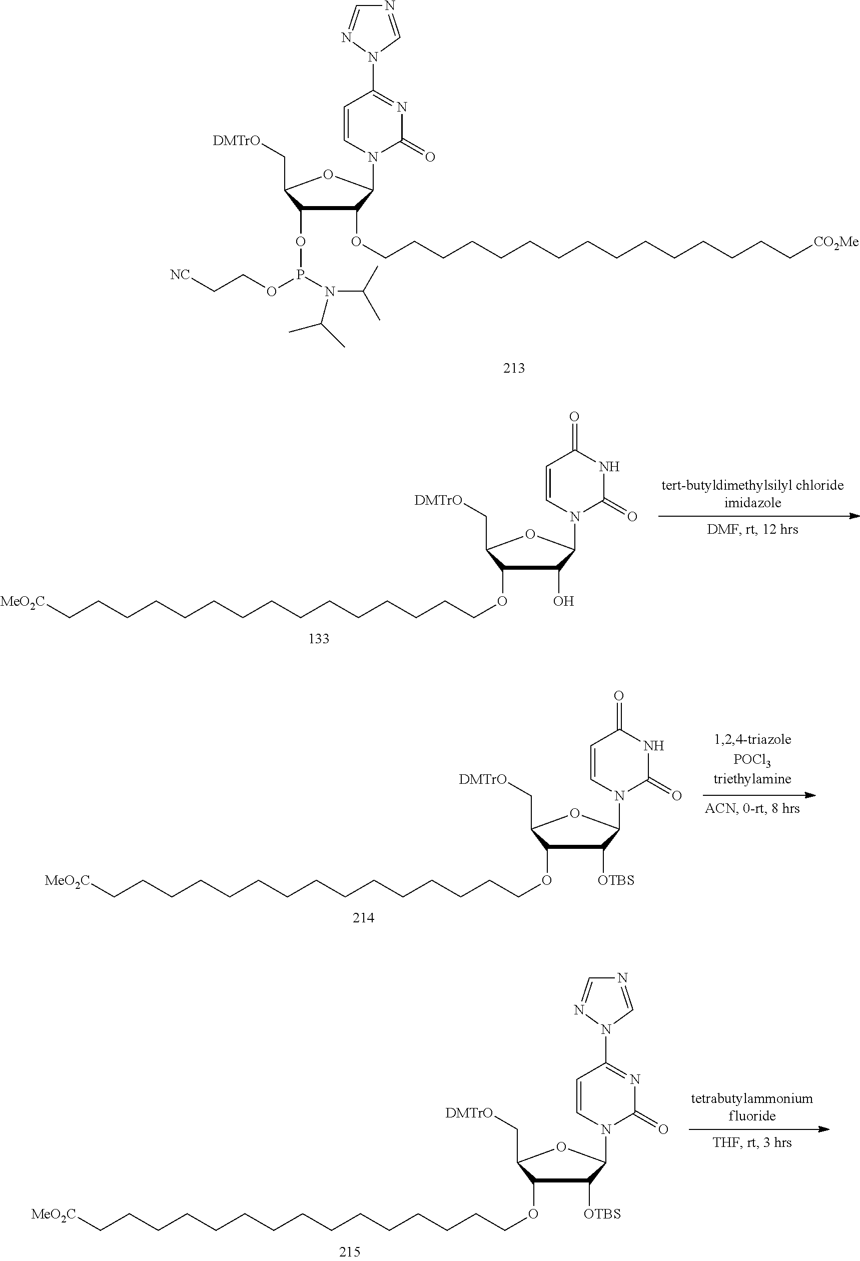

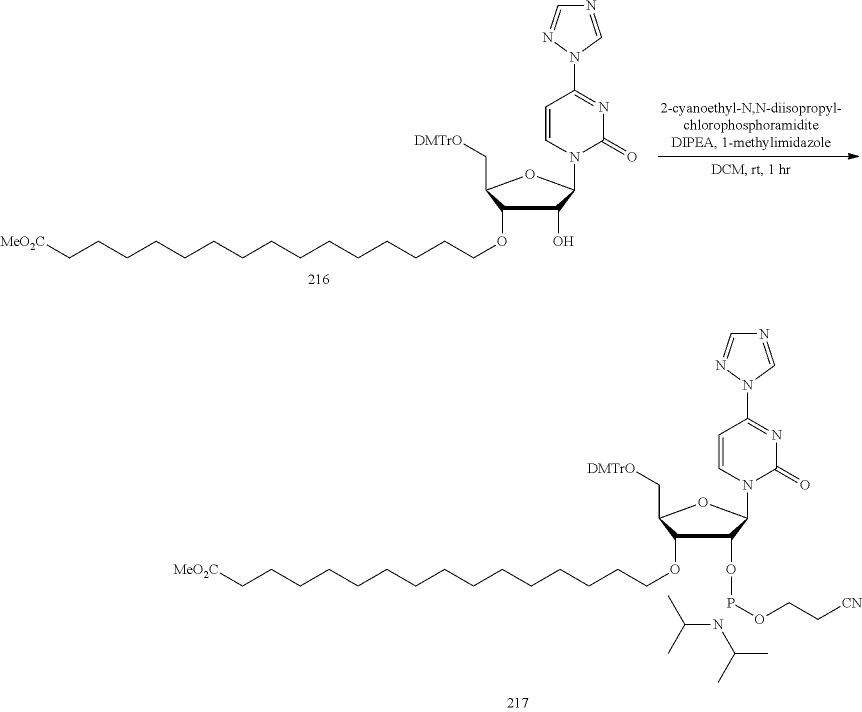

Compound 210: To a clear solution of Compound 134 (1.2 g, 1.47 mmol) in dimethylformamide (15 mL) was added imidazole (202.51 mg, 2.94 mmol) and stirred for 5 minutes. To the resulting solution was added tert-butyldimethylsilyl chloride (343.17 mg, 2.21 mmol) in a single portion and stirred at 22° C. for 12 hours. The reaction mixture was then diluted with ethylacetate (50 mL) and brine (50 mL). The organic layer was separated and further washed with brine (2×50 mL) and water (50 mL). The organic layer was then dried over anhydrous Na₂SO₄, filtered, and the filtrate was evaporated to dryness. The crude compound thus obtained was purified by combiflash chromatography (gradient: 0-50% ethyl acetate in hexane) to afford Compound 210 (1.12 g, 82% yield) as white foam. ¹H NMR (500 MHz, CDCl₃) δ 8.38 (s, 1H), 8.14 (d, J=8.1 Hz, 1H), 7.42-7.15 (m, 10H), 6.84 (dd, J=8.9, 3.8 Hz, 3H), 5.92 (d, J=1.6 Hz, 1H), 5.24 (d, J=8.1 Hz, 1H), 4.35 (dd, J=7.9, 4.8 Hz, 1H), 4.11 (d, J=7.9 Hz, 1H), 3.80 (d, J=1.2 Hz, 7H), 3.72 (dd, J=4.9, 1.7 Hz, 1H), 3.66 (s, 4H), 3.52 (dt, J=9.2, 6.6 Hz, 1H), 3.36 (dd, J=11.1, 2.3 Hz, 1H), 2.30 (t, J=7.6 Hz, 2H), 1.69-1.50 (m, 8H), 1.37-1.22 (m, 24H), 0.81 (s, 9H), 0.05 (s, 3H), −0.04 (s, 3H) ppm. 13C NMR (101 MHz, CDCl₃) δ 174.51, 163.10, 158.90, 149.97, 144.22, 140.41, 135.26, 135.10, 130.40, 128.47, 128.09, 127.38, 113.38, 113.34, 102.04, 88.35, 87.16, 82.89, 82.79, 71.01, 69.51, 60.90, 55.41, 51.59, 34.28, 30.00, 29.81, 29.79, 29.76, 29.74, 29.64, 29.60, 29.41, 29.30, 26.27, 25.76, 25.11, 18.19, −4.37, −4.87 ppm.

Compound 211: To a clear solution of Compound 210 (1.2 g, 1.29 mmol) in acetonitrile (30 mL) was added 1,2,4-triazole (2.00 g, 28.41 mmol) and triethylamine (2.89 g, 28.41 mmol, 3.98 mL). The reaction mixture was cooled to 0° C. and then phosphorus(V)oxychloride (594.02 mg, 3.87 mmol, 362.21 μL) was added slowly. Ice bath was removed after 15 minutes and stirring was continued for 8 hrs at 22° C. Volatile materials were removed under high vacuum and the residue was diluted in DCM (50 mL) and the organic layer was washed with water (30 mL) and brine (50 mL). DCM layer was separated, dried over anhydrous Na₂SO₄, filtered, and the filtrate was evaporated to dryness. Crude compound was purified by combiflash chromatography (Gradient: 20-60% ethyl acetate in hexane) to afford Compound 211 (0.99 g, 78% yield) as white solid. ¹H NMR (500 MHz, DMSO-d₆) δ 9.44 (s, 1H), 8.78 (d, J=7.2 Hz, 1H), 8.39 (s, 1H), 7.40-7.12 (m, 23H), 7.06 (d, J=8.8 Hz, 5H), 6.47 (d, J=7.2 Hz, 1H), 6.19 (s, 2H), 5.85 (s, 1H), 4.39 (dd, J=9.1, 4.7 Hz, 1H), 4.08 (d, J=9.1 Hz, 1H), 3.94 (d, J=4.8 Hz, 1H), 3.84 (s, OH), 3.75 (d, J=6.1 Hz, 7H), 3.55 (s, 5H), 3.31-3.28 (m, 1H), 2.25 (t, J=7.4 Hz, 2H), 1.50 (dt, J=24.9, 6.9 Hz, 4H), 1.21 (d, J=8.1 Hz, 26H), 0.72 (s, 10H), 0.01 (s, 3H), −0.10 (s, 3H) ppm.

Compound 212: To a solution of Compound 211 (0.99 g, 1.01 mmol) in THE (20 mL) at 22° C., tetrabutylammonium fluoride, 1M in THE (346.72 mg, 1.31 mmol, 383.97 μL), was added slowly in a single portion and then stirred for 3 hours. Volatile materials were removed in high vacuum pump and the crude residue thus obtained was purified by combiflash chromatography (gradient: 0-5% methanol in DCM) to afford Compound 212 (0.69 g, 79% yield) as white solid. ¹H NMR (400 MHz, CDCl₃) δ 9.24 (s, 1H), 8.86 (d, J=7.2 Hz, 1H), 8.10 (s, 1H), 7.44-7.27 (m, 9H), 6.87 (dd, J=9.0, 0.9 Hz, 4H), 6.56 (d, J=7.2 Hz, 1H), 6.01 (s, 1H), 4.49 (ddd, J=10.7, 9.2, 5.1 Hz, 1H), 4.24-4.04 (m, 2H), 3.94 (d, J=5.2 Hz, 1H), 3.82 (d, J=0.8 Hz, 6H), 3.78-3.73 (m, 1H), 3.66 (s, 3H), 3.63 (t, J=2.6 Hz, 2H), 2.61 (d, J=10.7 Hz, 1H), 2.30 (t, J=7.5 Hz, 2H), 1.79-1.61 (m, 4H), 1.42-1.08 (m, 24H) ppm. ¹³C NMR (101 MHz, CDCl₃) δ 174.51, 159.49, 158.91, 154.41, 154.08, 147.44, 144.17, 143.34, 135.62, 135.25, 130.34, 130.28, 128.43, 128.23, 127.40, 113.51, 94.82, 89.38, 87.36, 83.69, 82.20, 71.44, 67.62, 60.66, 53.57, 51.59, 34.27, 29.79, 29.74, 29.60, 29.40, 29.30, 26.18, 25.11 ppm.

Compound 213: To a clear solution of Compound 212 (0.23 g, 265.57 μmol) in dry DCM (10 mL) at 22° C. was added diisopropylethylamine (173.35 mg, 1.33 mmol, 233.62 L) and N-methylimidazole (33.04 mg, 398.36 μmol, 32.07 μL) slowly. The resulting solution was stirred for 5 minutes, after which 2-cyanoethyl-N,N-diisopropylchlorophosphoramidite (132.33 mg, 531.15 μmol, 124.84 μL) was added in a single portion. The reaction mixture was kept for 1 hour stirring at 22° C. and TLC was checked. The reaction mixture was diluted with DCM (50 mL) and washed with 10% sodium bicarbonate solution (2×50 mL). The organic layer was separated, dried over anhydrous Na₂SO₄, filtered, and the filtrate was evaporated to dryness. The crude mass obtained was purified by Combiflash chromatography (Gradient: 30-60% ethyl acetate in hexane) to afford Compound 213 (0.23 g, 81% yield) as white solid. ¹H NMR (500 MHz, CD₃CN) δ 9.17 (s, 1H), 8.72 (dd, J=35.4, 7.2 Hz, 1H), 8.13 (s, 1H), 7.64-7.17 (m, 9H), 6.89 (ddt, J=6.8, 5.4, 1.4 Hz, 4H), 6.44 (dd, J=24.2, 7.2 Hz, 1H), 5.88 (s, 1H), 4.79-4.41 (m, 1H), 4.31-4.01 (m, 2H), 3.97-3.45 (m, 18H), 2.73-2.45 (m, 2H), 2.27 (t, J=7.5 Hz, 2H), 1.73-1.50 (m, 4H), 1.40-1.05 (m, 35H) ppm. ¹³C NMR (126 MHz, CD₃CN) δ 174.82, 160.22, 159.86, 159.84, 154.96, 154.88, 148.55, 145.41, 144.07, 136.70, 136.54, 136.35, 136.29, 131.32, 131.28, 131.22, 131.17, 129.32, 129.02, 128.14, 114.21, 100.98, 95.04, 94.93, 91.54, 91.30, 87.85, 87.76, 83.10, 82.86, 81.65, 81.63, 71.83, 71.60, 70.67, 69.94, 69.86, 61.57, 61.28, 59.31, 59.24, 59.14, 59.08, 55.97, 55.95, 51.83, 44.13, 44.06, 43.96, 34.51, 30.64, 30.61, 30.35, 30.34, 30.30, 30.28, 30.25, 30.18, 29.97, 29.78, 26.87, 25.70, 25.14, 25.08, 25.04, 24.99, 24.88, 24.82, 21.19, 21.13 ppm. ³¹P NMR (202 MHz, CD₃CN) δ 151.23, 149.91 ppm.

Compound 214: To a clear solution of Compound 133 (1.3 g, 1.60 mmol) in dimethylformamide (15 mL) was added imidazole (219.38 mg, 3.19 mmol) and stirred for 5 minutes. To the resulting solution was added tert-butyldimethylsilyl chloride (371.77 mg, 2.39 mmol) in a single portion and stirred at 22° C. for 16 hours. The reaction mixture was then diluted with ethyl acetate (50 mL) and brine (50 mL). The organic layer was separated and further washed with brine (2×50 mL) and water (50 mL). The organic layer was then dried over anhydrous Na₂SO₄, filtered, and the filtrate was evaporated to dryness. The crude compound thus obtained was purified by combiflash chromatography (gradient: 0-50% ethyl acetate in hexane) to afford Compound 214 (1.29 g, 1.39 mmol, 87.03% yield) as white foam.

Compound 215: To a clear solution of Compound 214 (1.29 mmol) in acetonitrile (30 mL) was added 1,2,4-triazole (28.41 mmol) and triethylamine (28.41 mmol, 3.98 mL). The reaction mixture was cooled to 0° C. and then phosphorus(V)oxychloride (3.87 mmol, 362.21 μL) was added slowly. Ice bath was removed after 15 minutes and stirring was continued for 9 hours at 22° C. Volatile materials were removed under high vacuum and residue was diluted in DCM (60 mL), and the organic layer was washed with water (30 mL) and brine (2×50 mL). DCM layer was separated, dried over anhydrous Na₂SO₄, filtered, and the filtrate was evaporated to dryness. The crude compound was purified by combiflash chromatography (Gradient: 20-70% ethyl acetate in hexane) to afford 215 (0.92 g, 72% yield) as white solid.

Compound 216: To a solution of Compound 215 (0.92 g) in THE (20 mL) at 22° C., tetrabutylammonium fluoride, 1M in THE (1.31 mmol, 383.97 μL), was added slowly in a single portion and then stirred for 3 hours. Volatile materials were removed in high vacuum pump, and the crude residue thus obtained was purified by combiflash chromatography (gradient: 0-5% methanol in DCM) to afford Compound 216 (0.70 g, 79% yield) as white solid.

Compound 217: To a clear solution of Compound 216 (0.25 g) in dry DCM (10 mL) at 22° C. was added diisopropylethylamine (1.33 mmol, 233.62 μL) and N-methylimidazole (398.36 μmol, 32.07 μL) slowly. The resulting solution was stirred for 5 minutes, after which 2-cyanoethyl-N,N-diisopropylchlorophosphoramidite (531.15 μmol, 124.84 μL) was added in a single portion. The reaction mixture was kept for 1 hour stirring at 22° C. and TLC was checked. The reaction mixture was diluted with DCM (50 mL) and washed with 10% sodium bicarbonate solution (2×50 mL). The organic layer was separated, dried over anhydrous Na₂SO₄, filtered, and the filtrate was evaporated to dryness. The crude mass obtained was purified by Combiflash chromatography (Gradient: 30-80% ethyl acetate in hexane) to afford Compound 217 (0.24 g, 82% yield) as white foam. Synthesis of 2′-O-tri, hepta and nona-decyl w carboxymethyl ester Uridine Phosphoramidites

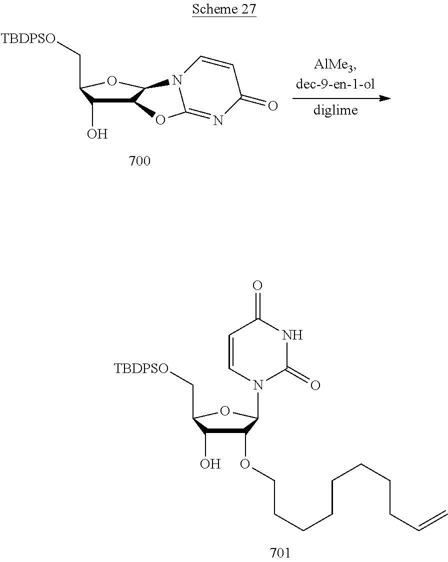

Compound 701: A 2M solution of trimethylaluminum (35.2 mL, 70.3 mmol) in heptane was added dropwise to a mixture of dec-9-en-1-ol (41.05 mL, 230.14 mmol) and anhydrous diglyme (24 mL). The resulting mixture was heated to 100° C. for 1 hour, cooled to room temperature followed by addition of 5′-OTBDPS anhydro uridine 700 (14.85 g, 31.96 mmol) in a single portion. The reaction mixture was heated at 125° C. overnight. The mixture was cooled to room temperature and partitioned between 10% H₃PO₄ (400 mL) and ethyl acetate (500 mL). The organic layer was separated, washed with brine, dried over anhydrous Na₂SO₄ and filtered. The volatiles were removed under vacuum and the residue was purified by ISCO automated column using 0-40% EtOAc in hexanes as eluent to give Compound 701 (10.5 g, 52%). ¹H NMR (500 MHz, DMSO-d6) δ 11.37 (d, J=2.2 Hz, 1H), 7.71 (d, J=8.1 Hz, 1H), 7.66-7.60 (m, 5H), 7.51-7.40 (m, 7H), 5.85 (d, J=4.5 Hz, 1H), 5.78 (ddt, J=17.0, 10.2, 6.7 Hz, 1H), 5.26 (dd, J=8.1, 2.2 Hz, 1H), 5.15 (s, 1H), 5.01-4.90 (m, 2H), 4.18 (t, J=5.1 Hz, 1H), 4.01-3.86 (m, 3H), 3.85-3.76 (m, 1H), 3.59 (dt, J=9.7, 6.5 Hz, 1H), 3.48 (dt, J=9.7, 6.6 Hz, 1H), 3.33 (s, 2H), 2.03-1.95 (m, 2H), 1.53-1.45 (m, 2H), 1.37-1.17 (m, 12H), 1.03 (s, 9H). ¹³C NMR (126 MHz, DMSO) δ 162.84, 150.30, 139.82, 138.79, 135.13, 134.96, 132.68, 132.17, 130.05, 130.00, 127.97, 114.56, 101.55, 86.45, 84.02, 80.90, 69.73, 68.08, 63.27, 39.52, 33.14, 28.98, 28.83, 28.72, 28.46, 28.23, 26.67, 25.33, 18.82. LRMS (ESI) calculated for C₃₅H₄₉N₂O₆Si [M+H]⁺ m/z=621.33, found 621.4.

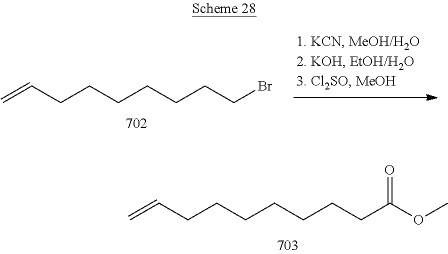

Compound 703: A solution of 9-bromonon-1-ene 702 (13.3 g, 62.24 mmol) in MeOH (105 mL) was combined with a solution of potassium cyanide (5.27 g, 80.9 mmol) in H₂O (25 mL). The resulting mixture was heated to reflux for 24 hours. The organic solvent was removed under reduced pressure and the aqueous residue was extracted with ethyl acetate, dried over anhydrous Na₂SO₄, filtered, and concentrated to give the crude nitrile. To a solution of potassium hydroxide (31.43 g, 560.18 mmol) in 100 mL of ethanol and 100 mL of water) was added the previously synthesized nitrile, and the resulting mixture was heated to reflux for 24 hours. The total volume of the mixture was reduced to half under reduced pressure and then extracted with Et₂O (100 mL). Concentrated HCl was added dropwise to the resulting aqueous layer until it reached acidic pH (1-2), followed by extraction with Et₂O (2×100 mL). The organic extracts were dried over anhydrous Na₂SO₄, filtered, and evaporated to dryness to afford crude carboxylic acid (9.81 g). Crude carboxylic acid was dissolved in anhydrous MeOH (130 mL) and thionyl chloride (6.77 mL, 93.36 mmol) was added dropwise at 0° C. The ice bath was removed, and the resulting mixture was stirred for 3 hours. The volatiles were removed under reduced pressure and the residue was filtered through a silica pad (5 cm) using EtOAc/hexane 2:8 as eluent to give Compound 703 (10.5 g, 91% over 3 steps). ¹H NMR (400 MHz, chloroform-d) δ 5.80 (ddt, J=16.9, 10.1, 6.7 Hz, 1H), 5.04-4.89 (m, 2H), 3.66 (s, 3H), 2.30 (t, J=7.5 Hz, 2H), 2.09-1.99 (m, 2H), 1.68-1.53 (m, 3H), 1.45-1.25 (m, 9H). LRMS (ESI) calculated for C₁₁H₂₁O₂[M+H]⁺ m/z=185.15, found 185.1.

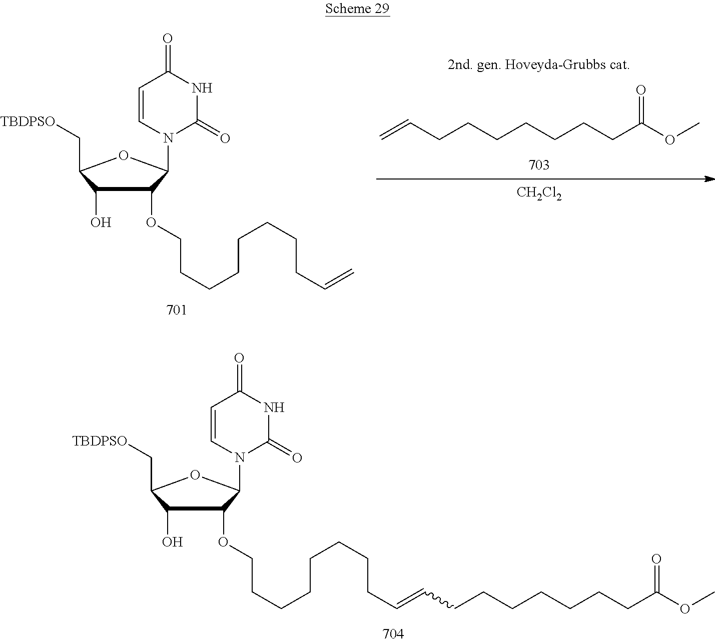

Compound 704: Compound 701 (5.42 g, 8.73 mmol) was dissolved in anhydrous DCM (175 mL) followed by the addition of methyl dec-9-enoate 703 (10.46 g, 56.74 mmol), benzoquinone (141.56 mg, 1.31 mmol) and second generation Hoveyda-Grubbs catalyst (547.04 mg, 873.0 μmol). The resulting mixture was stirred at reflux for 3.5 hours, cooled to room temperature, and the total volume of the reaction mixture was reduced to half under reduced pressure. The resulting solution was loaded into a 120 g silica column cartridge and purified by ISCO automated column using 0-60% EtOAc in hexanes as eluent to give Compound 704 (5.37 g, 79%) as a greenish oil. ¹H NMR (500 MHz, DMSO-d6) δ 11.37 (d, J=2.2 Hz, 1H), 7.71 (d, J=7.9 Hz, 1H), 7.67-7.58 (m, 5H), 7.52-7.38 (m, 7H), 5.85 (d, J=4.5 Hz, 1H), 5.39-5.29 (m, 2H), 5.25 (ddd, J=8.1, 2.3, 1.0 Hz, 1H), 5.17-5.12 (m, 1H), 4.18 (q, J=5.5 Hz, 1H), 3.97-3.86 (m, 3H), 3.83-3.76 (m, 1H), 3.61-3.55 (m, 4H), 3.52-3.40 (m, 2H), 3.31 (s, 2H), 2.27 (td, J=7.4, 4.1 Hz, 2H), 1.95-1.87 (m, 4H), 1.54-1.44 (m, 4H), 1.34-1.19 (m, 20H), 1.08-0.95 (m, 11H). ¹³C NMR (101 MHz, DMSO) δ 173.33, 170.32, 162.86, 150.31, 139.81, 135.15, 134.98, 132.66, 132.15, 130.08, 130.06, 130.02, 130.00, 128.00, 115.62, 101.55, 86.44, 84.02, 80.93, 71.28, 69.72, 69.57, 68.08, 63.27, 59.75, 58.05, 51.14, 39.52, 33.25, 31.95, 31.91, 28.99, 28.91, 28.87, 28.76, 28.48, 28.43, 28.30, 26.68, 25.35, 24.40, 20.76, 18.84, 14.09. LRMS (ESI) calculated for C₄₄H₆₅N₂O₈Si [M+H]⁺ m/z=777.44, found 777.5.

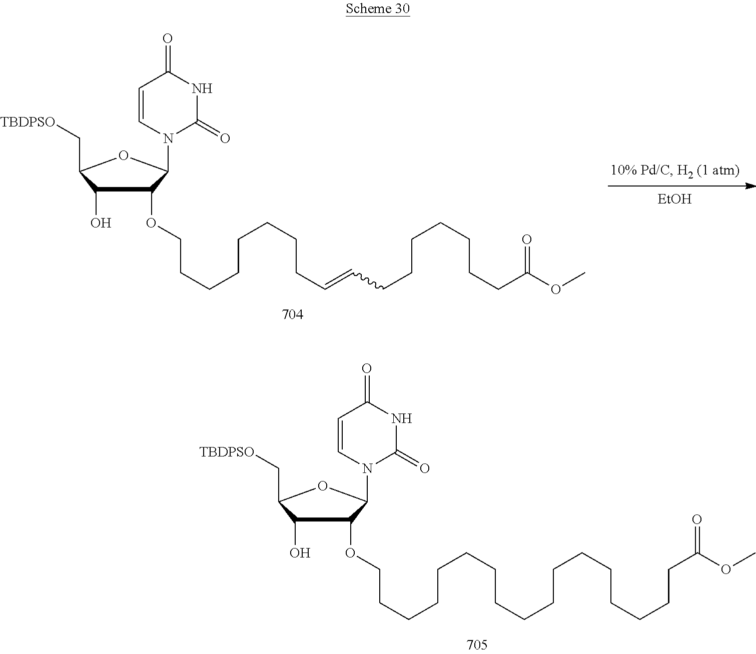

Compound 705: 10% Pd on carbon (735.42 mg, 0.69 mmol) was added to a stirred solution of nucleoside 704 (5.37 g, 6.91 mmol) in EtOH (170 mL). The flask was equipped with a three-way adapter connected to a balloon filled with Hydrogen. The flask was submitted to a sequence of vacuum-H₂ refill (×3) to saturate the solution. After 0.5 hour, the mixture was diluted with MeOH and filtered through a celite pad rinsing with more methanol. The filtrate was evaporated under reduced pressure to give crude Compound 705 (5.01 g, 93%). ¹H NMR (400 MHz, DMSO-d6) δ 11.37 (d, J=2.2 Hz, 1H), 7.71 (d, J=8.1 Hz, 1H), 7.67-7.59 (m, 5H), 7.52-7.39 (m, 7H), 5.85 (d, J=4.5 Hz, 1H), 5.25 (dd, J=8.1, 2.2 Hz, 1H), 5.16 (d, J=6.1 Hz, 1H), 4.18 (q, J=5.5 Hz, 1H), 3.97-3.86 (m, 3H), 3.83-3.76 (m, 1H), 3.62-3.54 (m, 4H), 3.52-3.40 (m, 2H), 2.27 (td, J=7.4, 2.4 Hz, 2H), 1.53-1.44 (m, 4H), 1.30-1.15 (m, 28H), 1.03 (d, J=1.3 Hz, 9H). ¹³C NMR (101 MHz, DMSO) δ 173.35, 162.85, 150.32, 139.82, 135.16, 134.98, 132.66, 132.15, 130.09, 130.03, 128.01, 101.55, 86.44, 84.03, 80.93, 69.71, 68.08, 63.28, 56.02, 51.15, 39.52, 33.26, 29.04, 29.02, 29.00, 28.96, 28.86, 28.76, 28.66, 28.44, 26.69, 25.36, 24.43, 18.84, 18.56. LRMS (ESI) calculated for C₄₄H₆₇N₂O₈Si [M+H]⁺ m/z=779.46, found 779.4.

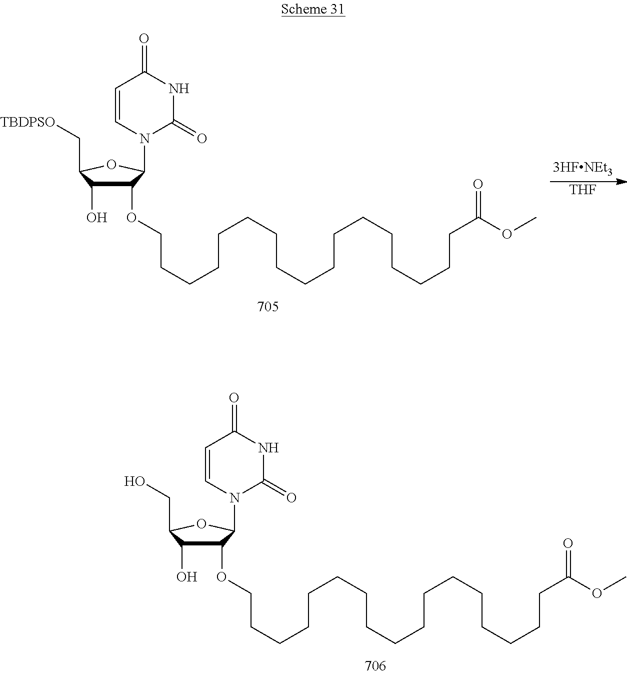

Compound 706: Triethylamine (3.59 mL, 25.72 mmol) and triethylamine trihydrofluoride (3.14 mL, 19.3 mmol) were added sequentially to a stirred solution of Compound 705 (5.01 g, 6.43 mmol) in THE (50 mL). The resulting mixture was heated at 45° C. for 4 hours, followed by removal of the volatiles under reduced pressure. The crude residue was purified by ISCO automated column using 0-100% EtOAc in hexane as eluent to give Compound 706 (1.46 g, 42%).

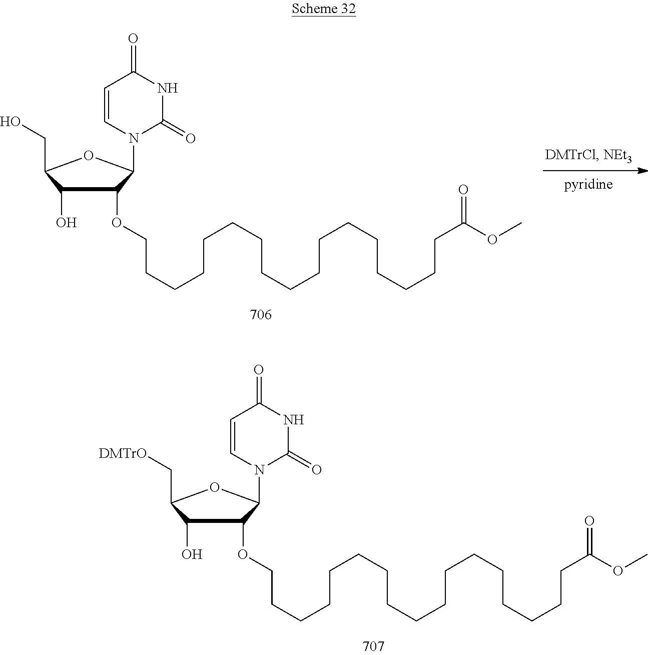

Compound 707: 4,4′-Dimethoxytrityl chloride (1.10 g, 3.24 mmol) and Triethylamine (0.45 mL, 3.24 mmol) were added to an stirred solution of nucleoside 706 (1.46 g, 2.70 mmol) in pyridine (20 mL). After 3 hours, the solvent was removed under reduced pressure, the residue was dissolved in EtOAc and washed with water, brine, dried over Na₂SO₄, filtered, and evaporated to dryness. The residue was purified by ISCO automated column using 0-50% EtOAc in hexanes as eluent to give Compound 707 (1.325 g, 58%)

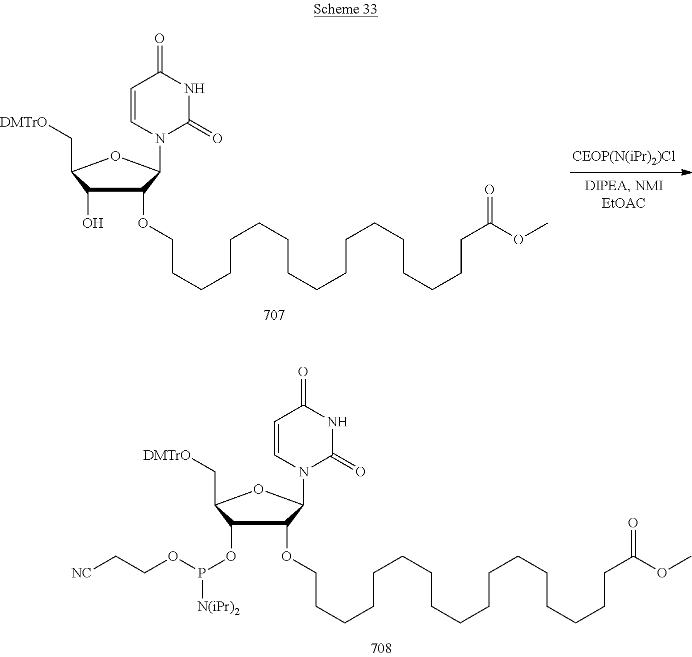

Compound 708: DIPEA, 2-cyanoethyl-N,N-diisopropylchlorophosphoramidite, and N-methylimidazole were added sequentially to a stirred solution of Compound 707 in anhydrous EtOAc at 0° C. The cold bath was removed, and the reaction mixture was stirred for 1 hour. The reaction was quenched with a solution of triethanolamine (2.7 M, 50 mL) in MeCN/toluene and stirred for 5 minutes. The mixture was diluted with ethyl acetate, transferred to a separatory funnel, layers separated, and the organic layer was washed sequentially with a 5% NaCl solution, and brine. The organic layer was dried over Na₂SO₄ and evaporated to dryness. The residue was pre-adsorbed on triethylamine pre-treated silica gel. The column was equilibrated with hexanes containing 1% NEt₃. The residue was purified by ISCO automated column using 0-40% EtOAc in hexanes as eluent to give Compound 708.

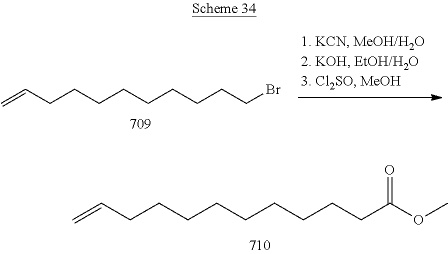

Compound 710: A solution of 11-bromoundec-1-ene (25.08 g, 103.25 mmol) in MeOH (180 mL) was combined with a solution of potassium cyanide (8.74 g, 134.2 mmol) in H₂O (45 mL). The resulting mixture was heated to reflux for 24 hours. The organic solvent was removed under reduced pressure and the aqueous residue was extracted with ethyl acetate, dried over anhydrous Na₂SO₄, filtered, and concentrated to give the crude nitrile. To a solution of potassium hydroxide (52.14 g, 929.3 mmol) in 150 mL of ethanol and 150 mL of water was added the previously synthesized nitrile, and the resulting mixture was heated to reflux for 24 hours. The total volume of the mixture was reduced to half under reduced pressure and then extracted with Et₂O (200 mL). Concentrated HCl was added dropwise to the resulting aqueous layer until it reached acidic pH (1-2), followed by extraction with Et₂O (2×200 mL). The organic extracts were dried over anhydrous Na₂SO₄, filtered, and evaporated to dryness to afford crude carboxylic acid (16.6 g). Crude carboxylic acid was dissolved in anhydrous MeOH (150 mL) and thionyl chloride (8.24 mL, 113.6 mmol) was added dropwise at 0° C. The ice bath was removed, and the resulting mixture was stirred for 3 hours. The volatiles were removed under reduced pressure and the residue was filtered through a silica pad (5 cm) using EtOAc/hexane 2:8 as eluent to give Compound 710 (17.5 g, 79% over 3 steps). ¹H NMR (500 MHz, DMSO-d₆) δ 5.79 (ddt, J=16.9, 10.2, 6.7 Hz, 1H), 5.03-4.90 (m, 2H), 3.57 (s, 3H), 2.28 (t, J=7.4 Hz, 2H), 2.04-1.96 (m, 2H), 1.55-1.46 (m, 2H), 1.37-1.21 (m, 13H).

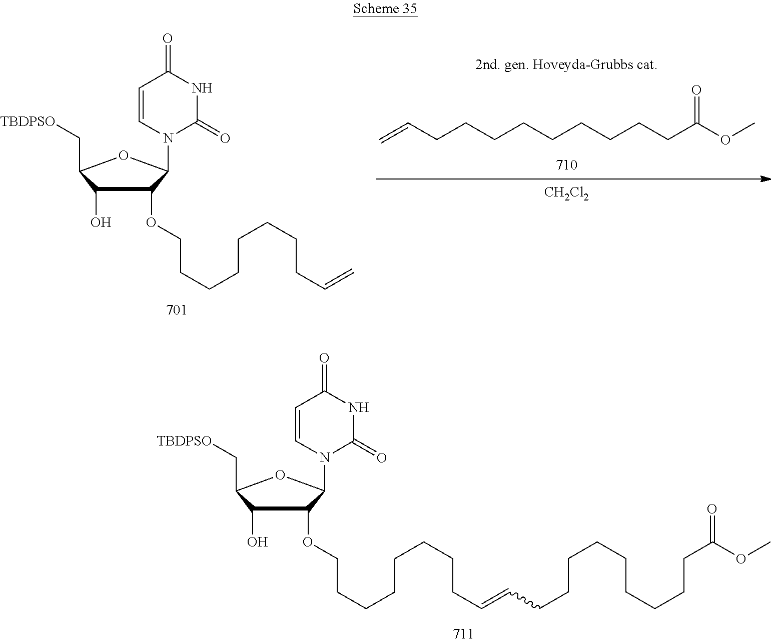

Compound 711: Compound 701 (4.44 g, 7.15 mmol) was dissolved in anhydrous DCM (145 mL) followed by the addition of methyl dec-9-enoate 710 (15.18 g, 71.5 mmol), benzoquinone (116 mg, 1.07 mmol) and second generation Hoveyda-Grubbs catalyst (448 mg, 0.715 mmol). The resulting mixture was stirred at reflux for 3.5 hours, cooled to room temperature, and the total volume of the reaction mixture was reduced to half under reduced pressure. The resulting solution was loaded into a 120 g silica column cartridge and purified by ISCO automated column using 0-60% EtOAc in hexanes as eluent to give Compound 711 (4.63 g, 80%) as a greenish oil. ¹H NMR (400 MHz, DMSO-d₆) δ 11.39 (d, J=2.2 Hz, 1H), 7.71 (d, J=8.1 Hz, 1H), 7.67-7.59 (m, 4H), 7.50-7.40 (m, 6H), 5.84 (d, J=4.4 Hz, 1H), 5.40-5.29 (m, 2H), 5.24 (dd, J=8.0, 2.2 Hz, 1H), 5.17 (d, J=6.2 Hz, 1H), 4.18 (q, J=5.5 Hz, 1H), 3.97-3.86 (m, 3H), 3.83-3.75 (m, 1H), 3.62-3.53 (m, 4H), 3.52-3.40 (m, 1H), 1.92 (q, J=6.4 Hz, 4H), 1.54-1.42 (m, 4H), 1.32-1.19 (m, 22H), 1.03 (s, 10H).

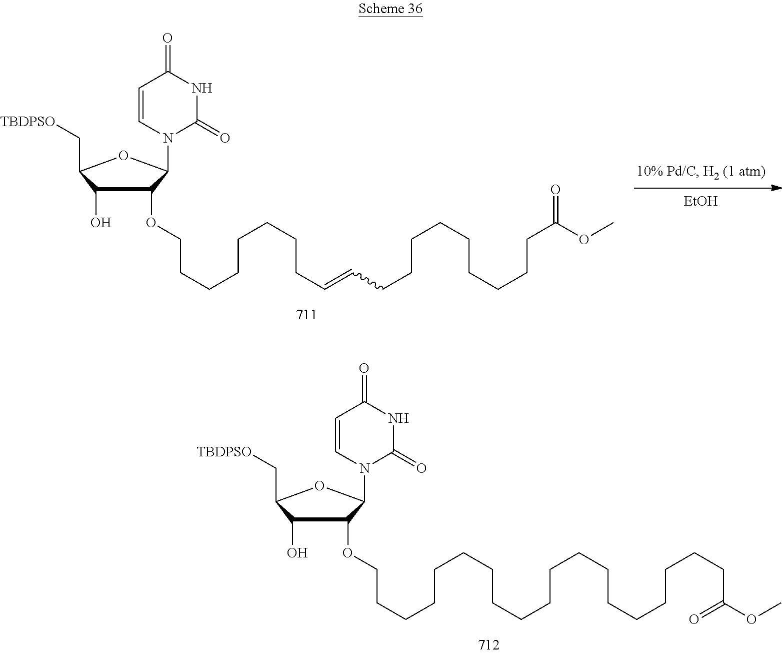

Compound 712: 10% Pd on carbon (612 mg, 0.575 mmol) was added to a stirred solution of nucleoside 711 (4.63 g, 5.75 mmol) in EtOH (150 mL). The flask was equipped with a three-way adapter connected to a balloon filled with Hydrogen. The flask was submitted to a sequence of vacuum-H₂ refill (×3) to saturate the solution. After 0.5 hour, the mixture was diluted with MeOH and filtered through a celite pad rinsing with more methanol. The filtrate was evaporated under reduced pressure to give crude 712 (4.52 g, 97%). ¹H NMR (400 MHz, DMSO-d6) δ 11.40-11.37 (m, 1H), 7.71 (d, J=8.1 Hz, 1H), 7.67-7.59 (m, 4H), 7.52-7.39 (m, 7H), 5.85 (d, J=4.4 Hz, 1H), 5.24 (dd, J=8.1, 2.2 Hz, 1H), 5.17 (d, J=6.1 Hz, 1H), 4.18 (q, J=5.4 Hz, 1H), 3.99-3.87 (m, 3H), 3.83-3.75 (m, 1H), 3.56 (s, 4H), 3.46 (ddt, J=14.0, 9.0, 6.6 Hz, 2H), 2.27 (t, J=7.4 Hz, 2H), 1.54-1.43 (m, 4H), 1.30-1.13 (m, 31H), 1.05-1.00 (m, 10H). ¹³C NMR (101 MHz, DMSO) δ 173.32, 162.85, 150.31, 139.80, 135.15, 134.97, 132.66, 132.14, 130.08, 130.02, 128.00, 101.55, 86.44, 84.02, 80.95, 69.71, 68.08, 63.27, 56.02, 54.91, 51.13, 39.52, 33.26, 29.04, 29.01, 28.96, 28.86, 28.76, 28.67, 28.45, 26.68, 25.36, 24.43, 18.84, 18.56. LRMS (ESI) calculated for C46H₇₁N₂O₈Si [M+H]⁺ m/z=807.49, found 807.5.

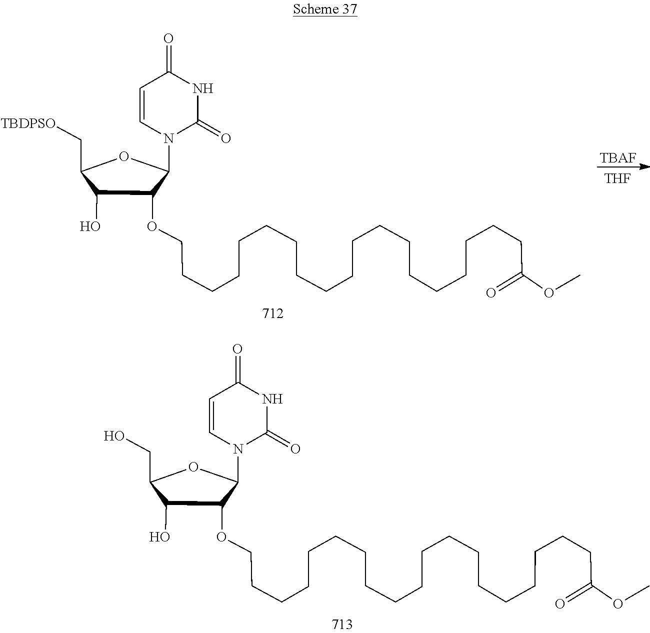

Compound 713: Tetrabutylammonium fluoride (1 M in THF) was added to a stirred solution of Compound 712 in THF. The mixture was stirred at room temperature for 3 hours before the volatiles were removed under reduced pressure. The residue was reconstituted in CH₂Cl₂ and partitioned with water. The layers were separated, and the aqueous portion was extracted with CH₂Cl₂ (3×20 mL). The combined organic extracts were dried over Na₂SO₄, and concentrated under reduced pressure. The residue was purified by flash column chromatography on silica gel (2:8 EtOAc/hexanes) to give Compound 713.

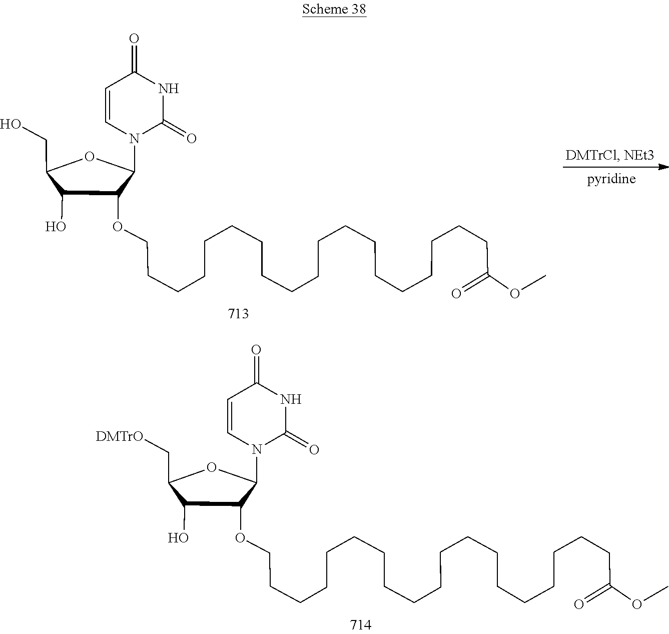

Compound 714: 4,4′-Dimethoxytrityl chloride (x g, x mmol) and Triethylamine (x mL, x mmol) were added to an stirred solution of nucleoside 713 (x g, x mmol) in pyridine (x mL). After 3 hours, the solvent was removed under reduced pressure, the residue was dissolved in EtOAc and washed with water, brine, dried over Na₂SO₄, filtered, and evaporated to dryness. The residue was purified by ISCO automated column using 0-50% EtOAc in hexanes as eluent to give Compound 714.

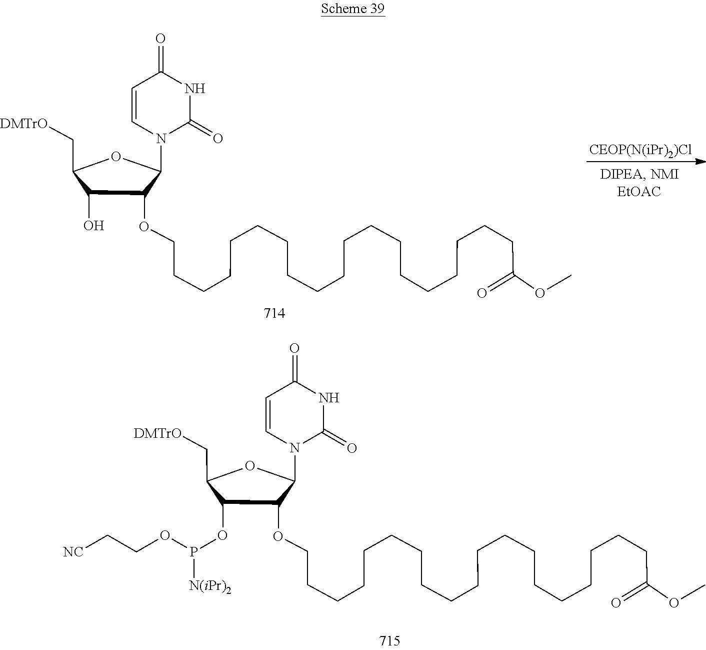

Compound 715: DIPEA, 2-cyanoethyl-N,N-diisopropylchlorophosphoramidite, and N-methylimidazole were added sequentially to a stirred solution of compound 714 in anhydrous EtOAc at 0° C. The cold bath was removed, and the reaction mixture was stirred for 1 hour. The reaction was quenched with a solution of triethanolamine (2.7 M, 50 mL) in MeCN/toluene and stirred for 5 minutes. The mixture was diluted with ethyl acetate, transferred to a separatory funnel, layers separated, and the organic layer was washed sequentially with a 5% NaCl solution, and brine. The organic layer was dried over Na₂SO₄ and evaporated to dryness. The residue was pre-adsorbed on triethylamine pre-treated silica gel. The column was equilibrated with hexanes containing 1% NEt₃. The residue was purified by ISCO automated column using 0-40% EtOAc in hexanes as eluent to give Compound 715.

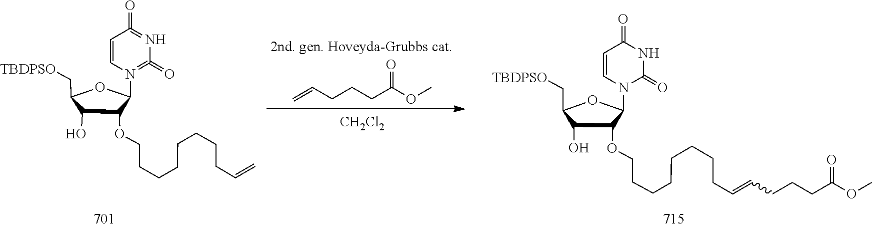

Compound 717: Compound 701 was dissolved in anhydrous DCM followed by the addition of methyl hex-5-enoate, benzoquinone and second generation Hoveyda-Grubbs catalyst. The resulting mixture was stirred at reflux for 3.5 hours, cooled to room temperature, and the total volume of the reaction mixture was reduced to half under reduced pressure. The resulting solution was loaded into a 120 g silica column cartridge and purified by ISCO automated column using 0-60% EtOAc in hexanes as eluent to give Compound 716 as a greenish oil.

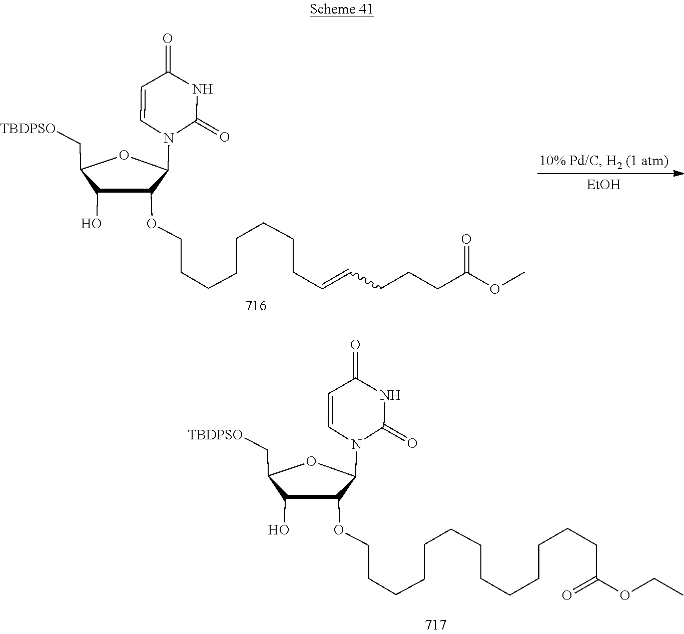

Compound 717: 10% Pd on carbon was added to a stirred solution of nucleoside 716 in EtOH. The flask was equipped with a three-way adapter connected to a balloon filled with Hydrogen. The flask was submitted to a sequence of vacuum-H₂ refill (×3) to saturate the solution. After 0.5 hour, the mixture was diluted with MeOH and filtered through a celite pad rinsing with more methanol. The filtrate was evaporated under reduced pressure to give crude 717.

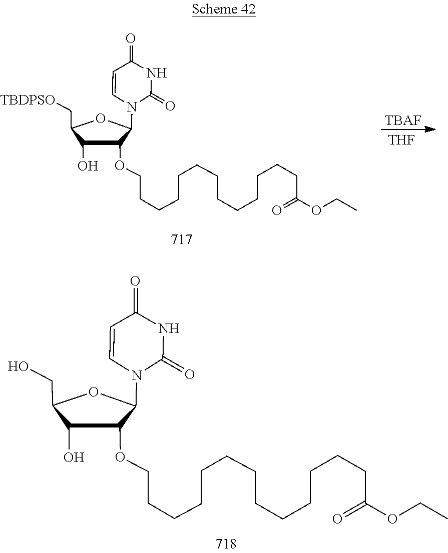

Compound 718: Tetrabutylammonium fluoride (1 M in THF) was added to a stirred solution of compound 717 in THF. The mixture was stirred at room temperature for 3 hours before the volatiles were removed under reduced pressure. The residue was reconstituted in CH₂Cl₂ and partitioned with water. The layers were separated and the aqueous portion was extracted with CH₂Cl₂ (3×20 mL). The combined organic extracts were dried over Na₂SO₄, concentrated under reduced pressure. The residue was purified by flash column chromatography on silica gel (2:8 EtOAc/hexanes) to give Compound 718.

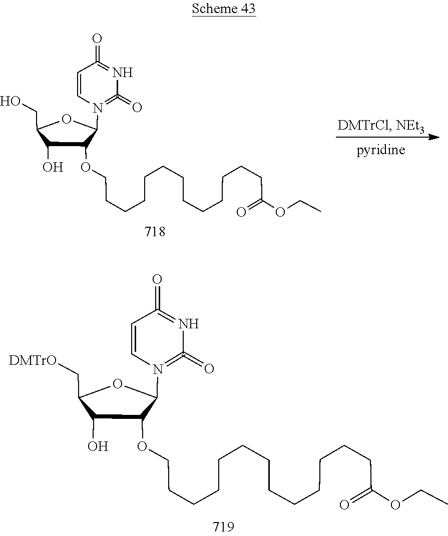

Compound 719: 4,4′-Dimethoxytrityl chloride and Triethylamine were added to a stirred solution of nucleoside 718 in pyridine. After 3 hours, the solvent was removed under reduced pressure, the residue was dissolved in EtOAc and washed with water, brine, dried over Na₂SO₄, filtered, and evaporated to dryness. The residue was purified by ISCO automated column using 0-50% EtOAc in hexanes as eluent to give Compound 719.

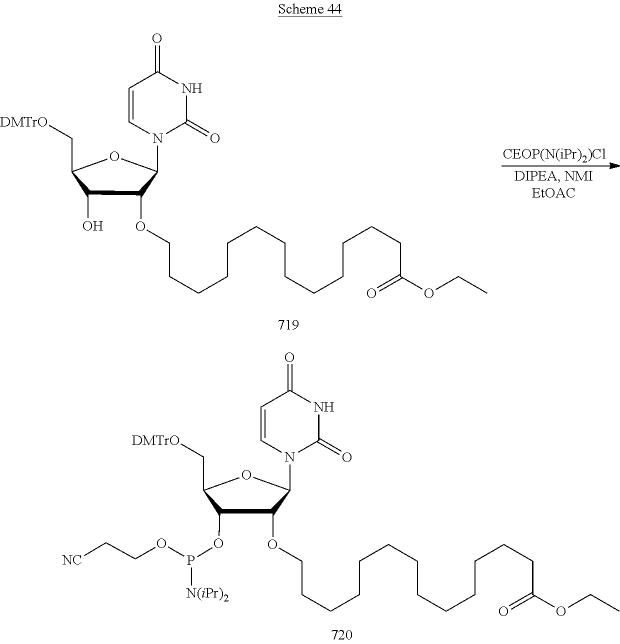

Compound 720: DIPEA, 2-cyanoethyl-N,N-diisopropylchlorophosphoramidite, and N-methylimidazole were added sequentially to a stirred solution of compound 719 in anhydrous EtOAc at 0° C. The cold bath was removed, and the reaction mixture was stirred for 1 hour. The reaction was quenched with a solution of triethanolamine (2.7 M, 50 mL) in MeCN/toluene and stirred for 5 minutes. The mixture was diluted with ethyl acetate, transferred to a separatory funnel, layers separated, and the organic layer was washed sequentially with a 5% NaCl solution, and brine. The organic layer was dried over Na₂SO₄ and evaporated to dryness. The residue was pre-adsorbed on triethylamine pre-treated silica gel. The column was equilibrated with hexanes containing 1% NEt₃. The residue was purified by ISCO automated column using 0-40% EtOAc in hexanes as eluent to give Compound 720.

### Synthesis of 2′, 3′-O-hexadecyl Uridine Phosphoramidites

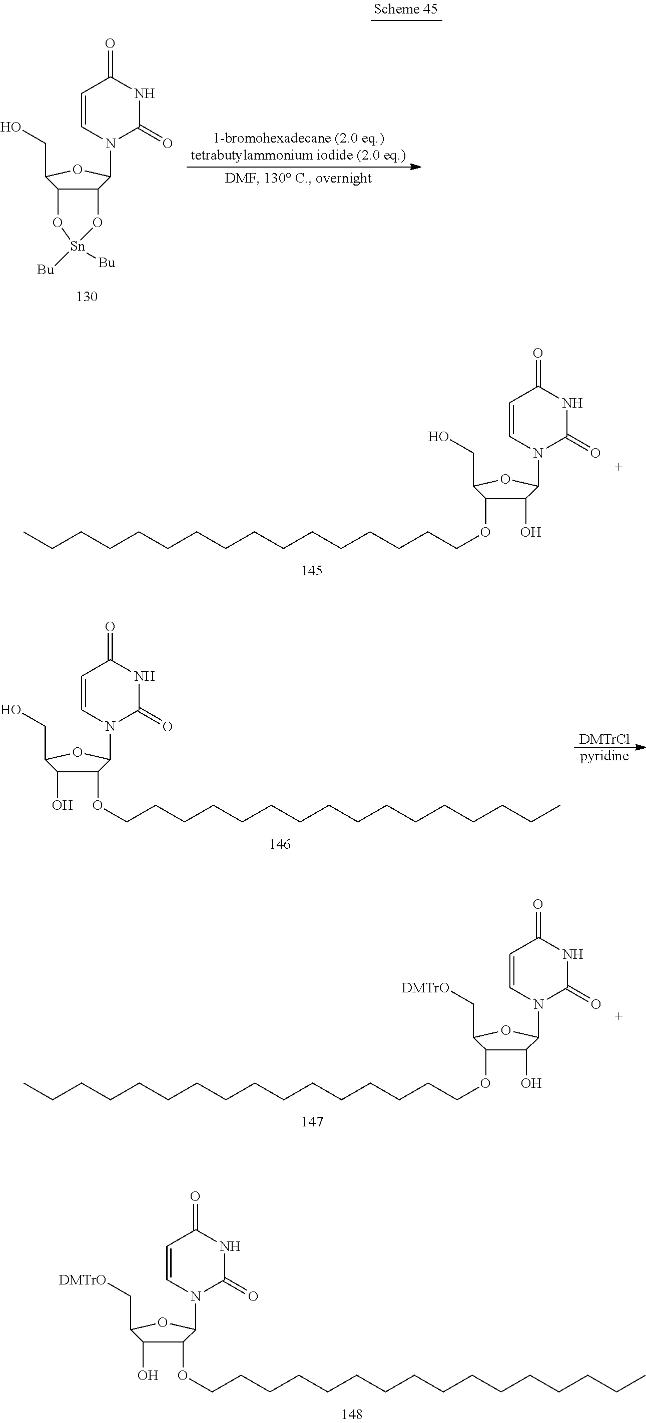

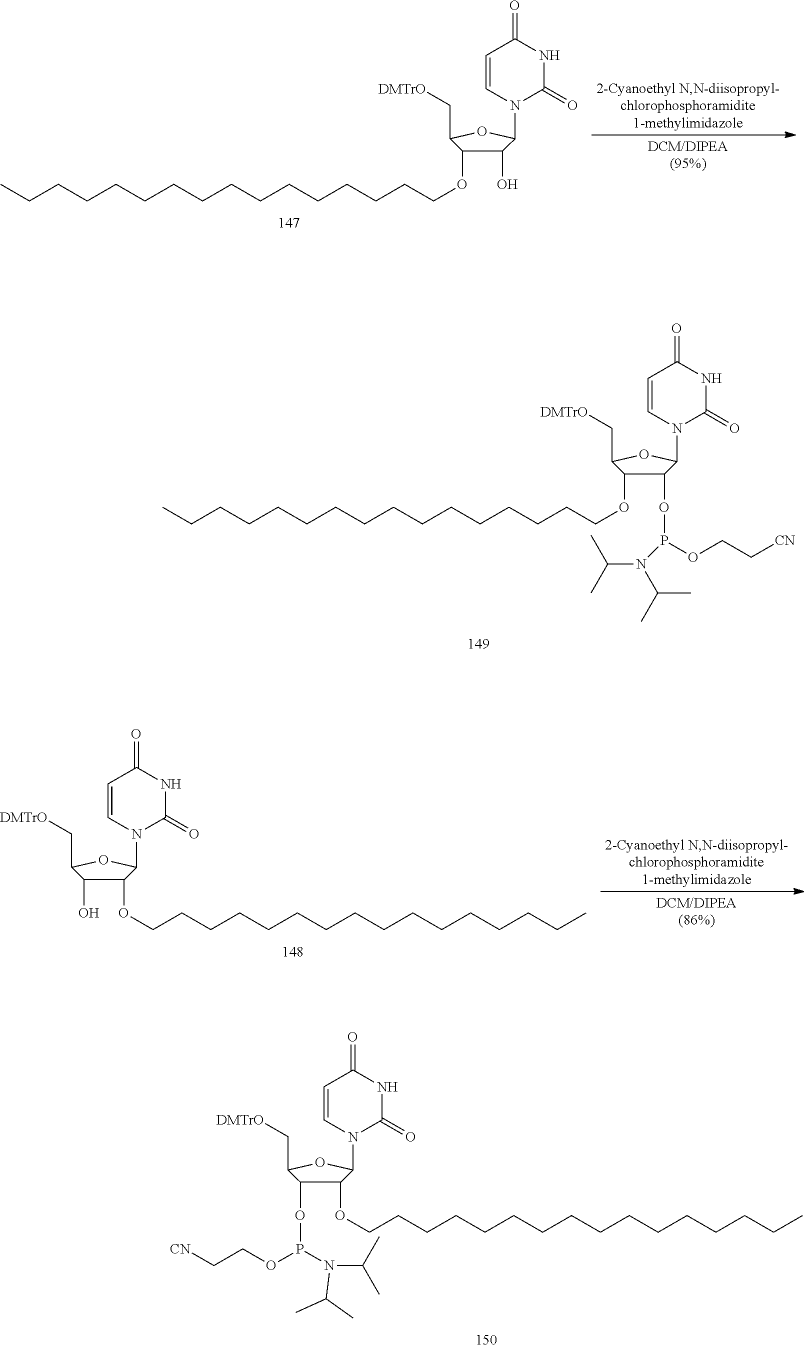

Compounds 145 and 146: To a solution of 2, 3′-O-dibutylstannylene uridine 130 (6.6 g, 13.89 mmol) in DMF (150 mL), 1-bromohexadecane (8.48 g, 27.78 mmol) and tetrabutylammonium iodide (10.26 g, 27.78 mmol) were added. The mixture was stirred at 130° C. in a reflux set-up overnight, forming a dark brown solution. The solution was eluted on silica (30% MeOH/DCM) and all UV active fractions were collected. The fractions were concentrated in vacuo and the product residue was eluted on silica (5% MeOH/DCM) to obtain a crude mixture of Compound 145 and Compound 146 (3.38 g).

Compound 147 and 148: Pyridine (10 mL) was added to a crude mixture of Compound 145 and Compound 146 (2.34 g, 4.99 mmol), and concentrated in vacuo to remove trace water. The mixture residue was placed under high vacuum and back-filled with argon 3 times. A solution of Compound 145 and Compound 146 in pyridine (42 mL) was treated with 4,4′-dimethoxytrityl chloride (1.86 g, 5.49 mmol) and stirred at room temperature overnight under argon. The reaction was quenched with MeOH (5 mL) and concentrated in vacuo. The product residue was dissolved in 3% TEA/DCM and washed with saturated NaHCO₃ (aq.) and brine. The organic layer was dried with Na₂SO₄ and concentrated in vacuo. A silica column was neutralized by eluting 3% TEA/DCM 3 times before loading the product residue. The product was purified on silica (40-60% ethylacetate in 3% TEA/hexanes). Compound 147 (1.32 g, 34%) and Compound 148 (660 mg, 17%) were separated and obtained as white solids. 147: ¹H NMR (500 MHz, DMSO-d₆) δ 11.3 (brs, 1H), 7.74 (d, 1H), 7.33 (d, 2H), 7.28 (t, 2H), 7.20-7.22 (m, 5H), 6.85-6.87 (m, 4H), 5.66 (d, 1H), 5.38 (d, 1H), 5.30 (d, 1H), 4.19-4.22 (m, 1H), 3.88-3.96 (m, 2H), 3.70 (s, 6H), 3.53-3.57 (m, 1H), 3.34-3.38 (m, 1H), 3.22-3.31 (m, 2H), 1.45-1.48 (m, 2H), 1.21-1.27 (m, 26H), 0.84 (t, 3H). ¹³C NMR (126 MHz, DMSO-d₆) δ 163.0, 158.1, 150.4, 144.6, 140.4, 135.3, 135.1, 129.7, 127.9, 127.7, 126.8, 113.2, 101.3, 89.4, 85.9, 80.4, 76.7, 72.0, 69.7, 62.3, 55.0, 52.0, 31.3, 29.2, 29.0, 29.0, 29.0, 28.9, 28.7, 25.5, 22.1, 13.9, 7.2.

Compound 149: Pyridine (8 mL) was added to Compound 147 (660 mg, 0.856 mmol) and concentrated in vacuo to remove trace water 3 times. The residue was placed under high vacuum and back-filled with argon 3 times. DCM (12 mL) was added to form a solution and placed in an ice bath with stirring. N,N-diisopropylethylamine (447 μL, 2.57 mmol) and 1-methylimidazole (13.7 μL, 0.171 mmol) were added and stirred for 20 minutes at 0° C. 2-cyanoethyl N,N-diisopropylchloro-phosphoramidite (382 μL, 1.71 mmol) was added, and the solution was removed from the ice bath and stirred at room temperature for 2 hours. The product mixture was washed with saturated NaHCO₃ (aq.) and extracted with 3% TEA/DCM. The organic layer was dried with Na₂SO₄ and concentrated in vacuo. A silica column was neutralized by eluting 3% TEA/DCM 3 times before loading the product residue. The product was purified on silica (50% ethylacetate in 3% TEA/hexanes). Compound 149 (790 mg, 95%) was obtained as a white solid. ¹H NMR (500 MHz, CD₃CN) δ 8.84 (brs, 1H), 7.77 (d, 0.5H), 7.74 (d, 0.5H), 7.44 (d, 2H), 7.25-7.35 (m, 7H), 6.84-6.94 (m, 4H), 5.91 (d, 0.5H), 5.86 (d, 0.5H), 4.48-4.51 (m, 1H), 4.04-4.12 (m, 2H), 3.80-3.90 (m, 2H), 3.78 (s, 6H), 3.58-3.76 (m, 4H), 3.34-3.36 (m, 1H), 2.59-2.69 (m, 2H), 1.48-1.58 (m, 2H), 1.24-1.31 (m, 28H), 1.18 (d, 9H), 1.15 (d, 3H), 0.89 (t, 3H) ³¹P NMR (202 MHz, CD₃CN) δ 150.69 (s), 151.38 (s).

Compound 150: Pyridine (6 mL) was added to Compound 148 (1.32 g, 1.71 mmol) and concentrated in vacuo to remove trace water 3 times. The residue was placed under high vacuum and back-filled with argon 3 times. DCM (12 mL) was added to form a solution and placed in an ice bath with stirring. N,N-diisopropylethylamine (894 μL, 5.14 mmol) and 1-methylimidazole (28 μL, 0.342 mmol) were added and stirred for 20 minutes at 0° C. 2-cyanoethyl N,N-diisopropylchloro-phosphoramidite (765 μL, 3.42 mmol) was added, and the solution was removed from the ice bath and stirred at room temperature for 2 hours. The product mixture was washed with saturated NaHCO₃ (aq.) and extracted with 3% TEA/DCM. The organic layer was dried with Na₂SO₄ and concentrated in vacuo. A silica column was neutralized by eluting 3% TEA/DCM 3 times before loading the product residue. The product was purified on silica (50% ethylacetate in 3% TEA/hexanes). Compound 150 (1.43 g, 86%) was obtained as a white solid. ¹H NMR (500 MHz, CD₃CN) δ 8.92 (brs, 1H), 7.81 (d, 0.6H), 7.72 (d, 0.4H), 7.43-7.47 (m, 2H), 7.23-7.36 (m, 8H), 6.86-6.93 (m, 3H), 5.86 (d, 0.5H), 5.85 (d, 0.6H), 5.18-5.27 (m, 1H), 4.46-4.50 (m, 0.6H), 4.40-4.44 (m, 0.4H), 4.05 (t, 0.6H), 4.02 (t, 0.4H), 3.82-3.93 (m, 1H), 3.77-3.79 (m, 6H), 3.58-3.71 (m, 4H), 3.33-3.39 (m, 1H), 2.64-2.69 (m, 1H), 2.53 (t, 1H), 1.49-1.60 (m, 2H), 1.23-1.37 (m, 28H), 1.17 (dd, 9H), 1.06 (d, 3H), 0.89 (t, 3H) ³¹P NMR (202 MHz, CD₃CN) δ 150.69 (s), 151.38 (s). ³¹P NMR (202 MHz, CD₃CN) δ 150.69 (s), 151.06 (s).

### Synthesis of 2′-O—C3-amide-C18 Conjugated Uridine Amidite

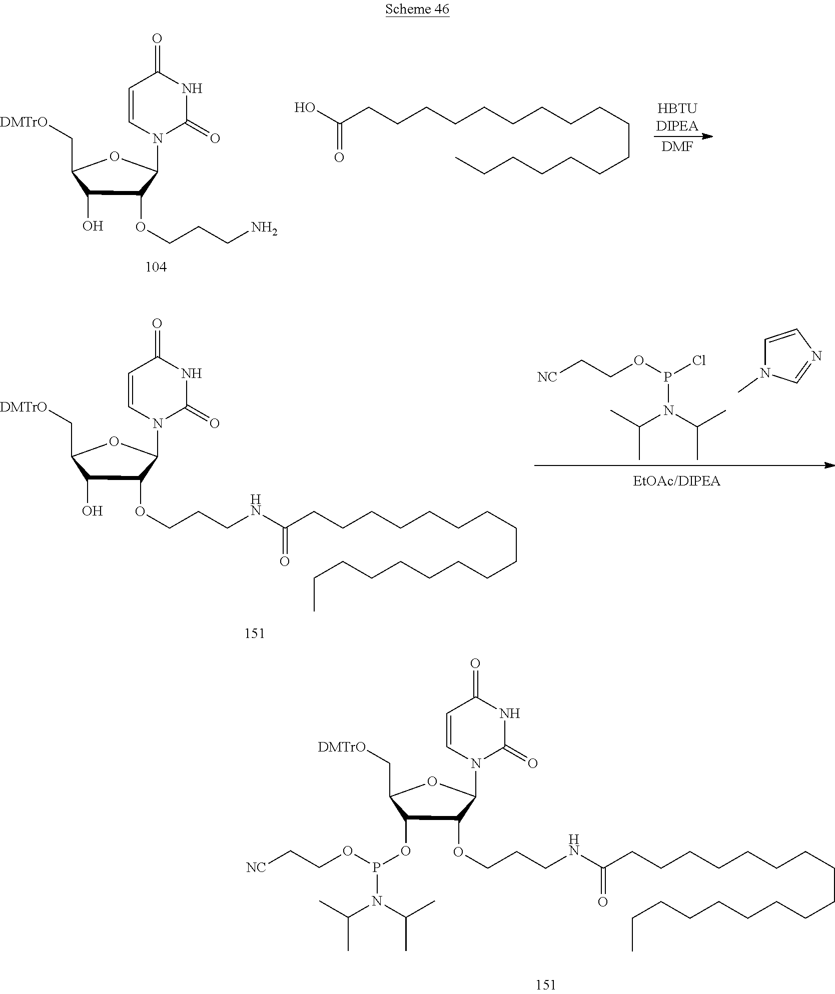

Compound 151: Compound 104 (6.0 g, 9.94 mmol), stearic acid (3.39 g, 11.9 mmol), and HBTU (4.6 g, 12.13 mmol) were combined in an empty flask equipped with a magnetic stirrer bar. The content of the flask was flushed with argon for 5 minutes followed by addition of DMF (25 mL) and DIPEA (5.2 mL, 29.8 mmol). After stirring for 20 hours, the reaction mixture was diluted with a saturated solution of NaHCO₃ and diethyl ether. The layers were separated, and the organic layer was washed with a saturated solution of NaHCO₃ and brine, and dried over Na₂SO₄. The volatiles were removed under reduced pressure, and the residue was purified by ISCO automated column using 0-6% MeOH in CH₂Cl₂ as eluent to give Compound 151 (5.5 g, 64%). ¹H NMR (500 MHz, chloroform-d) δ 8.39 (s, 1H), 8.04 (d, J=8.2 Hz, 1H), 7.41-7.36 (m, 2H), 7.32-7.27 (m, 6H), 6.86-6.81 (m, 4H), 5.89 (d, J=1.6 Hz, 1H), 5.81 (t, J=6.3 Hz, 1H), 5.26 (dd, J=8.1, 1.9 Hz, 1H), 4.51-4.42 (m, 1H), 4.08 (dt, J=7.9, 2.4 Hz, 1H), 3.91 (ddd, J=10.3, 6.1, 4.7 Hz, 1H), 3.86 (dd, J=5.2, 1.7 Hz, 1H), 3.80 (d, J=1.3 Hz, 6H), 3.72-3.64 (m, 2H), 3.62 (d, J=8.2 Hz, 1H), 3.55 (d, J=2.4 Hz, 2H), 3.26-3.17 (m, 1H), 2.21-2.13 (m, 2H), 1.91-1.70 (m, 2H), 1.67-1.59 (m, 2H), 1.31-1.21 (m, 28H), 0.88 (t, J=6.9 Hz, 3H).

Compound 152: Compound 151 (5.5 g, 6.32 mmol) was co-evaporated with acetonitrile (twice) and connected to the high vacuum line for 2 hours. The residue was dissolved in ethyl acetate (125 mL) and cooled to 0° C. To the previous solution, DIPEA (2.75 mL, 15.80 mmol), 2-cyanoethyl-N,N-diisopropylchlorophosphoramidite (3.53 mL, 15.80 mmol), and 1-methylimidazole (0.50 mL, 6.3 mmol) were added sequentially. The cold bath was removed, and the reaction mixture was stirred for 30 minutes. The reaction was quenched with a solution of triethanolamine (2.7 M, 17.5 mL) in MeCN/toluene and stirred for 5 minutes. The mixture was diluted with ethyl acetate, transferred to a separatory funnel, layers separated, and the organic layer was washed sequentially with a 5% NaCl solution (50 mL) and brine. The organic layer was dried over Na₂SO₄ and evaporated to dryness. The residue was pre-adsorbed on triethylamine pre-treated silica gel. The column was equilibrated with hexanes containing 1% NEt₃. The residue was purified by ISCO automated column using 0-60% EtOAc in hexanes as eluent to give Compound 152 (4.5 g, 67%). ¹H NMR (500 MHz, acetonitrile-d3) δ 8.95 (s, 1H), 7.77 (dd, J=48.2, 8.1 Hz, 1H), 7.46-7.40 (m, 2H), 7.35-7.27 (m, 6H), 6.90-6.84 (m, 4H), 6.39 (d, J=5.4 Hz, 1H), 5.84 (dd, J=7.6, 2.9 Hz, 1H), 5.20 (t, J=8.4 Hz, 1H), 4.45 (dddd, J=41.9, 10.0, 6.9, 5.0 Hz, 1H), 4.18-4.11 (m, 1H), 4.04-3.99 (m, 1H), 3.76 (d, J=3.1 Hz, 6H), 3.74-3.65 (m, 4H), 3.65-3.54 (m, 3H), 3.53-3.35 (m, 3H), 3.25-3.16 (m, 3H), 2.74 (t, J=5.9 Hz, 1H), 2.67 (td, J=5.9, 2.1 Hz, 1H), 2.54-2.50 (m, 2H), 2.08-2.02 (m, 2H), 1.70 (h, J=6.2 Hz, 2H), 1.54-1.47 (m, 2H), 1.29-1.22 (m, 28H), 1.18-1.01 (m, 12H), 0.87 (t, J=6.8 Hz, 3H). ³¹P NMR (202 MHz, CD₃CN) δ 149.59, 149.15.

### Synthesis of 2′-O—C3-amide-C14 Conjugated Uridine Amidite

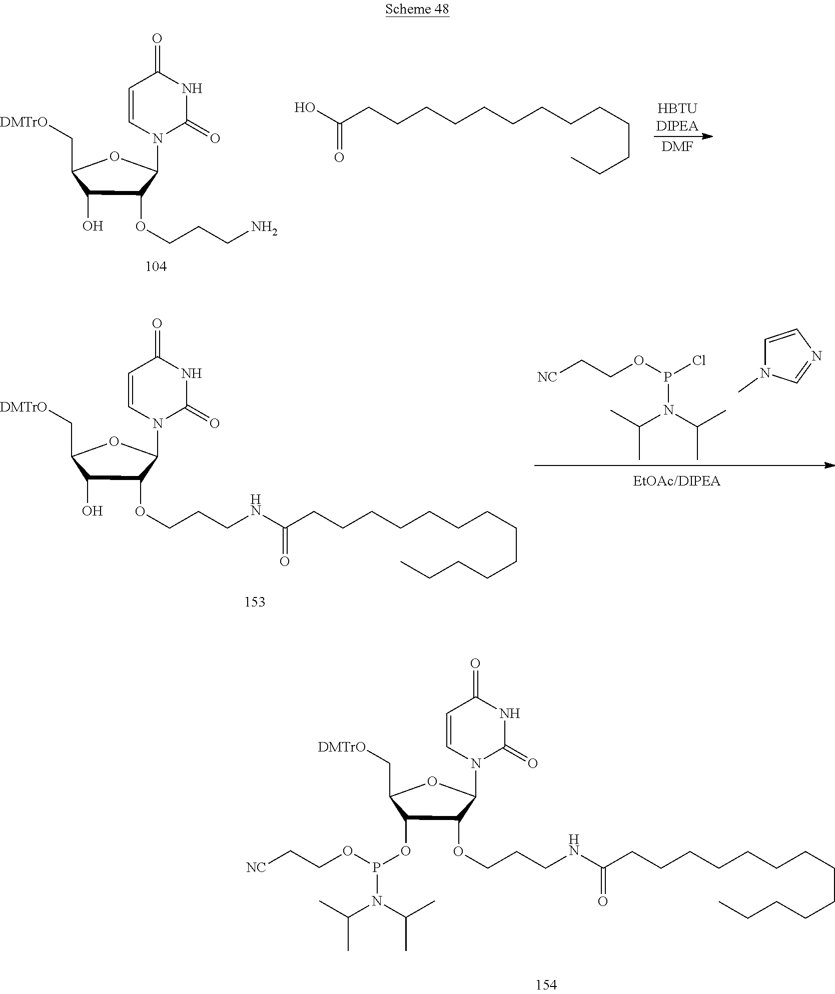

Compound 153: Compound 104 (5.0 g, 8.3 mmol), tetradecanoic acid (2.10 g, 9.19 mmol), and HBTU (3.83 g, 10.1 mmol) were combined in an empty flask equipped with a magnetic stirrer bar. The content of the flask was flushed with argon for 5 minutes followed by addition of DMF (25 mL) and DIPEA (4.3 mL, 24.8 mmol). After stirring for 20 hours, the reaction mixture was diluted with a saturated solution of NaHCO₃ and diethyl ether. The layers were separated, and the organic layer was washed with a saturated solution of NaHCO₃ and brine, and dried over Na₂SO₄. The volatiles were removed under reduced pressure, and the residue was purified by ISCO automated column using 0-6% MeOH in CH₂Cl₂ as eluent to give Compound 153 (3.93 g, 58%). ¹H NMR (400 MHz, chloroform-d) δ 8.94 (s, 1H), 7.44-7.23 (m, 9H), 6.91-6.78 (m, 4H), 5.95-5.85 (m, 2H), 5.32-5.22 (m, 1H), 4.46 (q, J=6.6 Hz, 1H), 4.08 (dt, J=8.0, 2.4 Hz, 1H), 3.98-3.89 (m, 1H), 3.86 (dd, J=5.2, 1.6 Hz, 1H), 3.80 (d, J=1.0 Hz, 6H), 3.72-3.52 (m, 4H), 2.20-2.13 (m, 2H), 1.89-1.53 (m, 5H), 1.31-1.19 (m, 20H), 0.87 (t, J=6.7 Hz, 3H).

Compound 154: Compound 153 (3.93 g, 4.83 mmol) was co-evaporated with acetonitrile (twice) and connected to the high vacuum line for 2 hours. The residue was dissolved in ethyl acetate (100 mL) and cooled to 0° C. To the previous solution, DIPEA (2.1 mL, 12.1 mmol), 2-cyanoethyl-N,N-diisopropylchlorophosphoramidite (2.69 mL, 12.1 mmol), and 1-methylimidazole (0.38 mL, 4.83 mmol) were added sequentially. The cold bath was removed, and the reaction mixture was stirred for 30 minutes. The reaction was quenched with a solution of triethanolamine (2.7 M, 14 mL) in MeCN/toluene and stirred for 5 minutes. The mixture was diluted with ethyl acetate, transferred to a separatory funnel, layers separated, and the organic layer was washed sequentially with a 5% NaCl solution (50 mL) and brine. The organic layer was dried over Na₂SO₄ and evaporated to dryness. The residue was pre-adsorbed on triethylamine pre-treated silica gel. The column was equilibrated with hexanes containing 1% NEt₃. The residue was purified by ISCO automated column using 0-60% EtOAc in hexanes as eluent to give Compound 154 (4.38 g, 89%). ¹H NMR (500 MHz, chloroform-d) δ 8.03 (dd, J=29.4, 8.2 Hz, 1H), 7.44-7.35 (m, 2H), 7.34-7.21 (m, 10H), 6.84 (ddd, J=8.9, 7.1, 3.1 Hz, 4H), 6.20 (q, J=6.3 Hz, 1H), 5.91 (dd, J=7.1, 2.0 Hz, 1H), 5.23 (dd, J=19.9, 8.1 Hz, 1H), 4.66-4.43 (m, 1H), 4.26-4.18 (m, 1H), 4.01 (ddd, J=11.6, 4.9, 2.0 Hz, 1H), 3.94-3.67 (m, 11H), 3.67-3.39 (m, 7H), 3.32 (tq, J=13.0, 6.1 Hz, 1H), 2.68-2.56 (m, 2H), 2.49-2.39 (m, 1H), 2.13 (q, J=7.9 Hz, 2H), 1.86-1.76 (m, 2H), 1.59 (s, 5H), 1.28-1.22 (m, 21H), 1.21-1.12 (m, 10H), 1.04 (d, J=6.8 Hz, 3H), 0.88 (t, J=6.9 Hz, 3H). ³¹P NMR (202 MHz, CDCl₃) δ 150.21, 149.86.

### Synthesis of 5′-amide-Lipophilic Conjugated 2′-OMe-Cytidine Amidite

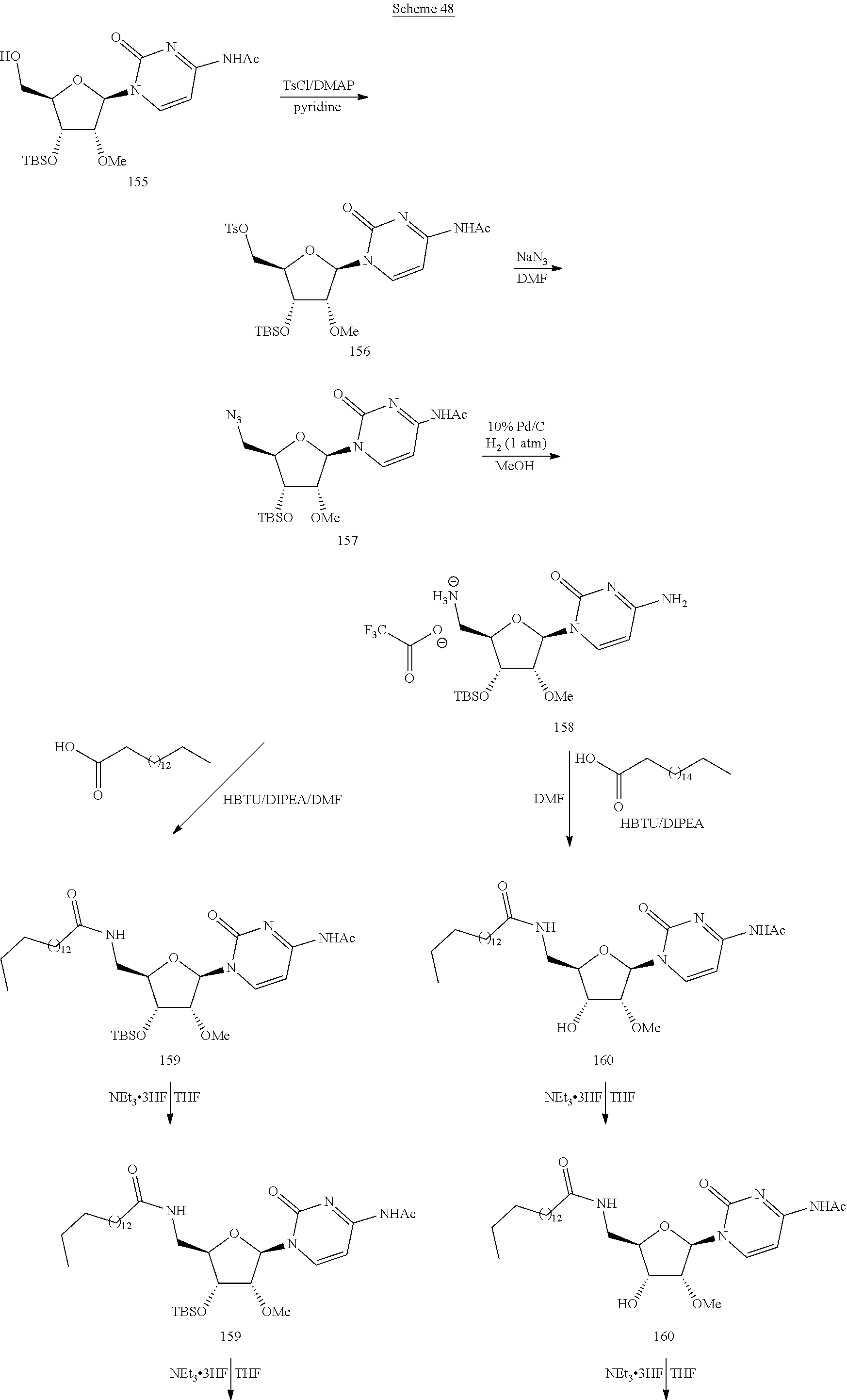

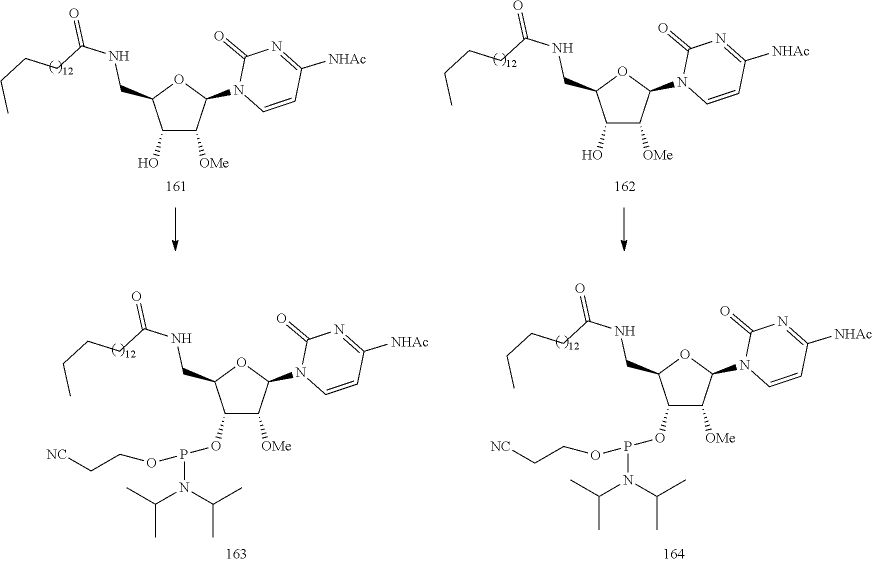 [SMILES: CCCCC(=O)NC[C@H]1O[C@@H](n2ccc(NC(C)=O)nc2=O)[C@H](OC)[C@@H]1O ; CCCCC(=O)NC[C@H]1O[C@@H](n2ccc(NC(C)=O)nc2=O)[C@H](OC)[C@@H]1O ; CCCCC(=O)NC[C@H]1O[C@@H](n2ccc(NC(C)=O)nc2=O)[C@H](OC)[C@@H]1OP(OCCC#N)N(C(C)C)C(C)C ; CCCCC(=O)NC[C@H]1O[C@@H](n2ccc(NC(C)=O)nc2=O)[C@H](OC)[C@@H]1OP(OCCC#N)N(C(C)C)C(C)C]

Compound 156: p-toluenesulfonyl chloride (20.7 g, 0.108 mol) was added to a stirred solution of Compound 155 (30.0 g, 72.5 mmol) and pyridine (29.3 mL, 0.363 mmol) in anhydrous CH₂Cl₂ (220 mL). The reaction mixture was heated to reflux for 48 hours. After cooling down, CH₂Cl₂ (200 mL) and a saturated aqueous solution of NaHCO₃ (500 mL) were added slowly and stirred vigorously for 1 hour. The mixture was transferred to a separatory funnel, the layers were separated, and the organic layer was washed with 1M HCl and brine. The organic layer was dried over Na₂SO₄, filtered, and evaporated to dryness to give crude tosylate Compound 156 (41.2 g). The crude tosylate was used in the next reaction without further purification.

Compound 157: Sodium azide (14.15 g, 0.217 mol) was added to a stirred solution of Compound 156 (41.2 g, 72.6 mmol) in DMF (360 mL). The resulting mixture was heated at 90° C. for 8 hours, cooled to room temperature, and combined with water (300 mL) and diethyl ether (200 mL). The mixture was transferred to a separatory funnel, the layers were separated, and the aqueous layer was extracted twice with diethyl ether. The organic layers were combined, dried over Na₂SO₄, and evaporated to dryness. The residue was purified by ISCO automated column using 0-60% EtOAc in hexanes as eluent to give Compound 157 (27.5 g, 86% over two steps). ¹H NMR (500 MHz, chloroform-d) δ 9.06 (s, 1H), 8.24 (d, J=7.5 Hz, 1H), 7.46 (d, J=7.5 Hz, 1H), 5.89 (s, 1H), 4.17 (dt, J=8.9, 2.8 Hz, 1H), 4.01 (dd, J=8.9, 4.8 Hz, 1H), 3.94 (dd, J=13.5, 2.8 Hz, 1H), 3.69-3.60 (m, 6H), 2.26 (s, 3H), 0.90 (s, 9H), 0.08 (s, 6H).

Compound 158: To a stirred solution of Compound 157 (17.0 g, 38.8 mmol) in methanol (300 mL), 10% Pd/C Degussa type (4.13 g, 3.88 mmol) was added. The flask was equipped with a 3-way adapter connected to a balloon filled with hydrogen, and to the vacuum line. The content of the flask was subjected to a sequence of vacuum/refill with hydrogen (three times). After 40 minutes, TFA (3 ml) was added, and the resulting mixture was filtered through a celite pad and the volatiles evaporated to dryness. The residue was purified by ISCO automated column using 0-10% of MeOH in CH₂Cl₂ as eluent to give Compound 158 (12.5 g, 77%). ¹H NMR (400 MHz, DMSO-d6) δ 10.98 (s, 1H), 8.15 (d, J=7.5 Hz, 1H), 8.03 (s, 3H), 7.25 (d, J=7.5 Hz, 1H), 5.87 (d, J=3.3 Hz, 1H), 4.21 (t, J=5.7 Hz, 1H), 4.12-4.06 (m, 1H), 4.03-3.93 (m, 1H), 3.40 (s, 3H), 3.30-3.17 (m, 1H), 3.15-3.03 (m, 1H), 2.11 (s, 3H), 0.88 (s, 9H), 0.09 (d, J=2.0 Hz, 6H). ¹⁹F NMR (376 MHz, DMSO) δ −73.75.

Compound 159: Compound 158 (5.1 g, 9.7 mmol), palmitic acid (2.74 g, 10.7 mmol), and HBTU (4.41 g, 11.6 mmol) were combined in an empty flask equipped with a magnetic stirrer bar. The content of the flask was flushed with argon for 5 minutes followed by addition of DMF (32 mL) and DIPEA (6.76 mL, 38.8 mmol). After stirring for 4 hours, the reaction mixture was diluted with a saturated solution of NaHCO₃ and diethyl ether. The layers were separated, and the organic layer was washed with a saturated solution of NaHCO₃ and brine and dried over Na₂SO₄. The volatiles were removed under reduced pressure and the residue was purified by ISCO automated column using 0-6% MeOH in CH₂Cl₂ as eluent to give Compound 159. (4.97 g, 78%). ¹H NMR (400 MHz, Chloroform-d) δ 8.64 (s, 1H), 7.78 (d, J=7.4 Hz, 1H), 7.46 (d, J=7.4 Hz, 1H), 5.47 (d, J=3.9 Hz, 1H), 4.23-4.19 (m, 1H), 4.18-4.09 (m, 2H), 3.84-3.75 (m, 1H), 3.46 (s, 3H), 3.44-3.36 (m, 1H), 2.28-2.20 (m, 5H), 1.64-1.59 (m, 2H), 1.31-1.23 (m, 24H), 0.94-0.86 (m, 12H), 0.09 (s, 6H).

Compound 160: Compound 158 (5.85 g, 11.1 mmol), stearic acid (3.47 g, 12.2 mmol), and HBTU (5.05 g, 13.3 mmol) were combined in an empty flask equipped with a magnetic stirrer bar. The content of the flask was flushed with argon for 5 minutes followed by addition of DMF (37 mL) and DIPEA (7.74 mL, 44.4 mmol). After stirring for 4 hours, the reaction mixture was diluted with a saturated solution of NaHCO₃ and diethyl ether. The layers were separated, and the organic layer was washed with a saturated solution of NaHCO₃ and brine, and dried over Na₂SO₄. The volatiles were removed under reduced pressure and the residue was purified by ISCO automated column using 0-6% MeOH in CH₂Cl₂ as eluent to give Compound 160. (3.87 g, 51%). ¹H NMR (400 MHz, Chloroform-d) δ 8.44 (s, 1H), 7.77 (d, J=7.5 Hz, 1H), 7.45 (d, J=7.4 Hz, 1H), 5.46 (d, J=3.9 Hz, 1H), 4.24-4.19 (m, 1H), 4.17-4.10 (m, 2H), 3.46 (s, 3H), 3.41-3.36 (m, 1H), 2.27-2.24 (m, 2H), 1.29-1.23 (m, 28H), 0.92-0.86 (m, 12H), 0.10-0.08 (m, 6H).

Compound 161: Triethylamine trihydrofluoride (3.5 mL, 21.7 mmol) was added to a stirred solution of Compound 159 (4.7 g, 7.2 mmol) in THE (50 mL) at 0° C. After stirring for 24 hours at room temperature, the volatiles were removed under reduced pressure, and the residue was purified by ISCO automated column using 0-6% MeOH in CH₂Cl₂ as eluent to give Compound 161 (3.49 g, 90%). ¹H NMR (400 MHz, DMSO-d6) δ 10.94 (s, 1H), 8.12 (d, J=7.5 Hz, 1H), 8.01 (t, J=5.9 Hz, 1H), 7.24 (d, J=7.5 Hz, 1H), 5.82 (d, J=3.3 Hz, 1H), 5.19 (d, J=5.7 Hz, 1H), 3.93-3.84 (m, 2H), 3.78 (t, J=3.9 Hz, 1H), 3.42 (s, 3H), 2.13-2.05 (m, 5H), 1.48 (s, 2H), 1.34-1.16 (m, 25H), 0.86 (t, J=6.6 Hz, 3H).

Compound 162: Triethylamine trihydrofluoride (2.66 mL, 16.5 mmol) was added to a stirred solution of Compound 160 (3.74 g, 5.51 mmol) in THE (50 mL) at 0° C. After stirring for 24 hours at room temperature, the volatiles were removed under reduced pressure and the residue was purified by ISCO automated column using 0-6% MeOH in CH₂Cl₂ as eluent to give Compound 162.

Compound 163/164: Standard phosphitylation of Compounds 161 and 162 gives compounds 163 and 164, respectively.

### Synthesis of 5′-amide-Lipophilic Conjugated 2′-OMe-Adenosine Amidite

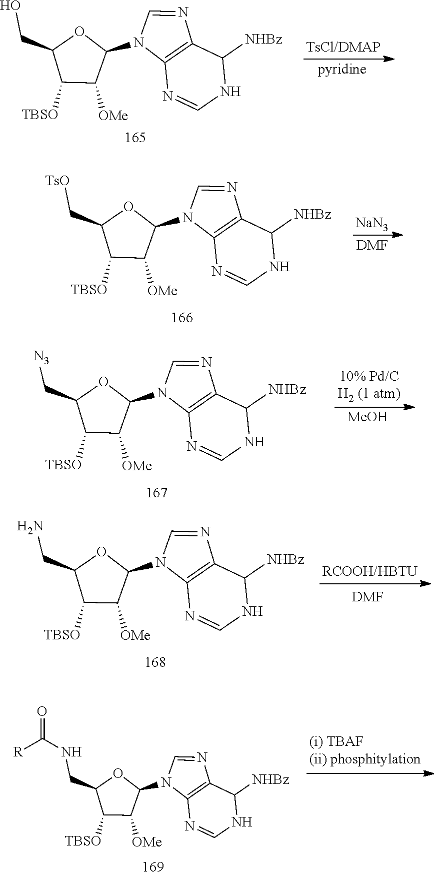

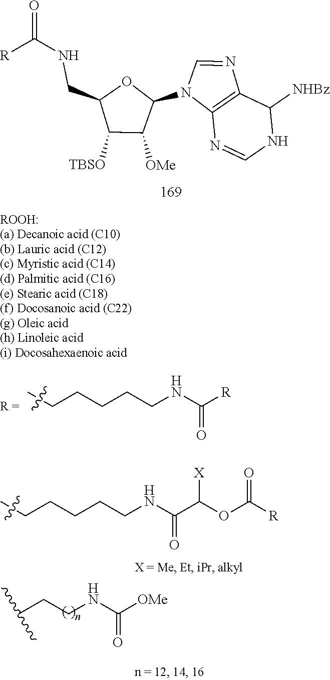

Compound 166: p-toluenesulfonyl chloride (34.3 g, 0.180 mmol) was added to a stirred solution of Compound 165 (30.0 g, 60.0 mmol) and pyridine (24.3 mL, 300 mmol) in anhydrous CH₂Cl₂ (180 mL). The reaction mixture was heated to reflux for 48 hours. After cooling down, CH₂Cl₂ (200 mL) and a saturated aqueous solution of NaHCO₃ (500 mL) were added slowly and stirred vigorously for 1 hour. The mixture was transferred to a separatory funnel, the layers were separated, and the organic layer was washed with 1M HCl and brine. The organic layer was dried over Na₂SO₄, filtered, and evaporated to dryness to give crude tosylate Compound 166. The crude tosylate was used in the next reaction without further purification.

Compound 167: Sodium azide (11.93 g, 183.5 mmol) was added to a stirred solution of crude Compound 166 (40.0 g, 61.2 mmol) in DMF (300 mL). The resulting mixture was heated at 90° C. for 8 hours, cooled to room temperature, and combined with water (300 mL) and diethyl ether (200 mL). The mixture was transferred to a separatory funnel, the layers separated, and the aqueous layer was extracted twice with diethyl ether. The organic layers were combined, dried over Na₂SO₄, and evaporated to dryness. The residue was purified by ISCO automated column using 0-8% MeOH in CH₂Cl₂ as eluent to give Compound 167 (29.8 g, 92%). ¹H NMR (500 MHz, chloroform-d, mixture of rotamers) δ 8.97 (s, 1H), 8.83-8.78 (m, 1H), 8.32-8.28 (m, 1H), 8.06-8.00 (m, 2H), 7.65-7.60 (m, 1H), 7.53 (dd, J=8.4, 7.0 Hz, 2H), 6.13 (d, J=3.4 Hz, 1H), 4.57-4.50 (m, 1H), 4.38 (dd, J=4.9, 3.5 Hz, 1H), 4.21 (dt, J=6.0, 4.0 Hz, 1H), 3.78 (dd, J=13.4, 3.9 Hz, 1H), 3.61 (dd, J=13.3, 4.3 Hz, 1H), 3.55-3.49 (m, 3H), 0.98-0.90 (m, 9H), 0.20-0.09 (m, 6H).

Compound 168: To a stirred solution of Compound 167 (13.58 g, 25.88 mmol) in methanol (130 mL), 10% Pd/C Degussa type (2.75 g, 2.59 mmol) was added. The flask was equipped with a 3-way adapter connected to a balloon filled with hydrogen, and to the vacuum line. The content of the flask was subjected to a sequence of vacuum/refill with hydrogen (three times). After 40 minutes, the reaction mixture was filtered through a celite pad and the volatiles evaporated to dryness. The residue was purified by ISCO automated column using 0-10% of MeOH in CH₂Cl₂ as eluent to give Compound 168 (9.4 g, 72%). ¹H NMR (500 MHz, chloroform-d) δ 8.99 (s, 1H), 8.79 (s, 1H), 8.28 (s, 1H), 8.03 (d, J=7.2 Hz, 2H), 7.65-7.59 (m, 1H), 7.57-7.50 (m, 2H), 6.07 (d, J=4.6 Hz, 1H), 4.56-4.45 (m, 2H), 4.15-4.08 (m, 1H), 3.43 (s, 3H), 3.14 (dd, J=13.6, 3.5 Hz, 1H), 2.96 (dd, J=13.6, 5.2 Hz, 1H), 0.95 (s, 9H), 0.14 (d, J=4.0 Hz, 6H).

Standard amide coupling of Compound 168 and lipid acids shown as RCOOH gives a variety of 5′-lipophilic conjugates of 2′-OMe-adenosine. These compounds can be converted to the phosphoramidite building blocks, as shown in Scheme 49 above.

### Synthesis of 5′-Amino Adenosine Lipid Amidites

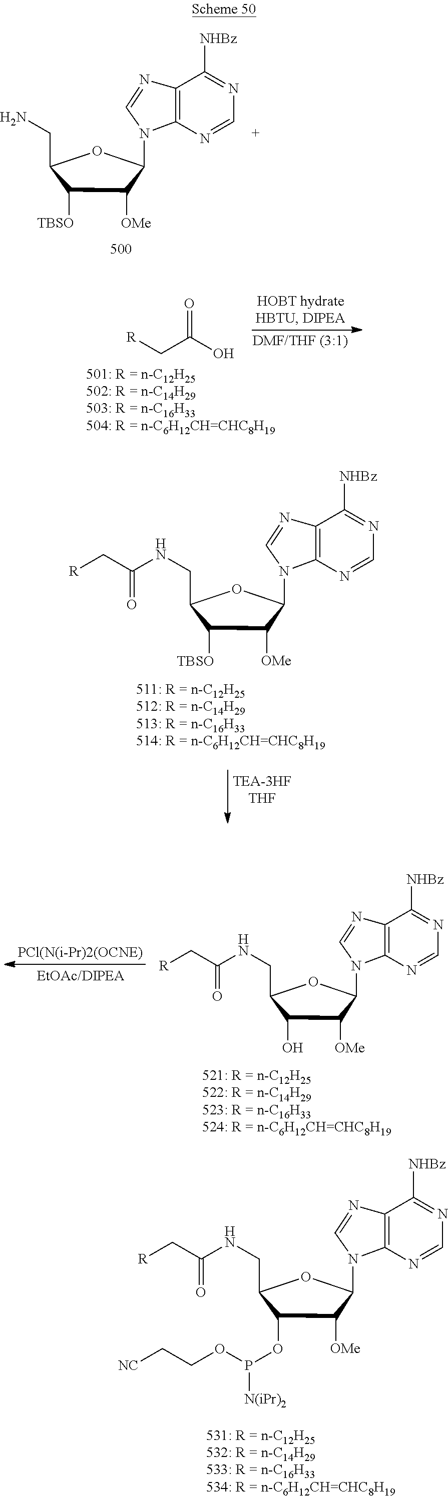

Compound 511: Compound 501 (1.26 g, 5.5 mmol) and HOBT hydrate (1.27 g, 8.3 mmol) were dissolved in anhydrous DMF (30 mL) and THE (10 ml) under an argon atmosphere and cooled to 0-5° C. in a water/ice bath. HBTU (2.45 g, 6.5 mmol) and N,N-diisopropylethylamine (3.0 mL, 17.1 mmol) were added and the solution stirred for 10 minutes. Compound 500 (2.3 g, 4.6 mmol) was added and the reaction was stirred at 0-5° C. for 2 hours. The reaction mixture was diluted with ethyl acetate (50 ml) and 5% NaCl (200 mL), and stirred for 5 minutes. The organic layer was isolated and washed with 10% H₃PO₄ (1×200 mL), 5% NaCl (1×200 mL), 4% NaHCO₃ (1×200 mL), and saturated NaCl (1×200 mL). The organic layer was dried over Na₂SO₄, filtered, and concentrated under reduced pressure at 25° C. to a foam. Purification was performed via silica gel flash chromatography, 80 g silica column, and ethyl acetate:hexanes (1:1 to 10:1 gradient). The fractions were concentrated under reduced pressure and chased with acetonitrile (twice). The fractions were dried under high vacuum overnight. Compound 511 was isolated as a white foam, with a 87% yield (2.86 g). ¹H NMR (400 MHz, DMSO-d₆) δ 11.23 (s, 1H), 8.77 (d, J=8.6 Hz, 2H), 8.13-7.96 (m, 3H), 7.64 (t, J=7.4 Hz, 1H), 7.54 (t, J=7.6 Hz, 2H), 6.11 (d, J=6.9 Hz, 1H), 4.72 (dd, J=6.9, 4.5 Hz, 1H), 4.54 (dd, J=4.6, 2.2 Hz, 1H), 4.01-3.88 (m, 1H), 3.55-3.42 (m, 1H), 3.39-3.29 (m, 1H), 3.27 (s, 3H), 2.08 (t, J=7.4 Hz, 2H), 1.48 (t, J=7.1 Hz, 2H), 1.20 (s, 20H), 0.91 (s, 9H), 0.83 (t, J=6.7 Hz, 3H), 0.12 (s, 6H). ¹³C NMR (101 MHz, DMSO-d₆) δ 172.42, 165.57, 152.12, 151.68, 150.58, 143.79, 132.43, 128.47, 128.41, 85.37, 84.69, 80.66, 70.96, 57.50, 40.54, 35.31, 31.28, 29.03, 29.00, 28.98, 28.87, 28.79, 28.70, 28.68, 25.60, 25.11, 22.08, 17.79, 13.90, −4.89.

Compound 512: Compound 512 was synthesized from Compound 500 and Compound 502 in an analogous fashion to Compound 511. Compound 512 was isolated as a glassy solid, with a 90% yield (3.05 g). ¹H NMR (400 MHz, DMSO-d₆) δ 11.23 (s, 1H), 8.77 (d, J=8.8 Hz, 2H), 8.05 (d, J=7.5 Hz, 3H), 7.64 (t, J=7.4 Hz, 1H), 7.54 (t, J=7.6 Hz, 2H), 6.11 (d, J=6.9 Hz, 1H), 4.72 (dd, J=7.0, 4.5 Hz, 1H), 4.53 (dd, J=4.5, 2.2 Hz, 1H), 3.99-3.92 (m, 1H), 3.55-3.42 (m, 1H), 3.36-3.27 (m, 1H), 3.26 (s, 3H) 2.08 (t, J=7.4 Hz, 2H), 1.53-1.41 (m, 2H), 1.30-1.15 (m, 24H), 0.91 (s, 9H), 0.87-0.78 (m, 3H), 0.12 (s, 6H). ¹³C NMR (101 MHz, DMSO-d₆) δ 172.41, 152.11, 151.68, 150.58, 143.79, 132.43, 128.47, 128.42, 126.05, 85.37, 84.70, 80.66, 70.96, 57.50, 40.54, 35.30, 31.27, 29.03, 29.01, 28.99, 28.96, 28.86, 28.78, 28.69, 28.67, 25.60, 25.11, 22.07, 17.79, 13.90, −4.89.

Compound 513: Compound 513 was synthesized from Compound 500 and Compound 503 in an analogous fashion to Compound 511. Compound 513 was isolated in 87% yield (3.05 g). ¹H NMR (400 MHz, DMSO-d₆) δ 11.24 (s, 1H), 8.77 (d, J=11.1 Hz, 2H), 8.09-7.99 (m, 3H), 7.67-7.59 (m, 1H), 7.59-7.49 (m, 2H), 6.11 (d, J=6.9 Hz, 1H), 4.73 (dd, J=7.0, 4.5 Hz, 1H), 4.53 (dd, J=4.5, 2.1 Hz, 1H), 3.99-3.91 (m, 1H), 3.55-3.43 (m, 1H), 3.38-3.22 (m, 4H), 2.08 (t, J=7.4 Hz, 2H), 1.54-1.42 (m, 2H), 1.30-1.12 (m, 28H), 0.91 (s, 9H), 0.86-0.78 (m, 3H), 0.11 (s, 6H). ¹³C NMR (101 MHz, DMSO-d₆) δ 172.40, 165.57, 151.68, 150.59, 143.80, 133.26, 132.44, 128.48, 128.42, 85.37, 84.71, 80.64, 70.96, 57.50, 40.54, 35.31, 31.30, 29.04, 29.01, 28.99, 28.89, 28.81, 28.72, 25.60, 25.12, 22.09, 17.79, 13.90, −4.89, −4.91.

Compound 514: Compound 514 was synthesized from Compound 500 and Compound 504 in an analogous fashion to Compound 511. Compound 514 was isolated as a white foam, with a 77% yield (2.08 g). ¹H NMR (400 MHz, DMSO-d₆) δ 11.23 (s, 1H), 8.77 (d, J=9.7 Hz, 2H), 8.05 (d, J=7.4 Hz, 3H), 7.64 (t, J=7.3 Hz, 1H), 7.54 (t, J=7.6 Hz, 2H), 6.11 (d, J=6.9 Hz, 1H), 5.35-5.22 (m, 2H), 4.73 (dd, J=7.0, 4.5 Hz, 1H), 4.54 (dd, J=4.6, 2.1 Hz, 1H), 4.00-3.90 (m, 1H), 3.55-3.42 (m, 1H), 3.39-3.20 (m, 4H), 2.08 (t, J=7.4 Hz, 2H), 2.01-1.85 (m, 4H), 1.55-1.41 (m, 2H), 1.41-1.09 (m, 20H), 0.91 (s, 9H), 0.87-0.77 (m, 3H), 0.12 (s, 6H). ¹³C NMR (101 MHz, DMSO-d₆) δ 172.37, 165.56, 152.10 151.66, 143.77, 132.41, 129.54, 129.52, 128.46, 128.40, 126.04, 85.37, 84.69, 80.64, 70.96, 57.49, 40.54, 35.29, 31.25, 29.06, 28.80, 28.69, 28.66, 28.56, 28.47, 26.57, 26.53, 25.58, 25.11, 22.06, 17.78, 13.87, −4.91.

Compound 521: Compound 511 (2.99 g, 3.9 mmol) was dissolved in anhydrous THF (12 mL) under an argon atmosphere. Triethylamine trihydrofluoride (2.6 mL, 15.7 mmol) was added and the reaction was stirred at room temperature for 19 hours, and then heated to 45° C. for 3 hours. The reaction mixture was cooled to room temperature and concentrated to an oil under reduced pressure. The oil was diluted with ethyl acetate (50 mL) and washed with 5% NaCl (2×150 mL) and saturated NaCl (1×150 mL). The organic layer was dried over Na₂SO₄, filtered, concentrated under reduced pressure at 25° C., and dried under high vacuum overnight. Compound 521 was isolated as a white foam, with a 97% yield (2.28 g). ¹H NMR (400 MHz, DMSO-d₆) δ 11.22 (s, 1H), 8.74 (d, J=15.4 Hz, 2H), 8.11-7.94 (m, 3H), 7.64 (t, J=7.4 Hz, 1H), 7.54 (t, J=7.6 Hz, 2H), 6.12 (d, J=6.2 Hz, 1H), 5.38 (s, 1H), 4.53 (t, J=5.5 Hz, 1H), 4.30 (t, J=4.0 Hz, 1H), 4.02-3.92 (m, 1H), 3.55-3.21 (m, 5H), 2.08 (t, J=7.4 Hz, 2H), 1.55-1.40 (m, J=6.8 Hz, 2H), 1.20 (d, J=4.7 Hz, 20H), 0.83 (t, J=6.7 Hz, 3H).

Compound 522: Compound 522 was synthesized from Compound 512 in an analogous fashion to Compound 521. Compound 522 was isolated in a 96% yield (2.42 g). ¹H NMR (400 MHz, DMSO-d₆) δ 11.22 (s, 1H), 8.74 (d, J=15.8 Hz, 2H), 8.10-7.94 (m, 3H), 7.64 (t, J=7.4 Hz, 1H), 7.54 (t, J=7.6 Hz, 2H), 6.12 (d, J=6.2 Hz, 1H), 5.38 (d, J=5.4 Hz, 1H), 4.53 (t, J=5.6 Hz, 1H), 4.32-4.27 (m, 1H), 4.02-3.94 (m, 1H), 3.52-3.24 (m, 5H), 2.12-2.02 (m, 2H), 1.53-1.40 (m, J=6.9 Hz, 2H), 1.20 (d, J=6.9 Hz, 24H), 0.83 (t, J=6.7 Hz, 3H).

Compound 523: Compound 523 was synthesized from Compound 513 in an analogous fashion to Compound 521. Compound 523 was isolated in a 100% yield (2.57 g). ¹H NMR (400 MHz, DMSO-d₆) δ 11.24 (s, 1H), 8.74 (d, J=12.6 Hz, 2H), 8.08-7.97 (m, 3H), 7.67-7.59 (m, 1H), 7.59-7.49 (m, 2H), 6.12 (d, J=6.2 Hz, 1H), 5.40 (s, 1H), 4.53 (dd, J=6.3, 4.9 Hz, 1H), 4.30 (dd, J=4.9, 3.3 Hz, 1H), 4.01-3.93 (m, 1H), 3.51-3.23 (m, 5H), 2.08 (t, J=7.4 Hz, 2H), 1.51-1.41 (m, 2H), 1.19 (d, J=7.9 Hz, 28H), 0.86-0.78 (m, 3H). ¹³C NMR (101 MHz, DMSO-d₆) δ 172.45, 165.58, 151.68, 150.55, 143.53, 133.27, 132.44, 128.48, 128.42, 85.62, 84.20, 81.58, 69.46, 57.51, 40.82, 35.33, 31.28, 29.04, 29.00, 28.94, 28.81, 28.70, 28.68, 25.24, 22.08, 13.92.

Compound 524: Compound 524 was synthesized from Compound 514 in an analogous fashion to Compound 521. Compound 524 was isolated as a white solid, with a 98% yield (1.67 g). ¹H NMR (400 MHz, DMSO-d₆) δ 9.58 (s, 1H), 8.70 (d, J=1.4 Hz, 1H), 8.33 (d, J=1.7 Hz, 1H), 8.08-7.99 (m, 2H), 7.69-7.62 (m, 1H), 7.58-7.52 (m, 2H), 7.47-7.38 (m, 1H), 6.04 (t, J=6.4 Hz, 1H), 4.71-4.54 (m, 2H), 4.41-4.26 (m, 1H), 3.99-3.63 (m, 5H), 3.44-3.29 (m, 4H), 2.83-2.67 (m, 2H), 2.34-2.16 (m, 3H), 1.67-1.52 (m, 2H), 1.35-1.17 (m, 36H), 0.88 (t, J=6.8 Hz, 3H).

Compound 531: Compound 521 (2.24 g, 3.7 mmol) was dissolved in anhydrous THF (20 mL) under an argon atmosphere. N,N-diisopropylethylamine (0.86 mL, 4.9 mmol) and 2-cyanoethyl N,N-diisopropylchlorophosphoramidite (1.1 mL, 4.9 mmol) were added and stirred at room temperature for 3 hours. Triethanolamine (3.7 mL, 10 mmol, 2.7 M solution in acetonitrile:toluene (4:9)) was added to the reaction mixture and stirred for 5 minutes. The reaction mixture was diluted with ethyl acetate (80 mL), concentrated under reduced pressure to 30 mL, diluted with ethyl acetate (50 mL), and then washed with 5% NaCl (3×100 mL) and saturated NaCl (1×100 mL). The organic layer was dried over Na₂SO₄, filtered, and concentrated to a foam under reduced pressure. Purification was carried out via silica gel flash chromatography, 80 g silica column, and ethyl acetate (+0.5% triethylamine):hexanes (1:1 to 100% ethyl acetate gradient). The fractions were concentrated under reduced pressure and chased with acetonitrile (2×). The fractions were dried under high vacuum overnight. Compound 531 was isolated as a white foam, with a 67% yield (2.00 g). ¹H NMR (400 MHz, acetonitrile-d₃) δ 8.70 (d, J=1.4 Hz, 1H), 8.33 (d, J=1.7 Hz, 1H), 8.08-7.99 (m, 2H), 7.69-7.62 (m, 1H), 7.55 (t, J=7.7 Hz, 2H), 7.48-7.40 (m, 1H), 6.04 (t, J=6.4 Hz, 1H), 4.71-4.54 (m, 2H), 4.41-4.26 (m, 1H), 3.99-3.63 (m, 5H), 3.44-3.29 (m, 4H), 2.83-2.67 (m, 2H), 2.34-2.16 (m, 3H), 1.67-1.52 (m, 2H), 1.35-1.17 (m, 32H), 0.88 (t, J=6.8 Hz, 3H). ¹³C NMR (101 MHz, acetonitrile-d₃) δ 174.21, 174.15, 152.70, 151.40, 144.57, 144.48, 134.89, 133.66, 129.70, 129.21, 126.33, 119.73, 119.66, 88.57, 85.59, 82.48, 72.19, 60.24, 60.07, 59.43, 59.23, 59.12, 59.07, 58.64, 44.35, 44.23, 44.18, 44.05, 41.61, 41.46, 37.07, 37.02, 32.70, 30.45, 30.43, 30.41, 30.30, 30.19, 30.14, 30.10, 30.07, 26.56, 26.51, 25.12, 25.04, 24.99, 24.96, 24.93, 23.46, 21.15, 21.12, 21.08, 21.05, 14.47. ³¹P NMR (162 MHz, acetonitrile-d₃) δ 150.87, 149.79.

Compound 532: Compound 532 was synthesized from Compound 522 in an analogous fashion to Compound 531. Compound 532 was isolated as a white foam, with a 81% yield (2.56 g). ¹H NMR (400 MHz, acetonitrile-d₃) δ 9.56 (s, 1H), 8.71 (d, J=1.3 Hz, 1H), 8.33 (d, J=1.6 Hz, 1H), 8.07-7.96 (m, 2H), 7.66 (t, J=7.4 Hz, 1H), 7.56 (t, J=7.6 Hz, 2H), 7.46-7.38 (m, 1H), 6.04 (t, J=6.3 Hz, 1H), 4.71-4.53 (m, 2H), 4.41-4.25 (m, 1H), 3.99-3.63 (m, 5H), 3.44-3.30 (m, 4H), 2.82-2.67 (m, 2H), 2.31-2.18 (m, 3H), 1.65-1.52 (m, 2H), 1.35-1.18 (m, 35H), 0.89 (t, J=6.8 Hz, 3H). ¹³C NMR (101 MHz, Acetonitrile-d₃) δ 174.21, 174.14, 152.70, 151.41, 144.57, 144.48, 134.90, 133.67, 129.72, 129.21, 126.34, 126.29, 119.66, 88.58, 85.59, 85.49, 85.46, 72.02, 60.25, 60.07, 59.43, 59.24, 59.12, 59.08, 58.64, 44.36, 44.24, 44.18, 44.06, 41.60, 41.46, 37.08, 37.02, 32.71, 30.46, 30.44, 30.43, 30.40, 30.30, 30.18, 30.15, 30.10, 30.07, 26.56, 26.51, 25.13, 25.05, 25.00, 24.96, 24.93, 23.46, 21.15, 21.12, 21.09, 21.05, 14.48. ³¹P NMR (162 MHz, Acetonitrile-d₃) δ 150.87, 149.80.

Compound 533: Compound 533 was synthesized from Compound 523 in an analogous fashion to Compound 531. Compound 533 was isolated in a 89% yield (2.95 g). ¹H NMR (400 MHz, acetonitrile-d₃) δ 9.63 (s, 1H), 8.69 (d, J=1.4 Hz, 1H), 8.33 (d, J=1.5 Hz, 1H), 8.07-7.97 (m, 2H), 7.70-7.60 (m, 1H), 7.58-7.51 (m, 2H), 7.48-7.40 (m, 1H), 6.04 (t, J=6.6 Hz, 1H), 4.71-4.52 (m, 2H), 4.41-4.25 (m, 1H), 3.99-3.64 (m, 5H), 3.44-3.29 (m, 4H), 2.82-2.69 (m, 2H), 2.37-2.15 (m, 3H), 1.65-1.52 (m, 2H), 1.45-1.16 (m, 39H), 0.94-0.84 (m, 3H). ¹³C NMR (101 MHz, acetonitrile-d₃) δ 174.20, 174.13, 166.46, 152.68, 151.41, 151.39, 144.57, 144.47, 134.89, 133.65, 129.69, 129.21, 126.33, 126.28, 119.71, 119.64, 88.57, 85.58, 85.49, 85.45, 82.51, 82.48, 72.19, 60.24, 60.07, 59.43, 59.23, 59.12, 59.07, 58.64, 44.35, 44.23, 44.18, 44.05, 41.62, 41.47, 37.08, 37.02, 32.71, 30.48, 30.46, 30.44, 30.43, 30.41, 30.30, 30.19, 30.15, 30.11, 30.08, 26.56, 26.51, 25.13, 25.05, 25.00, 24.97, 24.94, 23.46, 21.15, 21.12, 21.08, 21.05, 14.49. ³¹P NMR (162 MHz, acetonitrile-d₃) δ 150.87, 149.79.

Compound 534: Compound 534 was synthesized from Compound 524 in an analogous fashion to Compound 531. Compound 534 was isolated as a white foam, with a 77% yield (1.65 g). ¹H NMR (400 MHz, acetonitrile-d₃) δ 9.56 (s, 1H), 8.71 (d, J=1.4 Hz, 1H), 8.33 (d, J=1.7 Hz, 1H), 8.07-7.98 (m, 2H), 7.69-7.62 (m, 1H), 7.60-7.51 (m, 2H), 7.48-7.33 (m, 1H), 6.04 (t, J=6.4 Hz, 1H), 5.38-5.27 (m, 2H), 4.71-4.54 (m, 2H), 4.41-4.26 (m, 1H), 3.99-3.63 (m, 5H), 3.45-3.29 (m, 4H), 2.84-2.67 (m, 2H), 2.34-2.17 (m, 3H), 2.09-1.92 (m, 3H), 1.66-1.52 (m, 2H), 1.39-1.18 (m, 32H), 0.94-0.83 (m, 3H). ¹³C NMR (101 MHz, acetonitrile-d₃) δ 174.11, 152.70, 151.40, 144.56, 134.90, 133.67, 130.83, 130.76, 129.71, 129.21, 118.34, 88.57, 44.36, 44.23, 44.18, 44.05, 41.62, 41.47, 37.07, 37.01, 32.69, 30.52, 30.50, 30.25, 30.10, 30.07, 30.04, 29.91, 27.86, 26.56, 26.51, 25.13, 25.05, 25.00, 24.97, 24.93, 23.45, 14.48, 2.01, 1.19. ³¹P NMR (162 MHz, acetonitrile-d₃) δ 150.86, 149.79.

### Synthesis of Sterically Hindered Ester-Containing Lipid

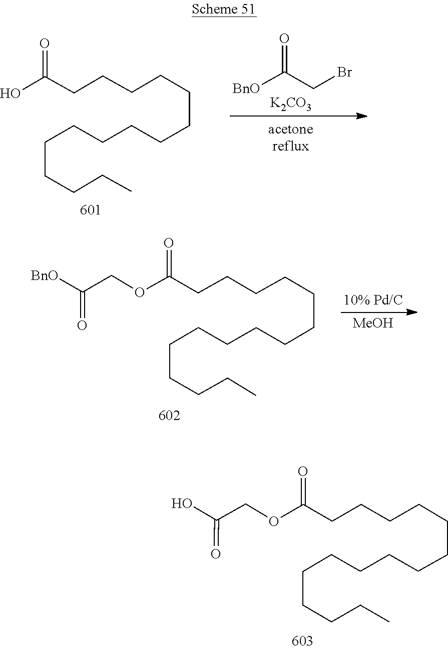

Compound 603: Palmitic acid 601 (3.53 g, 13.1 mmol) and potassium carbonate (3.71 g, 26.85 mmol) were added to a stirred solution of benzyl 2-bromoacetate (3.0 g, 13.1 mmol, 2.05 mL) in acetone (250 mL). After heating at reflux for 24 hours, the reaction mixture was cooled to room temperature and filtrated to remove the excess of K₂CO₃. The filtrate was evaporated under reduced pressure, and the residue was partitioned between diethyl ether and (50 mL) and water (50 mL). The organic fraction was dried over MgSO₄, filtered and evaporated under reduced pressure to give the crude benzyl ester 602 (5.2 g). The residue was dissolved in a 4:1 mixture of ethyl acetate/methanol (100 mL), followed by addition of 10% Pd/C (0.75 g, 0.71 mmol). The flask was equipped with a three-way adapter connected to a rubber balloon filled with Hydrogen, and to the vacuum line. The flask was placed under vacuum for 20 seconds, followed by refilling with Hydrogen. The sequence was repeated two more times. After 4 hours, the reaction mixture was filtered through a celite pad, the filtride was rinsed with ethyl acetate (×3) and methanol (×2). The combined filtrate was evaporated under reduced pressure. The residue was purified by ISCO automated column using 0-20% EtOAc in hexanes (the hexanes contained 1% of acetic acid) as eluent to give Compound 603 (2.22 g, 51%)¹H NMR (500 MHz, CDCl₃) δ 4.67 (s, 2H), 2.42 (t, J=7.5 Hz, 2H), 1.66 (p, J=7.5 Hz, 2H), 1.38-1.23 (m, 23H), 0.88 (t, J=6.9 Hz, 3H).

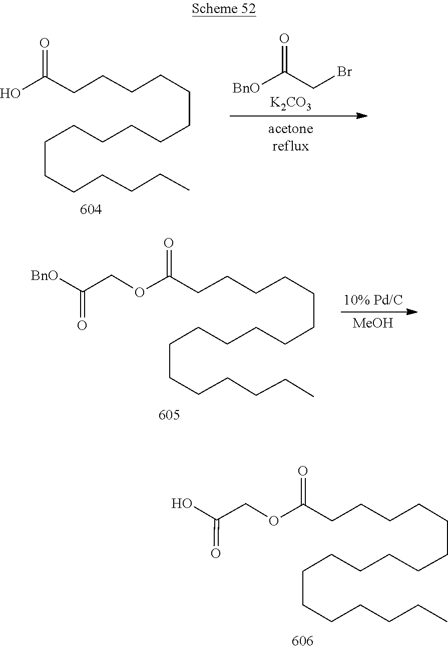

Compound 606: Stearic acid 604 (2.0 g, 7.03 mmol) and potassium carbonate (1.99 g, 14.41 mmol) were added to a stirred solution of benzyl 2-bromoacetate (1.61 g, 7.03 mmol) in acetone (250 mL). After heating at reflux for 24 hours, the reaction mixture was cooled to room temperature and filtrated to remove the excess of K₂CO₃. The filtrate was evaporated under reduced pressure, and the residue was partitioned between diethyl ether and water (50 mL). The organic fraction was dried over MgSO₄, filtered and evaporated under reduced pressure to give the crude benzyl ester 605 (3.0 g). The residue was dissolved in a 1:1 mixture of ethyl acetate/methanol (100 mL), followed by addition of 10% Pd/C (738 mg, 0.693 mmol). The flask was equipped with a three-way adapter connected to a rubber balloon filled with Hydrogen, and to the vacuum line. The flask was placed under vacuum for 20 seconds, followed by refilling with Hydrogen. The sequence was repeated two more times. After 4 hours, the reaction mixture was filtered through a celite pad, the filtride was rinsed with ethyl acetate (×3) and methanol (×2). The combined filtrate was evaporated under reduced pressure. The residue was purified by ISCO automated column using 0-20% EtOAc in hexanes (the hexanes contained 1% of acetic acid) as eluent to give Compound 606 (1.5 g, 62% over 2 steps). ¹H NMR (400 MHz, DMSO-d6) δ 4.53 (s, 2H), 2.35 (t, J=7.4 Hz, 2H), 1.59-1.49 (m, 2H), 1.23 (s, 28H), 0.85 (t, J=6.7 Hz, 3H).

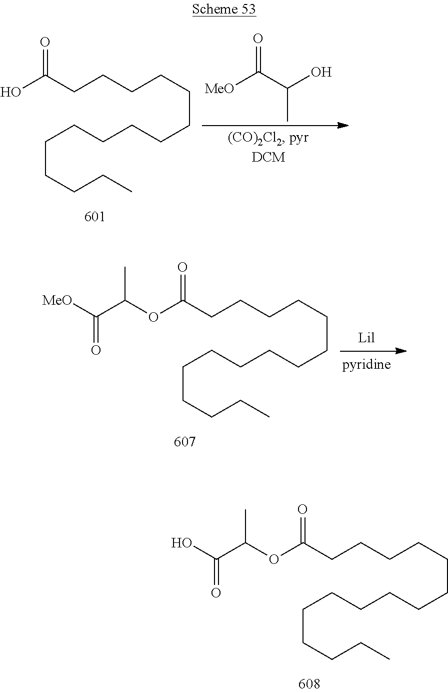

Compound 608: Palmitic acid 601 (2.66 g, 10.37 mmol) was dissolved in dry DCM (100 mL) under Argon and cooled to 0° C. Oxalyl chloride (2 M, 10.37 mL, 20.73 mmol) was added followed by DMF (one drop). The ice bath was removed, and the reaction mixture was stirred at room temperature. When the evolution of gas stopped (about 2 hours), the mixture was concentrated in vacuo to give crude palmitoyl chloride. In another flask, methyl 2-hydroxypropanoate (0.9 mL, 9.42 mmol) was dissolved in dry DCM (60 mL) followed by addition of pyridine (3.81 mL, 47.1 mmol). The reaction mixture was cooled to 0° C., followed by dropwise addition of a solution of the palmitoyl chloride in DCM (10 mL) via cannula. The ice bath was removed, and the reaction was stirred overnight. The reaction was quenched with deionized water (50 mL) and stirred vigorously for 30 minutes. The biphasic mixture was transferred to a separatory funnel. The layers were partitioned and separated. The organic layer was saved while the aqueous layer was extracted with dichloromethane (150 mL×2). The organics were combined and washed with 1 M aqueous hydrochloric acid, saturated aqueous sodium bicarbonate, brine, dried (sodium sulfate), filtered and concentrated. The crude residue was purified by ISCO automated column using 0-10% EtOAc in hexanes as eluent to give Compound 607 (2.28 g, 70%). ¹H NMR (500 MHz, chloroform-d) δ 5.10 (q, J=7.1 Hz, 1H), 3.74 (s, 3H), 2.37 (hept, J=7.7 Hz, 2H), 1.64 (h, J=7.1 Hz, 2H), 1.48 (d, J=7.1 Hz, 3H), 1.36-1.23 (m, 24H), 0.88 (t, J=6.8 Hz, 3H). Lithium Iodide (3.89 g, 29.05 mmol) was added to a stirred solution of Compound 607 (2 g, 5.84 mmol) in anhydrous pyridine (30 mL). After stirring for 24 hours at reflux, the mixture was evaporated. The residual oil was suspended with a mixture of 1 M HCl and EtOAc. The layers were separated, and the aqueous layer was extracted with EtOAc (×3). The organic extracts were combined, washed with a saturated aqueous solution of sodium thiosulfate, brine, dried over Na₂SO₄ and pre-adsorbed in silica gel. The residue was purified by ISCO automated column using 0-20% MeOH in CH₂Cl₂ as eluent to give Compound 608 (1.01 g, 52%). ¹H NMR (400 MHz, DMSO-d6) δ 12.94 (s, 1H), 4.88 (q, J=7.1 Hz, 1H), 2.32 (t, J=7.3 Hz, 2H), 1.57-1.47 (m, 2H), 1.37 (d, J=7.1 Hz, 3H), 1.24 (s, 24H), 0.88-0.83 (m, 3H).

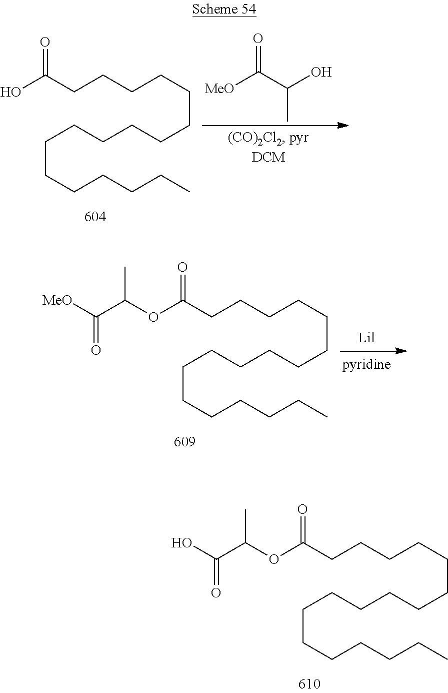

Compound 610: Stearic acid 604 (2.95 g, 10.37 mmol) was dissolved in dry DCM (100 mL) under Argon and cooled to 0° C. Oxalyl chloride (2 M, 10.37 mL, 20.73 mmol) was added followed by DMF (one drop). The ice bath was removed, and the reaction mixture was stirred at room temperature. When the evolution of gas stopped (about 2 hours), the mixture was concentrated in vacuo to give crude stearyl chloride. In another flask, methyl 2-hydroxypropanoate (0.981 g, 9.42 mmol, 0.9 mL) was dissolved in dry DCM (60 mL) followed by addition of pyridine (3.81 mL, 47.12 mmol). The reaction mixture was cooled to 0° C., followed by dropwise addition of a solution of the stearyl chloride in DCM (10 mL) via cannula. The ice bath was removed, and the reaction was stirred overnight. The reaction was quenched with deionized water (50 mL) and stirred vigorously for 30 minutes. The biphasic mixture was transferred to a separatory funnel. The layers were partitioned and separated. The organic layer was saved while the aqueous layer was extracted with dichloromethane (150 mL×2). The organics were combined and washed with 1 M aqueous hydrochloric acid, saturated aqueous sodium bicarbonate, brine, dried (sodium sulfate), filtered and concentrated. The crude residue was purified by ISCO automated column using 0-10% EtOAc in hexanes as eluent to give Compound 609 (3.09 g, 88%). ¹H NMR (500 MHz, chloroform-d) δ 5.10 (q, J=7.1 Hz, 1H), 3.75 (s, 3H), 2.38 (td, J=7.6, 6.2 Hz, 2H), 1.64 (q, J=7.4 Hz, 2H), 1.48 (d, J=7.0 Hz, 3H), 1.32-1.23 (m, 28H), 0.88 (t, J=6.9 Hz, 3H). Lithium Iodide (5.58 g, 41.7 mmol) was added to a stirred solution of compound 609 (3.09 g, 8.34 mmol) in anhydrous pyridine (40 mL). After stirring for 24 hours at reflux, the mixture was evaporated. The residual oil was suspended with a mixture of 1 M HCl and EtOAc. The layers were separated, and the aqueous layer was extracted with EtOAc (×3). The organic extracts were combined, washed with a saturated aqueous solution of sodium thiosulfate, brine, dried over Na₂SO₄ and pre-adsorbed in silica gel. The residue was purified by ISCO automated column using 0-20% MeOH in CH₂Cl₂ as eluent to give Compound 610 (1.29 g, 43%). ¹H NMR (400 MHz, DMSO-d6) δ 12.94 (s, 1H), 2.32 (t, J=7.3 Hz, 2H), 1.59-1.47 (m, 2H), 1.37 (d, J=7.1 Hz, 3H), 1.23 (s, 28H), 0.85 (t, J=6.7 Hz, 3H).

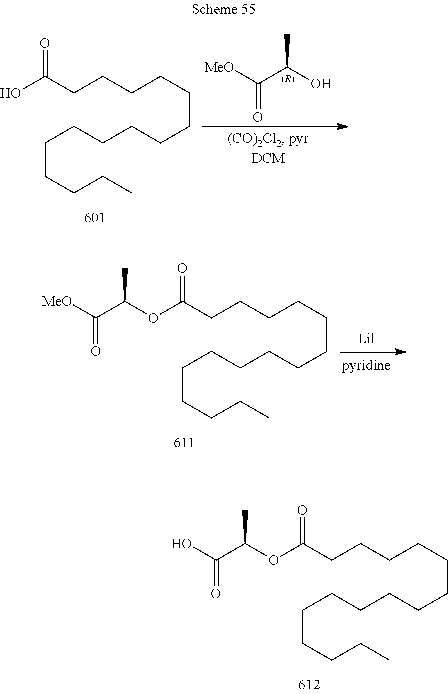

Compound 612: Palmitic acid 601 (2.66 g, 10.37 mmol) was dissolved in dry DCM (100 mL) under Argon and cooled to 0° C. Oxalyl chloride (1.79 mL, 20.73 mmol) was added followed by DMF (one drop). The ice bath was removed, and the reaction mixture was stirred at room temperature. When the evolution of gas stopped (about 2 hours), the mixture was concentrated in vacuo to give crude palmitoyl chloride. In another flask, methyl-(R)-lactate (0.9 mL, 9.42 mmol) was dissolved in dry DCM (60 mL) followed by addition of pyridine (3.81 mL, 47.1 mmol). The reaction mixture was cooled to 0° C., followed by dropwise addition of a solution of the palmitoyl chloride in DCM (10 mL) via cannula. The ice bath was removed, and the reaction was stirred overnight. The reaction was quenched with deionized water (50 mL) and stirred vigorously for 30 minutes. The biphasic mixture was transferred to a separatory funnel. The layers were partitioned and separated. The organic layer was saved while the aqueous layer was extracted with dichloromethane (150 mL×2). The organics were combined and washed with 1 M aqueous hydrochloric acid, saturated aqueous sodium bicarbonate, brine, dried (sodium sulfate), filtered and concentrated. The crude residue was purified by ISCO automated column using 0-10% EtOAc in hexanes as eluent to give Compound 611 (3.02 g, 93%). ¹H NMR (400 MHz, chloroform-d) δ 5.10 (q, J=7.1 Hz, 1H), 3.75 (s, 3H), 2.38 (td, J=7.5, 4.3 Hz, 2H), 1.70-1.60 (m, 2H), 1.48 (d, J=7.1 Hz, 3H), 1.38-1.22 (m, 26H), 0.91-0.85 (m, 3H). Lithium Iodide (5.90 g, 44.1 mmol) was added to a stirred solution of Compound 611 (3.02 g, 8.82 mmol) in anhydrous pyridine (47 mL). After stirring for 24 hours at reflux, the mixture was evaporated. The residual oil was suspended with a mixture of 1 M HCl and EtOAc. The layers were separated, and the aqueous layer was extracted with EtOAc (×3). The organic extracts were combined, washed with a saturated aqueous solution of sodium thiosulfate, brine, dried over Na₂SO₄ and pre-adsorbed in silica gel. The residue was purified by ISCO automated column using 0-20% MeOH in CH₂Cl₂ as eluent to give Compound 612 (1.2 g, 41%). ¹H NMR (400 MHz, chloroform-d) δ 5.11 (q, J=7.1 Hz, 1H), 2.38 (td, J=7.5, 3.0 Hz, 2H), 1.69-1.60 (m, 2H), 1.53 (d, J=7.1 Hz, 3H), 1.35-1.23 (m, 24H), 0.94-0.84 (m, 3H). LRMS (ESI) calculated for C₁₉H₃₅O₄[M−H]⁻ m/z=327.26, found 327.2. Enantiomeric excess: 100%.

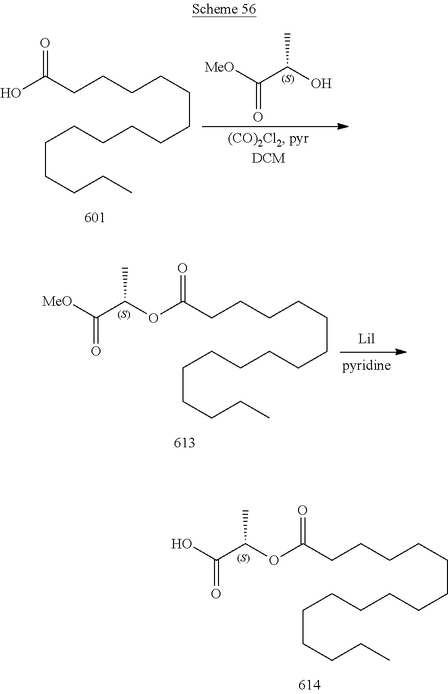

Compound 614: Palmitic acid 601 (2.66 g, 10.37 mmol) was dissolved in dry DCM (100 mL) under Argon and cooled to 0° C. Oxalyl chloride (1.79 mL, 20.73 mmol) was added followed by DMF (one drop). The ice bath was removed, and the reaction mixture was stirred at room temperature. When the evolution of gas stopped (about 2 hours), the mixture was concentrated in vacuo to give crude palmitoyl chloride. In another flask, methyl-(S)-lactate (0.9 mL, 9.42 mmol) was dissolved in dry DCM (60 mL) followed by addition of pyridine (3.81 mL, 47.1 mmol). The reaction mixture was cooled to 0° C., followed by dropwise addition of a solution of the palmitoyl chloride in DCM (10 mL) via cannula. The ice bath was removed, and the reaction was stirred overnight. The reaction was quenched with deionized water (50 mL) and stirred vigorously for 30 minutes. The biphasic mixture was transferred to a separatory funnel. The layers were partitioned and separated. The organic layer was saved while the aqueous layer was extracted with dichloromethane (150 mL×2). The organics were combined and washed with 1 M aqueous hydrochloric acid, saturated aqueous sodium bicarbonate, brine, dried (sodium sulfate), filtered and concentrated. The crude residue was purified by ISCO automated column using 0-70% EtOAc in hexanes as eluent to give Compound 613 (3.2 g, 99%). ¹H NMR (400 MHz, chloroform-d) δ 5.10 (q, J=7.1 Hz, 1H), 3.74 (s, 3H), 2.46-2.31 (m, 2H), 1.70-1.59 (m, 2H), 1.48 (d, J=7.1 Hz, 3H), 1.34-1.22 (m, 24H), 0.91-0.85 (m, 3H). Lithium Iodide (6.25 g, 46.7 mmol) was added to a stirred solution of Compound 613 (3.2 g, 9.34 mmol) in anhydrous pyridine (30 mL). After stirring for 24 hours at reflux, the mixture was evaporated. The residual oil was suspended with a mixture of 1 M HCl and EtOAc. The layers were separated, and the aqueous layer was extracted with EtOAc (×3). The organic extracts were combined, washed with a saturated aqueous solution of sodium thiosulfate, brine, dried over Na₂SO₄ and pre-adsorbed in silica gel. The residue was purified by ISCO automated column using 0-20% MeOH in CH₂Cl₂ as eluent to give Compound 614 (1.81 g, 59%). ¹H NMR (400 MHz, chloroform-d) δ 5.12 (q, J=7.1 Hz, 1H), 2.42-2.35 (m, 2H), 1.70-1.60 (m, 2H), 1.53 (d, J=7.1 Hz, 3H), 1.32-1.24 (m, 24H), 0.90-0.85 (m, 3H). LRMS (ESI) calculated for C₁₉H₃₅O₄[M−H]⁻ m/z=327.26, found 327.3. Enantiomeric excess: 100%.

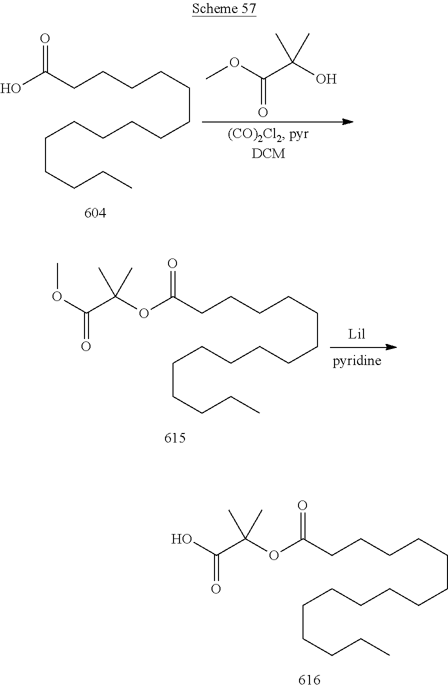

Compound 616: Palmitic acid 601 (2.46 g, 9.59 mmol) was dissolved in dry DCM (100 mL) and cooled to 0° C. Oxalyl chloride (1.66 mL, 20.3 mmol) was added followed by DMF (one drop). The ice bath was removed, and the reaction mixture was stirred at room temperature. When the evolution of gas stopped (about 2 hours), the mixture was concentrated in vacuo to give crude palmitoyl chloride. In another flask, methyl 2-hydroxy-2-methyl-propanoate (1.03 g, 8.72 mmol) was dissolved in dry DCM (60 mL) followed by addition of pyridine (3.5 mL, 43.6 mmol). The reaction mixture was cooled to 0° C., followed by dropwise addition of a solution of the palmitoyl chloride in DCM (20 mL) via cannula. The ice bath was removed, and the reaction was stirred overnight. The reaction was quenched with an aqueous saturated solution of NH₄Cl. The biphasic mixture was transferred to a separatory funnel and the layers were separated. The aqueous layer was extracted with dichloromethane (150 mL×2). The combined organics layers were combined and washed with 1 M aqueous hydrochloric acid, saturated aqueous sodium bicarbonate, brine, dried over Na₂SO₄, filtered and concentrated. The crude residue was purified by ISCO automated column using 0-10% EtOAc in hexanes as eluent to give Compound 615 (1.78 g, 57%). ¹H NMR (500 MHz, chloroform-d) δ 3.72 (s, 3H), 2.30 (t, J=7.5 Hz, 2H), 1.61 (p, J=7.4 Hz, 2H), 1.54 (s, 7H), 1.33-1.24 (m, 24H), 0.88 (t, J=6.9 Hz, 3H). LRMS (ESI) calculated for C₂₁H₄₁O₄[M+H]⁺ m/z=357.29, found 357.3. Lithium Iodide (3.34 g, 24.9 mmol) was added to a stirred solution of Compound 615 (1.78 g, 4.99 mmol) in anhydrous pyridine (25 mL). After stirring for 24 hours at reflux, the volatiles were removed under reduced pressure. The residual oil was suspended with a mixture of 1 M HCl and EtOAc. The layers were separated, and the aqueous layer was extracted with EtOAc (×3). The organic extracts were combined, washed with a saturated aqueous solution of sodium thiosulfate, brine, dried over Na₂SO₄ and pre-adsorbed in silica gel. The residue was purified by ISCO automated column using 0-20% MeOH in CH₂Cl₂ as eluent to give Compound 616 (1.23 g, 72%). ¹H NMR (400 MHz, chloroform-d) δ 2.31 (t, J=7.5 Hz, 2H), 1.66-1.55 (m, 8H), 1.37-1.20 (m, 25H), 0.91-0.84 (m, 3H). LRMS (ESI) calculated for C₂₀H₃₇O₄[M−H]⁻ m/z=341.28, found 341.3.

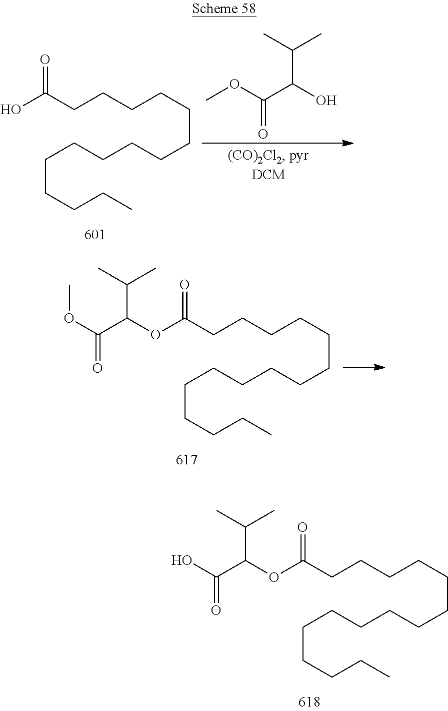

Compound 618: Palmitic acid 601 (2.19 g, 8.54 mmol) was dissolved in dry DCM (100 mL) and cooled to 0° C. Oxalyl chloride (1.47 mL, 17.1 mmol) was added followed by DMF (one drop). The ice bath was removed, and the reaction mixture was stirred at room temperature. When the evolution of gas stopped (about 2 hours), the mixture was concentrated in vacuo to give crude palmitoyl chloride. In another flask, methyl 2-hydroxy-3-methyl-butanoate (1.08 g, 7.76 mmol) was dissolved in dry DCM (60 mL) followed by addition of pyridine (3.14 mL, 38.8 mmol). The reaction mixture was cooled to 0° C., followed by dropwise addition of a solution of the palmitoyl chloride in DCM (10 mL) via cannula. The ice bath was removed, and the reaction was stirred overnight. The reaction was quenched with an aqueous saturated solution of NH₄Cl. The biphasic mixture was transferred to a separatory funnel and the layers were separated. The aqueous layer was extracted with dichloromethane (150 mL×2). The combined organics layers were combined and washed with 1 M aqueous hydrochloric acid, saturated aqueous sodium bicarbonate, brine, dried over Na₂SO₄, filtered and concentrated. The crude residue was purified by ISCO automated column using 0-10% EtOAc in hexanes as eluent to give Compound 617 (2.18 g, 75%). ¹H NMR (500 MHz, chloroform-d) δ 4.84 (d, J=4.6 Hz, 1H), 3.74 (s, 3H), 2.40 (td, J=7.5, 2.5 Hz, 2H), 2.22 (heptd, J=6.9, 4.6 Hz, 1H), 1.65 (p, J=7.5 Hz, 2H), 1.35-1.24 (m, 24H), 0.98 (dd, J=9.7, 6.9 Hz, 6H), 0.88 (t, J=6.9 Hz, 3H). LRMS (ESI) calculated for C₂₂H₄₃O₄ [M+H]+m/z=371.31, found 371.3. Lithium Iodide (3.94 g, 29.4 mmol) was added to a stirred solution of Compound 617 (2.18 g, 5.88 mmol) in anhydrous pyridine (25 mL). After stirring for 24 hours at reflux, the volatiles were removed under reduced pressure. The residual oil was suspended with a mixture of 1 M HCl and EtOAc. The layers were separated, and the aqueous layer was extracted with EtOAc (×3). The organic extracts were combined, washed with a saturated aqueous solution of sodium thiosulfate, brine, dried over Na₂SO₄ and pre-adsorbed in silica gel. The residue was purified by ISCO automated column using 0-20% MeOH in CH₂Cl₂ as eluent to give Compound 618 (1.59 g, 75%). ¹H NMR (400 MHz, chloroform-d) δ 4.90 (d, J=4.3 Hz, 1H), 2.45-2.37 (m, 2H), 2.28 (pd, J=6.9, 4.3 Hz, 1H), 1.66 (p, J=7.5 Hz, 2H), 1.36-1.22 (m, 24H), 1.03 (dd, J=6.9, 5.9 Hz, 6H), 0.92-0.84 (m, 2H). LRMS (ESI) calculated for C₂₁H₃₉O₄[M−H]⁻ m/z=355.29, found 355.3.

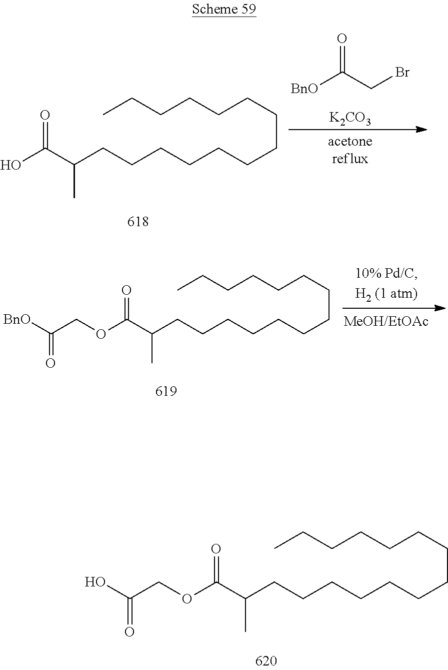

Compound 620: 2-methylhexadecanoic acid 618 (2.42 g, 8.95 mmol), and Potassium carbonate (2.54 g, 18.34 mmol) were added to a stirred solution of benzyl bromoacetate (1.48 mL, 9.40 mmol) in acetone (250 mL). After refluxing for 24 h, the reaction mixture was cooled to room temperature and filtrated to remove the excess of K₂CO₃. The filtrate was evaporated under reduced pressure. The residue was a white solid which was partitioned between diethyl ether and (50 mL) and water (50 mL). The organic fraction was dried over magnesium sulfate, filtered and evaporated under reduced pressure to give the crude benzyl ester. The residue was pre-adsorbed in silica gel and purified using 0% to 8% gradient EtOAc/hexane to give Compound 619 (2.03 g, 54%). ¹H NMR (400 MHz, chloroform-d) δ 7.41-7.31 (m, 5H), 5.19 (s, 2H), 4.65 (s, 2H), 2.53 (h, J=7.0 Hz, 1H), 1.76-1.64 (m, 1H), 1.55 (s, 1H), 1.46-1.37 (m, 1H), 1.35-1.21 (m, 25H), 1.18 (d, J=7.0 Hz, 3H), 0.91-0.85 (m, 3H). LRMS (ESI) calculated for C₂₆H₄₂O₄Na [M+Nₐ]⁺ m/z=441.31, found 441.3. Compound 618 (2.03 g, 4.85 mmol) was dissolved in a 4:1 mixture of ethyl acetate/methanol (80 mL), followed by addition of 10% Pd/C (516 mg, 0.484 mmol). The flask was equipped with a three-way adapter connected to a rubber balloon filled with Hydrogen, and to the vacuum line. The flask was placed under vacuum for 20 seconds, followed by refilling with Hydrogen. The sequence was repeated two more times. After 4 hours, the reaction mixture was filtered through a celite pad, the filtride was rinsed with ethyl acetate (×3) and methanol (×2). The combined filtrate was evaporated under reduced pressure. The residue was purified by ISCO automated column using 0-60% EtOAc in hexanes (the hexanes contained 1% of acetic acid) as eluent to give Compound 620 (1.13 g, 70%). ¹H NMR (400 MHz, chloroform-d) δ 4.66 (d, J=1.0 Hz, 2H), 2.54 (h, J=7.0 Hz, 1H), 1.76-1.64 (m, 1H), 1.51-1.39 (m, 1H), 1.36-1.17 (m, 28H), 0.92-0.84 (m, 2H).). LRMS (ESI) calculated for C₁₉H₃₅O₄[M−H]⁻ m/z=327.26, found 327.2.

### Cleavable Ceramide-Type Linkers

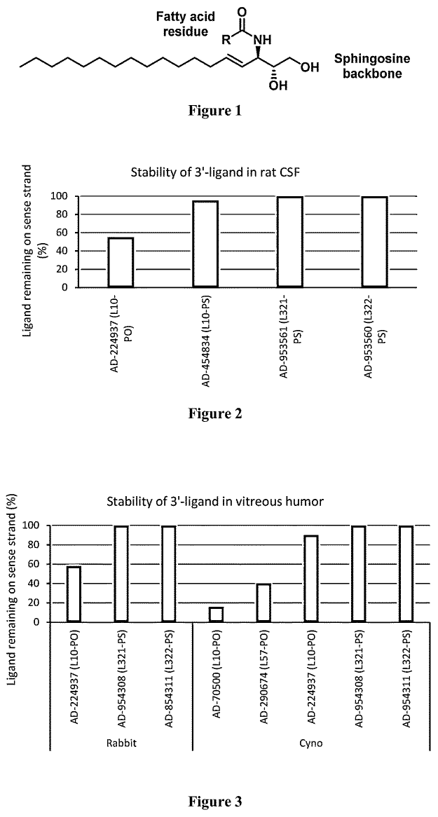

Ceramidases (CDases) are key enzymes of sphingolipid metabolism that regulate the formation and degradation of ceramides. A ceramide is composed of sphingosine bone and a fatty acid residue, as shown in FIG. 1. The enzymatic degradation of ceramides by cleavage of the amide bond, is controlled by three families of CDases (acid, neutral, and alkaline) which are distinguished by their pH optima, subcellular location, primary structure, mechanism, and function.

2′-O-ceramide-type nucleosides phosphoramidates can be synthesized using strategy based on the mechanism and the structural requirements of human neutral CDases. The synthesized monomers nucleosides is introduced strategically into siRNA and once in the body, will be cleaved selectively by CDases, releasing the fatty acid and the oligonucleotide chain.

The synthetic procedure for 2′-O-ceramide-type nucleosides phosphoramidates can be shown as Scheme 60. Compound 901 is commercially available or can be prepared in 2 steps from uridine. Cross metathesis of the terminal alkene at the 2′-position of the nucleoside with a derivate of (S)-allylglycine gave compound 902. Hydrogenation of the internal alkene followed by formation of the phosphoramidate afforded compound 903.

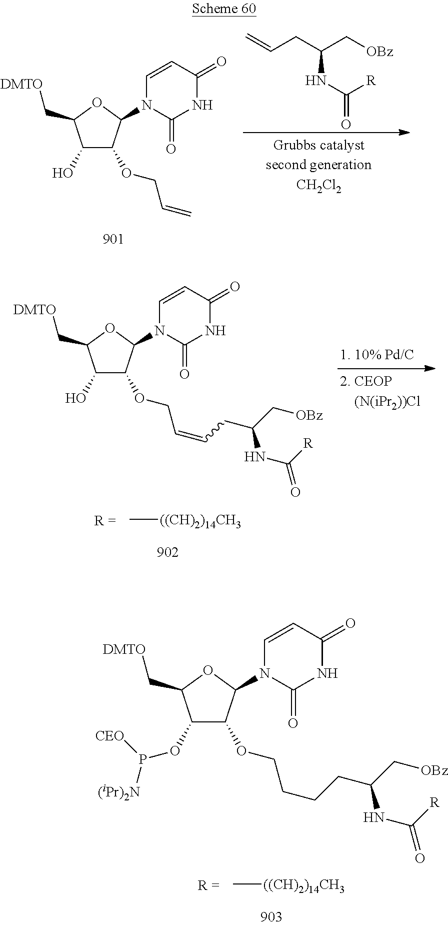

### Example 2. Post-Synthetic Conjugation of Lipophilic Moieties to siRNA

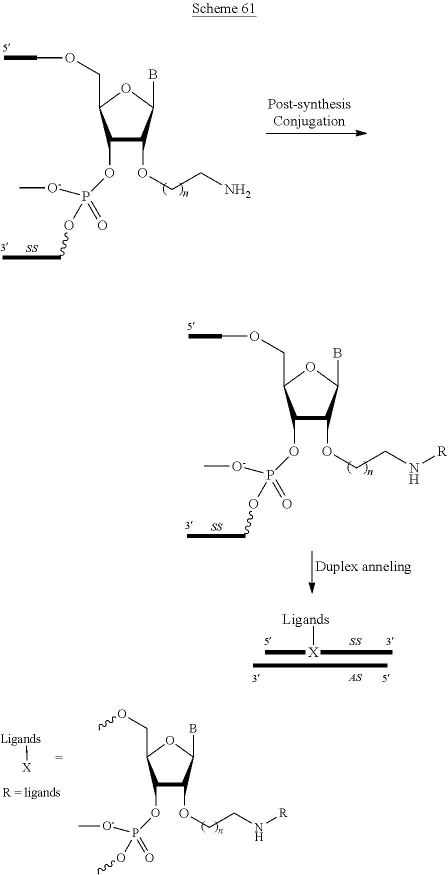

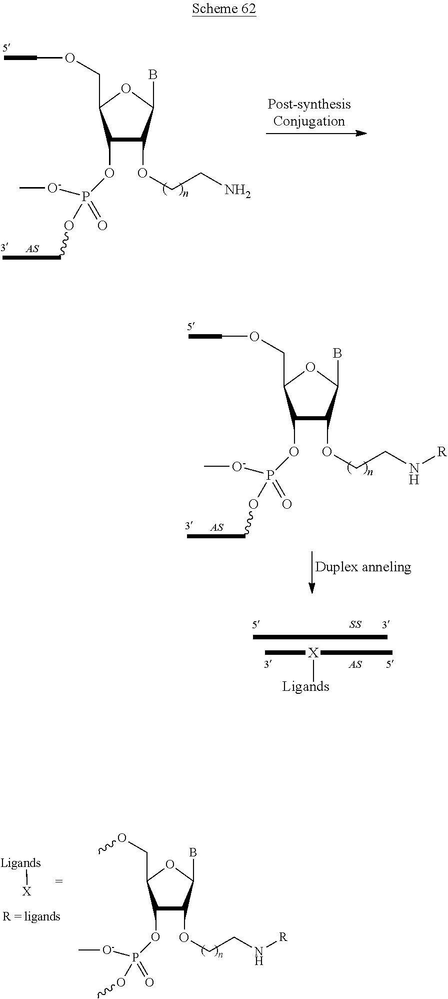

Various ligands, including various lipophilic moieties was conjugated to siRNA agents via post-synthesis conjugation methods, as shown in Schemes 61 and 62. Amino derivative of sense or antisense strand of siRNA was reacted either with NHS esters of lipophilic ligands or carboxylic acids under peptide coupling conditions. These singles strands were then purified and combined with other strands to make siRNA duplexes.

### Example 3. Synthesis of siRNA Conjugates Having Terminal Acid Functionality

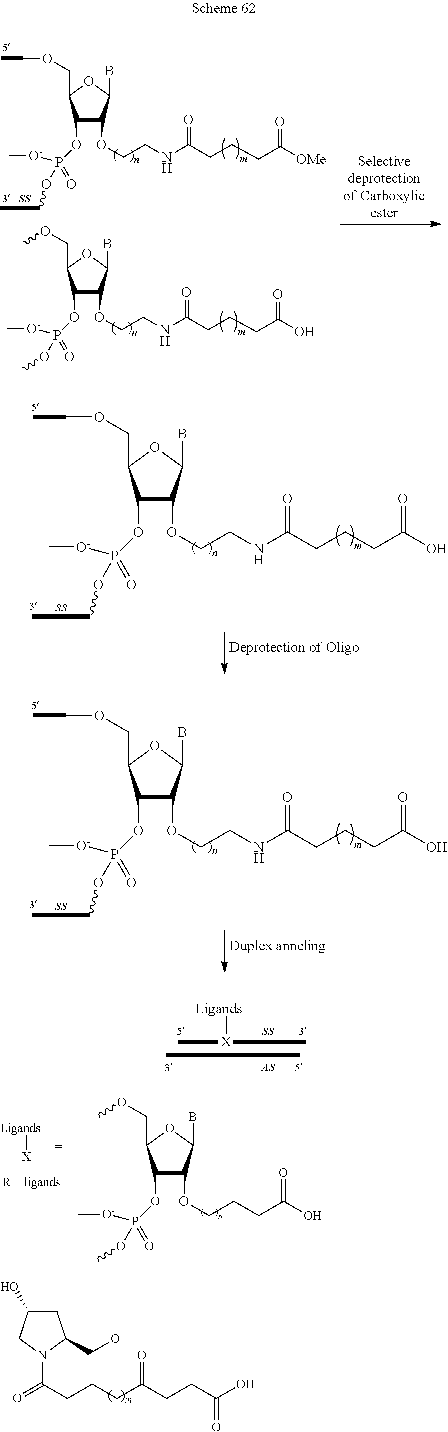

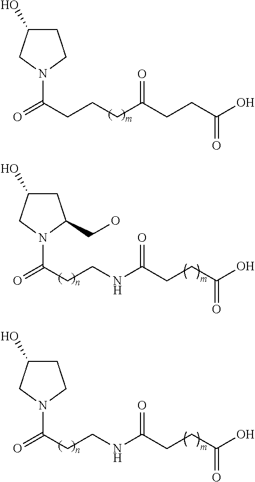

Various ligands, including various lipophilic having carboxylic moieties was conjugated to siRNA agents at terminals and internal positions via on column or post-synthetic conjugation, as shown in Scheme 62.

Solid supported single strands containing lipophilic moieties having terminal esters were first treated with 20% piperidine in water overnight followed by 2:1 NH40H in ethanol for 15 hours at room temperature to generate single strands having terminal carboxylic acids. These single strands were combined with corresponding antisense strands to generate siRNA duplexes for various assays (see, e.g., Tables 11, 12, 18, and 19).

### Example 4. Synthesis of siRNA Conjugates Having Lipophilic Groups Attached to Phosphate Backbone

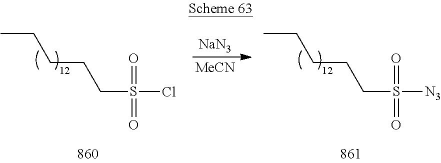

Compound 861: Sodium azide (2.57 g, 39.53 mmol) was added to a stirred solution of hexadecane-1-sulfonyl chloride (10.08 g, 30.4 mmol) in MeCN (100 mL). After stirring at room temperature for 10 hours, the reaction mixture was diluted with EtOAc (200 mL) and washed with water (50 mL). The organic phase was dried over Na₂SO₄ and evaporated to dryness. The residue was purified by ISCO automated column using 0-5% EtOAc in hexanes as eluent to give Compound 861 (7.71 g, 76%). ¹H NMR (400 MHz, chloroform-d) δ 3.33-3.28 (m, 2H), 1.96-1.87 (m, 2H), 1.51-1.41 (m, 2H), 1.33-1.23 (m, 24H), 0.92-0.86 (m, 3H).

Reaction between compound 861 and an oligonucleotide (sense or antisense strand). During the solid-phase synthesis of an oligonucleotide (Scheme 64), a solution of Compound 861 (0.5 M in acetonitrile) was used to oxidize the P(III) phosphite ester intermediate 862 to produce a sulfonyl phosphoramidite Compound 863. This oxidation step is used instead of common oxidizing reagents (I₂ or sulfurizing reagent) and can be performed at any stage of the oligonucleotide synthesis that involve oxidation of a P(III) phosphite. At the end of the synthesis, the oligo is fully deprotected using standard conditions, and cleaved from the solid support to give oligonucleotide 864 containing the sulfonylphosphoramidate.

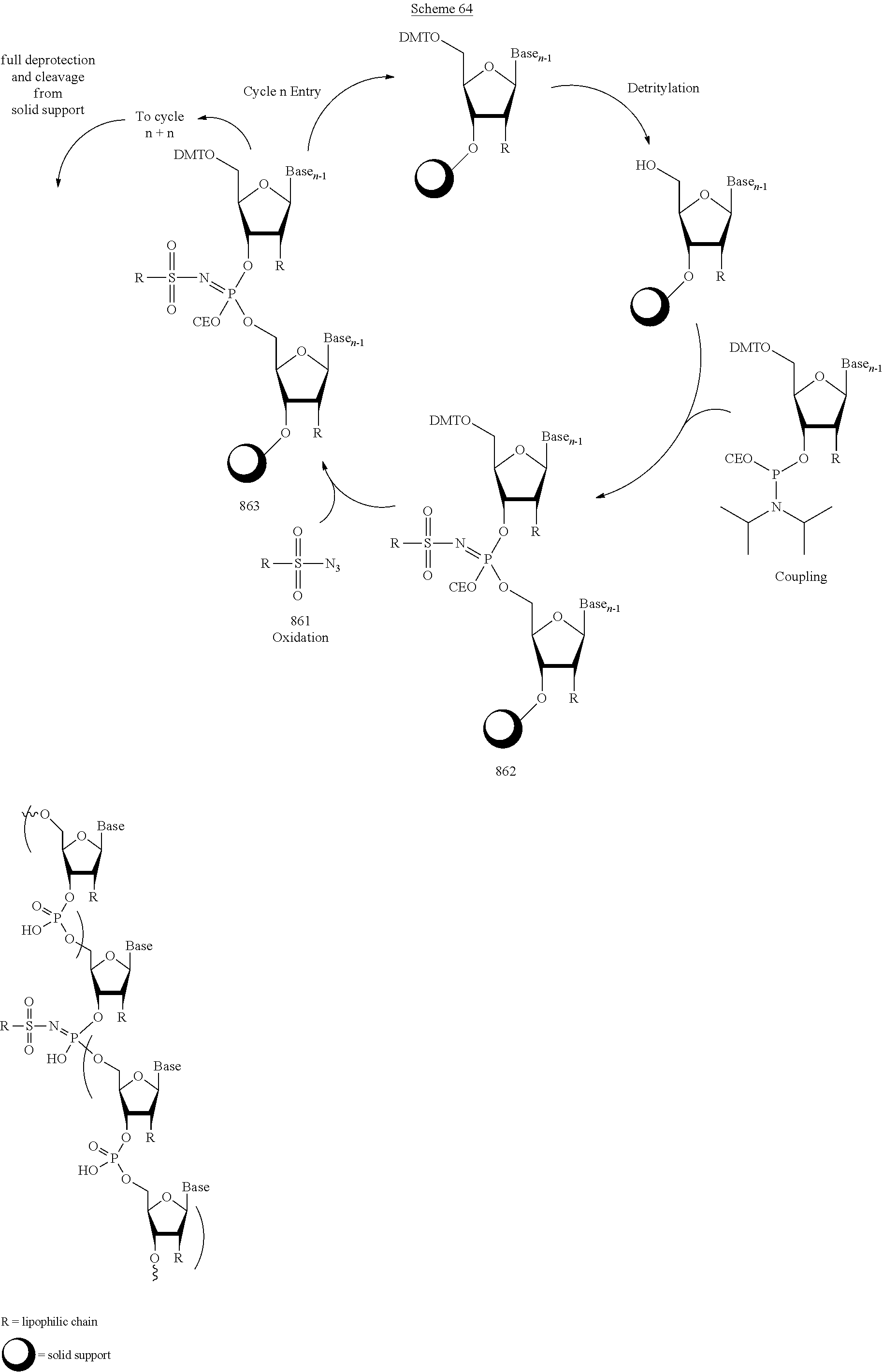

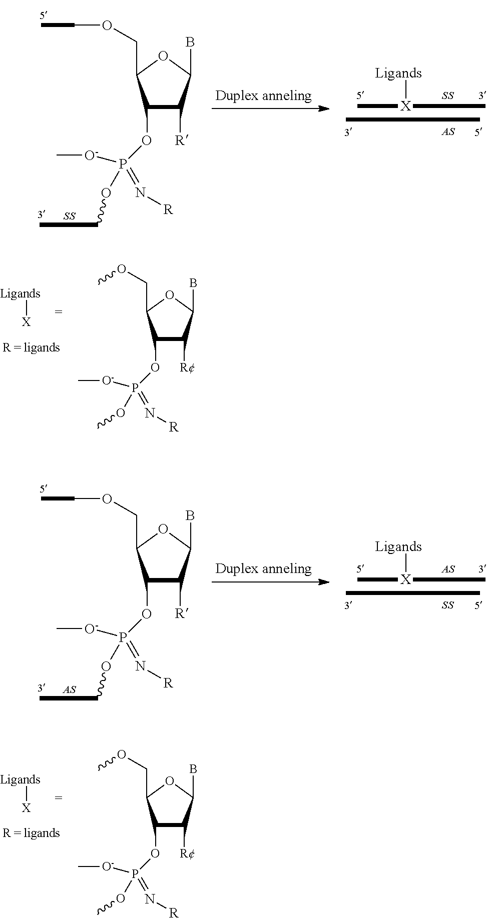

TABLE 1
siRNAs having phosphate backbone modified with lipophilic moiety

| Duplex | Oligo | | | | Molecular | Exact |
|---|---|---|---|---|---|---|
| Id | Id | strand | target | oligoSeq | Weight | Mass |
| AD- | A- | sense | TTR | asascagY158GfuUf | 7217.13 | 7213.25 |
| 1033233 | 1840408 | | | ugcucuausasa | | |
| | A- | antis | TTR | VPuUfauaGfagcaagaAf | 7699.98 | 7696.19 |
| | 555715 | | | cAfcuguususu | | |
| AD- | A- | sense | SOD1 | csasuuuY158AfaUfCfCf | 7121.08 | 7117.24 |
| 1427063 | 2248662 | | | ucacucuasasa | | |
| | A- | antis | SOD1 | VPusUfsuagAfgUfGfag | 7851.15 | 7847.15 |
| | 444402 | | | gaUfuAfaaaugsasg | | |

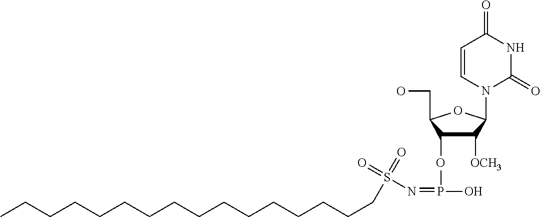

Y158
Upper and lower case letters in italics indicate 2′-deoxy-2′-fluoro (2′-F), and 2′-O-methyl (2′-
OMe) sugar modifications, respectively, to adenosine, cytidine, guanosine and uridine; s
indicates phosphorothioate (PS) linkage; VP indicates vinyl phosphonate.

### Example 5: Synthesis of Monomers for Nucleobase Modified Lipophilic Conjugates

A variety of lipids can be conjugated with aminolinker at C5 position of pyrimidine as shown below and the building block phosphoramidites can be incorporated into siRNAs.

### Synthesis of C5-Lipid Conjugated Uridine Phosphoramidite

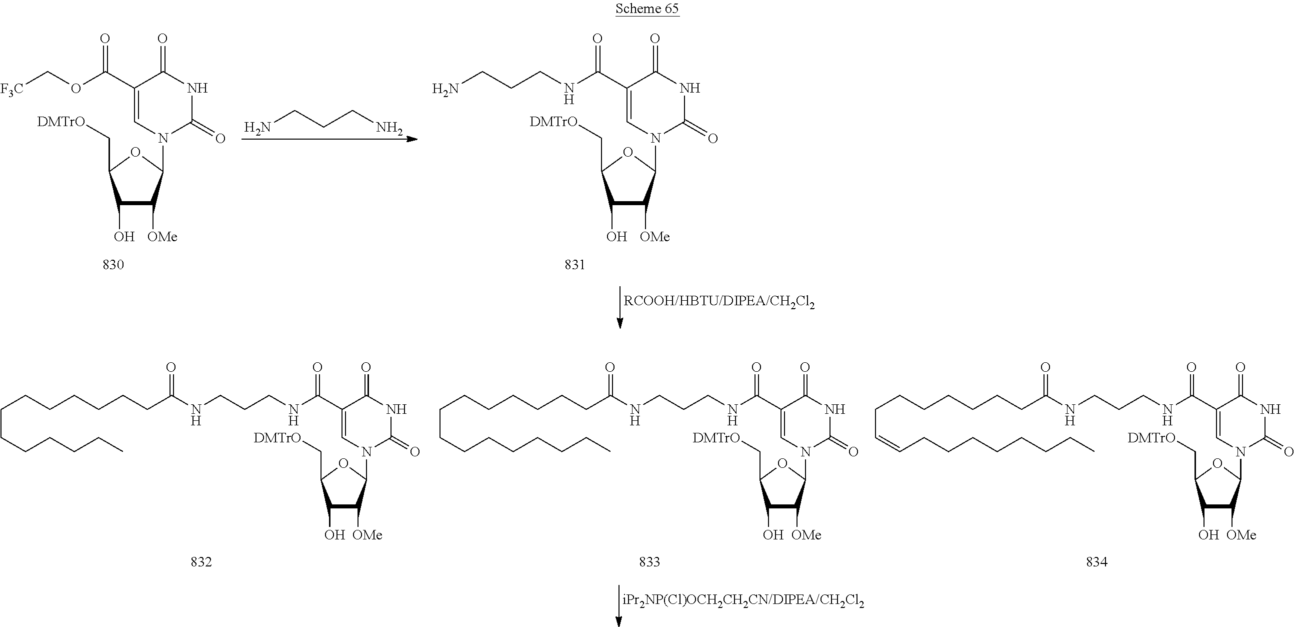

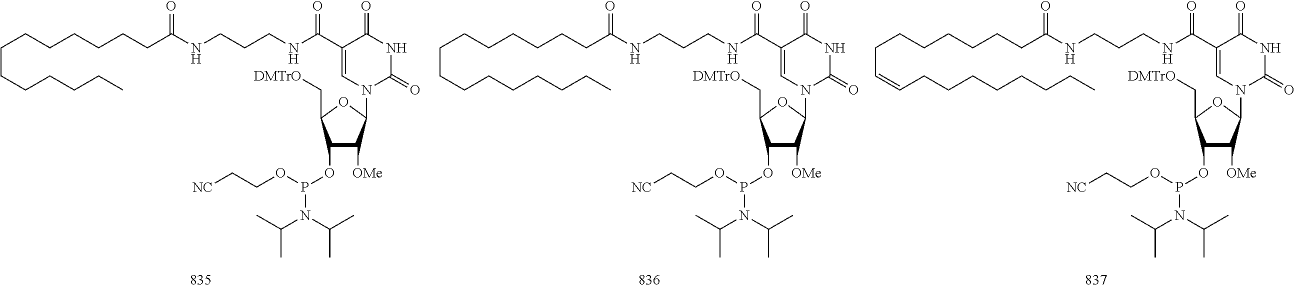

Compound 831: 1,3-Diaminopropane (81.0 g, 1.09 mol) was added to a solution of Compound 830 (15.0 g, 21.9 mmol) in MeOH (120 mL) at room temperature. The reaction mixture was stirred at room temperature for 15 hours. The reaction mixture was diluted with DCM, and this solution was washed with H₂O and brine, dried over Na₂SO₄, and concentrated in vacuo to afford the crude material (14.5 g). This crude was used directly for the next coupling reaction. ESI-MS; 661.3 (M+H).

Compound 832: Compound 831 (5.0 g, 7.57 mmol) was added to a reaction flask, along with myristic acid (3.46 g, 15.1 mmol) and HBTU (3.44 g, 9.08 mmol). The solids were dissolved in CH₂Cl₂ (150 mL) and diisopropylethylamine (2.93 g, 22.7 mmol) was added via a syringe. The reaction was stirred at room temperature overnight. The reaction was checked by TLC (EtOAc) to confirm the consumption of the starting material. The reaction was diluted with CH₂Cl₂ then washed by saturated NaHCO₃ solution. The organic layer was separated, dried over anhydrous Na₂SO₄ and concentrated. The crude residue was purified by flash chromatography on silica gel (0% to 100% EtOAc/Hexane) to give Compound 832 (4.98 g, 5.72 mmol, 76%). ¹H NMR (500 MHz, chloroform-d) δ 8.68 (s, 1H), 7.52-7.42 (m, 2H), 7.42-7.33 (m, 5H), 7.19 (d, J=7.3 Hz, 1H), 6.86-6.77 (m, 5H), 6.31 (t, J=6.2 Hz, 1H), 5.91 (d, J=3.3 Hz, 1H), 4.20 (t, J=6.0 Hz, 1H), 4.05 (ddd, J=6.8, 4.7, 2.8 Hz, 1H), 3.94 (dd, J=5.6, 3.3 Hz, 1H), 3.77 (s, 6H), 3.71 (hept, J=6.6 Hz, 3H), 3.56 (s, 3H), 3.50 (dd, J=11.0, 2.9 Hz, 1H), 3.46-3.29 (m, 4H), 1.25-1.23 (m, 24H), 0.88 (t, J=6.9 Hz, 3H).

Compound 833: Compound 833 was obtained (3.62 g, 53.2%) by using palmitic acid in a similar manner as described above for the synthesis of Compound 832. ¹H NMR (500 MHz, chloroform-d) δ 8.67 (s, 1H), 8.62 (t, J=6.4 Hz, 1H), 7.52-7.43 (m, 2H), 7.43-7.33 (m, 5H), 7.27-7.15 (m, 2H), 6.84-6.79 (m, 4H), 6.32 (t, J=6.2 Hz, 1H), 5.91 (d, J=3.4 Hz, 1H), 4.21 (t, J=6.0 Hz, 1H), 4.05 (ddd, J=6.8, 4.7, 2.8 Hz, 1H), 3.94 (dd, J=5.6, 3.3 Hz, 1H), 3.77 (s, 6H), 3.71 (p, J=6.7 Hz, 1H), 3.56 (s, 3H), 3.50 (dd, J=11.0, 2.9 Hz, 1H), 3.45-3.32 (m, 3H), 1.24 (d, J=9.7 Hz, 28H), 0.88 (t, J=6.9 Hz, 3H).

Compound 834: Compound 834 was obtained (5.29 g, 79.5%) by using oleic acid in a similar manner as described above for the synthesis of Compound 832. ¹H NMR (500 MHz, chloroform-d) δ 8.66 (d, J=9.4 Hz, 2H), 7.46 (tt, J=6.1, 1.3 Hz, 2H), 7.37 (ddd, J=9.0, 4.7, 2.2 Hz, 4H), 7.30-7.23 (m, 3H), 7.22-7.14 (m, 1H), 6.82 (dt, J=8.9, 1.6 Hz, 4H), 6.37 (dt, J=20.2, 6.0 Hz, 1H), 5.92 (d, J=3.3 Hz, 1H), 5.34 (td, J=3.7, 2.0 Hz, 4H), 4.20 (dd, J=7.5, 4.7 Hz, 1H), 4.05 (ddd, J=7.0, 4.7, 2.9 Hz, 1H), 3.95 (dd, J=5.6, 3.3 Hz, 1H), 3.77 (s, 6H), 3.56 (s, 3H), 3.53-3.45 (m, 1H), 2.01 (d, J=6.0 Hz, 4H), 1.34-1.11 (m, 24H), 0.88 (t, J=7.0, 2.4 Hz, 3H).

Compound 835: To a 200 mL round bottom flask was added Compound 832 (4.98 g, 5.72 mmol) in anhydrous EtOAc (80 mL) under argon and cooled in an ice bath. Then N, N-diisopropylaminocyanoethyl phosphonamidic chloride (1.49 g, 6.29 mmol) was added followed by DIPEA (2.22 g, 17.2 mmol). The reaction mixture was stirred at room temperature overnight. Then the reaction mixture was quenched with brine, extracted with EtOAc. The organic layer was separated, dried over anhydrous Na₂SO₄ and concentrated to crude oil. Flash chromatography on silica gel (0% to 60% EtOAc in hexane) to give Compound 835 (2.22 g, 2.07 mmol, 36.25%). ¹H NMR (500 MHz, acetonitrile-d₃) δ 8.65 (t, J=6.2 Hz, 1H), 8.49 (s, 1H), 7.53-7.44 (m, 2H), 7.44-7.33 (m, 4H), 7.33-7.26 (m, 2H), 7.20 (td, J=7.1, 1.3 Hz, 1H), 6.91-6.81 (m, 4H), 6.49 (t, J=6.0 Hz, 1H), 5.91 (d, J=4.5 Hz, 1H), 4.28-4.14 (m, 2H), 3.98 (t, J=4.7 Hz, 1H), 3.88-3.77 (m, 1H), 3.75 (s, 6H), 3.63-3.49 (m, 2H), 3.45 (s, 3H), 3.40-3.21 (m, 4H), 3.12 (qd, J=6.4, 3.7 Hz, 2H), 2.65 (dt, J=6.4, 5.5 Hz, 2H), 1.62-0.96 (m, 36H), 0.88 (t, J=6.9 Hz, 3H). ³¹P NMR (202 MHz, acetonitrile-d₃) δ 150.37, 150.26

Compound 836: Compound 833 was obtained (2.16 g, 48.8%) in a similar manner as described above for the synthesis of Compound 836. ¹H NMR (400 MHz, acetonitrile-d₃) δ 8.65 (t, J=6.3 Hz, 1H), 8.59 (s, 1H), 7.54-7.44 (m, 2H), 7.45-7.32 (m, 4H), 7.28 (dd, J=8.3, 6.9 Hz, 2H), 7.25-7.12 (m, 1H), 6.86 (dt, J=8.2, 1.5 Hz, 4H), 6.50 (t, J=6.1 Hz, 1H), 5.89 (dd, J=19.7, 4.4 Hz, 1H), 4.31 (dt, J=9.1, 5.4 Hz, 1H), 4.19 (ddd, J=6.7, 4.5, 2.3 Hz, 1H), 4.11-4.02 (m, 1H), 3.75 (d, J=1.9 Hz, 6H), 3.72-3.47 (m, 4H), 3.50-3.37 (m, 4H), 3.39-3.00 (m, 6H), 2.81-2.70 (m, 1H), 2.45 (t, J=6.0 Hz, 2H), 1.66-1.09 (m, 40H), 0.92-0.81 (m, 3H). ³¹P NMR (202 MHz, acetonitrile-d₃) δ 150.37, 150.25

Compound 837: Compound 834 was obtained (1.42 g, 22.1%) in a similar manner as described above for the synthesis of Compound 837. ¹H NMR (400 MHz, acetonitrile-d₃) δ 8.66 (q, J=6.5 Hz, 1H), 8.61-8.45 (m, 1H), 7.59-7.14 (m, 10H), 6.85 (dt, J=8.8, 2.1 Hz, 4H), 6.50 (t, J=6.1 Hz, 1H), 5.89 (dd, J=19.7, 4.4 Hz, 1H), 5.34 (t, J=5.0 Hz, 3H), 4.39-3.96 (m, 4H), 3.75 (d, J=1.9 Hz, 6H), 3.68-3.02 (m, 13H), 2.76 (t, J=6.0 Hz, 1H), 2.54-2.24 (m, 2H), 1.99 (dd, J=11.0, 5.0 Hz, 4H), 1.69-0.96 (m, 34H), 0.87 (t, J=6.5 Hz, 3H). ³¹P NMR (162 MHz, acetonitrile-d₃) δ 150.46, 150.34.

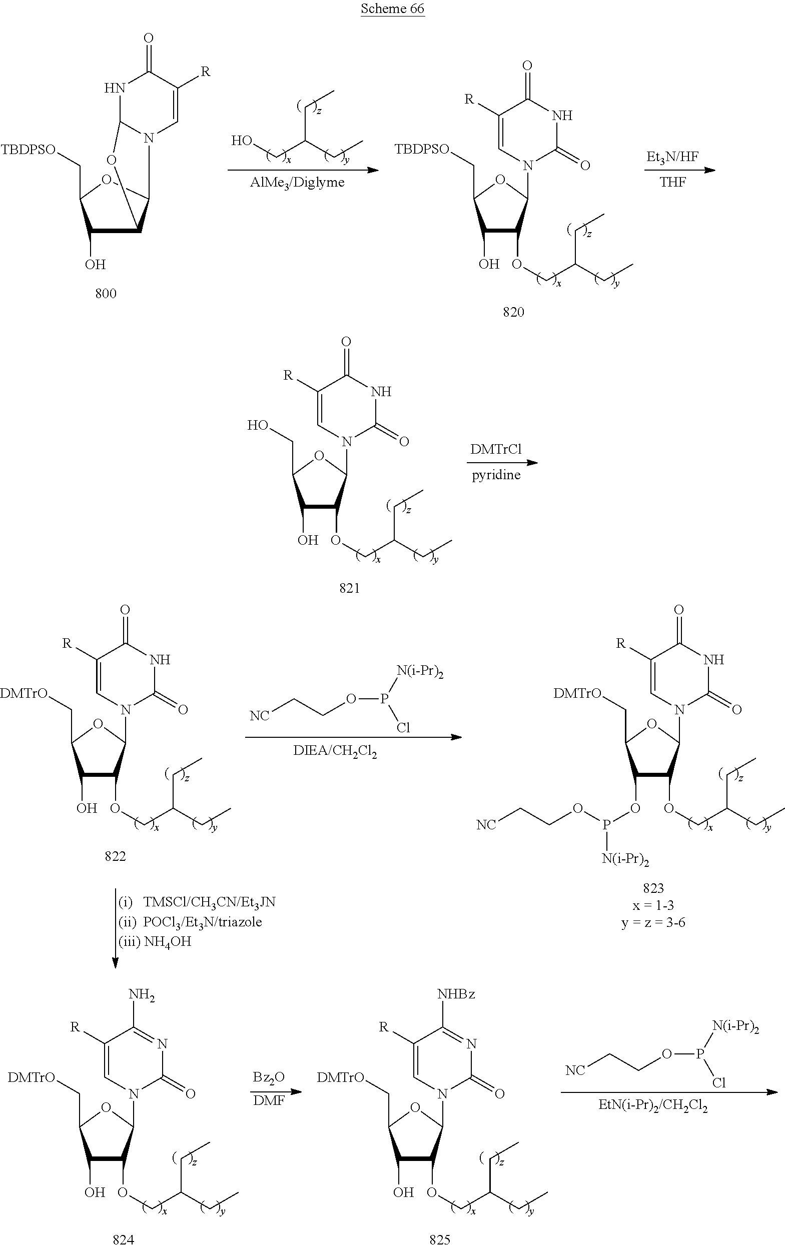

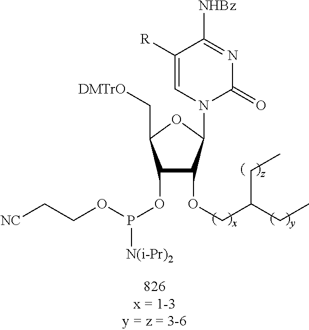

As shown in Scheme 66, protected anhydro nucleoside 800 can be ring-opened by any branched alkyl alcohols to give Compound 820. Removal of the protecting group at 5′-position gives Compound 822. 5′-position of free nucleoside 822 is protected by DMTr group to give Compound 823 and the secondary hydroxyl group at 3′-position is phosphitylated to give Compound 824. Compound 825 can be converted to cytosine derivative using standard triazole conditions to give Compound 826. The exocyclic amino group is protected by benzoyl group to give Compound 826 and subsequent phosphitylation gives Compound 827. Some examples of branched alkyl nucleoside at 2′ position include, but not limited to those shown below:

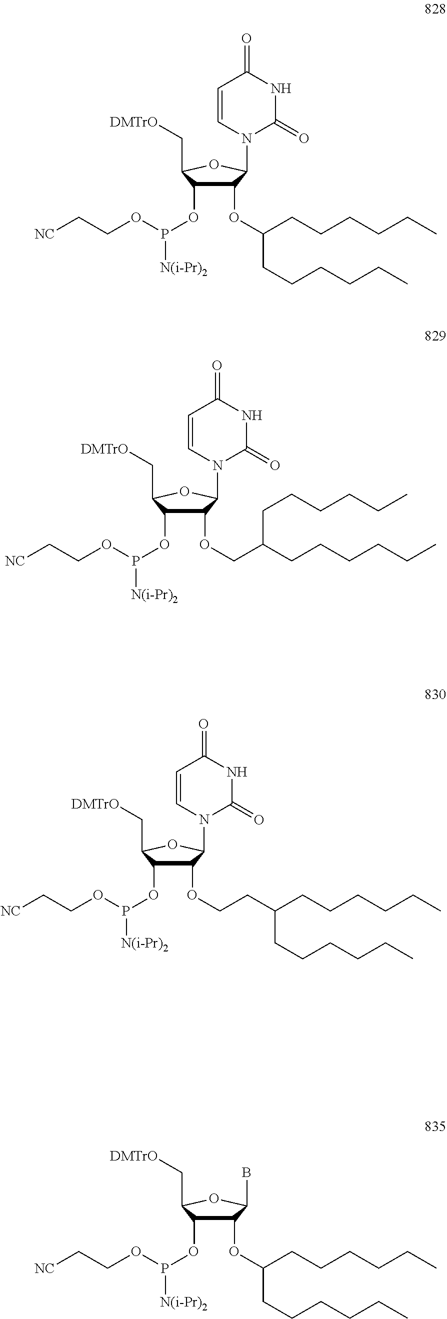

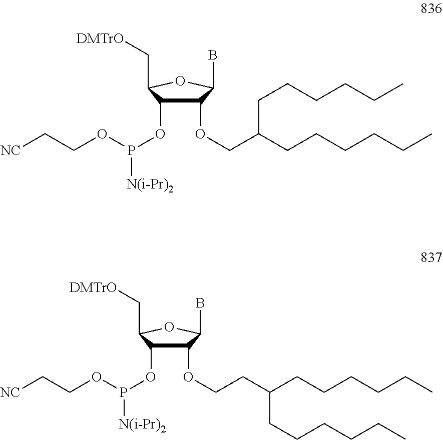

### Example 6. Metabolic Stability Determination of siRNA Conjugates in Various Matrices

Stability of ligands in cerebral spinal fluid (CSF): Stability of ligands were assessed by incubating 50 μL of rat derived CSF (BioIVT, Cat. RAT00CSFXZN), with 12.5 L of siRNA (0.1 mg/mL) in a 96-well plate for 24 hours at 37° C. with gentle shaking. After which, protein was digested by adding 25 μL of a proteinase K solution containing 0.0875 mg proteinase K in 4.1% Tween 20, 0.3% Triton X-100, 24.7 mM Tris-HCl, pH 8.0 and incubating for 1 hour at 50° C. with gentle shaking. Samples were then diluted with 450 μL lysis buffer (Phenomenex, Cat. AL0-8579) that was adjusted to pH 5.5 using ammonium hydroxide in preparation for solid phase extraction.

Stability of ligands in brain homogenate: Stability of ligands were assessed by incubating 50 μL of rat brain homogenate (BioIVT, Cat. S05966) with 12.5 μL of siRNA (0.1 mg/mL) in a 96-well plate for 24 hours at 37° C. with gentle shaking. After which, protein was digested by adding 25 μL of a proteinase K solution containing 0.0875 mg proteinase K in 4.1% Tween 20, 0.3% Triton X-100, 24.7 mM Tris-HCl, pH 8.0 and incubating for 1 hour at 50° C. with gentle shaking. Samples were then diluted with 450 μL lysis buffer (Phenomenex, Cat. AL0-8579) that was adjusted to pH 5.5 using ammonium hydroxide in preparation for solid phase extraction.

Stability of ligands in vitreous humor: Stability of ligands were assessed by incubating 50 μL of rabbit derived (BioIVT, Cat. RAB00VITHUMPZN) or cynomologous monkey derived (BioIVT, Cat. NHP01HUMPZN) vitreous humor with 12.5 μL of siRNA (0.1 mg/mL) in a 96-well plate for 24 hours at 37° C. with gentle shaking. After which, protein was digested by adding 25 μL of a proteinase K solution containing 0.0875 mg proteinase K in 4.1% Tween 20, 0.3% Triton X-100, 24.7 mM Tris-HCl, pH 8.0 and incubating for 1 h at 50° C. with gentle shaking. Samples were then diluted with 450 μL lysis buffer (Phenomenex, Cat. AL0-8579) that was adjusted to pH 5.5 using ammonium hydroxide in preparation for solid phase extraction.

Solid Phase Extraction: Solid phase extraction was then performed using Clarity OTX solid phase extraction plates (Phenomenex, Cat. 8E-S103-EGA). The plate was first conditioned by passing 1 mL methanol through it using a positive pressure manifold, followed by 1.9 mL equilibration buffer (50 mM ammonium acetate with 2 mM sodium azide, pH 5.5), then the samples were loaded onto the column. The column was then washed with 1.5 mL wash buffer (50 mM ammonium acetate in 50% acetonitrile, pH 5.5) 5 times. Samples were eluted with 0.6 mL elution buffer (10 mM EDTA, 100 mM ammonium bicarbonate, 10 mM DTT in 40% acetonitrile and 10% THF, pH 8.8) and dried using nitrogen flow (TurboVap, 65 psi N₂ at 40° C.).

Analytical Method: After SPE, samples were reconstituted in 120 μL water, and analyzed using liquid chromatography combined with mass spectrometry detection on a Thermo QExactive by electrospray ionization (ESI). Samples were injected (30 μL) and separated using an XBridge BEH C8 XP Column 130 A, 2.5 m, 2.1×30 mm (Waters, Cat. 176002554) maintained at 80° C. Mobile phase A was 16 mM triethylamine and 200 mM hexafluoroisopropanol and mobile phase B was methanol, and a gradient of 0-65% mobile phase B over 6.2 minutes was employed at 1 mL/min. The ESI source was operated in negative ion mode, with full scan, using spray voltage=2800 V, sheath gas flow=65 units, auxiliary gas flow=20 units, sweep gas flow=4 units, capillary temperature=300° C., and auxiliary gas heated to 300° C. Promass software was used to deconvolute the signal. Stability studies of siRNA conjugates in CSF

TABLE 2
siRNA conjugates for stability studies

| | | | | | | Molecular |
|---|---|---|---|---|---|---|
| Duplex | Oligo | | | | Molecular | Weight |
| Id | Id | Strand | Target | Oligo Seq | Weight | found |
| AD- | A- | sense | SOD1 | csasuuuuAfaUfCfCfucacucu | 7506.36 | 7502.51 |
| 224937 | 444399 | | | aaaL10 | | |
| | A- | antis | SOD1 | usUfsuagAfgUfGfaggaUfuA | 7775.15 | 7771.17 |
| | 268862 | | | faaaugsasg | | |
| AD- | A- | sense | SOD1 | csasuuuuAfaUfCfCfucacucu | 7522.43 | 7518.49 |
| 454834 | 809914 | | | aaasL10 | | |
| | A- | antis | SOD1 | usUfsuagAfgUfGfaggaUfuA | 7775.15 | 7771.17 |
| | 268862 | | | faaaugsasg | | |
| AD- | A- | sense | SOD1 | csasuuuuAfaUfCfCfucacucu | 7364.25 | 7360.36 |
| 953560 | 1700504 | | | aasasL322 | | |
| | A- | antis | SOD1 | VPusUfsuagAfgUfGfaggaUf | 7851.15 | 7847.15 |
| | 444402 | | | uAfaaaugsasg | | |
| AD- | A- | sense | SOD1 | csasuuuuAfaUfCfCfucacucu | 7251.10 | 7247.28 |
| 953561 | 1700503 | | | aasasL321 | | |
| | A- | antis | SOD1 | VPusUfsuagAfgUfGfaggaUf | 7851.15 | 7847.15 |
| | 444402 | | | uAfaaaugsasg | | |

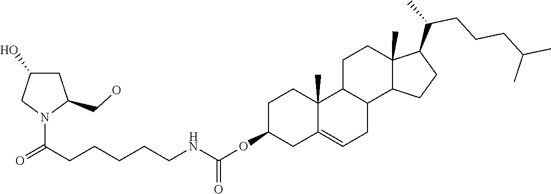 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C]

L10 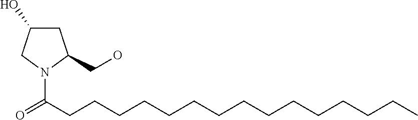 [SMILES: CCCCCCCCCCCCCCCC(=O)N1C[C@H](O)C[C@H]1CO]
L321 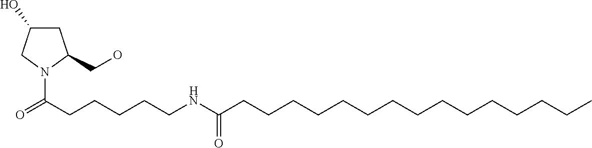 [SMILES: CCCCCCCCCCCCCCCC(=O)NCCCCCC(=O)N1C[C@H](O)C[C@H]1CO]
L322
Upper and lower case letters in italics indicate 2′-deoxy-2′-fluoro (2′-F), and 2′-O-methyl (2′-
OMe) sugar modifications, respectively, to adenosine, cytidine, guanosine and uridine; s
indicates phosphorothioate (PS) linkage; VP indicates vinyl phosphonate.

FIG. 2 shows the stability of the siRNAs conjugated with various lipophilic monomers (listed in Table 2 above) in rat CSF after incubating the siRNA duplexes with rat CSF for 24 hours.

Stability Studies of siRNA Conjugates In Vitreous Fluid

TABLE 3
siRNA conjugates for stability studies

| | | | | | | Molecular |
|---|---|---|---|---|---|---|
| Duplex | Oligo | | | | Molecular | Weight |
| Id | Id | Strand | Target | Oligo Seq | Weight | found |
| AD- | A-140611 | sense | h/c | usgsggauUfuCfAfUfguaa | 7704.51 | 7700.58 |
| 70500 | | | TTR | ccaagaL10 | | |
| | A-131902 | antis | h/c | VPusCfsuugGFuuAfcaug | 7633.01 | 7628.1 |
| | | | TTR | AfaAfucccasusc | | |
| AD- | A-444399 | sense | SOD1 | csasuuuuAfaUfCfCfucac | 7506.36 | 7502.51 |
| 224937 | | | | ucuaaaL10 | | |
| | A-268862 | antis | SOD1 | usUfsuagAfgUfGfaggaUf | 7775.15 | 7771.17 |
| | | | | uAfaaaugsasg | | |
| AD- | A-515644 | sense | h/c | usgsggauUfuCfAfUfguaa | 7558.33 | 7554.50 |
| 290674 | | | TTR | ccaagaL57 | | |
| | A-131092 | antis | h/c | VPusCfsuugGfuuAfcaug | 7633.01 | 7628.10 |
| | | | TTR | AfaAfucccasusc | | |
| AD- | A- | sense | mTTR | asascaguGfuUfCfUfugcu | 7347.15 | 7343.29 |
| 954308 | 1700512 | | | cuauasasL321 | | |
| | A-555715 | antis | TTR | VPuUfauaGfagcaagaAfc | 7699.98 | 7696.19 |
| | | | | Afcuguususu | | |
| AD- | A- | sense | mTTR | asascaguGfuUfCfUfugcu | 7460.30 | 7456.37 |
| 954311 | 1700513 | | | cuauasasL322 | | |
| | A-555715 | antis | TTR | VPuUfauaGfagcaagaAfc | 7699.98 | 7969.19 |
| | | | | Afcuguususu | | |

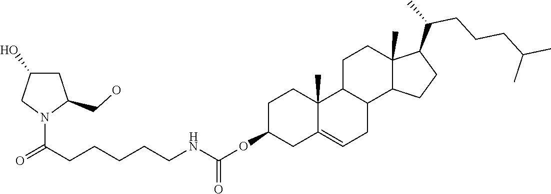 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C]

L10

[symbol]

L57 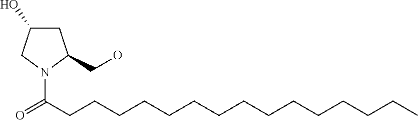 [SMILES: CCCCCCCCCCCCCCCC(=O)N1C[C@H](O)C[C@H]1CO]
L321 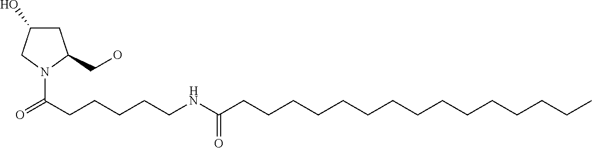 [SMILES: CCCCCCCCCCCCCCCC(=O)NCCCCCC(=O)N1C[C@H](O)C[C@H]1CO]
L322
Upper and lower case letters in italics indicate 2′-deoxy-2′-fluoro (2′-F), and 2′-O-methyl (2′-
OMe) sugar modifications, respectively, to adenosine, cytidine, guanosine and uridine; s
indicates phosphorothioate (PS) linkage; VP indicates vinyl phosphonate. [symbol]
indicates data missing or illegible when filed

FIG. 3 shows the stability of the siRNAs conjugated with various lipophilic monomers (listed in Table 3 above) in the vitreous humor of rabbit and cyno (NHP), respectively, for 24 hours. The remaining amounts of ligand-conjugated siRNA duplexes were plotted in the figure.

TABLE 4
siRNA conjugates for stability studies in vitreous fluid of rabbit and NHP

| | | | | | | Molecular |
|---|---|---|---|---|---|---|
| Duplex | Oligo | | | | Molecular | Weight |
| Id | Id | Strand | Target | Oligo Seq | Weight | Found |
| AD- | A- | sense | SOD1 | csasuuuuAfaUfCfCfu | 7506.36 | 7502.51 |
| 224937 | 444399 | | | cacucuaaaL10 | | |
| | A- | antis | SOD1 | usUfsuagAfgUfGfagg | 7775.15 | 7771.17 |
| | 268862 | | | aUfuAfaaaugsasg | | |
| AD- | A- | sense | SOD1 | csasuuuuAfaUfCfCfu | 7364.25 | 7360.36 |
| 953560 | 1700504 | | | cacucuaasasL322 | | |
| | A- | antis | SOD1 | VPusUfsuagAfgUfGf | 7851.15 | 7847.15 |
| | 444402 | | | aggaUfuAfaaaugsasg | | |
| AD- | A- | sense | mTTR | Q362sasacaguGfuUf | 7289.07 | 7285.24 |
| 954303 | 1700507 | | | CfUfugcucuausasa | | |
| | A- | antis | TTR | VPuUfauaGfagcaaga | 7699.98 | 7696.19 |
| | 555715 | | | AfcAfcuguususu | | |
| AD- | A- | sense | mTTR | Q363sasacaguGfuUf | 7317.12 | 7313.28 |
| 954304 | 1700508 | | | CfUfugcucuausasa | | |
| | A- | antis | TTR | VPuUfauaGfagcaaga | 7699.98 | 7696.19 |
| | 555715 | | | AfcAfcuguususu | | |
| AD- | A- | sense | mTTR | Q365sasacaguGfuUf | 7343.16 | 7339.29 |
| 954305 | 1700510 | | | CfUfugcucuausasa | | |
| | A- | antis | TTR | VPuUfauaGfagcaaga | 7699.98 | 7696.19 |
| | 555715 | | | AfcAfcuguususu | | |
| AD- | A- | sense | mTTR | Q361sasacaguGfuUf | 7261.01 | 7257.21 |
| 954306 | 1700506 | | | CfUfugcucuausasa | | |
| | A- | antis | TTR | VPuUfauaGfagcaaga | 7699.98 | 7696.19 |
| | 555715 | | | AfcAfcuguususu | | |
| AD- | A- | sense | mTTR | Q364sasacaguGfuUf | 7345.18 | 7341.31 |
| 954307 | 1700509 | | | CfUfugcucuausasa | | |
| | A- | antis | TTR | VPuUfauaGfagcaaga | 7699.98 | 7696.19 |
| | 555715 | | | AfcAfcuguususu | | |
| AD- | A- | sense | mTTR | asascaguGfuUfCfUfu | 7347.15 | 7343.29 |
| 954308 | 1700512 | | | gcucuauasasL321 | | |
| | A- | antis | TTR | VPuUfauaGfagcaaga | 7699.98 | 7696.19 |
| | 555715 | | | AfcAfcuguususu | | |
| AD- | A- | sense | mTTR | Q366sasacaguGfuUf | 7401.28 | 7397.37 |
| 954309 | 1700511 | | | CfUfugcucuausasa | | |
| | A- | antis | TTR | VPuUfauaGfagcaaga | 7699.98 | 7696.19 |
| | 555715 | | | AfcAfcuguususu | | |
| AD- | A- | sense | mTTR | Q370sascaguGfuUfCf | 7167.05 | 7163.27 |
| 954310 | 1700514 | | | Ufugcucuausasa | | |
| | A- | antis | TTR | VPuUfauaGfagcaaga | 7699.98 | 7696.19 |
| | 555715 | | | AfcAfcuguususu | | |

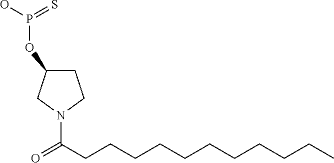

Q361s 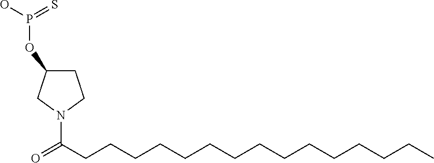
Q362s 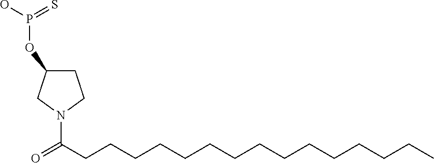
Q363s 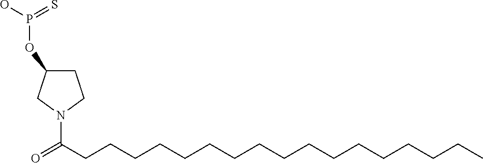
Q364s 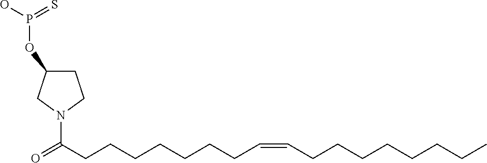
Q365s 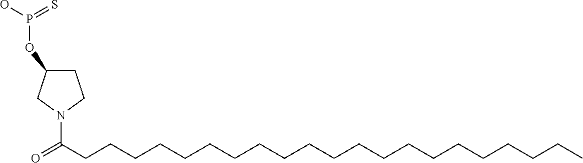
Q366s 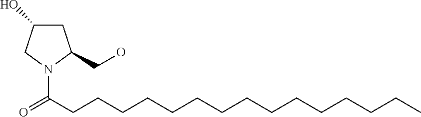 [SMILES: CCCCCCCCCCCCCCCC(=O)N1C[C@H](O)C[C@H]1CO]
L321 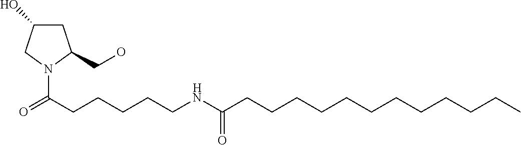 [SMILES: CCCCCCCCCCCCC(=O)NCCCCCC(=O)N1C[C@H](O)C[C@H]1CO]
L322 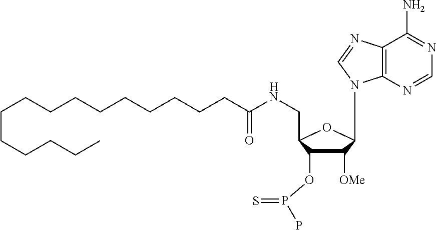
Q370
Upper and lower case letters in italics indicate 2′-deoxy-2′-fluoro (2′-F), and 2′-O-methyl (2′-
OMe) sugar modifications, respectively, to adenosine, cytidine, guanosine and uridine; s
indicates phosphorothioate (PS) linkage; VP indicates vinyl phosphonate.

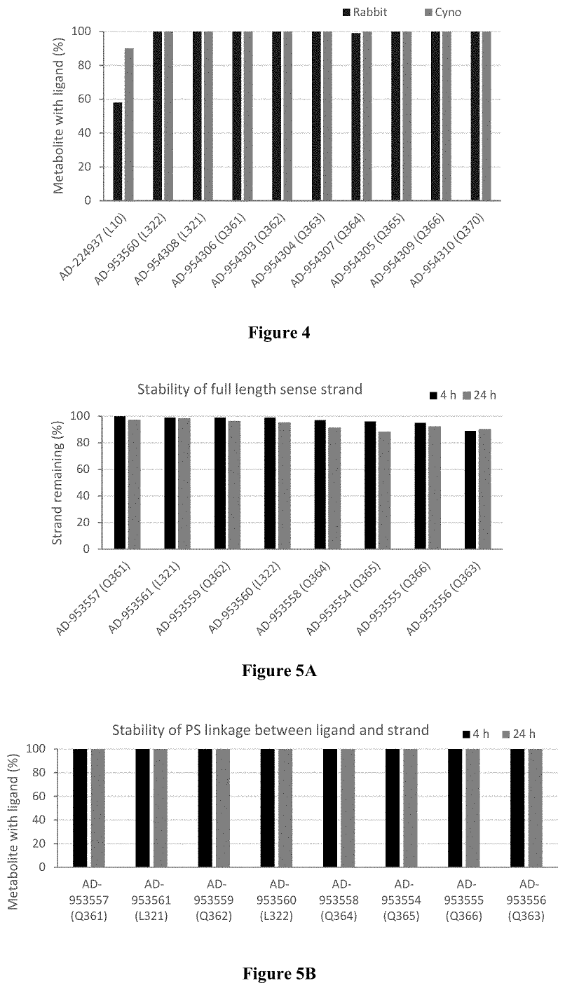

FIG. 4 shows the stability of the siRNAs conjugated with various lipophilic monomers (listed in Table 4 above) in the vitreous humor of rabbit and cyno (NHP) for 24 hours. The remaining amounts of ligand-conjugated siRNA duplexes were plotted in the figure.

TABLE 5
siRNA conjugates for metabolic stability studies in rat brain homogenate

| | | | | | | Molecular |
|---|---|---|---|---|---|---|
| Duplex | | | | | Molecular | Weight |
| Id | Oligo Id | Strand | Target | Oligo Seq | Weight | Found |
| AD- | A- | sense | SOD1 | Q361scsauuuuAfaUfCfCfuc | 7164.964 | 7161.211 |
| 953557 | 1700497 | | | acucuasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | Q362scsauuuuAfaUfCfCfuc | 7193.024 | 7189.242 |
| 953559 | 1700498 | | | acucuasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | Q363scsauuuuAfaUfCfCfuc | 7221.074 | 7217.274 |
| 953556 | 1700499 | | | acucuasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | Q364scsauuuuAfaUfCfCfuc | 7249.134 | 7245.305 |
| 953558 | 1700500 | | | acucuasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | Q365scsauuuuAfaUfCfCfuc | 7247.114 | 7243.289 |
| 953554 | 1700501 | | | acucuasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | Q366scsauuuuAfaUfCfCfuc | 7305.234 | 7301.368 |
| 953555 | 1700502 | | | acucuasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | csasuuuuAfaUfCfCfucacuc | 7251.109 | 7247.284 |
| 953561 | 1700503 | | | uaasasL321 | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | csasuuuuAfaUfCfCfucacuc | 7364.259 | 7360.368 |
| 953560 | 1700504 | | | uaasasL322 | | |
| | A- | antis | SOD1 | VPusUfsuagAfgUfGfaggaU | 7851.156 | 7847.154 |
| | 444402 | | | fuAfaaaugsasg | | |

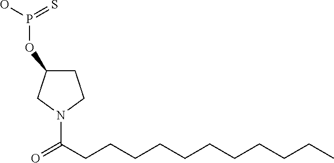

Q361s 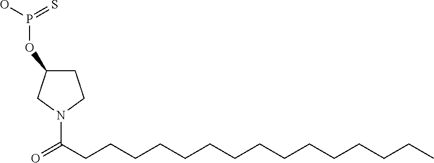
Q362s 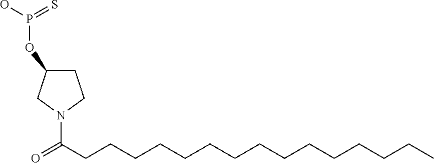
Q363s 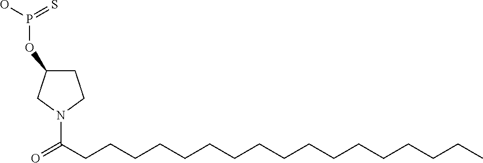
Q364s 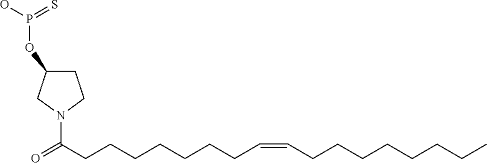
Q365s 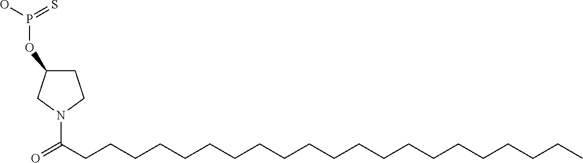
Q366s 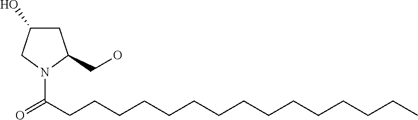 [SMILES: CCCCCCCCCCCCCCCC(=O)N1C[C@H](O)C[C@H]1CO]
L321 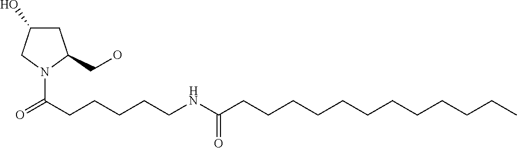 [SMILES: CCCCCCCCCCCCC(=O)NCCCCCC(=O)N1C[C@H](O)C[C@H]1CO]
L322
Upper and lower case letters in italics indicate 2′-deoxy-2′-fluoro (2′-F), and 2′-O-methyl (2′-
OMe) sugar modifications, respectively, to adenosine, cytidine, guanosine and uridine; s
indicates phosphorothioate (PS) linkage; VP indicates vinyl phosphonate.

FIGS. 5A and 5B show the stability of the siRNAs conjugated with various lipophilic monomers (listed in Table 5 above) in rat brain homogenate for 4 hours and 24 hours, respectively. The remaining amounts of ligand-conjugated siRNA duplexes were plotted in FIG. 5A. FIG. 5B shows the stability of PS linkages.

TABLE 6
siRNAs conjugated with esterase cleavable conjugates for stability studies in vitreous fluid

| | | | | | | Molecular |
|---|---|---|---|---|---|---|
| Duplex | | | | | Molecular | Weight |
| Id | Oligo Id | Strand | Target | Oligo Seq | Weight | Found |
| AD- | A- | sense | TTR | asascag(Uhd)GfuUfCfUfu | 7140.02 | 7136.26 |
| 307571 | 594427 | | | gcucuausasa | | |
| | A- | antis | TTR | VPuUfauaGfagcaagaAfcAf | 7699.98 | 7696.19 |
| | 555715 | | | cuguususu | | |
| AD- | A- | sense | TTR | asascagY132GfuUfCfUfug | 7325.25 | 7321.36 |
| 890095 | 1543023 | | | cucuausasa | | |
| | A- | antis | TTR | VPuUfauaGfagcaagaAfcAf | 7699.98 | 7696.19 |
| | 555715 | | | cuguususu | | |
| AD- | A- | sense | TTR | asascagY133GfuUfCfUfug | 7353.30 | 7349.39 |
| 890096 | 1543024 | | | cucuausasa | | |
| | A- | antis | TTR | VPuUfauaGfagcaagaAfcAf | 7699.98 | 7696.19 |
| | 555715 | | | cuguususu | | |
| AD- | A- | sense | TTR | asascagY134GfuUfCfUfug | 7311.22 | 7307.35 |
| 890097 | 1543025 | | | cucuausasa | | |
| | A- | antis | TTR | VPuUfauaGfagcaagaAfcAf | 7699.98 | 7696.19 |
| | 555715 | | | cuguususu | | |
| AD- | A- | sense | TTR | asascagY135GfuUfCfUfug | 7339.28 | 7335.38 |
| 890094 | 1543026 | | | cucuausasa | | |
| | A- | antis | TTR | VPuUfauaGfagcaagaAfcAf | 7699.98 | 7696.19 |
| | 555715 | | | cuguususu | | |

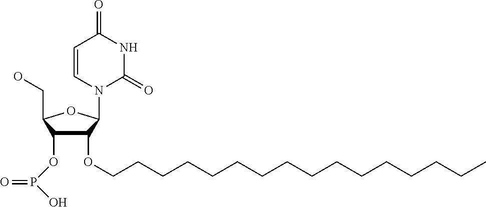

(Uhd) 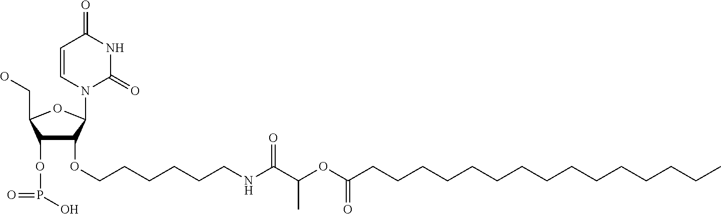
Y132 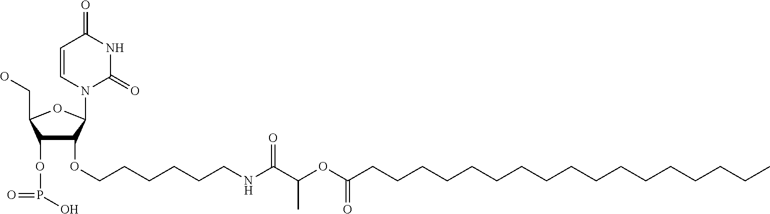
Y133 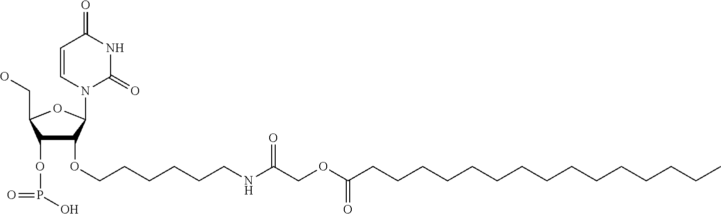
Y134 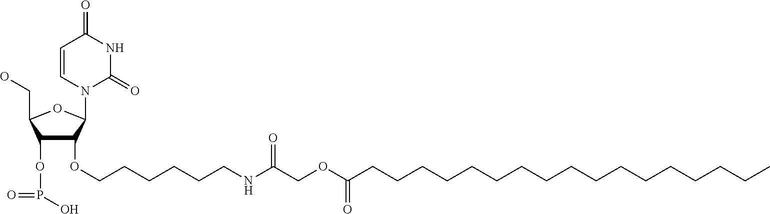
Y135
Upper and lower case letters in italics indicate 2′-deoxy-2′-fluoro (2′-F), and 2′-O-methyl (2′-
OMe) sugar modifications, respectively, to adenosine, cytidine, guanosine and uridine; s
indicates phosphorothioate (PS) linkage; VP indicates vinyl phosphonate.

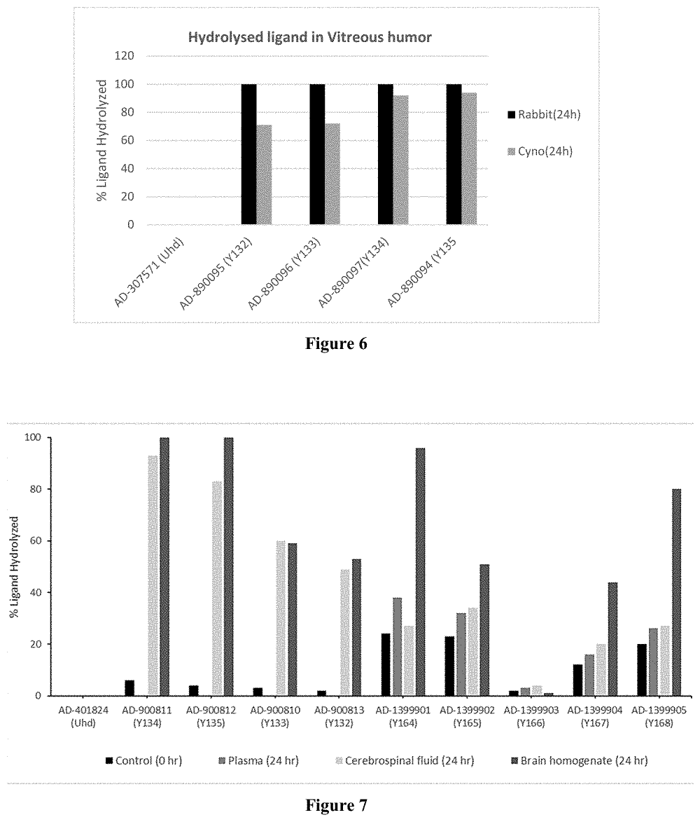

FIG. 6 shows the stability of the siRNA conjugates having esterase cleavable conjugates (listed in Table 6 above) in the vitreous humor of rabbit and cyno (NHP) for 24 hours. The percentage of the hydrolyzed ligand-conjugated siRNA duplexes were plotted in the figure.

TABLE 7
siRNA conjugated with esterase cleavable conjugates for stability studies in Plasma, CSF and brain homogenate

| | | | | | | Molecular |
|---|---|---|---|---|---|---|
| Duplex | | | | | Molecular | Weight |
| Id | Oligo Id | Strand | Target | Oligo Seq | Weight | Found |
| AD- | A- | sense | SOD1 | csasuuu(Uhd)AfaUfCfC | 7043.97 | 7040.25 |
| 401824 | 637448 | | | fucacucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuY132AfaUfCfC | 7229.20 | 7225.36 |
| 900813 | 1543019 | | | fucacucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuY133AfaUfCfC | 7257.26 | 7253.39 |
| 900810 | 1543020 | | | fucacucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuY134AfaUfCfC | 7215.18 | 7211.34 |
| 900811 | 1543021 | | | gaUfuAfaaaugsasg | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuY135AfaUfCfC | 7243.23 | 7239.37 |
| 900812 | 1543022 | | | fucacucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuY164AfaUfCfC | 7229.20 | 7225.36 |
| 1399901 | 2208106 | | | fucacucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuY165AfaUfCfC | 7229.20 | 7225.36 |
| 1399902 | 2208107 | | | fucacucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuY166AfaUfCfC | 7243.23 | 7239.37 |
| 1399903 | 2208108 | | | fucacucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuY167AfaUfCfC | 7257.25 | 7253.39 |
| 1399904 | 2208109 | | | fucacucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuY168AfaUfCfC | 7229.20 | 7225.36 |
| 1399905 | 2208110 | | | fucacucuasasa | | |
| | A- | antis | SOD1 | VPusUfsuagAfgUfGfag | 7851.15 | 7847.15 |
| | 444402 | | | gaUfuAfaaaugsasg | | |

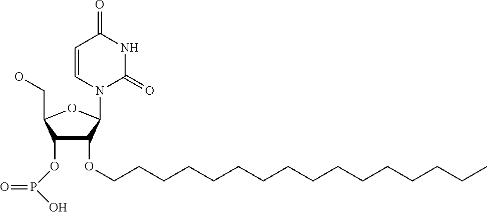

(Uhd) 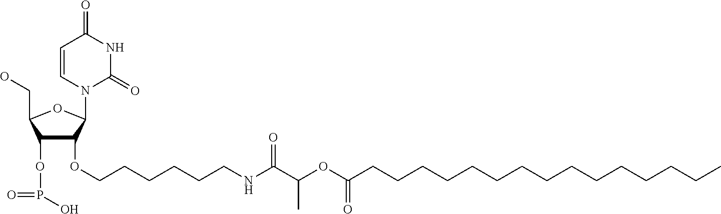
Y132 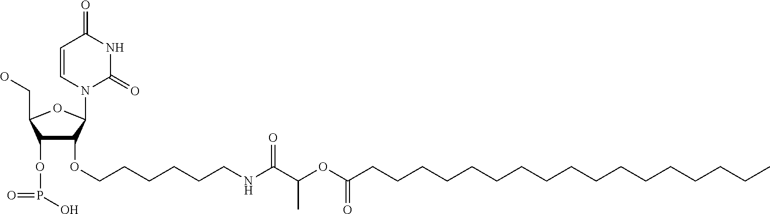
Y133 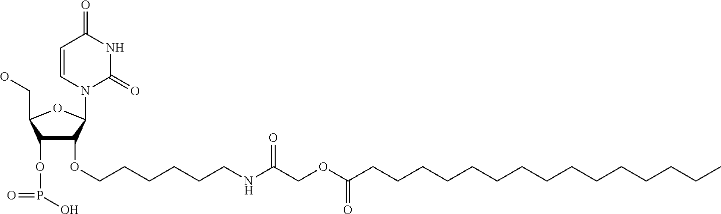
Y134 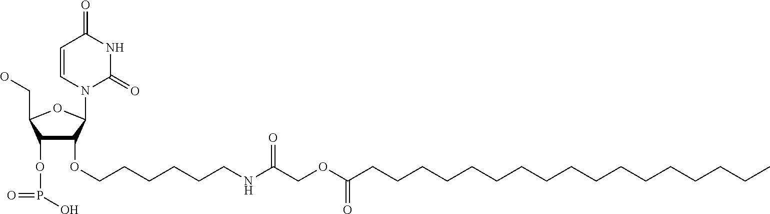
Y135 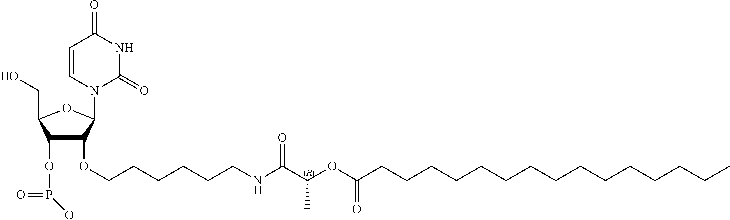
Y164 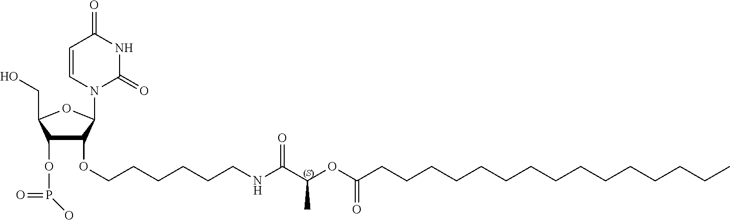
Y165 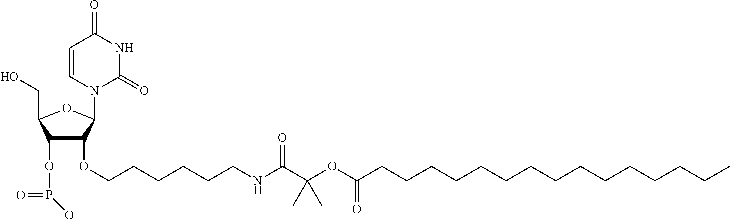
Y166 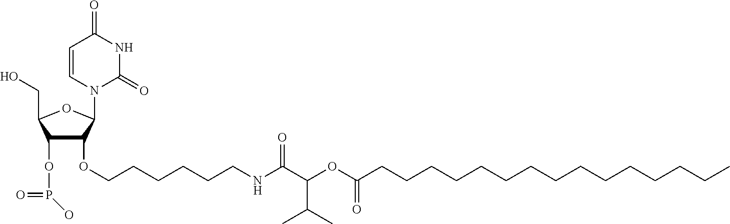
Y167 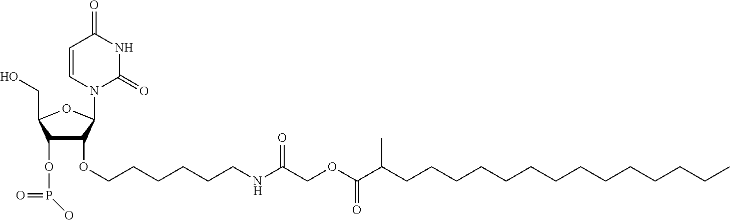
Y168
Upper and lower case letters in italics indicate 2′-deoxy-2′-fluoro (2′-F), and 2′-O-methyl (2′-
OMe) sugar modifications, respectively, to adenosine, cytidine, guanosine and uridine; s
indicates phosphorothioate (PS) linkage; VP indicates vinyl phosphonate.

FIG. 7 shows the stability of the siRNA conjugates having esterase cleavable conjugates (listed in Table 7 above) in rat plasma, CSF and brain homogenate for 24 hours. The percentage of the hydrolyzed ligand-conjugated siRNA duplexes were plotted.

### Example 7. Co-Relation of Hydrophobicity and Activity in CNS Tissues

To evaluate the role of hydrophobicity in uptake and activity of lipids conjugates in CNS tissue, a number of shorter lipids were introduced in the sense or antisense strand (Table 8), instead of a single longer lipid chain. Based on the hydrophobicity measurements by EMSA assay, it was determined that, for the siRNA conjugates having a number of shorter lipid chains introduced, a hydrophobicity similar to that of siRNA conjugates having a single long chain can be achieved. Protein binding characteristics of siRNA conjugates were measured by EMSA assay as given below.

EMSA Assay protocol for Kd Determination: Bio Rad 10% Criterion TBE polyacrylamide gel was equilibrated with a pre-run in 1×TBE at 100V for 20 minutes, in a Criterion gel electrophoresis tank. Each sample well was flushed with 20 μL of 1×TBE electrophoresis buffer (Bio Rad) before and after the pre-run. Samples were prepared at duplicate for two gels per siRNA duplex (total quadruplicate). Duplexes at a stock concentration of 10 μM in 1×PBS were diluted to a final concentration of 0.5 μM (20 μL total volume) containing 1×PBS and increasing concentrations of non-denatured human serum albumin (HSA) solution (Calbiochem). Human serum albumin concentration ranged from 0 μM to 1000 μM in increments of 100 for max of 1 mM, and 0 μM to 2000 μM in varying increments for max 2 mM. The samples were mixed, centrifuged for 30 seconds at 3000 RPM, and subsequently incubated at room temperature for 10 minutes.

Once incubation was complete, 4 μL of 6×EMSA Gel-loading solution (LifeTechnologies) was added to each sample, centrifuged for 30 seconds at 3000 RPM, and 12 μL of each sample was loaded on the gel. The gel electrophoresis was first run at 50V for 20 minutes to allow the entire sample to be fully loaded on the gel. Then the gel electrophoresis was run at 100V for 1 hour. At the completion of electrophoresis, the gel was removed from the casing and placed in 50 mL of 1×TBE. To stain, 5 μL of SYBR Gold (LifeTechnologies) was added to the container and the gel was incubated at room temperature for 10 minutes, on a platform rocker. The gel was rinsed with 50 mL of 1×TBE and placed in an additional 50 mL of the buffer.

Bio Rad ChemiDoc MP Imaging System was used to image the gel using the following parameters: the imaging application was set to SYBR Gold, the size was set to Bio-Rad criterion gel, the exposure was set to automatic for intense bands, the highlight saturated pixels where turned one and the color was set to gray. The detection, molecular weight analysis, and output were all disabled. Once a clean photo of the gel was obtained, Image Lab 5.2 (Bio Rad) was used to process the image. The lanes and bands where manually set to measure band intensity. Band intensities of each sample where normalized to that of the duplex without human serum albumin (control at 0 μM) to obtain the fraction of bound siRNA relative to the concentration of HSA. Binding affinity dissociation constant was calculated on GraphPad Prism 7, using nonlinear regression curve fit with the one site specific binding with Hill slope equation.

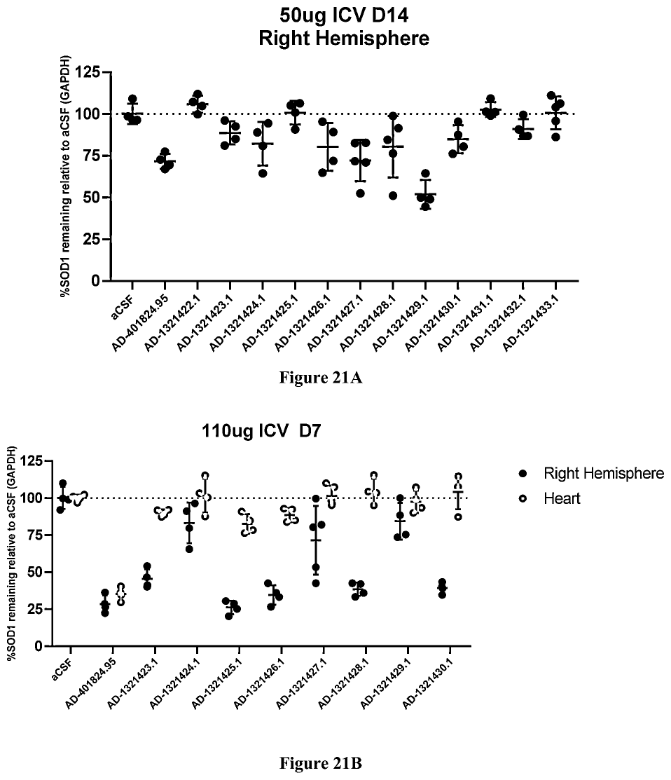

Similar in vivo activity was obtained by introducing multiple shorter lipids in the duplex structure instead of introducing a single lipid chain in the siRNA conjugates. Activity of siRNA conjugate depends on the position of different shorter lipids in the sequence. By utilizing these designs, the systemic exposure siRNA conjugates to liver, kidney and heart can be limited (FIG. 21B)

TABLE 8
Kd and duplex information for siRNA conjugates
used for protein binding assay

| | Location of | | |
|---|---|---|---|
| Duplex ID | Modification | Modification Count | K_{D} Value (μM) |
| AD-1321422 | S6, 16, 17 | C6 × 3 | 1176 ± 446 |
| AD-1321423 | S6, 16, 21 | C6 × 3 | 745.5 ± 156.9 |
| AD-1321424 | S1, 6, 16 | C6 × 3 | 882 ± 97.7 |
| AD-1321425 | S1, 6, 16, 21 | C6 × 4 | 372.1 ± 47.1 |
| AD-1321426 | S1, 6, 15, 16, 21 | C3 × 5 | 1766 ± 1224 |
| AD-1321427 | S6, 17 | C6 × 2 | ≥2 mM HSA |
| AD-1321428 | S6, 16 | C6 × 2 | ≥2 mM HSA |
| AD-1321429 | S1, 6, 16, 17, 21 | C6 × 5 | 189.5 ± 24.4 |
| AD-1321430 | S6, 16, 17 | C6 × 2 + C3 × 1 | 1312 ± 49.4 |
| AD-1321431 | S6, 17; AS16 | C6 × 3 | 1284 ± 46.7 |
| AD-1321432 | S6, 16; AS16 | C6 × 3 | 1313 ± 70.9 |
| AD-1321433 | S6, 16, 17; AS16 | C6 × 3 + C3 × 1 | ≥2 mM HSA |
| AD-401824 | S6 | C16 | 176.4 ± 17 |

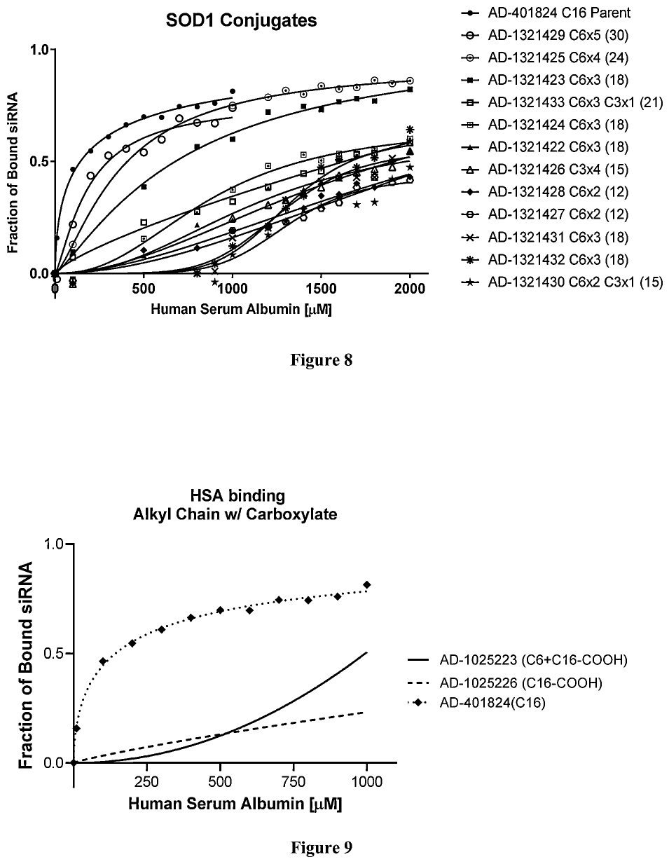

Hydrophobicity of siRNA conjugates is critical for the activity and distribution of the siRNA to different CNS tissues and also plays a major role in systemic exposure of siRNA conjugates after intrathecal administration. By examining the protein binding characteristics of number of conjugates, it was found (see Tables 8-9 and FIGS. 8-9) that the conjugates having alkyl chains with exposed carboxylic were active in CNS tissues although less active in heart.

TABLE 9
Kd and duplex information for conjugates
used for protein binding assay

| Series | Duplex Number | n | K_{D} Value (μM HSA) |
|---|---|---|---|
| Alkyl chain | AD-401824 | 16 | 176.4 ± 17 |
| Alkyl Chain with | AD-1025226 | 16 | No binding (≥1 mM HSA) |
| Carboxylate | AD-1025223 | 16 | No binding (≥1 mM HSA) |

### Example 8. mRNA Knockdown in Mouse Eyes Using Lipophilic Conjugated siRNAs

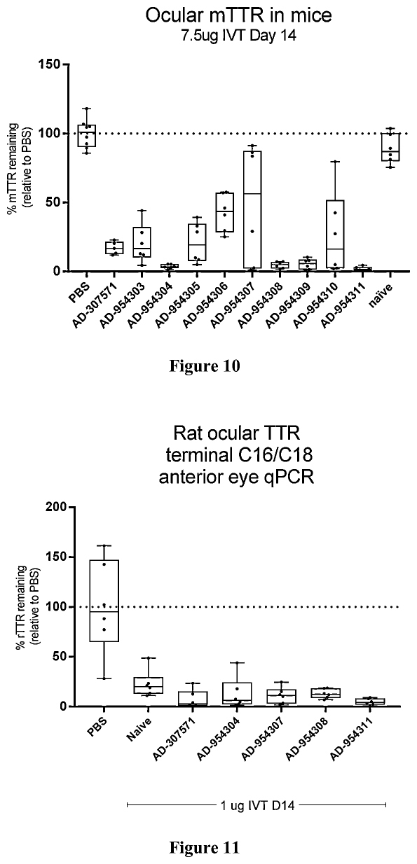

TTR gene silencing was studied with siRNA conjugates listed in Table 10 by qPCR in mouse eyes following intravitreal administration of a single 7.5 μg or 1 μg dose of siRNA duplexes, with the mice sacrificed on day 14, and the results were compared to PBS control. The results are shown in FIGS. 10-11.

TABLE 10
5′-3′ lipophilic siRNA conjugates for in vivo ocular studies

| | | | | | | Molecular |
|---|---|---|---|---|---|---|
| Duplex | Oligo | | | | Molecular | Weight |
| Id | Id | Strand | Target | Oligo Seq | Weight | Found |
| AD- | A- | sense | TTR | asascag(Uhd)GfuUfCfUfu | 7140.02 | 7136.26 |
| 307571 | 594427 | | | gcucuausasa | | |
| | A- | antis | TTR | VPuUfauaGfagcaagaAfcA | 7699.98 | 7696.16 |
| | 555715 | | | fcuguususu | | |
| AD- | A- | sense | mTTR | Q361sasacaguGfuUfCfUfu | 7261.01 | 7257.21 |
| 954306 | 1700506 | | | gcucuausasa | | |
| | A- | antis | TTR | VPuUfauaGfagcaagaAfcA | 7699.98 | 7696.19 |
| | 555715 | | | fcuguususu | | |
| AD- | A- | sense | mTTR | Q362sasacaguGfuUfCfUfu | 7289.07 | 7285.24 |
| 954303 | 1700507 | | | gcucuausasa | | |
| | A- | antis | TTR | VPuUfauaGfagcaagaAfcA | 7699.98 | 7696.19 |
| | 555715 | | | fcuguususu | | |
| AD- | A- | sense | mTTR | Q363sasacaguGfuUfCfUfu | 7317.12 | 7313.28 |
| 954304 | 1700508 | | | gcucuausasa | | |
| | A- | antis | TTR | VPuUfauaGfagcaagaAfcA | 7699.98 | 7696.19 |
| | 555715 | | | fcuguususu | | |
| AD- | A- | sense | mTTR | Q364sasacaguGfuUfCfUfu | 7345.18 | 7341.31 |
| 954307 | 1700509 | | | gcucuausasa | | |
| | A- | antis | TTR | VPuUfauaGfagcaagaAfcA | 7699.98 | 7696.19 |
| | 555715 | | | fcuguususu | | |
| AD- | A- | sense | mTTR | Q365sasacaguGfuUfCfUfu | 7343.16 | 7339.29 |
| 954305 | 1700510 | | | gcucuausasa | | |
| | A- | antis | TTR | VPuUfauaGfagcaagaAfcA | 7699.98 | 7696.19 |
| | 555715 | | | fcuguususu | | |
| AD- | A- | sense | mTTR | Q366sasacaguGfuUfCfUfu | 7401.28 | 7397.37 |
| 954309 | 1700511 | | | gcucuausasa | | |
| | A- | antis | TTR | VPuUfauaGfagcaagaAfcA | 7699.98 | 7696.19 |
| | 555715 | | | fcuguususu | | |
| AD- | A- | sense | mTTR | asascaguGfuUfCfUfugcuc | 7347.15 | 7343.29 |
| 954308 | 1700512 | | | uauasasL321 | | |
| | A- | antis | TTR | VPuUfauaGfagcaagaAfcA | 7699.98 | 7696.19 |
| | 555715 | | | fcuguususu | | |
| AD- | A- | sense | mTTR | asascaguGfuUfCfUfugcuc | 7460.30 | 7456.37 |
| 954311 | 1700513 | | | uauasasL322 | | |
| | A- | antis | TTR | VPuUfauaGfagcaagaAfcA | 7699.98 | 7696.19 |
| | 555715 | | | fcuguususu | | |
| AD- | A- | sense | mTTR | Q370sascaguGfuUfCfUfu | 7167.05 | 7163.27 |
| 954310 | 1700514 | | | gcucuausasa | | |
| | A- | antis | TTR | VPuUfauaGfagcaagaAfcA | 7699.98 | 7696.19 |
| | 555715 | | | fcuguususu | | |

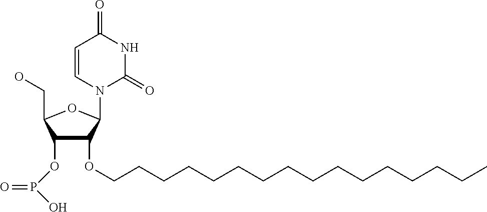

(Uhd) 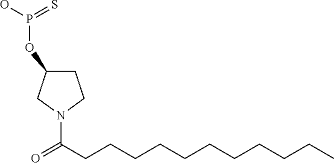
Q361s 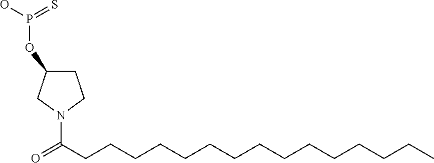
Q362s 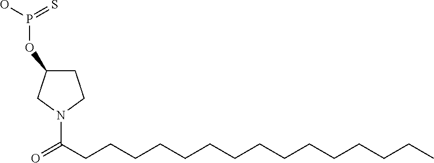
Q363s 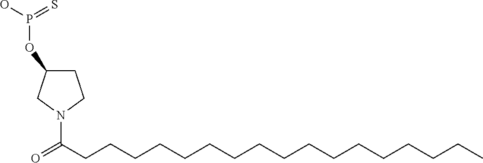
Q364s 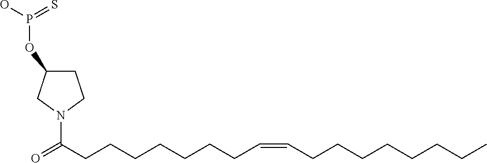
Q365s 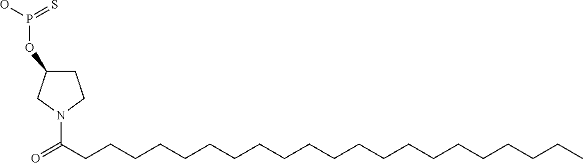
Q366s 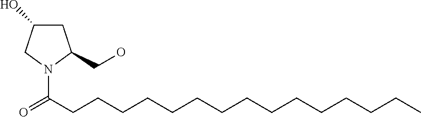 [SMILES: CCCCCCCCCCCCCCCC(=O)N1C[C@H](O)C[C@H]1CO]
L321 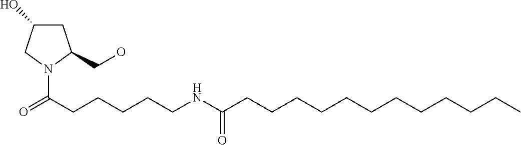 [SMILES: CCCCCCCCCCCCC(=O)NCCCCCC(=O)N1C[C@H](O)C[C@H]1CO]
L322 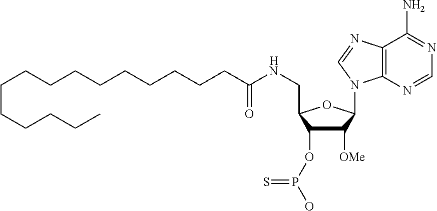
Q370
*Upper and lower case letters in italics indicate 2′-deoxy-2′-fluoro (2′-F), and 2′-O-methyl
(2′-OMe) sugar modifications, respectively, to adenosine, cytidine, guanosine and uridine; s
indicates phosphorothioate (PS) linkage; VP indicates vinyl phosphonate; Nhd indicates 2′-
O-hexadecyl.

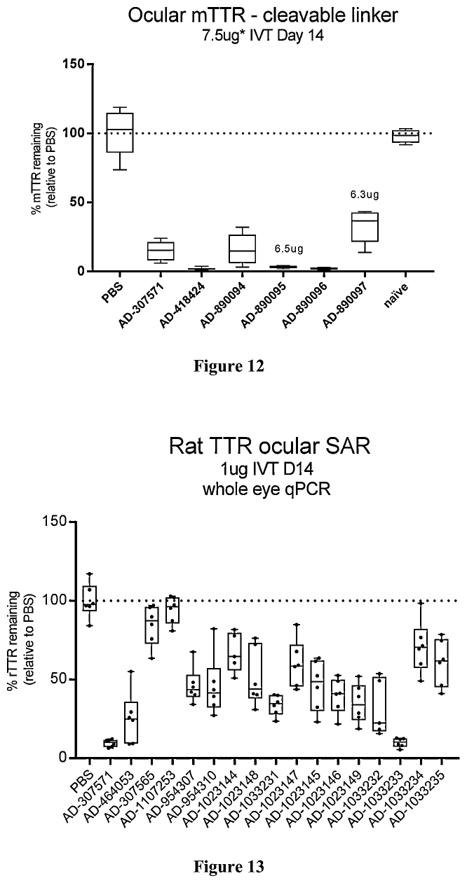

TTR gene silencing was also studied with siRNA conjugates listed in Table 11 by qPCR in mouse eyes following intravitreal administration of a single 7.5 μg dose of siRNA duplexes, with the mice sacrificed on day 14, and the results were compared to PBS control. The results are shown in FIG. 12. The siRNA duplexes listed below were conjugated with esterase cleavable conjugates.

TABLE 11
Esterase cleavable lipophilic siRNA conjugates of TTR sequence.

| | | | | | | Molecular |
|---|---|---|---|---|---|---|
| Duplex | Oligo | | | | Molecular | Weight |
| Id | Id | Strand | Target | Oligo Seq | Weight | Found |
| AD- | A- | sense | TTR | asascag(Uhd)GfuUfCfUfug | 7140.02 | 7136.26 |
| 307571 | 594427 | | | cucuausasa | | |
| | A- | antis | TTR | VPuUfauaGfagcaagaAfcAf | 7699.98 | 7696.16 |
| | 555715 | | | cuguususu | | |
| AD- | A- | sense | TTR | asascagY84GfuUfCfUfugcu | 7279.23 | 7275.36 |
| 418424 | 637431 | | | cuausasa | | |
| | A- | antis | TTR | VPuUfauaGfagcaagaAfcAf | 7699.98 | 7696.19 |
| | 555715 | | | cuguususu | | |
| AD- | A- | sense | TTR | asascagY132GfuUfCfUfugc | 7325.25 | 7321.36 |
| 890095 | 1543023 | | | ucuausasa | | |
| | A- | antis | TTR | VPuUfauaGfagcaagaAfcAf | 7699.98 | 7696.19 |
| | 555715 | | | cuguususu | | |
| AD- | A- | sense | TTR | asascagY133GfuUfCfUfugc | 7353.30 | 7349.39 |
| 890096 | 1543024 | | | ucuausasa | | |
| | A- | antis | TTR | VPuUfauaGfagcaagaAfcAf | 7699.98 | 7696.19 |
| | 555715 | | | cuguususu | | |
| AD- | A- | sense | TTR | asascagY134GfuUfCfUfugc | 7311.22 | 7307.35 |
| 890097 | 1543025 | | | ucuausasa | | |
| | A- | antis | TTR | VPuUfauaGfagcaagaAfcAf | 7699.98 | 7696.19 |
| | 555715 | | | cuguususu | | |
| AD- | A- | sense | TTR | asascagY135GfuUfCfUfugc | 7339.28 | 7335.38 |
| 890094 | 1543026 | | | ucuausasa | | |
| | A- | antis | TTR | VPuUfauaGfagcaagaAfcAf | 7699.98 | 7696.19 |
| | 555715 | | | cuguususu | | |

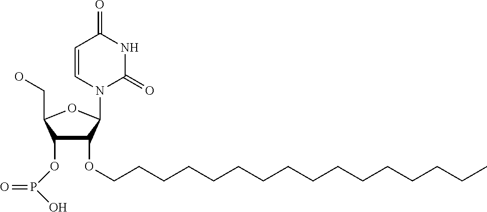

(Uhd) 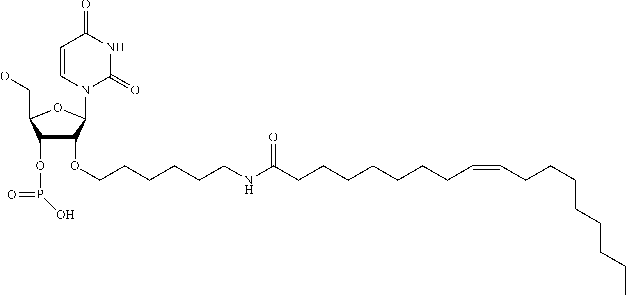
Y84 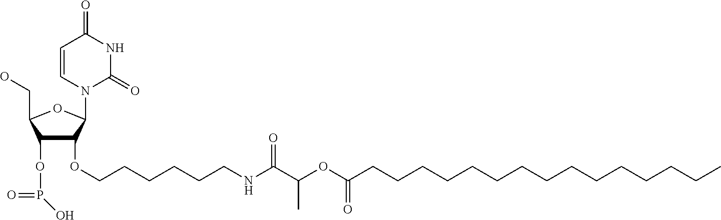
Y132 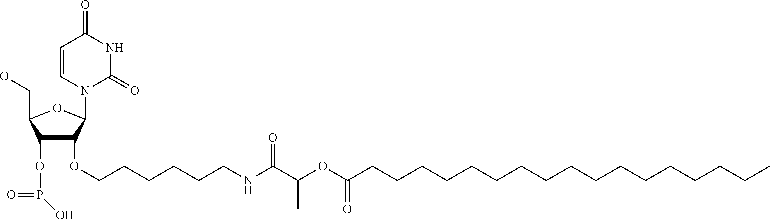
Y133 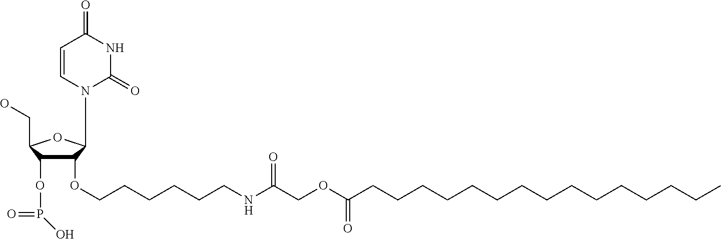
Y134 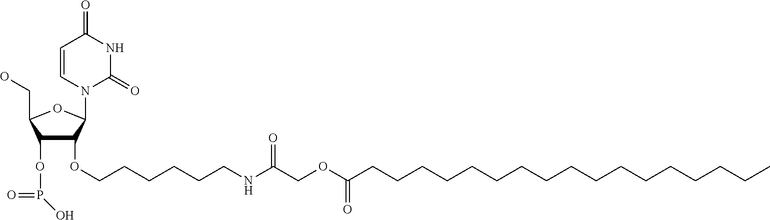
Y135
*Upper and lower case letters in italics indicate 2′-deoxy-2′-fluoro (2′-F), and 2′-O-methyl
(2′-OMe) sugar modifications, respectively, to adenosine, cytidine, guanosine and uridine; s
indicates phosphorothioate (PS) linkage; VP indicates vinyl phosphonate; Nhd indicates 2′-
O-hexadecyl.

TTR gene silencing was also studied with siRNA conjugates listed in Table 12 by qPCR in rat eyes following intravitreal administration of a single 1 μg dose of siRNA duplexes, with the rat sacrificed on day 14, and the results were compared to PBS control. The results are shown in FIG. 13.

TABLE 12
Lipophilic siRNA conjugates for in vivo study in rat (5′, 3′, internal, and terminal carboxylic acid)

| | | | | | | Molecular |
|---|---|---|---|---|---|---|
| | | | | | Molecular | Weight |
| Duplex Id | Oligo Id | Strand | Target | Oligo Seq | Weight | Found |
| AD- | A- | sense | m/rTTR | Q3 77sasacaguGfuUfCfUfug | 7347.09 | 7343.255 |
| 1023144 | 1812977 | | | cucuausasa | | |
| | A- | antis | TTR | VPuUfauaGfagcaagaAfcAfc | 7699.98 | 7696.19 |
| | 555715 | | | uguususu | | |
| AD- | A- | sense | m/rTTR | Q378sasacaguGfuUfCfUfug | 7375.15 | 7371.28 |
| 1023148 | 1812978 | | | cucuausasa | | |
| | A- | antis | TTR | VPuUfauaGfagcaagaAfcAfc | 7699.98 | 7696.19 |
| | 555715 | | | uguususu | | |
| AD- | A- | sense | m/rTTR | Q379sasacaguGfuUfCfUfug | 7403.20 | 7399.31 |
| 1033231 | 1812979 | | | cucuausasa | | |
| | A- | antis | TTR | VPuUfauaGfagcaagaAfcAfc | 7699.98 | 7696.19 |
| | 555715 | | | uguususu | | |
| AD- | A- | sense | m/rTTR | asascagY152GfuUfCfUfugc | 7170.01 | 7166.23 |
| 1023147 | 1812980 | | | ucuausasa | | |
| | A- | antis | TTR | VPuUfauaGfagcaagaAfcAfc | 7699.98 | 7696.19 |
| | 555715 | | | uguususu | | |
| AD- | A- | sense | m/rTTR | asascagY153GfuUfCfUfugc | 7170.01 | 7166.23 |
| 1023145 | 1812981 | | | ucuausasa | | |
| | A- | antis | TTR | VPuUfauaGfagcaagaAfcAfc | 7699.98 | 7696.19 |
| | 555715 | | | uguususu | | |
| AD- | A- | sense | m/rTTR | asascagY154GfuUfCfUfugc | 7283.17 | 7279.32 |
| 1023146 | 1812982 | | | ucuausasa | | |
| | A- | antis | TTR | VPuUfauaGfagcaagaAfcAfc | 7699.98 | 7696.19 |
| | 555715 | | | uguususu | | |
| AD- | A- | sense | m/rTTR | asascagY155GfuUfCfUfugc | 7311.22 | 7307.35 |
| 1023149 | 1812983 | | | ucuausasa | | |
| | A- | antis | TTR | VPuUfauaGfagcaagaAfcAfc | 7699.98 | 7696.19 |
| | 555715 | | | uguususu | | |
| AD- | A- | sense | m/rTTR | asascagY156GfuUfCfUfugc | 7339.28 | 7335.38 |
| 1033232 | 1812984 | | | ucuausasa | | |
| | A- | antis | TTR | VPuUfauaGfagcaagaAfcAfc | 7699.98 | 7696.19 |
| | 555715 | | | uguususu | | |
| AD- | A- | sense | m/rTTR | asascagY158GfuUfCfUfugc | 7217.13 | 7213.25 |
| 1033233 | 1840408 | | | ucuausasa | | |
| | A- | antis | TTR | VPuUfauaGfagcaagaAfcAfc | 7699.98 | 7696.19 |
| | 555715 | | | uguususu | | |
| AD- | A- | sense | mTTR | asascagQ382GfuUfCfUfugc | 7056.94 | 7053.22 |
| 1033234 | 1866827 | | | ucuausasa | | |
| | A- | antis | TTR | VPuUfauaGfagcaagaAfcAfc | 7699.98 | 7696.19 |
| | 555715 | | | uguususu | | |
| AD- | A- | sense | mTTR | asascagQ383GfuUfCfUfugc | 7084.99 | 7081.25 |
| 1033235 | 1866828 | | | ucuausasa | | |
| | A- | antis | TTR | VPuUfauaGfagcaagaAfcAfc | 7699.98 | 7696.19 |
| | 555715 | | | uguususu | | |

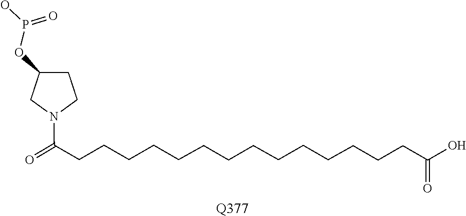

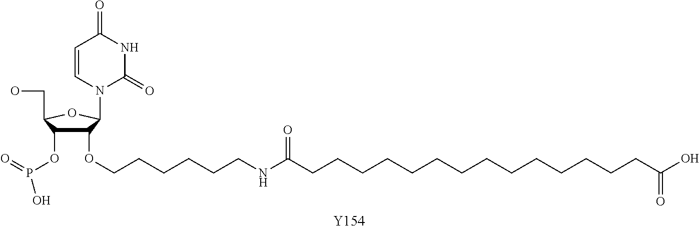

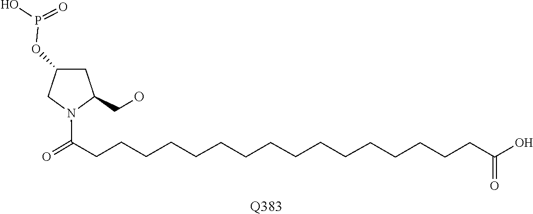

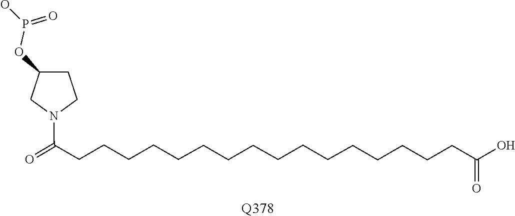

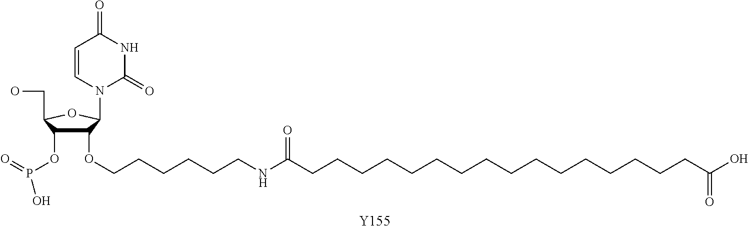

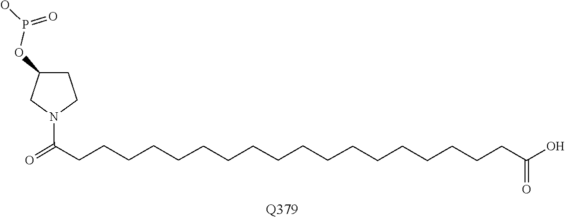

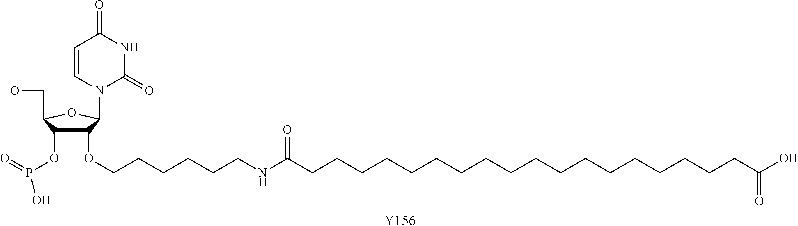

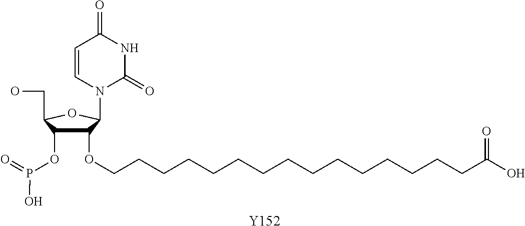

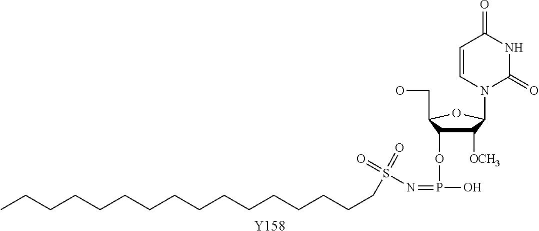

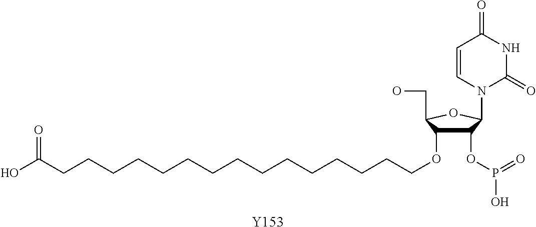

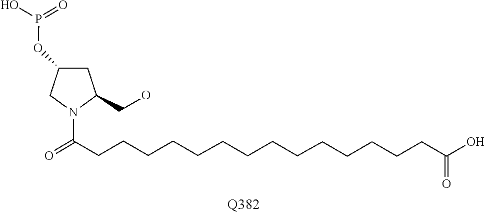

* Upper and lower case letters in italics indicate 2′-deoxy-2′-fluoro (2′-F), and 2′-O-methyl (2′-OMe) sugar modifications, respectively, to adenosine, cytidine, guanosine and uridine;
s indicates phosphorothioate (PS) linkage;
VP indicates vinyl phosphonate;
Nhd indicates 2′-O-hexadecyl.

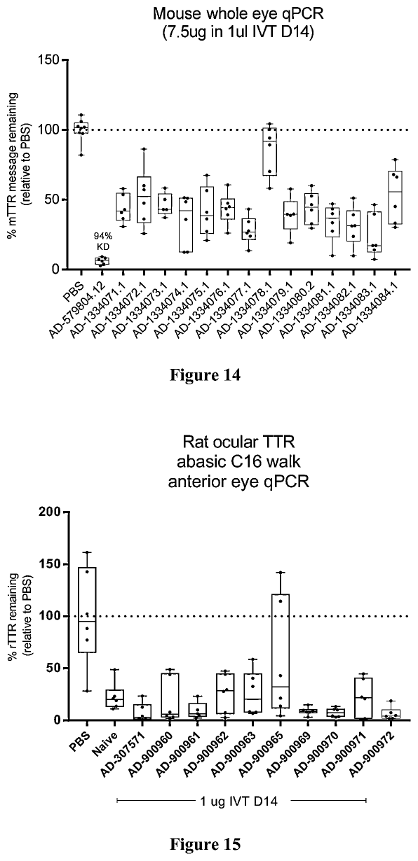

TTR gene silencing was also studied with siRNA conjugates listed in Table 13 by qPCR in mice eyes following intravitreal administration of a single 7.5 μg dose of siRNA duplexes, with the mice sacrificed on day 14, and the results were compared to PBS control. The results are shown in FIG. 14. The siRNA duplexes listed below were conjugated with multiple shorter lipid molecules.

TABLE 13
Lipophilic siRNA conjugates having multiple shorter lipid distributed along sense
and antisense strand of a TTR sequence

| | | | | | | Molecular |
|---|---|---|---|---|---|---|
| Duplex | | | | | Molecular | Weight |
| Id | Oligo Id | Strand | Target | Oligo Seq | Weight | Found |
| AD- | A- | sense | TTR | asascag(Uhd)GfuUfCfUfug | 7140.02 | 7136.26 |
| 579804 | 594427 | | | cucuausasa | | |
| | A- | antis | TTR | VPusUfsauaGfagcaagaAfc | 7732.11 | 7728.14 |
| | 555713 | | | Afcuguususu | | |
| AD- | A- | sense | TTR | (Ahs)ascag(Uh)GfuUfCfUf | 7210.16 | 7206.34 |
| 1334071 | 2219775 | | | ugcuc(Uh)ausas(Ah) | | |
| | A- | antis | TTR | VPusUfsauaGfagcaagaAfc | 7732.11 | 7728.14 |
| | 555713 | | | Afcuguususu | | |
| AD- | A- | sense | TTR | asascag(Uh)uGfuUfCfUfug | 7530.35 | 7526.38 |
| 1334072 | 2219776 | | | cu(Ch)(Uh)ausas(Ah) | | |
| | A- | antis | TTR | VPusUfsauaGfagcaagaAfc | 7732.11 | 7728.14 |
| | 555713 | | | Afcuguususu | | |
| AD- | A- | sense | TTR | asascag(Uh)(Gh)uUfCfUfu | 7222.19 | 7218.36 |
| 1334073 | 2219777 | | | g(Ch)(Uh)cuausasa | | |
| | A- | antis | TTR | VPusUfsauaGfagcaagaAfc | 7732.11 | 7728.14 |
| | 555713 | | | Afcuguususu | | |
| AD- | A- | sense | TTR | asascag(Uh)GfuUfCfUfug | 7140.02 | 7136.26 |
| 1334074 | 2219778 | | | (Ch)(Uh)cuausasa | | |
| | A- | antis | TTR | VPusUfsauaGfagcaagaAfc | 7732.11 | 7728.14 |
| | 555713 | | | Afcuguususu | | |
| AD- | A- | sense | TTR | asascag(Uh)GfuUfCfUfug( | 7140.02 | 7136.26 |
| 1334083 | 2219778 | | | Ch)(Uh)cuausasa | | |
| | A- | antis | TTR | VPuUfauaGfagcaagaAfc(A | 7782.15 | 7778.29 |
| | 2219785 | | | h)cuguususu | | |
| AD- | A- | sense | TTR | asascag(Uh)GfuUfCfUfug( | 7140.02 | 7136.26 |
| 1334084 | 2219778 | | | Ch)(Uh)cuausasa | | |
| | A- | antis | TTR | VPuUfauaGfagcaagaAfc(A | 7740.07 | 7736.24 |
| | 2219786 | | | pr)cuguususu | | |
| AD- | A- | sense | TTR | asascag(Uh)GfuUfCfUfugc | 7069.88 | 7066.18 |
| 1334075 | 2219779 | | | u(Ch)uausasa | | |
| | A- | antis | TTR | VPusUfsauaGfagcaagaAfc | 7732.11 | 7728.14 |
| | 555713 | | | Afcuguususu | | |
| AD- | A- | sense | TTR | asascag(Uh)GfuUfCfUfugc | 7069.88 | 7066.18 |
| 1334081 | 2219779 | | | u(Ch)uausasa | | |
| | A- | antis | TTR | VPuUfauaGfagcaagaAfc(A | 7782.15 | 7778.29 |
| | 2219785 | | | h)cuguususu | | |
| AD- | A- | sense | TTR | asascag(Uh)GfuUfCfUfugc | 7069.88 | 7066.18 |
| 1334082 | 2219779 | | | u(Ch)uausasa | | |
| | A- | antis | TTR | VPuUfauaGfagcaagaAfc(A | 7740.07 | 7736.24 |
| | 2219786 | | | pr)cuguususu | | |
| AD- | A- | sense | TTR | (Aprs)ascag(Upr)GfuUfCfU | 7041.83 | 7038.15 |
| 1334076 | 2219780 | | | fugcu(Cpr)(Upr)ausasa | | |
| | A- | antis | TTR | VPusUfsauaGfagcaagaAfc | 7732.11 | 7728.14 |
| | 555713 | | | Afcuguususu | | |
| AD- | A- | sense | TTR | (Aprs)ascag(Upr)GfuUfCfU | 7041.84 | 7038.15 |
| 1334077 | 2219781 | | | fugcuc(Upr)ausas(Apr) | | |
| | A- | antis | TTR | VPusUfsauaGfagcaagaAfc | 7732.11 | 7728.14 |
| | 555713 | | | Afcuguususu | | |
| AD- | A- | sense | TTR | (Aprs)ascag(Upr)GfuUfCfU | 7013.78 | 7010.12 |
| 1334078 | 2219782 | | | fugcuc(Upr)ausasa | | |
| | A- | antis | TTR | VPusUfsauaGfagcaagaAfc | 7732.11 | 7728.14 |
| | 555713 | | | Afcuguususu | | |
| AD- | A- | sense | TTR | (Aprs)ascag(Uh)GfuUfCfUf | 7083.91 | 7080.20 |
| 1334079 | 2219783 | | | ugcu(Cpr)(Upr)ausasa | | |
| | A- | antis | TTR | VPusUfsauaGfagcaagaAfc | 7732.11 | 7728.14 |
| | 555713 | | | Afcuguususu | | |
| AD- | A- | sense | TTR | (Aprs)ascag(Uh)GfuUfCfUf | 7097.94 | 7094.22 |
| 1334080 | 2219784 | | | ugcu(Ch)uausasa | | |
| | A- | antis | TTR | VPusUfsauaGfagcaagaAfc | 7732.11 | 7728.14 |
| | 555713 | | | Afcuguususu | | |

Symbols

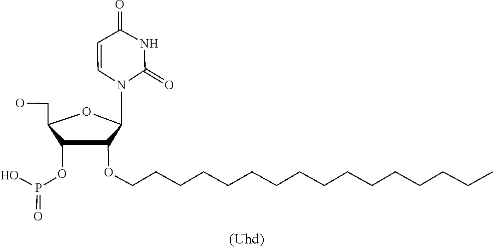

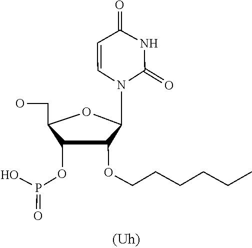

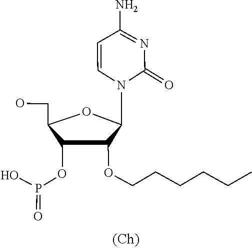

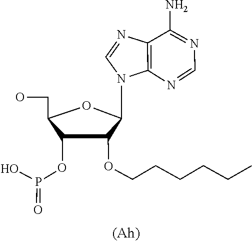

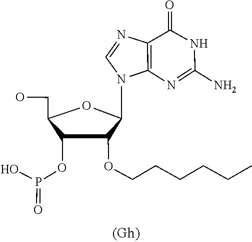

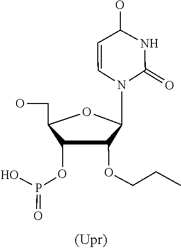

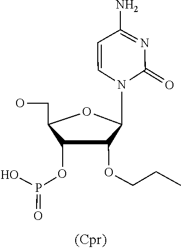

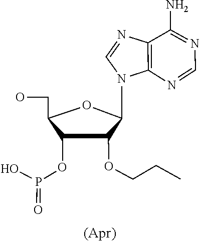

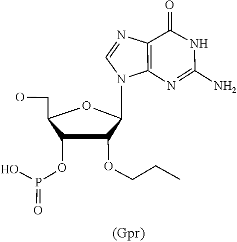

TTR gene silencing was also studied with siRNA conjugates listed in Table 14 by qPCR in rat eyes following intravitreal administration of a single 1 μg dose of siRNA duplexes, with the rat sacrificed on day 14, and the results were compared to PBS control. The results are shown in FIG. 15. The siRNA duplexes listed below were conjugated with esterase cleavable conjugates.

TABLE 14
Lipophilic siRNA conjugates for in vivo evaluation in rat (abasic walk)

| | | | | | | Molecular |
|---|---|---|---|---|---|---|
| Duplex | | | | | Molecular | Weight |
| Id | Oligo Id | Strand | Target | Oligo Seq | Weight | Found |
| AD- | A- | sense | TTR | asascag(Uhd)GfuUfCf | 7140.023 | 7136.262 |
| 307571 | 594427 | | | Ufugcucuausasa | | |
| | A- | antis | TTR | VPuUfauaGfagcaagaAf | 7699.985 | 7696.194 |
| | 555715 | | | cAfcuguususu | | |
| AD- | A- | sense | TTR | asascaQ367uGfuUfCfU | 6987.924 | 6984.228 |
| 900960 | 1700680 | | | fugcucuausasa | | |
| | A- | antis | TTR | VPusUfsauaGfagcaaga | 7732.116 | 7728.149 |
| | 555713 | | | AfcAfcuguususu | | |
| AD- | A- | sense | TTR | asascagQ367GfuUfCfU | 7026.963 | 7023.25 |
| 900961 | 1700681 | | | fugcucuausasa | | |
| | A- | antis | TTR | VPusUfsauaGfagcaaga | 7732.116 | 7728.149 |
| | 555713 | | | AfcAfcuguususu | | |
| AD- | A- | sense | TTR | asascaguQ367uUfCfUf | 6999.96 | 6996.248 |
| 900962 | 1700682 | | | ugcucuausasa | | |
| | A- | antis | TTR | VPusUfsauaGfagcaaga | 7732.116 | 7728.149 |
| | 555713 | | | AfcAfcuguususu | | |
| AD- | A- | sense | TTR | asascaguGfQ367UfCfU | 7026.963 | 7023.25 |
| 900963 | 1700683 | | | fugcucuausasa | | |
| | A- | antis | TTR | VPusUfsauaGfagcaaga | 7732.116 | 7728.149 |
| | 555713 | | | AfcAfcuguususu | | |
| AD- | A- | sense | TTR | asascaguGfuUfQ367Uf | 7039.984 | 7036.254 |
| 900965 | 1700685 | | | ugcucuausasa | | |
| | A- | antis | TTR | VPusUfsauaGfagcaaga | 7732.116 | 7728.149 |
| | 555713 | | | AfcAfcuguususu | | |
| AD- | A- | sense | TTR | asascaguGfuUfCfUfug | 7027.948 | 7024.234 |
| 900969 | 1700689 | | | Q367ucuausasa | | |
| | A- | antis | TTR | VPusUfsauaGfagcaaga | 7732.116 | 7728.149 |
| | 555713 | | | AfcAfcuguususu | | |
| AD- | A- | sense | TTR | asascaguGfuUfCfUfugc | 7026.963 | 7023.25 |
| 900970 | 1700690 | | | Q367cuausasa | | |
| | A- | antis | TTR | VPusUfsauaGfagcaaga | 7732.116 | 7728.149 |
| | 555713 | | | AfcAfcuguususu | | |
| AD- | A- | sense | TTR | asascaguGfuUfCfUfugc | 7027.948 | 7024.234 |
| 900971 | 1700691 | | | uQ367uausasa | | |
| | A- | antis | TTR | VPusUfsauaGfagcaaga | 7732.116 | 7728.149 |
| | 555713 | | | AfcAfcuguususu | | |
| AD- | A- | sense | TTR | asascaguGfuUfCfUfugc | 7026.963 | 7023.25 |

| | | |
|---|---|---|
| 900972 | 1700692 | ucQ367ausasa |

| | | | | | |
|---|---|---|---|---|---|
| A- | antis | TTR | VPusUfsauaGfagcaaga | 7732.116 | 7728.149 |

| | |
|---|---|
| 555713 | AfcAfcuguususu |

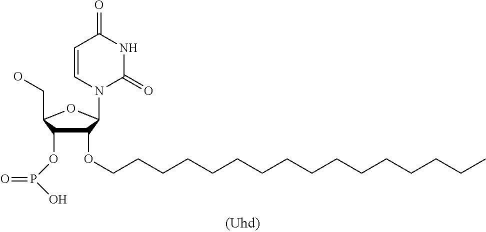

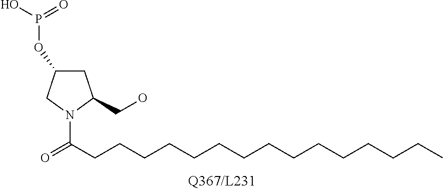

### Example 9. Positional Impact of Abasic Lipophilic Modification (Q367) Across the siRNA Sequence

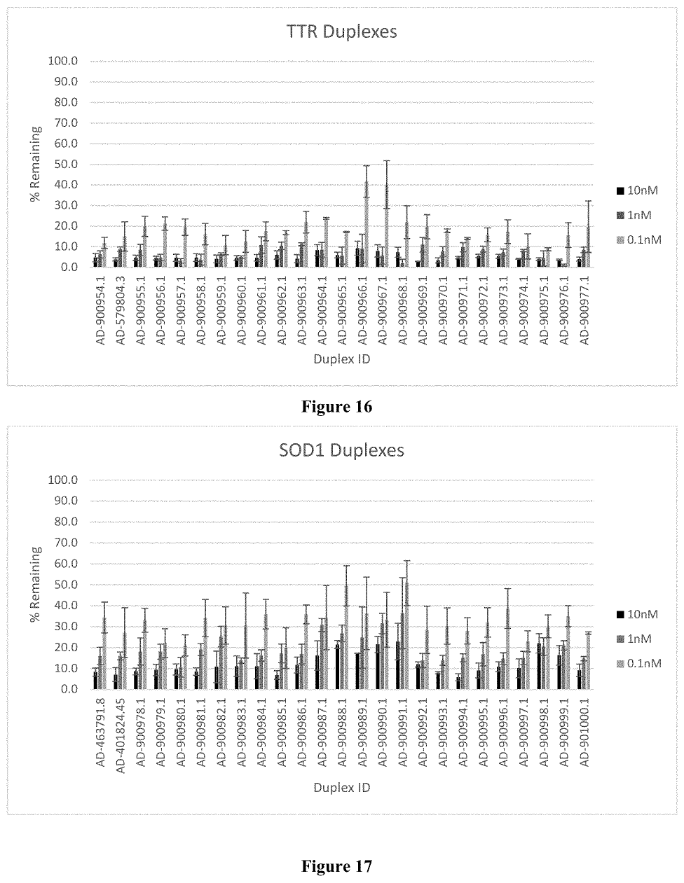

The effect of the position of the lipophilic modification across the entire siRNA sequence on the sense strand was evaluated in primary mouse hepatocytes using siRNA conjugates modified by Q367 ligand, as compared to the control duplex AD-900954 (shown in Table 15). Cells were incubated with each siRNA conjugate at 0.1, 1, and 10 nM concentrations for free uptake (without transfection agent) and TTR mRNA was measured after 24 hours. Values are plotted as a fraction of untreated control cells. The results are shown in FIG. 16.

TABLE 15
Abasic lipophilic ligand walk across the sense strand of a TTR sequence

| | | | | | | Molecular |
|---|---|---|---|---|---|---|
| Duplex | | | | | Molecular | Weight |
| Id | Oligo Id | Strand | Target | Oligo Seq | Weight | Found |
| AD- | A- | sense | TTR | asascaguGfuUfCfUfugcucu | 6929.626 | 6926.027 |
| 900954 | 331806 | | | ausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascag(Uhd)GfuUfCfUfug | 7140.023 | 7136.262 |
| 579804 | 594427 | | | cucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | Q3 67sasacaguGfuUfCfUfu | 7347.151 | 7343.291 |
| 900955 | 1700675 | | | gcucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | Q3 67sascaguGfuUfCfUfug | 7003.918 | 7000.223 |
| 900956 | 1700676 | | | cucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asQ3 67scaguGfuUfCfUfug | 7003.918 | 7000.223 |
| 900957 | 1700677 | | | cucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asasQ367aguGfuUfCfUfug | 7027.948 | 7024.234 |
| 900958 | 1700678 | | | cucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascQ367guGfuUfCfUfug | 7003.923 | 7000.223 |
| 900959 | 1700679 | | | cucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaQ367uGfuUfCfUfugc | 6987.924 | 6984.228 |
| 900960 | 1700680 | | | ucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascagQ367GfuUfCfUfugc | 7026.963 | 7023.25 |
| 900961 | 1700681 | | | ucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguQ367uUfCfUfugcu | 6999.96 | 6996.248 |
| 900962 | 1700682 | | | cuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfQ367UfCfUfugc | 7026.963 | 7023.25 |
| 900963 | 1700683 | | | ucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuQ367CfUfugcu | 7038.999 | 7035.27 |
| 900964 | 1700684 | | | cuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfQ3 67Ufugcu | 7039.984 | 7036.254 |
| 900965 | 1700685 | | | cuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfQ3 67ugcu | 7038.999 | 7035.27 |
| 900966 | 1700686 | | | cuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfQ367gc | 7026.963 | 7023.25 |
| 900967 | 1700687 | | | ucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfuQ367c | 6987.924 | 6984.228 |
| 900968 | 1700688 | | | ucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfugQ36 | 7027.948 | 7024.234 |
| 900969 | 1700689 | | | 7ucuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfugcQ3 6 | 7026.963 | 7023.25 |
| 900970 | 1700690 | | | 7cuausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfugcuQ3 | 7027.948 | 7024.234 |
| 900971 | 1700691 | | | 67uausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfugcucQ | 7026.963 | 7023.25 |
| 900972 | 1700692 | | | 367ausasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfugcucu | 7003.923 | 7000.223 |
| 900973 | 1700693 | | | Q367usasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfugcucu | 7026.958 | 7023.25 |
| 900974 | 1700694 | | | aQ367sasa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfugcucu | 7003.918 | 7000.223 |
| 900975 | 1700695 | | | ausQ367sa | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfugcucu | 7003.923 | 7000.223 |
| 900976 | 1700696 | | | ausasQ367 | | |
| | A- | antis | TTR | VPusUfsauaGfagcaagaAfc | 7732.116 | 7728.149 |
| | 555713 | | | Afcuguususu | | |
| AD- | A- | sense | TTR | asascaguGfuUfCfUfugcucu | 8109.006 | 8104.708 |
| 900977 | 1700697 | | | auasasL231 | | |
| | A- | antis | TTR | VPusUfsauaGfagcaagaAfc | 7732.116 | 7728.149 |
| | 555713 | | | Afcuguususu | | |

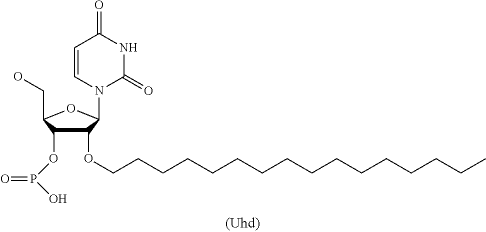

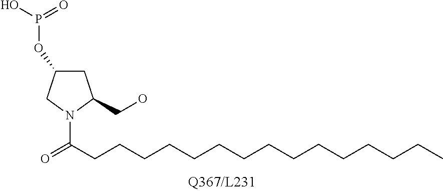

* Upper and lower case letters in italics indicate 2′-deoxy-2′-fluoro (2′-F), and 2′-O-methyl (2′-OMe) sugar modifications, respectively, to adenosine, cytidine, guanosine and uridine;
s indicates phosphorothioate (PS) linkage;
VP indicates vinyl phosphonate;
Nhd indicates 2′-O-hexadecyl.

The effect of the position of the lipophilic modification across the entire SOD1 siRNA sequence on the sense strand was also evaluated in primary mouse hepatocytes using siRNA conjugates modified by Q367 ligand, as compared to the control duplex AD-463791 (shown in Table 16). Cells were incubated with each siRNA conjugate at 0.1, 1, and 10 nM concentrations for free uptake (without transfection agent) and SOD1 mRNA was measured after 24 hours. Values are plotted as a fraction of untreated control cells. The results are shown in FIG. 17.

TABLE 16
Abasic lipophilic ligand walk across a sense strand of a SOD1 sequence

| | | | | | | Molecular |
|---|---|---|---|---|---|---|
| Duplex | | | | | Molecular | Weight |
| Id | Oligo Id | strand | Target | Oligo Seq | Weight | Found |
| AD- | A- | sense | SOD1 | csasuuu(Uhd)AfaUfCfCfuc | 7043.97 | 7040.25 |
| 401824 | 637448 | | | acucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfucacuc | 6833.57 | 6830.02 |
| 463791 | 899929 | | | uasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | Q367scsauuuuAfaUfCfCfuc | 7251.10 | 7247.28 |
| 900978 | 1700698 | | | acucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | Q367sasuuuuAfaUfCfCfuca | 6931.89 | 6928.22 |
| 900979 | 1700699 | | | cucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csQ367suuuuAfaUfCfCfuca | 6907.87 | 6904.21 |
| 900980 | 1700700 | | | cucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasQ367uuuAfaUfCfCfuca | 6930.91 | 6927.24 |
| 900981 | 1700701 | | | cucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuQ367uuAfaUfCfCfuca | 6930.91 | 6927.24 |
| 900982 | 1700702 | | | cucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuQ367uAfaUfCfCfuca | 6930.91 | 6927.24 |
| 900983 | 1700703 | | | cucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuQ367AfaUfCfCfuca | 6930.91 | 6927.24 |
| 900984 | 1700704 | | | cucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuQ367aUfCfCfucac | 6919.91 | 6916.23 |
| 900985 | 1700705 | | | ucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfQ367UfCfCfuca | 6907.87 | 6904.21 |
| 900986 | 1700706 | | | cucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaQ367CfCfucac | 6942.95 | 6939.26 |
| 900987 | 1700707 | | | ucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfQ367Cfucac | 6943.93 | 6940.24 |
| 900988 | 1700708 | | | ucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfQ367ucac | 6943.93 | 6940.24 |
| 900989 | 1700709 | | | ucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfQ367ca | 6930.91 | 6927.24 |
| 900990 | 1700710 | | | cucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfuQ367a | 6931.90 | 6928.22 |
| 900991 | 1700711 | | | cucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfucQ367 | 6907.87 | 6904.21 |
| 900992 | 1700712 | | | cucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfucaQ | 6931.90 | 6928.22 |
| 900993 | 1700713 | | | 367ucuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfucacQ | 6930.91 | 6927.24 |
| 900994 | 1700714 | | | 367cuasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfucacuQ | 6931.90 | 6928.22 |
| 900995 | 1700715 | | | 367uasasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfucacuc | 6930.91 | 6927.24 |
| 900996 | 1700716 | | | Q367asasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfucacuc | 6907.87 | 6904.21 |
| 900997 | 1700717 | | | uQ367sasa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfucacuc | 6907.87 | 6904.21 |
| 900998 | 1700718 | | | uasQ367sa | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfucacuc | 6907.87 | 6904.21 |
| 900999 | 1700719 | | | uasasQ367 | | |
| | A- | antis | SOD1 | VPusUfsuagAfgUfGfaggaU | 7851.15 | 7847.15 |
| | 444402 | | | fuAfaaaugsasg | | |
| AD- | A- | sense | SOD1 | csasuuuuAfaUfCfCfucacuc | 8012.95 | 8008.70 |
| 901000 | 1700720 | | | uaasasL231 | | |
| | A- | antis | SOD1 | VPusUfsuagAfgUfGfaggaU | 7851.15 | 7847.15 |

| | |
|---|---|
| 444402 | fuAfaaaugsasg |

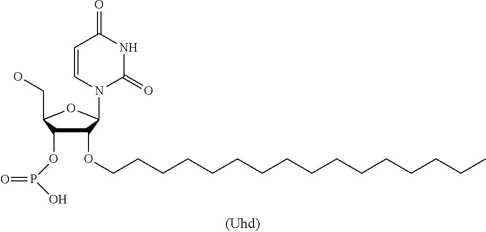

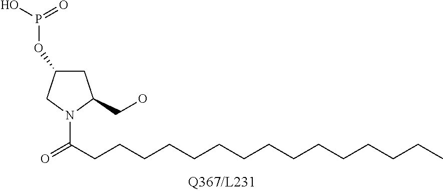

* Upper and lower case letters in italics indicate 2′-deoxy-2′-fluoro (2′-F), and 2′-O-methyl (2′-OMe) sugar modifications, respectively, to adenosine, cytidine, guanosine and uridine;
s indicates phosphorothioate (PS) linkage;
VP indicates vinyl phosphonate.

### Example 10. mRNA Knockdown in CNS Using Lipophilic Conjugated siRNAs

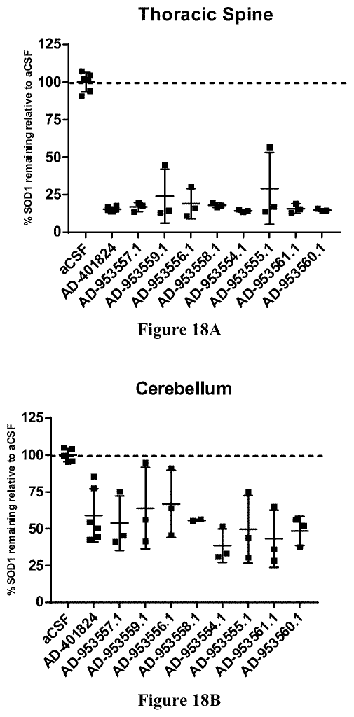

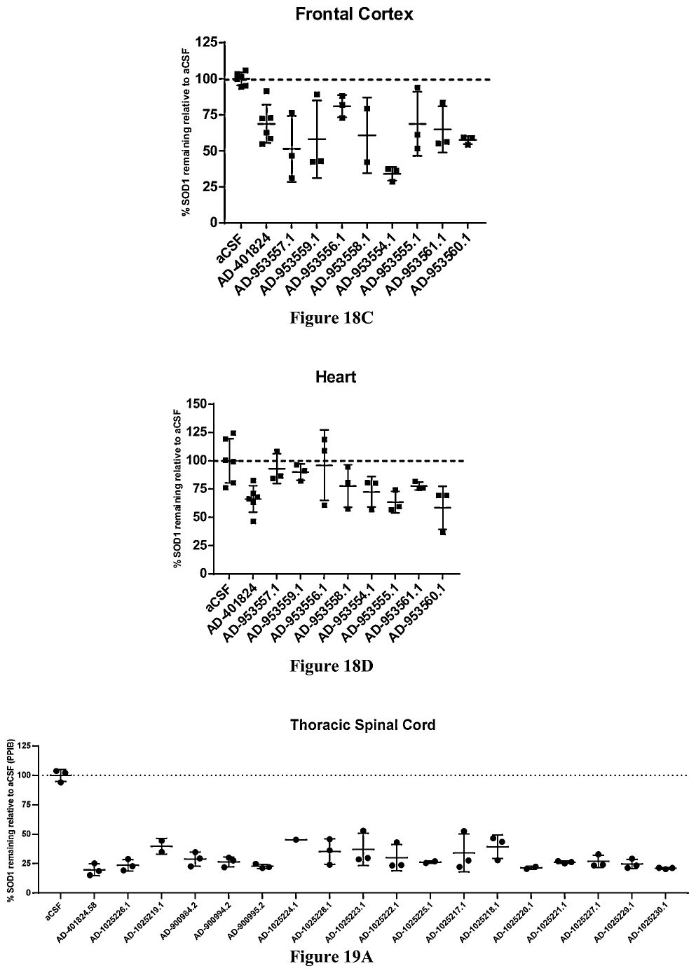

SOD6 gene silencing was studied with siRNA conjugates listed in Table 17 by qPCR in rat brain (cerebellum and frontal cortex), spinal cord (thoracic spinal cord), and heart following intrathecal administration of a single 0.9 mg dose of siRNA duplexes, with the rat sacrificed on day 14, and the results were compared to artificial CSF dosed control. The results are shown in FIG. 18.

TABLE 17
Lipophilic siRNA conjugates of SOD1 sequence (5′, 3′ and internal)

| | | | | | | Molecular |
|---|---|---|---|---|---|---|
| | | | | | Molecular | Weight |
| Duplex Id | Oligo Id | Strand | Target | Oligo Seq | Weight | found |
| AD- | A-637448 | sense | SOD1 | csasuuu(Uhd)AfaUfCf | 7043.97 | 7040.25 |
| 401824 | | | | Cfucacucuasasa | | |
| | A-444402 | antis | SOD1 | VPusUfsuagAfgUfGfa | 7851.15 | 7847.15 |
| | | | | ggaUfuAfaaaugsasg | | |
| AD- | A- | sense | SOD1 | Q361scsauuuuAfaUfCf | 7164.96 | 7161.21 |
| 953557 | 1700497 | | | Cfucacucuasasa | | |
| | A-444402 | antis | SOD1 | VPusUfsuagAfgUfGfa | 7851.15 | 7847.15 |
| | | | | ggaUfuAfaaaugsasg | | |
| AD- | A- | sense | SOD1 | Q362scsauuuuAfaUfCf | 7193.02 | 7189.24 |
| 953559 | 1700498 | | | Cfucacucuasasa | | |
| | A-444402 | antis | SOD1 | VPusUfsuagAfgUfGfa | 7851.15 | 7847.15 |
| | | | | ggaUfuAfaaaugsasg | | |
| AD- | A- | sense | SOD1 | Q363scsauuuuAfaUfCf | 7221.07 | 7217.27 |
| 953556 | 1700499 | | | Cfucacucuasasa | | |
| | A-444402 | antis | SOD1 | VPusUfsuagAfgUfGfa | 7851.15 | 7847.15 |
| | | | | ggaUfuAfaaaugsasg | | |
| AD- | A- | sense | SOD1 | Q364scsauuuuAfaUfCf | 7249.13 | 7245.30 |
| 953558 | 1700500 | | | Cfucacucuasasa | | |
| | A-444402 | antis | SOD1 | VPusUfsuagAfgUfGfa | 7851.15 | 7847.15 |
| | | | | ggaUfuAfaaaugsasg | | |
| AD- | A- | sense | SOD1 | Q365scsauuuuAfaUfCf | 7247.11 | 7243.28 |
| 953554 | 1700501 | | | Cfucacucuasasa | | |
| | A-444402 | antis | SOD1 | VPusUfsuagAfgUfGfa | 7851.15 | 7847.15 |
| | | | | ggaUfuAfaaaugsasg | | |
| AD- | A- | sense | SOD1 | Q366scsauuuuAfaUfCf | 7305.23 | 7301.36 |
| 953555 | 1700502 | | | Cfucacucuasasa | | |
| | A-444402 | antis | SOD1 | VPusUfsuagAfgUfGfa | 7851.15 | 7847.15 |
| | | | | ggaUfuAfaaaugsasg | | |
| AD- | A- | sense | SOD1 | csasuuuuAfaUfCfCfuc | 7251.10 | 7247.28 |
| 953561 | 1700503 | | | acucuaasasL321 | | |
| | A-444402 | antis | SOD1 | VPusUfsuagAfgUfGfa | 7851.15 | 7847.15 |
| | | | | ggaUfuAfaaaugsasg | | |
| AD- | A- | sense | SOD1 | csasuuuuAfaUfCfCfuc | 7364.25 | 7360.36 |
| 953560 | 1700504 | | | acucuaasasL322 | | |
| | A-444402 | antis | SOD1 | VPusUfsuagAfgUfGfa | 7851.15 | 7847.15 |
| | | | | ggaUfuAfaaaugsasg | | |

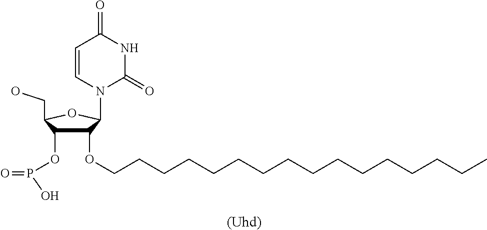

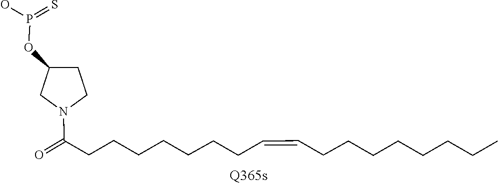

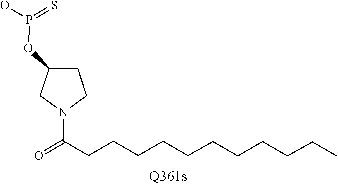

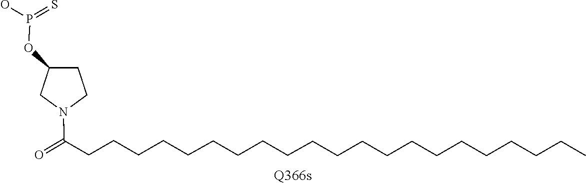

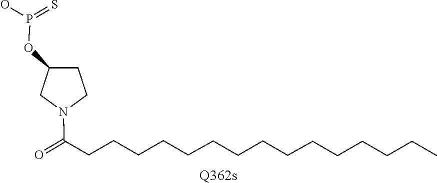

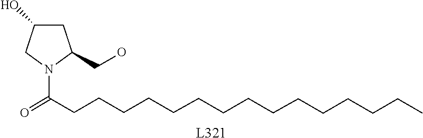

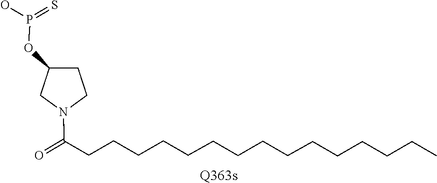

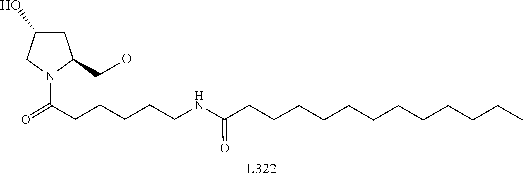

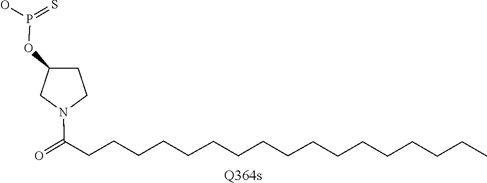

Upper and lower case letters in italics indicate 2′-deoxy-2′-fluoro (2′-F), and 2′-O-methyl (2′-OMe) sugar modifications, respectively, to adenosine, cytidine, guanosine and uridine;
s indicates phosphorothioate (PS) linkage;
VP indicates vinyl phosphonate.

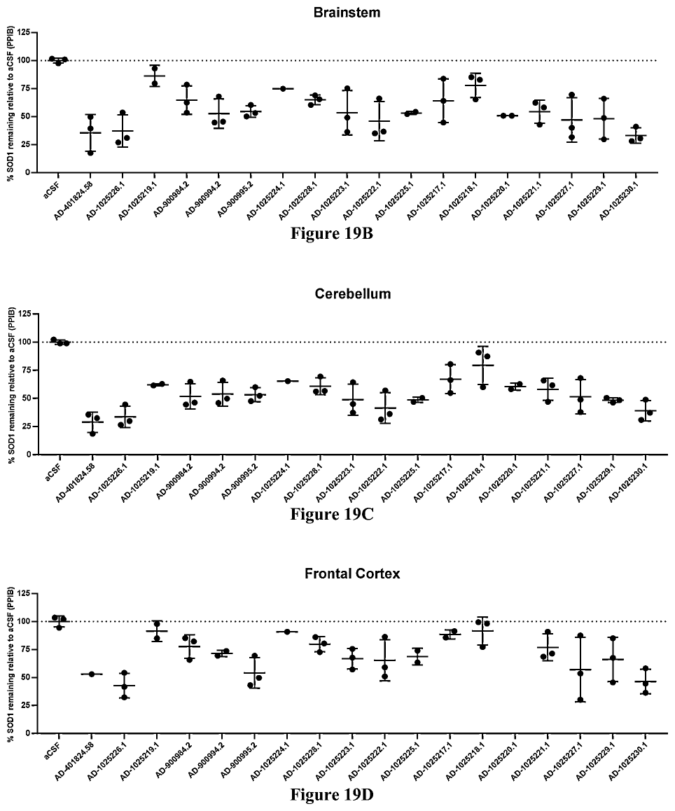

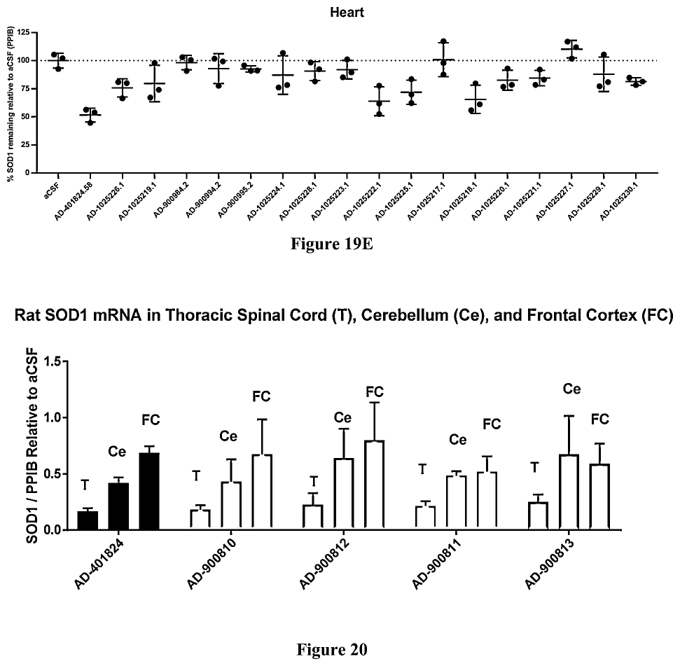

SOD1 gene silencing was also studied with siRNA conjugates listed in Table 18 by qPCR in rat brain (brain stem, cerebellum and frontal cortex), spinal cord (thoracic spinal cord), and heart following intrathecal administration of a single 0.9 mg dose of siRNA duplexes, with the rat sacrificed on day 14, and the results were compared to artificial CSF dosed control. The results are shown in FIG. 19.

TABLE 18
Lipophilic siRNA conjugates of SOD1 sequence for rat study

| Duplex | | | | | | Molecular |
|---|---|---|---|---|---|---|
| Id | Oligo Id | Strand | Target | OligoSeq | Molecular Weight | Weight Found |
| AD- | A- | sense | SOD1 | csasuuu(Uhd)AfaUfCfCfuc | 7043.97 | 7040.25 |
| 401824 | 637448 | | | acucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | csasuuuQ367AfaUfCfCfuc | 6930.91 | 6927.24 |
| 900984 | 1700704 | | | acucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | csasuuuuAfaUfCfCfucacQ | 6930.91 | 6927.24 |
| 900994 | 1700714 | | | 367cuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | csasuuuuAfaUfCfCfucacu | 6931.90 | 6928.22 |
| 900995 | 1700715 | | | Q367uasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | csasuuuY152AfaUfCfCfuc | 7073.96 | 7070.22 |
| 1025226 | 1866829 | | | acucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | csasuuuY154AfaUfCfCfuc | 7187.12 | 7183.31 |
| 1025223 | 1866830 | | | acucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | csasuuuY155AfaUfCfCfuc | 7215.17 | 7211.34 |
| 1025222 | 1866831 | | | acucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | csasuuuY156AfaUfCfCfuc | 7243.23 | 7239.37 |
| 1025225 | 1866832 | | | acucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | csasuuuQ382AfaUfCfCfuc | 6960.89 | 6957.21 |
| 1025224 | 1866833 | | | acucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | csasuuuQ383AfaUfCfCfuc | 6988.94 | 6985.24 |
| 1025228 | 1866834 | | | acucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | Q377scsauuuuAfaUfCfCfu | 7251.05 | 7247.24 |
| 1025227 | 1866835 | | | cacucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | Q378scsauuuuAfaUfCfCfu | 7279.11 | 7275.27 |
| 1025229 | 1866836 | | | cacucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | Q379scsauuuuAfaUfCfCfu | 7307.16 | 7303.31 |
| 1025230 | 1866837 | | | cacucuasasa | | |
| | A- | antis | SOD1 | VPusUfsuagAfgUfGfagga | 7851.15 | 7847.15 |
| | 444402 | | | UfuAfaaaugsasg | | |
| AD- | A- | sense | SOD1 | Q363sgscaaagGfuGfGfAfa | 7591.42 | 7587.45 |
| 1025217 | 1875194 | | | augaagasasa | | |
| | A- | antis | SOD1 | VPusUfsucuUfcAfUfuucc | 7446.77 | 7442.97 |
| | 1287139 | | | AfcCfuuugcscsc | | |
| AD- | A- | sense | SOD1 | Q363sasaagguGfgAfAfAfu | 7615.45 | 7611.46 |
| 1025220 | 1875195 | | | gaagaaasgsa | | |
| | A- | antis | SOD1 | VPusCfsuuuCfuUfCfauuu | 7447.76 | 7443.95 |
| | 1287141 | | | CfcAfccuuusgsc | | |
| AD- | A- | sense | SOD1 | Q363sgsacuugGfgCfAfAfa | 7560.36 | 7556.41 |
| 1025218 | 1875196 | | | gguggaasasa | | |
| | A- | antis | SOD1 | VPusUfsuucc(Agn)ccuuug | 7496.92 | 7493.07 |
| | 1136073 | | | CfcCfaagucsasu | | |
| AD- | A- | sense | SOD1 | Q363sasggaugAfaGfAfGfa | 7561.35 | 7557.39 |
| 1025221 | 1875197 | | | ggcaugususa | | |
| | A- | antis | SOD1 | VPusAfsacaUfgCfCfucucU | 7493.84 | 7490.01 |
| | 1286811 | | | fuCfauccususu | | |
| AD- | A- | sense | SOD1 | csasuuQ367uAfaUfCfCfuc | 6587.68 | 6584.17 |
| 1025219 | 1875239 | | | acucusasa | | |
| | A- | antis | SOD1 | VPusUfsuagAfgUfGfagga | 7851.15 | 7847.15 |
| | 444402 | | | UfuAfaaaugsasg | | |

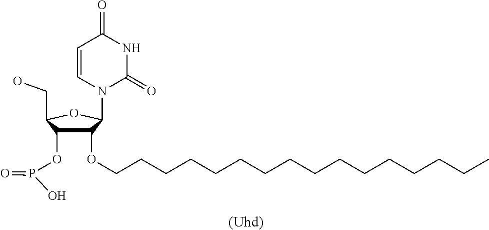

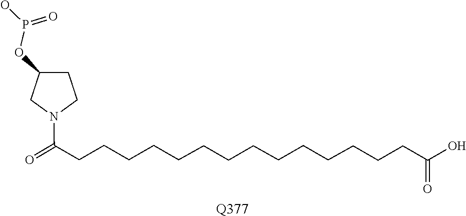

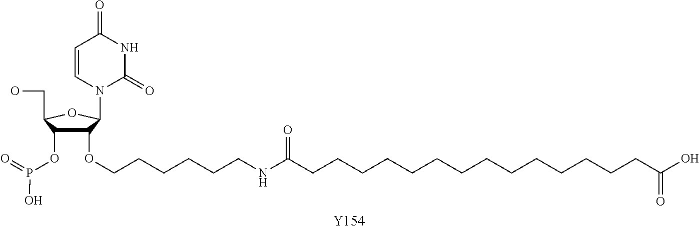

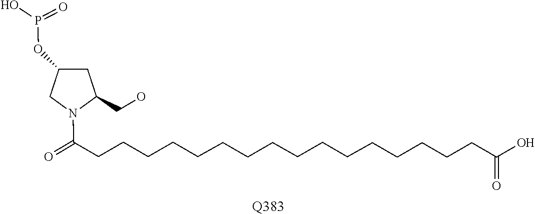

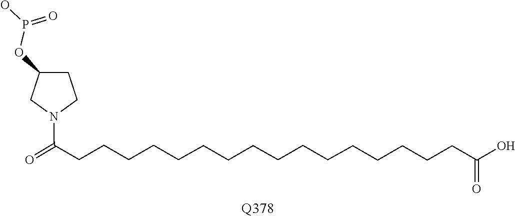

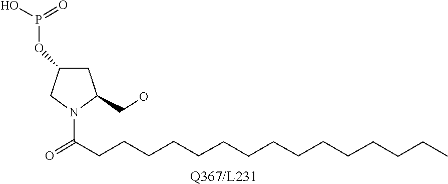

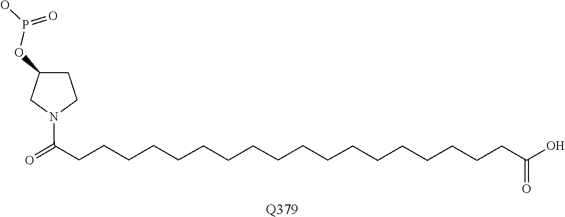

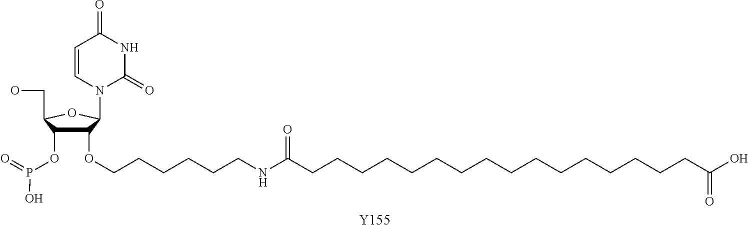

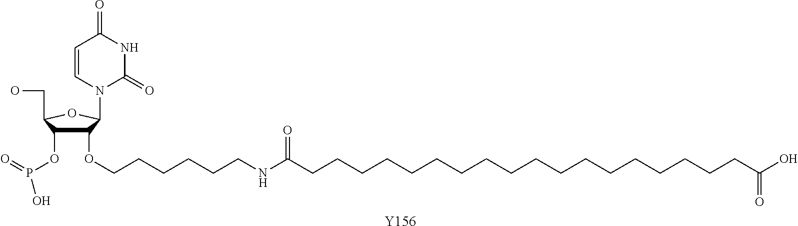

Upper and lower case letters in italics indicate 2′-deoxy-2′-fluoro (2′-F), and 2′-O-methyl (2′-OMe) sugar modifications, respectively, to adenosine, cytidine, guanosine and uridine;
s indicates phosphorothioate (PS) linkage;
VP indicates vinyl phosphonate.

SOD1 gene silencing was also studied with siRNA conjugates listed in Table 19 by qPCR in rat brain (cerebellum and frontal cortex), spinal cord (thoracic spinal cord), and heart following intrathecal administration of a single 0.9 mg dose of siRNA duplexes, with the rat sacrificed on day 7, and the results were compared to artificial CSF dosed control. The results are shown in FIG. 20.

TABLE 19
Esterase cleavable lipophilic siRNA conjugates of SOD1 sequence

Molecular

| Duplex | Oligo | | | | Molecular | Weight |
|---|---|---|---|---|---|---|
| Id | Id | Strand | Target | Oligo Seq | Weight | Found |
| AD- | A- | sense | SOD1 | csasuuu(Uhd)AfaUfCfCfuca | 7043.97 | 7040.25 |
| 401824 | 637448 | | | cucuasasa | | |
| | A- | antis | SOD1 | VPusUfsuagAfgUfGfaggaUf | 7851.15 | 7847.15 |
| | 444402 | | | uAfaaaugsasg | | |
| AD- | A- | sense | SOD1 | csasuuuY132AfaUfCfCfucac | 7229.20 | 7225.36 |
| 900813 | 154301 | | | ucuasasa | | |
| | 9 | | | | | |
| | A- | antis | SOD1 | VPusUfsuagAfgUfGfaggaUf | 7851.15 | 7847.15 |
| | 444402 | | | uAfaaaugsasg | | |
| AD- | A- | sense | SOD1 | csasuuuY133AfaUfCfCfucac | 7257.26 | 7253.39 |
| 900810 | 154302 | | | ucuasasa | | |
| | 0 | | | | | |
| | A- | antis | SOD1 | VPusUfsuagAfgUfGfaggaUf | 7851.15 | 7847.15 |
| | 444402 | | | uAfaaaugsasg | | |
| AD- | A- | sense | SOD1 | csasuuuY134AfaUfCfCfucac | 7215.18 | 7211.34 |
| 900811 | 154302 | | | ucuasasa | | |
| | 1 | | | | | |
| | A- | antis | SOD1 | VPusUfsuagAfgUfGfaggaUf | 7851.15 | 7847.15 |
| | 444402 | | | uAfaaaugsasg | | |
| AD- | A- | sense | SOD1 | csasuuuY135AfaUfCfCfucac | 7243.23 | 7239.37 |
| 900812 | 154302 | | | ucuasasa | | |
| | 2 | | | | | |
| | A- | antis | SOD1 | VPusUfsuagAfgUfGfaggaUf | 7851.15 | 7847.15 |
| | 444402 | | | uAfaaaugsasg | | |

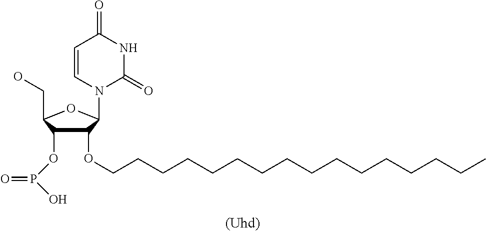

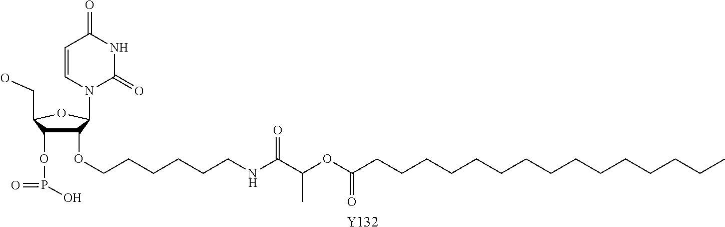

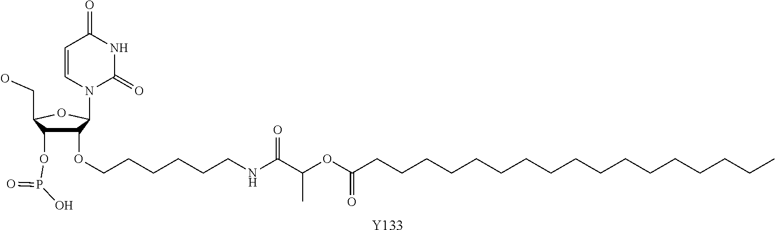

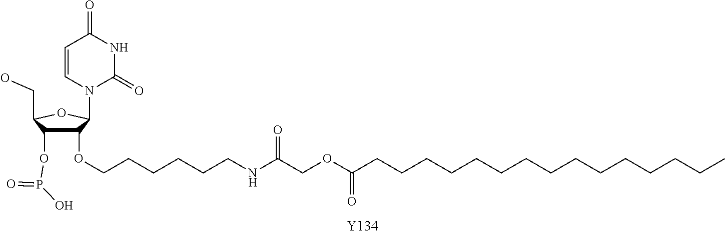

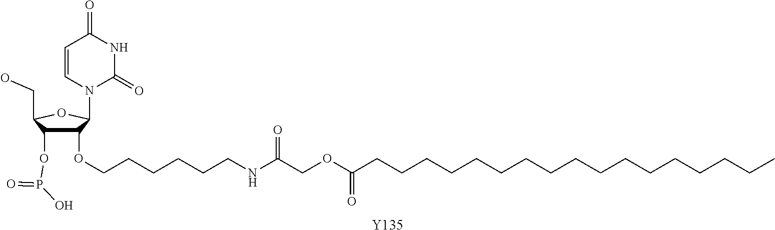

Upper and lower case letters in italics indicate 2′-deoxy-2′-fluoro (2′-F), and 2′-O-methyl (2′-OMe) sugar modifications, respectively, to adenosine, cytidine, guanosine and uridine;
s indicates phosphorothioate (PS) linkage;
VP indicates vinyl phosphonate.

SOD1 gene silencing was also studied with siRNA conjugates listed in Table 20 by qPCR in mice brain and heart following ICV administration of a single 50 or 150 ug dose of siRNA duplexes, with the mice sacrificed on day 14 or day 7, and the results were compared to artificial CSF dosed control. The results are shown in FIG. 21.

TABLE 20
Lipophilic siRNA conjugates of SOD1 sequence for mice ICV experiment

| Duplex | | | | | Molecular | Exact |
|---|---|---|---|---|---|---|
| Id | Oligo Id | strand | target | oligoSeq | Weight | Mass |
| AD- | A- | sense | SOD1 | csasuuu(Uhd)AfaUfCfCfuca | 7043.976 | 7040.254 |
| 401824 | 637448 | | | cucuasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | csasuuu(Uh)AfaUfCfCfucac | 7043.973 | 7040.261 |
| 1321422 | 2219765 | | | (Uh)(Ch)uasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | csasuuu(Uh)AfaUfCfCfucac | 7043.978 | 7040.256 |
| 1321423 | 2219766 | | | (Uh)cuasas(Ah) | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | (Chs)asuuu(Uh)AfaUfCfCfu | 7043.975 | 7040.254 |
| 1321424 | 2219767 | | | cac(Uh)cuasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | (Chs)asuuu(Uh)AfaUfCfCfu | 7114.113 | 7110.331 |
| 1321425 | 2219768 | | | cac(Uh)cuasas(Ah) | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | (Cprs)asuuu(Upr)AfaUfCfC | 6973.847 | 6970.175 |
| 1321426 | 2219769 | | | fuca(Cpr)(Upr)cuasas(Apr) | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | csasuuu(Uh)AfaUfCfCfucac | 6973.84 | 6970.182 |
| 1321427 | 2219770 | | | u(Ch)uasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | csasuuu(Uh)AfaUfCfCfucac | 6973.84 | 6970.182 |
| 1321431 | 2219770 | | | u(Ch)uasasa | | |
| | A- | antis | SOD1 | VPusUfsuagAfgUfGfaggaU | 7933.328 | 7929.252 |
| | 2219774 | | | fu(Ah)aaaugsasg | | |
| AD- | A- | sense | SOD1 | csasuuu(Uh)AfaUfCfCfucac | 6973.84 | 6970.178 |
| 1321428 | 2219771 | | | (Uh)cuasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | csasuuu(Uh)AfaUfCfCfucac | 6973.84 | 6970.178 |
| 1321432 | 2219771 | | | (Uh)cuasasa | | |
| | A- | antis | SOD1 | VPusUfsuagAfgUfGfaggaU | 7933.328 | 7929.252 |
| | 2219774 | | | fu(Ah)aaaugsasg | | |
| AD- | A- | sense | SOD1 | (Chs)asuuu(Uh)AfaUfCfCfu | 7184.246 | 7180.415 |
| 1321429 | 2219772 | | | cac(Uh)(Ch)uasas(Ah) | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | csasuuu(Uh)AfaUfCfCfucac | 7001.896 | 6998.209 |
| 1321430 | 2219773 | | | (Uh)(Cpr)uasasa | | |
| | A- | antis | SOD1 | VPusUfsuagAfgUfGfaggaU | 7851.156 | 7847.154 |
| | 444402 | | | fuAfaaaugsasg | | |
| AD- | A- | sense | SOD1 | csasuuu(Uh)AfaUfCfCfucac | 7001.896 | 6998.209 |
| 1321433 | 2219773 | | | (Uh)(Cpr)uasasa | | |
| | A- | antis | SOD1 | VPusUfsuagAfgUfGfaggaU | 7933.328 | 7929.252 |
| | 2219774 | | | fu(Ah)aaaugsasg | | |

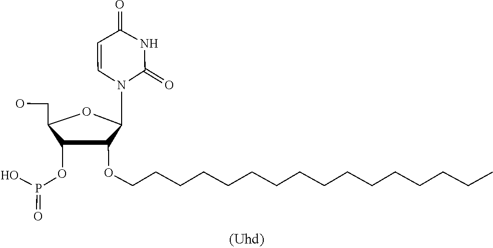

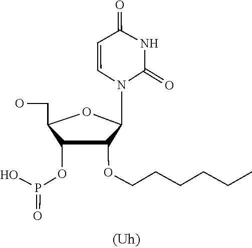

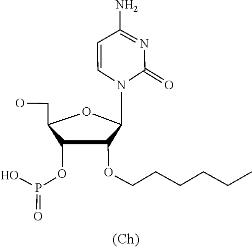

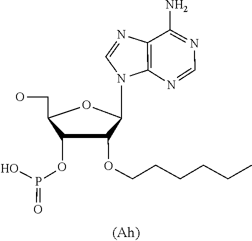

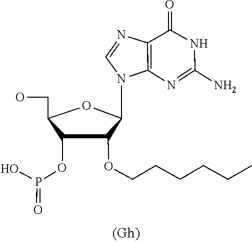

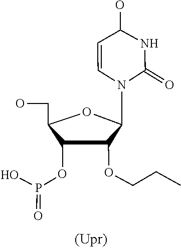

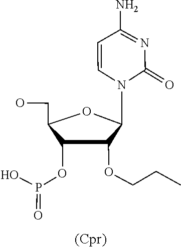

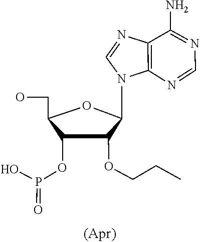

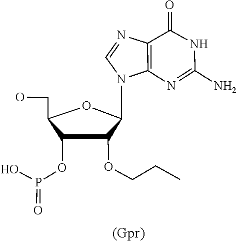

1. A compound comprising:
an antisense strand which is complementary to a target gene;
a sense strand which is complementary to said antisense strand; and
one or more lipophilic monomers, wherein the lipophilic monomer is selected from the group consisting of: 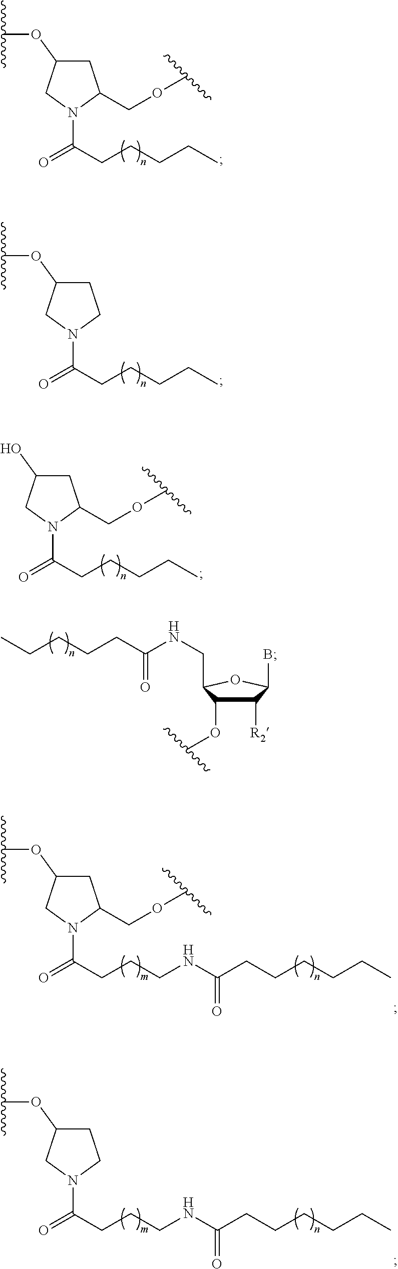 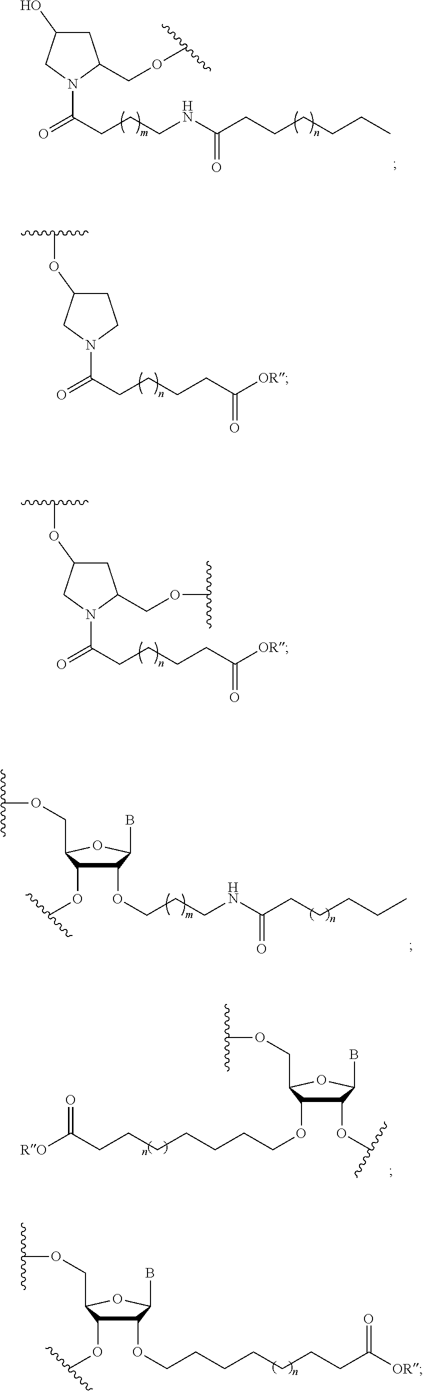 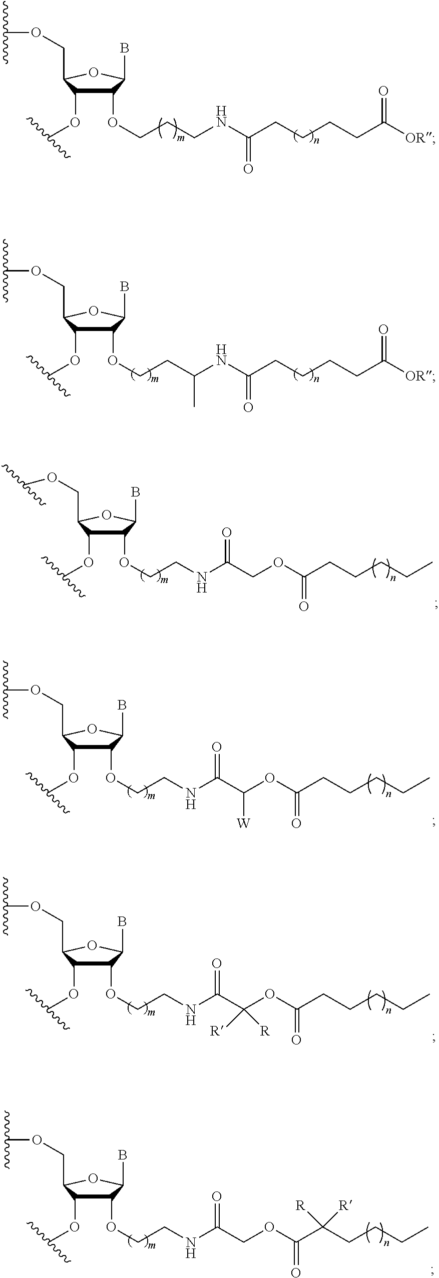 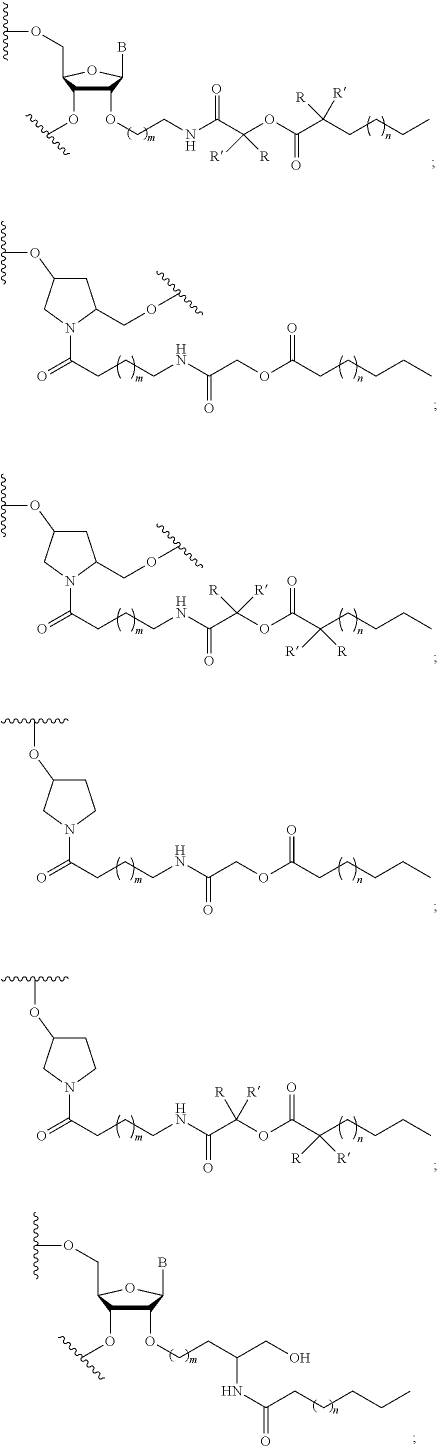 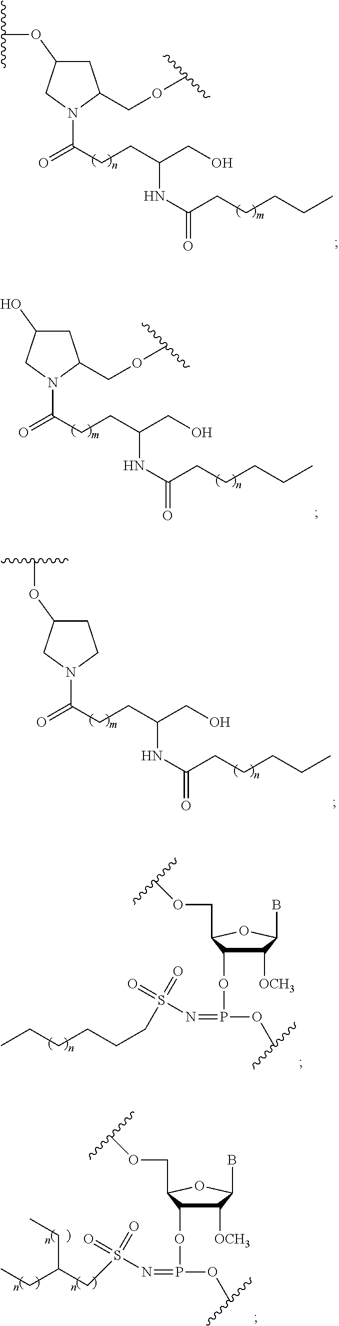 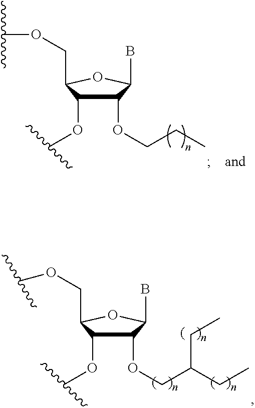
wherein:
m is an integer of 0-8;
n is an integer of 1-21;
R₂′ is H, OH, F, OMe, O-methoxyalkyl, O-allyl, O—N-methylacetamido, O-dimethylaminoethoxyethyl, or O-aminopropyl;
B is a modified or unmodified nucleobase;
W is an alkyl group; and
R, R′, and R″ are each independently H or an alkyl group.

2. The compound of claim 0, wherein said sense and antisense strands are each 15 to 30 nucleotides in length.

3. The compound of claim 0, wherein said sense and antisense strands are each 19 to 25 nucleotides in length.

4. The compound of claim 0, wherein said sense and antisense strands are each 21 to 23 nucleotides in length.

5. The compound of claim 4, wherein the sense strand is 21 nucleotides in length, and the antisense strand is 23 nucleotides in length, wherein the strands form a double-stranded region of 21 consecutive base pairs having a 2-nucleotide long single-stranded overhangs at the 3′-end.

6. The compound of claim 0, wherein said compound comprises a single-stranded overhang on at least one of the termini.

7. The compound of claim 6, wherein said single-stranded overhang is 1, 2 or 3 nucleotides in length.

8. The compound of claim 1, wherein the sense and the antisense strands comprise less than ten 2′-fluoro modified nucleotides.

9. The compound of claim 1, wherein the sense and antisense strands comprise at least 50%, at least 60%, or least 70% of 2′-OMe modified nucleotides.

10. The compound of claim 1, wherein the lipophilic monomer is selected from the group consisting of 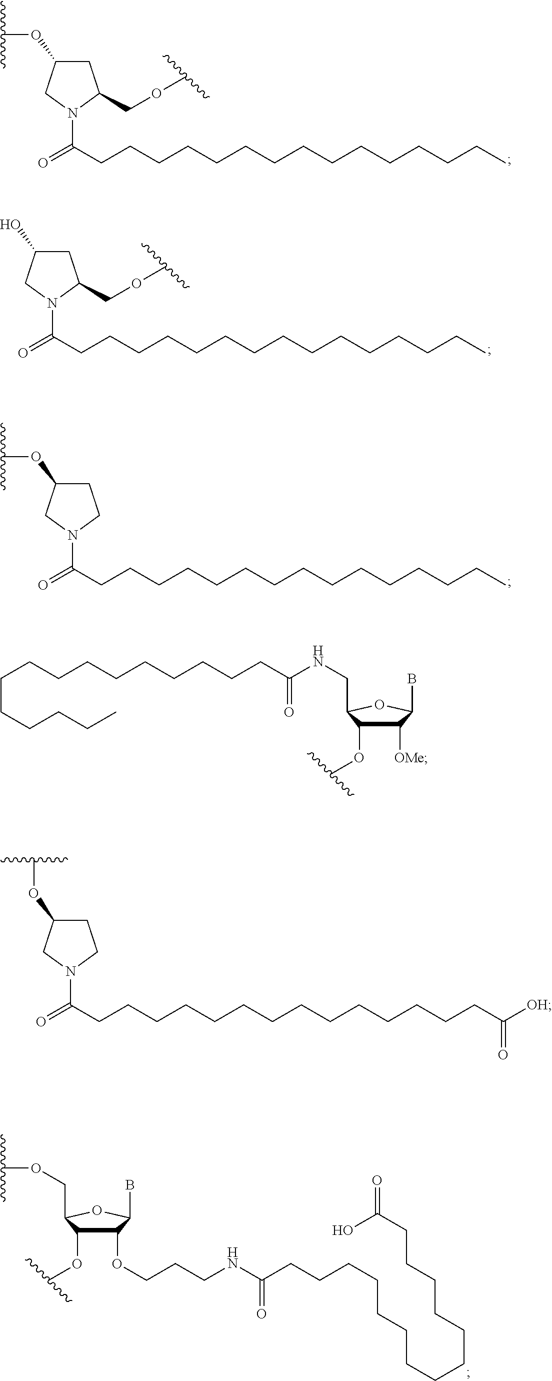 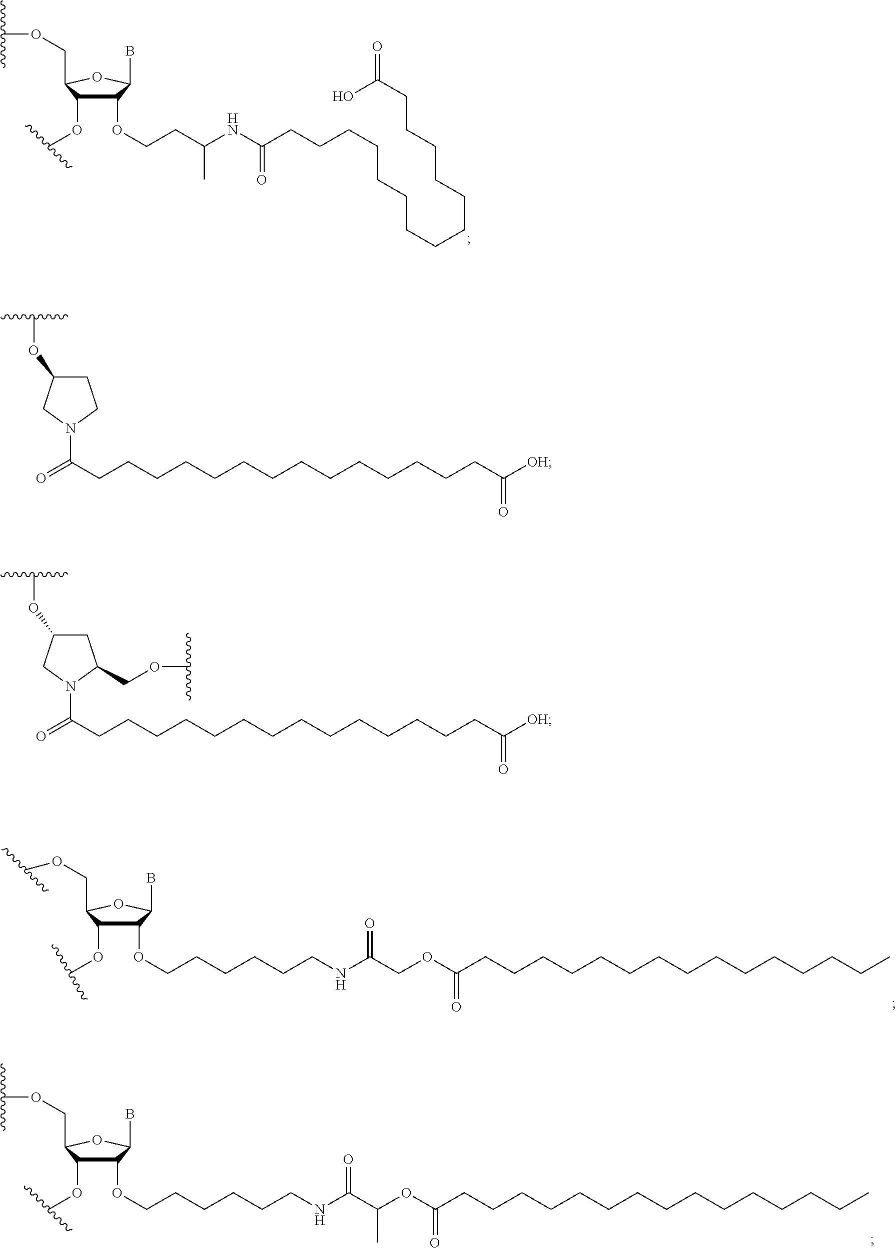 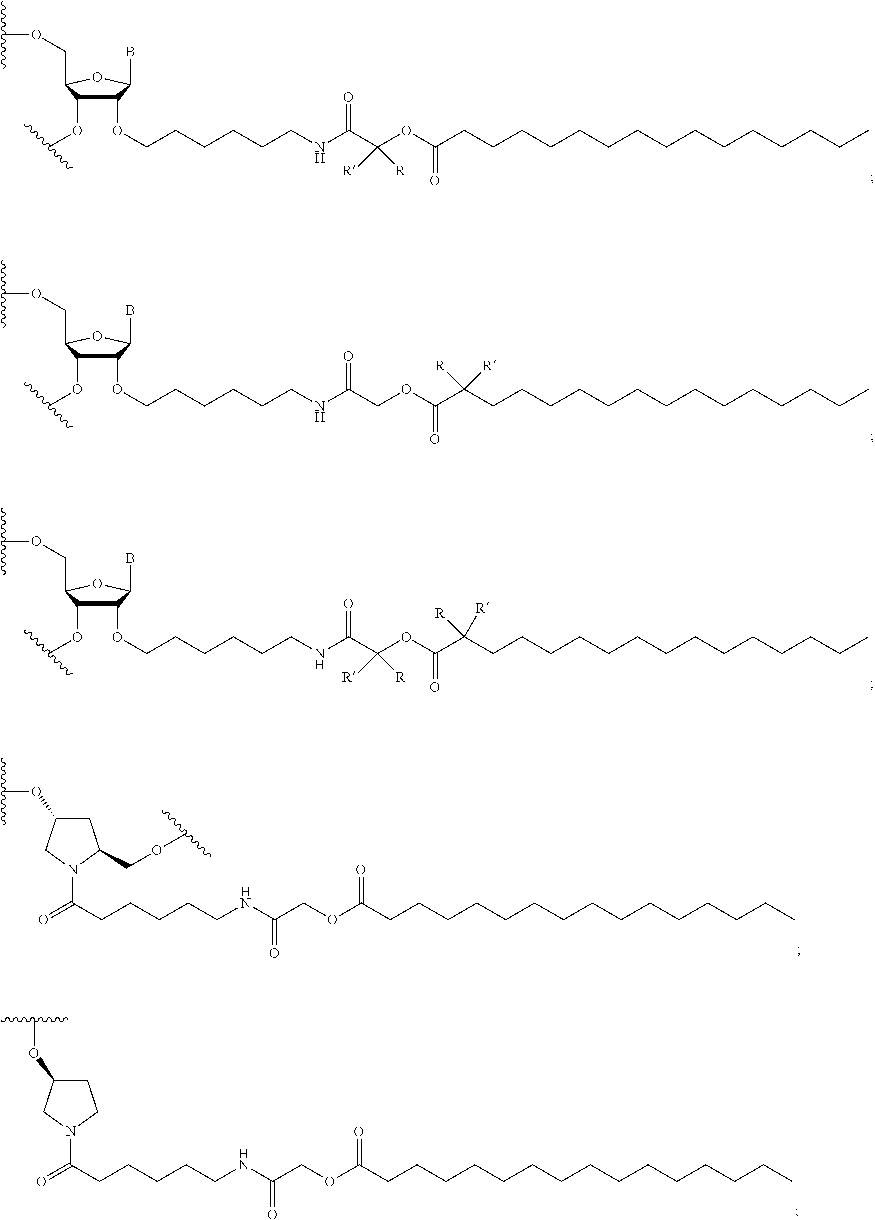 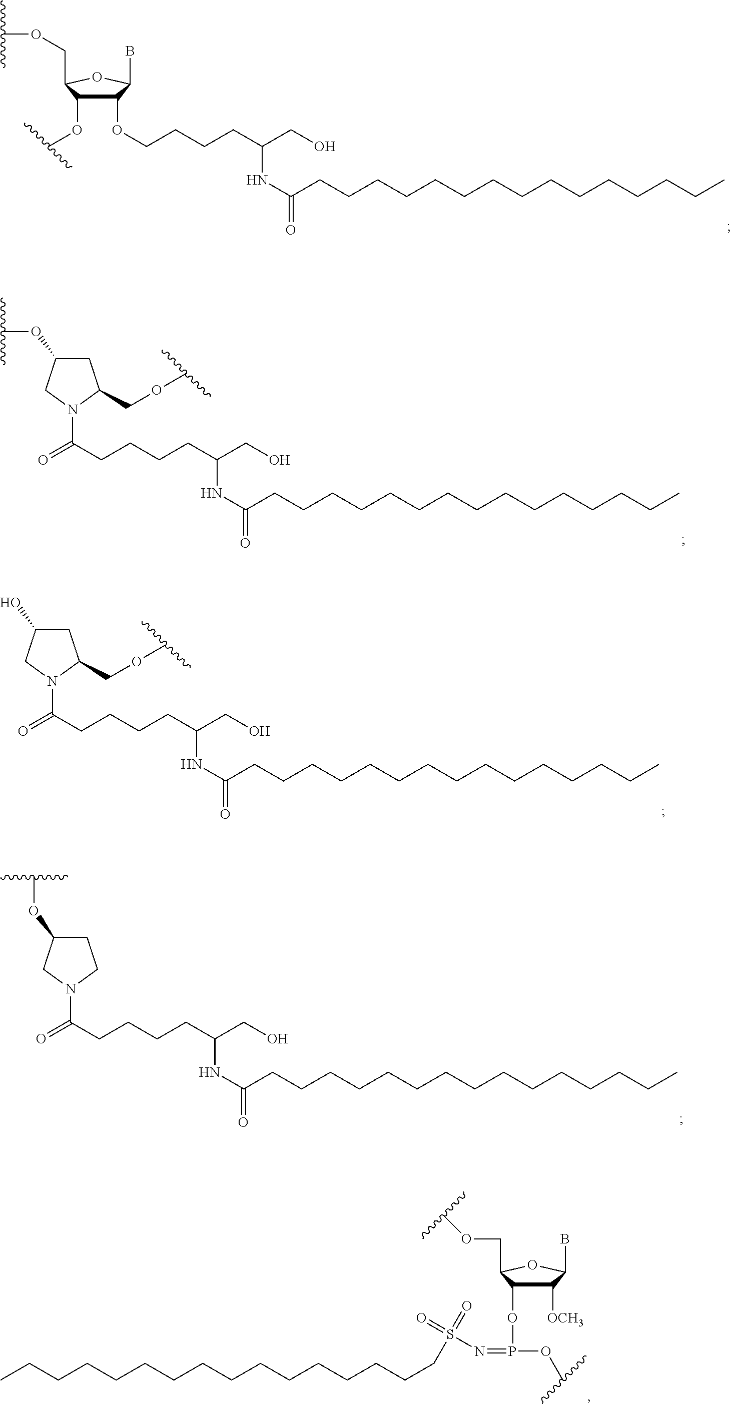 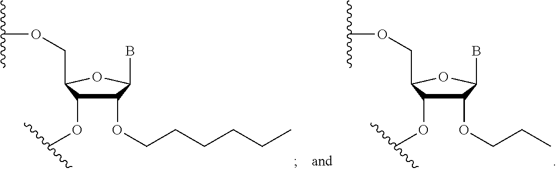
wherein R and R′ are each independently H, methyl, ethyl, isopropyl, or t-butyl.

11. The compound of claim 1, wherein the sense strand comprises at least one phosphorothioate linkage at the 3′-end.

12. The compound of claim 1, wherein the sense strand comprises at least two phosphorothioate linkages at the 3′-end.

13. The compound of claim 11, wherein one of the phosphorothioate linkages is located between the lipophilic monomer and the first nucleotide from the 3′-end of the sense strand.

14. The compound of claim 1, further comprising a phosphate or phosphate mimic at the 5′-end of the antisense strand.

15. The compound of claim 14, wherein the phosphate mimic is a 5′-vinyl phosphonate (VP).

16. The compound of claim 1, wherein the antisense strand comprises at least one GNA in the seed region.

17. The compound of claim 16, wherein the seed region is at position 5-7 from the 5′-end of the antisense strand.

18. The compound of claim 1, further comprising a targeting ligand that targets a receptor which mediates delivery to a CNS tissue.

19. The compound of claim 18, wherein the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, and LDL receptor ligand.

20. The compound of claim 1, further comprising a targeting ligand that targets a receptor which mediates delivery to an ocular tissue.

21. The compound of claim 20, wherein the targeting ligand is selected from the group consisting of trans-retinol, RGD peptide, LDL receptor ligand, and carbohydrate based ligands.

22. The compound of claim 21, wherein the targeting ligand is a RGD peptide and the RGD peptide is H-Gly-Arg-Gly-Asp-Ser-Pro-Lys-Cys-OH or Cyclo(-Arg-Gly-Asp-D-Phe-Cys).

23-37. (canceled)

38. A compound comprising:
an antisense strand which is complementary to a target gene;
a sense strand which is complementary to said antisense strand; and
one or more lipophilic monomers containing a lipophilic moiety conjugated to the sense strand and/or antisense strand via a carrier selected from the group consisting of: 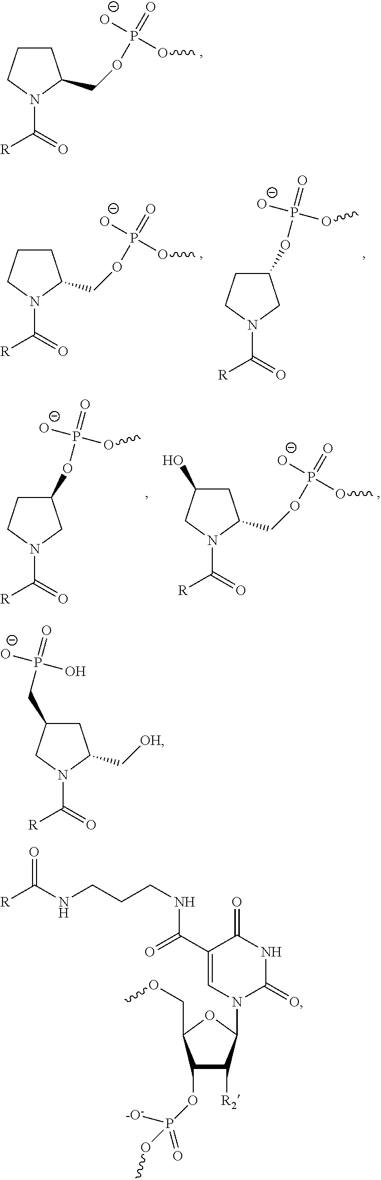 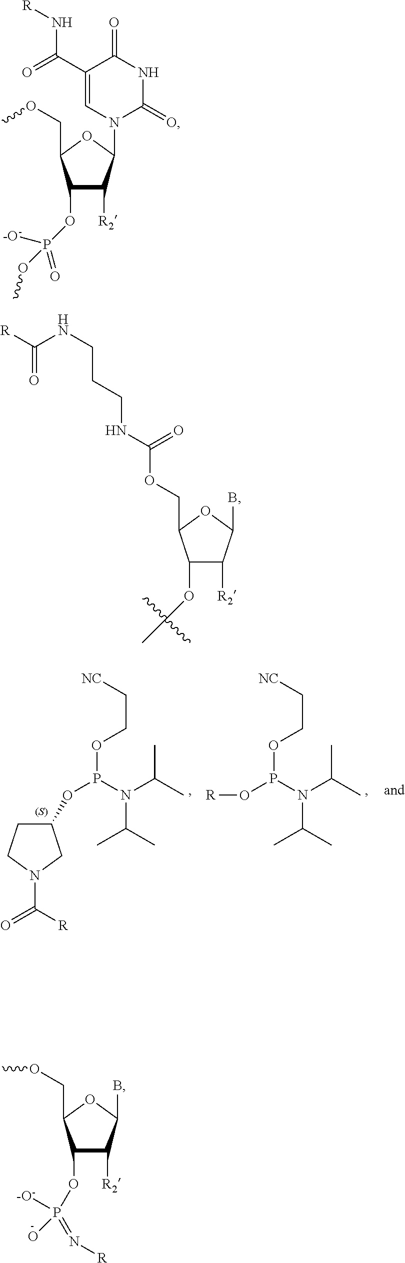
wherein:
R is the lipophilic moiety.
R₂′ is H, OH, F, OMe, O-methoxyalkyl, O-allyl, O—N-methylacetamido, O-dimethylaminoethoxyethyl, or O-aminopropyl; and
B is a modified or unmodified nucleobase.

39. A compound comprising:
an antisense strand which is complementary to a target gene;
a sense strand which is complementary to said antisense strand; and
one or more lipophilic monomers containing a lipophilic moiety conjugated to an internal position of the sense strand and/or antisense strand via a carrier selected from the group consisting of: 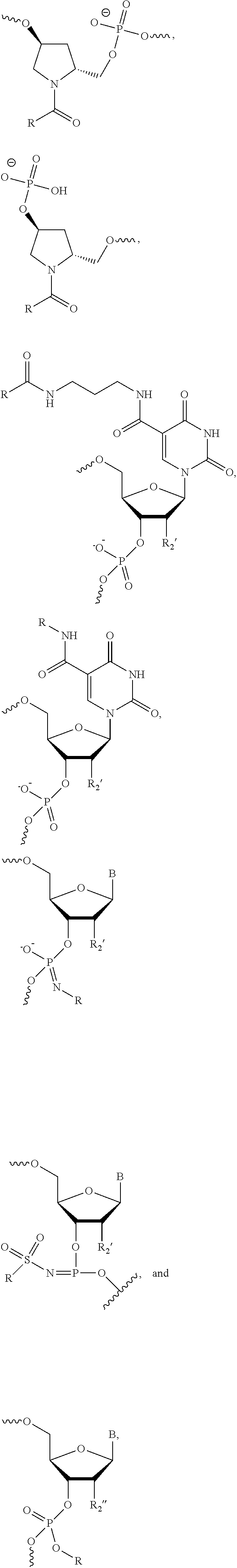
wherein:
R is the lipophilic moiety;
n is an integer of 1-21;
R₂′ is H, OH, F, OMe, O-methoxyalkyl, O-allyl, O—N-methylacetamido, O-dimethylaminoethoxyethyl, or O-aminopropyl; and
B is a modified or unmodified nucleobase.
